(19) United States
(12) Patent Application Publication
(10) Pub. No.: US 2004/0242572 A1
(43) Pub. Date: Dec. 2, 2004

(54) New carboxamide compounds having melanin concentrating hormone antagonistic activity, pharmaceutical preparations comprising these compounds and process for their manufacture

(75) Inventors: Dirk Stenkamp, Biberach (DE); Martin Lenter, Ulm (DE); Heike-Andrea Wieland, Bad Soden (DE); Klaus Rudolf, Warthausen (DE); Stephan George Mueller, Warthausen (DE); Ralf R.H. Lotz, Schemmerhofen (DE); Kirsten Arndt, Biberach (DE); Philipp Lustenberger, Warthausen (DE); Thorsten Lehmann-Lintz, Ochsenhausen (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/647,156

(22) Filed: Aug. 22, 2003

Related U.S. Application Data
(60) Provisional application No. 60/408,224, filed on Sep. 4, 2002.

(30) Foreign Application Priority Data
Aug. 24, 2002 (DE) 10238865

(51) Int. Cl. A61K 31/54; A61K 31/537; A61K 31/495; A61K 31/426; A61K 31/421; A61K 31/4172; A61K 31/325; A61K 31/16

(52) U.S. Cl. 514/227.2; 514/237.5; 514/252.12; 514/317; 514/365; 514/374; 514/400; 514/408; 514/478; 514/616; 544/59; 544/162; 544/399; 546/229; 548/146; 548/215; 548/338.5; 548/567; 560/157

(57) ABSTRACT

The present invention relates to carboxamide compounds of general formula I

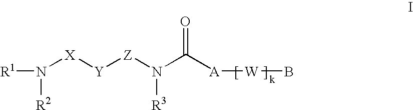

wherein the groups and residues A, B, W, X, Y, Z, R¹, R², R³ and k have the meanings given in claim 1. Moreover the invention relates to process for preparing the above mentioned carboxamides as well as pharmaceutical compositions containing at least one carboxamide according to the invention. In view of their MCH-receptor antagonistic activity the pharmaceutical compositions according to the invention are suitable for the treatment of metabolic disorders and/or eating disorders, particularly obesity, bulimia, anorexia, hyperphagia and diabetes.

### RELATED APPLICATIONS

The priority benefit of DE 102 38 865.2, filed Aug. 24, 2002 and U.S. Provisional Application No. 60/408,224, filed Sep. 4, 2002 are hereby claimed, both of which are incorporated by reference herein.

The present invention relates to new carboxamide compounds, processes for preparing them and the physiologically acceptable salts thereof as well as their use as MCH antagonists and their use in preparing a pharmaceutical preparation which is suitable for the prevention and/or treatment of symptoms and/or diseases caused by MCH or causally connected with MCH in some other way. The invention further relates to the use of a compound according to the invention for influencing eating behaviour and for reducing body weight and/or for preventing an increase in the body weight of a mammal. The invention also relates to compositions and medicaments containing a compound according to the invention, and a process for preparing them.

### BACKGROUND TO THE INVENTION

The intake of food and its conversion in the body is an essential part of life for all living creatures. Therefore, deviations in the intake and conversion of food generally lead to problems and also illness. The changes in the lifestyle and nutrition of humans, particularly in industrialised countries, have promoted obesity in recent decades. In affected people, obesity leads directly to restricted mobility and a reduction in the quality of life. There is the additional factor that obesity often leads to other diseases such as, for example, diabetes, dyslipidaemia, high blood pressure, arteriosclerosis and coronary heart disease. Moreover, high body weight alone puts an increased strain on the support and mobility apparatus, which can lead to chronic pain and diseases such as arthritis or osteoarthritis. Thus, obesity is a serious health problem for society.

The term obesity means an excess of adipose tissue. In this connection, obesity is fundamentally to be seen as the increased level of body fat which leads to a health risk. In the last analysis it is not precisely possible to draw a distinction between normal individuals and those suffering from obesity, but the health risk accompanying obesity is presumed to rise continuously as the level of body fat increases. For simplicity's sake, in the present invention, individuals with a Body Mass Index (BMI), which is defined as the body weight measured in kilograms divided by the height (in meters) squared, above a value of 25 and more particularly above 30 are preferably regarded as suffering from obesity.

Apart from physical activity and a change in nutrition, there is currently no convincing treatment option for effectively reducing body weight. However, as obesity is a major risk factor in the development of serious and even life-threatening diseases, it is all the more important to have access to pharmaceutical active substances for the prevention and/or treatment of obesity. One approach which has been proposed very recently is the therapeutic use of MCH antagonists (cf. inter alia WO 01/21577, WO 01/82925).

Melanin-concentrating hormone (MCH) is a cyclic neuropeptide consisting of 19 amino acids. It is synthesised predominantly in the hypothalamus in mammals and from there travels to other parts of the brain by the projections of hypothalamic neurones. Its biological activity is mediated in humans through two different glycoprotein-coupled receptors (GPCRs) from the family of rhodopsin-related GPCRs, namely the MCH receptors 1 and 2 (MCH-1R, MCH-2R).

Investigations into the function of MCH in animal models have provided good indications for a role of the peptide in regulating the energy balance, i.e. changing metabolic activity and food intake [1,2]. For example, after intraventricular administration of MCH in rats, food intake was increased compared with control animals. Additionally, transgenic rats which produce more MCH than control animals, when given a high-fat diet, responded by gaining significantly more weight than animals without an experimentally altered MCH level. It was also found that there is a positive correlation between phases of increased desire for food and the quantity of MCH mRNA in the hypothalamus of rats. However, experiments with MCH knock-out mice are particularly important in showing the function of MCH. Loss of the neuropeptide results in lean animals with a reduced fat mass, which take in significantly less food than control animals.

The anorectic effects of MCH are mediated in rodents through the G_{∀s}-coupled MCH-1R [3-6]. Unlike primates, ferrets and dogs, no second receptor has hitherto been found in rodents. After losing the MCH-1R, knock-out mice have a lower fat mass, an increased energy conversion and, when fed on a high fat diet, do not put on weight, compared with control animals. Another indication of the importance of the MCH-MCH-1R system in regulating the energy balance results from experiments with a receptor antagonist (SNAP-7941) [3]. In long term trials the animals treated with the antagonist lose significant amounts of weight.

In addition to its anorectic effect, the MCH-1R antagonist SNAP-7941 also achieves additional anxiolytic and antidepressant effects in behavioural experiments on rats [3]. Thus, there are clear indications that the MCH-MCH-1R system is involved not only in regulating the energy balance but also in affectivity.

### Literature

1. Qu, D., et al., A role for melanin-concentrating hormone in the central regulation of feeding behaviour. Nature, 1996. 380(6571): p. 243-7.

2. Shimada, M., et al., Mice lacking melanin-concentrating hormone are hypophagic and lean. Nature, 1998. 396(6712): p. 670-4.

3. Borowsky, B., et al., Antidepressant, anxiolytic and anorectic effects of a melanin-concentrating hormone-1 receptor antagonist. Nat Med, 2002. 8(8): p. 825-30.

4. Chen, Y., et al., Targeted disruption of the melanin-concentrating hormone receptor-1 results in hyperphagia and resistance to diet-induced obesity. Endocrinology, 2002. 143(7): p. 2469-77.

5. Marsh, D. J., et al., Melanin-concentrating hormone 1 receptor-deficient mice are lean, hyperactive, and hyperphagic and have altered metabolism. Proc Natl Acad Sci USA, 2002. 99(5): p. 3240-5.

6. Takekawa, S., et al., T-226296: A novel, orally active and selective melanin-concentrating hormone receptor antagonist. Eur J Pharmacol, 2002. 438(3): p. 129-35.

In the patent literature certain amine compounds are proposed as MCH antagonists. Thus, WO 01/21577 (Takeda) describes compounds of formula

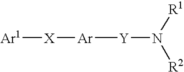

wherein Ar¹ denotes a cyclic group, X denotes a spacer, Y denotes a bond or a spacer, Ar denotes an aromatic ring which may be fused with a non-aromatic ring, R¹ and R² independently of one another denote H or a hydrocarbon group, while R¹ and R² together with the adjacent N atom may form an N-containing hetero ring and R² with Ar may also form a spirocyclic ring, R together with the adjacent N atom and Y may form an N-containing hetero ring, as MCH antagonists for the treatment of obesity.

Moreover WO 01/82925 (Takeda) also describes compounds of formula

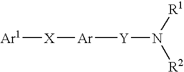

wherein Ar¹ denotes a cyclic group, X and Y represent spacer groups, Ar denotes an optionally substituted fused polycyclic aromatic ring, R¹ and R² independently of one another represent H or a hydrocarbon group, while R¹ and R² together with the adjacent N atom may form an N-containing heterocyclic ring and R² together with the adjacent N atom and Y may form an N-containing hetero ring, as MCH antagonists for the treatment of obesity.

Other amine-compounds having an MCH-antagonistic activity are proposed in WO 02/057233 (Schering Corp.). The compounds come under general formula

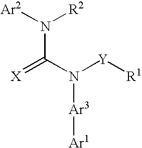

wherein Ar¹, Ar², Ar³ denote inter alia aryl or heteroaryl, X O, S or N—CN, Y denotes a single bond or C₁₋₄-alkylene and R¹ and R² are as herein defined.

Also a MCH-antagonistic activity is described in WO 02/051809 (Schering Corp.) in connection with piperidine derivatives of formula

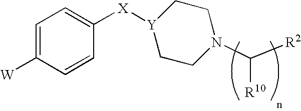

wherein W denotes a specifically defined aminocarbonyl or carbonylamino group, X denotes —CHR⁸, —CO, —C(═NOR⁹) or —CR⁸═, Y denotes CH, C(OH), C(C₁₋₄-alkoxy) or in the case of a C double bond, R² denotes a substituted aryl or heteroaryl group, R¹⁰ denotes H, C₁₋₆-alkyl or aryl and the other groups are as herein defined.

Carboxamides as antagonists of the human 11CBy receptors are proposed in WO 02/10146 (Smithkline Beecham). The compounds are examples of the general structural formula

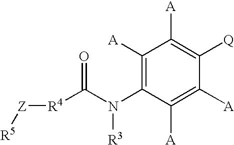

wherein A denotes H, alkyl, alkoxy, alkenyl, acyl, halogen, OH, CN or CF₃, R³ denotes H, methyl or ethyl, R⁴ denotes an optionally substituted aromatic carbocyclic or heterocyclic ring, Z denotes O, S, NH, CH₂ or a single bond, R⁵ denotes an optionally substituted aromatic, saturated or unsaturated carbocyclic or heterocyclic ring, Q denotes the group —X—Y—NR¹(R²), while according to different configurations X may denote O, S or N, Y may denote an alkylene or a cycloalkylene group which may also be substituted, and R¹ and R² may represent alkyl or phenyl-alkyl, while R¹ and R², R¹ and Y or R¹ and X may also be connected to one another to form a cyclic system, as described.

Other compounds with MCH-antagonistic properties are proposed in the published applications WO 03/035055, WO 03/033480, WO 02/06245, WO 02/04433, WO 01/87834, WO 01/21169 and JP 2001/226269.

Quinazolinone compounds of general formula

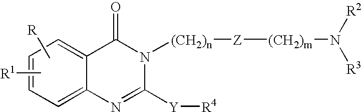

are described in WO 01/23365 (Merck), wherein Z denotes a bond or phenylene, and in WO 01/23364 (Merck), wherein Z denotes cyclohexylene. Moreover Y represents a bond or C₂₋₄-alkenyl and R⁴ denotes aryl, cycloalkyl, phenylalkyl or a heterocyclic system. These compounds are described as GPlbIX inhibitors, particularly as inhibitors of this receptor with the von Willebrand factor (vWF) ligand.

Aromatic compounds which may contain an amide bridge and an amine group are also proposed in the literature for other indications. Thus, compounds of general formula Ar-A-E, wherein Ar denotes an optionally substituted aromatic mono- or bicyclic group, A denotes an amide or amine bridge and E denotes inter alia a phenyl group which is substituted in the para position via a spacer group B with a substituted aminoalkylene group, are described in WO 99/01127 (Smithkline Beecham Corp.). These compounds are proposed as CCR5 receptor ligands for the treatment inter alia of asthma, atopical diseases and rheumatoid arthritis.

WO 01/72712 (Cor Therapeutics Inc.) describes isoquinoline compounds of the following formula

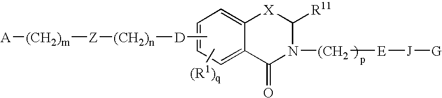

wherein A denotes an optionally substituted amino or amidino group, Z denotes a bond or an alkyl, cycloalkyl, alkenyl, alkynyl or aryl spacer group, m and n denote 0 to 3, D denotes a bond or a specified bridge, X denotes NR¹² or CHR¹², p denotes 0 to 3, E also denotes a bond, in addition to the specified ether, amine, amide and carboxyl groups, J denotes a bond, a cycloalkylene, phenylene, naphthylene or heteroaryl group, G denotes more closely defined amide, imino or amidino groups and the other groups are as hereinbefore defined. These compounds are proposed as inhibitors of the isolated factor Xa as well as blood clotting and are therefore proposed as antithrombotic and thrombolytic active substances.

DE 197 18 181 A1 (Boehringer Ingelheim) proposes disubstituted bicyclic heterocycles of formula

Rₐ-A-Het-Ar-E

wherein Rₐ may denote one of a number of more closely defined amino groups or optionally also an R₄—SO₂—NR₅ or an R₄—SO₂ group having the meanings given for R₄ and R₅, A denotes a phenylene-C₁₋₃-alkylene group, an n-C₂₋₆-alkylene group or a C₅₋₇-cycloalkylene-C₁₋₃-alkylene group which may be substituted as specified, Het denotes an optionally substituted benzimidazole, indole, tetrahydroquinolinone or quinazolinone group, Ar denotes an optionally substituted phenylene, naphthylene, thienylene, thiazolylene, pyridinylene, pyrimidinylene, pyrazinylene or pyridazinylene group and E denotes a cyano or R_{b}NH—C(═NH) group, wherein R_{b} denotes H, OH, C₁₋₃-alkyl or a group which can be cleaved in vivo. These compounds are proposed as thrombin-inhibiting and thrombin-time prolonging active substances.

### SUMMARY OF THE INVENTION

The present invention provides new carboxamide compounds, particularly those which are effective as MCH antagonists. The invention also sets out to provide new carboxamide compounds which can be used to influence the eating habits of mammals and achieve a reduction in body weight, particularly in mammals and/or prevent an increase in body weight. The present invention further sets out to provide new pharmaceutical compositions which are suitable for the prevention and/or treatment of symptoms and/or diseases caused by MCH or otherwise causally connected to MCH. In particular, this invention provides pharmaceutical compositions for the treatment of metabolic disorders such as obesity and/or diabetes as well as diseases and/or disorders which are associated with obesity and diabetes. Other objectives of the present invention are concerned with demonstrating advantageous uses of the compounds according to the invention. The invention also sets out to provide a process for preparing the carboxamide compounds according to the invention. Other aims of the present invention will be immediately apparent to one skilled in the art from the foregoing remarks and those that follow.

A first object of the present invention comprises carboxamide compounds of general formula I

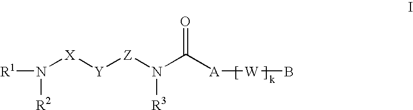

wherein

R¹, R² independently of one another denote H, a C₁₋₈-alkyl or C₃₋₇-cycloalkyl group optionally substituted by the group R¹¹ or a phenyl group optionally mono- or polysubstituted by the group R¹² and/or monosubstituted by nitro, or

R¹ and R² form a C₂₋₈-alkylene bridge wherein

one or two —CH₂-groups may be replaced independently of one another by —CH═N— or —CH═CH— and/or

one or two —CH₂-groups may be replaced independently of one another by —O—, —S—, —CO—, —C(═CH₂)— or —NR¹³— so that heteroatoms are not directly connected to one another,

while in the alkylene bridge defined above one or more H atoms may be replaced by R¹⁴, and/or

the alkylene bridge defined above may be substituted by one or two identical or different carbo- or heterocyclic groups Cy in such a way that the bond between the alkylene bridge and the group Cy is formed

via a single or double bond,

via a common C atom forming a spirocyclic ring system,

via two common, adjacent C and/or N atoms forming a fused bicyclic ring system or

via three or more C and/or N atoms forming a bridged ring system,

R³ denotes H, C₁₋₆-alkyl, C₃₋₇-cycloalkyl, C₃₋₇-cycloalkyl-C₁₋₄-alkyl-, C₃₋₇-cycloalkenyl, C₃₋₇-cycloalkenyl-C₁₋₄-alkyl-, phenyl, phenyl-C₁₋₄-alkyl-, C₁₋₃-alkoxy-C₂₋₆-alkyl-, amino-C₂₋₆-alkyl-, C₁₋₃-alkyl-amino-C₂₋₆-alkyl- or di-(C₁₋₃-alkyl)-amino-C₂₋₆-alkyl-,

X denotes a single bond or a C₁₋₈-alkylene bridge wherein

one or two —CH₂-groups may be replaced independently of one another by —CH═CH— or —C≡C— and/or

one or two —CH₂-groups may be replaced independently of one another by —O—, —S—, —(SO)—, —(SO₂)—, —CO— or —NR⁴— in such a way that in each case two O, S or N atoms or one O atom and an S atom are not directly connected with one another,

while one or two C atoms independently of one another may be substituted by a hydroxy, ω-hydroxy-C₁₋₃-alkyl-, ω-(C₁₋₃-alkoxy)-C₁₋₃-alkyl- and/or C₁₋₃-alkoxy group and/or in each case with one or two identical or different C₁₋₆-alkyl groups, and/or

the alkylene bridge may be connected to R¹ so as to include the N atom connected to R¹ and X, forming a heterocyclic group,

Z denotes a C₁₋₄-alkylene bridge, wherein two adjacent C atoms with an additional C₁₋₄-alkylene bridge may be connected to one another, while in group Z a —CH₂-group may be replaced by —O— or —NR⁵—,

and one or two C atoms of the alkylene bridge may be substituted independently of one another with a hydroxy, ω-hydroxy-C₁₋₃-alkyl-,

ω-(C₁₋₃-alkoxy)-C₁₋₃-alkyl-, C₁₋₃-alkoxy group, amino-C₁₋₃-alkyl-, C₁₋₃-alkyl-amino-C₁₋₃-alkyl- or di-(C₁₋₃-alkyl)-amino-C₁₋₃-alkyl- and/or with one or two identical or different C₁₋₆-alkyl groups, and/or

R³ may be connected to Z so as to include the N atom connected to R³, forming a heterocyclic group,

A, Y independently of one another have one of the meanings given for Cy,

while R¹ may be connected to Y so as to include the group X and the N atom connected to R¹ and X, forming a heterocyclic group fused to Y, and/or

R³ may be connected to Y so as to include the group Z and the N atom connected to R³ and Z forming a saturated or partially unsaturated heterocyclic group fused to Y, or

A and R³ may be connected to one another in such a way that the group

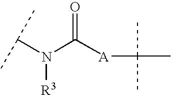

of formula I denotes a group of partial formula II

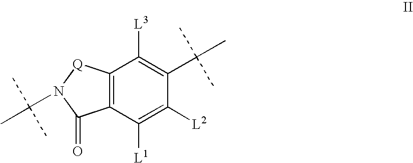

and

Q denotes a group, selected from the partial formulae IIIa to IIIg

—CR⁶R⁷— IIIa

—CR⁶═CR⁷— IIIb

—N═CR⁸— IIIc

—N═N— IIId

—CO—NR⁹— IIIe

—CR⁸═N— IIIf

—CO— IIIg

L¹, L², L³ independently of one another have one of the meanings given for R²⁰,

B denotes C₁₋₆-alkyl, C₁₋₆-alkenyl, C₁₋₆-alkynyl, C₃₋₇-cycloalkyl-C₁₋₃-alkyl-, C₃₋₇-cycloalkenyl-C₁₋₃-alkyl-, C₃₋₇-cycloalkyl-C₁₋₃-alkenyl- or C₃₋₇-cycloalkyl-C₁₋₃-alkynyl-, wherein one or more C atoms may be mono- or polysubstituted by halogen and/or monosubstituted by hydroxy or cyano and/or cyclic groups may be mono- or polysubstituted by R²⁰, or

has one of the meanings given for Cy, while the bond to the group W or optionally directly to the group A is formed via a C atom of the carbocyclic moiety or of the optionally fused-on phenyl or pyridine ring or via an N or C atom of the heterocyclic moiety,

while when k=0the group B and the group A may be connected to one another via a common C atom forming a spirocyclic ring system or

via two common, adjacent atoms forming a fused, bicyclic ring system,

W denotes a single bond, —O—, a C₁₋₄-alkylene, C₂₋₄-alkenylene, C₂₋₄-alkynylene, C₁₋₄-alkylenoxy, Oxy-C₁₋₄-alkylene, C₁₋₃-alkylene-oxy-C₁₋₃-alkylene-, imino, N—(C₁₋₃-alkyl)-imino-, imino-C₁₋₄-alkylene-, N—(C₁₋₃-alkyl)-imino-C₁₋₄-alkylene-, C₁₋₄-alkylene-imino- or C₁₋₄-alkylene-N—(C₁₋₃-alkyl)-imino-group,

while one or two C atoms independently of one another may be substituted by a hydroxy, ω-hydroxy-C₁₋₃-alkyl-, ω-(C₁₋₃-alkoxy)-C₁₋₃-alkyl- and/ or C₁₋₃-alkoxy group and/or with one or two identical or different C₁₋₆-alkyl groups, and/or

W with the definitions alkylene, oxyalkylene and alkyleneoxyalkylene may also be connected to B via a double bond,

k denotes 0 or 1,

Cy denotes a carbo- or heterocyclic group selected from one of the following definitions

a saturated 3- to 7-membered carbocyclic group,

an unsaturated 5- to 7-membered carbocyclic group,

a phenyl group,

a saturated 4- to 7-membered or unsaturated 5- to 7-membered heterocyclic group with an N, O or S atom as heteroatom,

a saturated or unsaturated 5- to 7-membered heterocyclic group with two or more N atoms or with one or two N atoms and an O or S atom as heteroatoms,

an aromatic heterocyclic 5- or 6-membered group with one or more identical or different heteroatoms selected from N, O and/or S,

while the above mentioned 4-, 5-, 6- or 7-membered groups may be connected via two common, adjacent C atoms, fused with a phenyl or pyridine ring, and

in the above mentioned 5-, 6- or 7-membered groups one or two non-adjacent —CH₂ groups may be replaced by a —CO—, —C(═CH₂)—, —(SO)— or —(SO₂)— group, and

the above mentioned saturated 6- or 7-membered groups may also be present as bridged ring systems with an imino, N—(C₁₋₄-alkyl)-imino, methylene, C₁₋₄-alkyl-methylene- or di-(C₁₋₄-alkyl)-methylene-bridge, and

the above mentioned cyclic groups may be mono- or polysubstituted at one or more C atoms with R²⁰, and in the case of a phenyl group also additionally monosubstituted by nitro, and/or substituted by R²¹ at one or more N atoms,

R⁴, R⁵ independently of one another have one of the meanings given for R¹⁶,

R⁶, R⁷,

R⁸, R⁹ independently of one another denote H, a C₁₋₆-alkyl, ω-C₁₋₃-alkoxy-C₁₋₃-alkyl- or ω-hydroxy-C₁₋₃-alkyl-group and R⁶, R⁷, R⁸ independently of one another also denote halogen,

R¹¹ denotes R¹⁵—O—, R¹⁵—O—CO—, R¹⁶R¹⁷N—, R¹⁸R¹⁹N—CO— or Cy-,

R¹² has one of the meanings given for R²⁰,

R¹³ has one of the meanings given for R¹⁷,

R¹⁴ denotes halogen, C₁₋₆-alkyl, R¹⁵—O—, R¹⁵—O—CO—, R¹⁶R¹⁷N—, R¹⁸R¹⁹N—CO—, R¹⁵—O—C₁₋₃-alkyl-, R¹⁵—O—CO—C₁₋₃-alkyl-, R¹⁶R¹⁷N—C₁₋₃-alkyl-, R¹⁸R¹⁹N—CO—C₁₋₃-alkyl- or Cy-C₁₋₃-alkyl-,

R¹⁵ denotes H, C₁₋₄-alkyl, C₃₋₇-cycloalkyl, C₃₋₇-cycloalkyl-C₁₋₃-alkyl-, phenyl, phenyl-C₁₋₃-alkyl- or pyridinyl,

R¹⁶ denotes H, C₁₋₆-alkyl, C₃₋₇-cycloalkyl, C₃₋₇-cycloalkyl-C₁₋₃-alkyl-, C₄₋₇-cycloalkenyl, C₄₋₇-cycloalkenyl-C₁₋₃-alkyl-, ω-hydroxy-C₂₋₃-alkyl-, ω-(C₁₋₃-alkoxy)-C₂₋₃-alkyl-, amino-C₁₋₆-alkyl-, C₁₋₃-alkyl-amino-C₁₋₆-alkyl- or di-(C₁₋₃-alkyl)-amino-C₁₋₆-alkyl-,

R¹⁷ has one of the meanings given for R¹⁶ or denotes phenyl, phenyl-C₁₋₃-alkyl-, pyridinyl, dioxolan-2-yl, C₁₋₃-alkylcarbonyl, hydroxycarbonyl-C₁₋₃-alkyl-, C₁₋₄-alkoxycarbonyl, C₁₋₃-alkylcarbonylamino-C₂₋₃-alkyl-, C₁₋₃-alkylsulphonyl- or C₁₋₃-alkylsulphonylamino-C₂₋₃-alkyl-,

R¹⁸, R¹⁹ independently of one another denote H or C₁₋₆-alkyl,

R²⁰ denotes halogen, hydroxy, cyano, C₁₋₄-alkyl, C₃₋₇-cycloalkyl, hydroxy-C₁₋₃-alkyl, R²²—C₁₋₃-alkyl- or has one of the meanings given for R²²,

R²¹ denotes C₁₋₃-alkyl, ω-hydroxy-C₂₋₃-alkyl, phenyl, phenyl-C₁₋₃-alkyl-, C₁₋₃-alkyl-carbonyl, carboxy, C₁₋₄-alkoxy-carbonyl, C₁₋₃-alkylsulphonyl, phenylcarbonyl or phenyl-C₁₋₃-alkyl-carbonyl,

R²² denotes pyridinyl, phenyl, phenyl-C₁₋₃-alkoxy-, C₁₋₃-alkoxy, C₁₋₃-alkylthio-, carboxy, H—CO—, C₁₋₃-alkylcarbonyl, C₁₋₄-alkoxycarbonyl, aminocarbonyl, C₁₋₃-alkylaminocarbonyl, di-(C₁₋₃-alkyl)-aminocarbonyl, C₁₋₃-alkyl-sulphonyl, C₁₋₃-alkyl-sulphinyl, C₁₋₃-alkyl-sulphonylamino-, amino-, C₁₋₃-alkylamino-, di-(C₁₋₃-alkyl)-amino-, phenyl-C₁₋₃-alkylamino- or N—(C₁₋₃-alkyl)-phenyl-C₁₋₃-alkylamino-, acetylamino-, propionylamino-, phenylcarbonyl, phenylcarbonylamino-, phenylcarbonylmethylamino-, hydroxyalkylaminocarbonyl, (4-morpholinyl)carbonyl, (1-pyrrolidinyl)carbonyl, (1-piperidinyl)carbonyl, (hexahydro-1-azepinyl)carbonyl, (4-methyl-1-piperazinyl)carbonyl, methylenedioxy, aminocarbonylamino- or alkylaminocarbonylamino-,

while in the groups and residues A, B, W, X, Y, Z, R¹ to R⁹ and R¹¹ to R²² in each case one or more C atoms may be mono- or polysubstituted by F and/or in each case one or two C atoms independently of one another may be monosubstituted by Cl or Br, and/or in each case one or more phenyl rings independently of one another additionally have one, two or three substituents selected from the group F, Cl, Br, I, C₁₋₄-alkyl, C₁₋₄-alkoxy, difluoromethyl, trifluoromethyl, hydroxy, amino, C₁₋₃-alkylamino-, di-(C₁₋₃-alkyl)-amino-, acetylamino-, aminocarbonyl, CN, difluoromethoxy, trifluoromethoxy, amino-C₁₋₃-alkyl-, C₁₋₃-alkylamino-C₁₋₃-alkyl- and di-(C₁₋₃-alkyl)-amino-C₁₋₃-alkyl- and/or may be monosubstituted by nitro, and

the H atom of any carboxy group present or an H atom bound to an N atom may be replaced in each case by a group which can be cleaved in vivo,

the tautomers, diastereomers, enantiomers, mixtures thereof and the salts thereof.

The invention also relates to the compounds in the form of the individual optical isomers, mixtures of the individual enantiomers or racemates, in the form of the tautomers and in the form of the free bases or the corresponding acid addition salts with pharmacologically acceptable acids. The subject of the invention also includes the compounds according to the invention, including their salts, wherein one or more hydrogen atoms are replaced by deuterium.

The invention further relates to a process for preparing carboxamide compounds of formula I

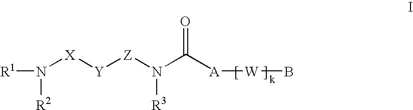

wherein A, B, W, X, Y, Z, R¹, R², R³ and k have one of the meanings given hereinbefore, where

if A denotes a group R³ which is not connected to the group A:

a) in the event that A denotes a nitrogen-heterocyclic group connected to the carboxamide group via a nitrogen atom which may also have in addition to the nitrogen atom one or more heteroatoms selected from N, O and S, at least one amine compound of formula I-1

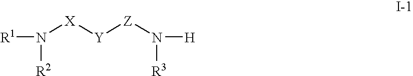

wherein R¹, R², R³, X, Y and Z have the meanings given hereinbefore,

is reacted with CDT (1,1′-carbonyldi-(1,2,4-triazole)) and at least one secondary amine compound of formula I-2

A[W]ₖB I-2

wherein A, B, W and k have the meanings given hereinbefore and the group A has the sec. amine function,

in a solvent or mixture of solvents in the presence of at least one base, and

b) for the other cases at least one carboxylic acid compound of formula I-3

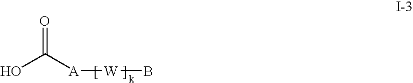

wherein A, B, W and k have the meanings given hereinbefore,

is reacted with TBTU (2-(1H-benzotriazol-1-yl)-1,1,3,3-tetramethyluronium-tetrafluoroborate) and at least one amine compound of formula I-1

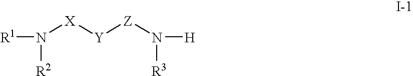

wherein R¹, R², R³, X, Y and Z have the meanings given hereinbefore,

in a solvent or mixture of solvents in the presence of at least one base, and

if B is a group R³ connected to the group A:

a) in the event of a group Q having the meaning —CR⁶R⁷— (IIIa), while R⁶ and R⁷ are as hereinbefore defined, an amine compound of formula Ia.1

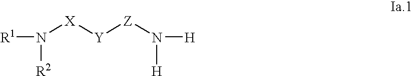

wherein R¹, R², X, Y and Z have the meanings specified, is reacted with an o-bromomethyl-benzoic acid ester derivative of formula Ia.2

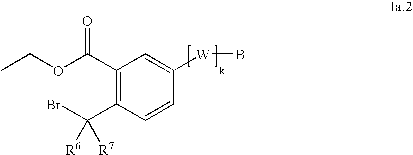

wherein R⁶, R⁷, W, B and k have the meanings specified,

b) in the event of a group Q having the meaning —CR⁶═CR⁷— (IIIb), wherein R⁶ and R⁷ are as hereinbefore defined, an isoquinolinone derivative of formula Ib.2

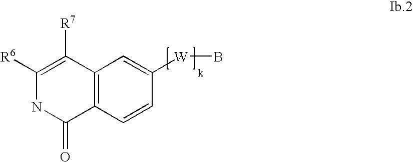

wherein R⁶, R⁷, W, B and k have the meanings specified, is reacted with an electrophilic compound of formula Ib.3

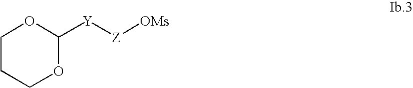

wherein Y and Z have the meanings specified and OMs denotes a suitable leaving group, preferably mesylate, to obtain an isoquinoline derivative of formula Ib.4

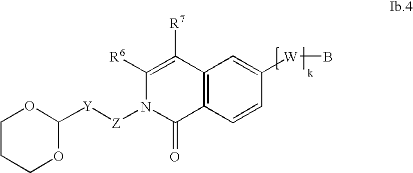

wherein R⁶, R⁷, W, B, Y, Z and k have the meanings specified, and the isoquinoline derivative of formula Ib.4 is further derivatised by known methods to form the compound of formula I,

c) in the event of a group Q having the meaning —N═CR⁸— (IIIc), wherein R⁸ is as hereinbefore defined, a phthalazinone derivative of formula Ic.4

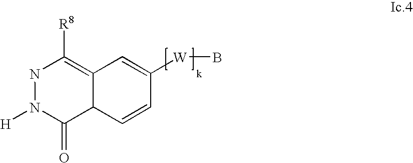

wherein R⁸, W, B and k have the meanings specified, is reacted with an electrophilic compound of formula Ic.5

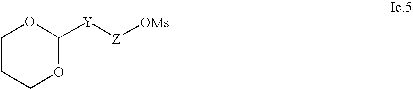

wherein Y and Z have the meanings specified and OMs denotes a leaving group, preferably mesylate, to form a phthalazinone derivative of formula Ic.6

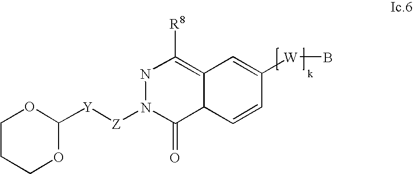

wherein R⁸, W, B, Y, Z and k have the meanings specified, and the phthalazinone derivative of formula Ic.6 thus obtained is further derivatised by known methods to form the compound of formula I wherein Q denotes —N═CR⁸— (IIIc),

d) in the event of a group Q having the meaning —N═N— (IIId) an o-amino-benzamide derivative of formula Id.1

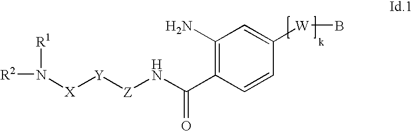

wherein R¹, R², W, B, X, Y, Z and k have the meanings specified, is reacted in the presence of a suitable nitrite compound and an acid to form the compound of formula I wherein Q denotes —N═N—,

e) in the event of a group Q having the meaning —CO—NR⁹— (IIIe), wherein R⁹ is as hereinbefore defined, an o-amino-benzamide derivative of formula Ie.1

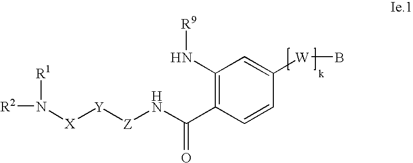

wherein R¹, R², R⁹, W, B, X, Y, Z and k have the meanings specified, is reacted in the presence of CDI (carbonyldiimidazole) to form the compound of formula I wherein Q denotes —CO—NR⁹—,

f) in the event of a group Q having the meaning —CR⁸═N— (IIIf), wherein R⁸ is as hereinbefore defined, an o-amino-benzamide derivative of formula If.1

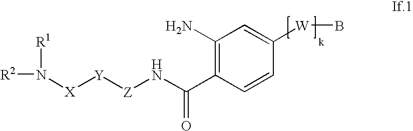

wherein R¹, R², W, B, X, Y, Z and k have the meanings specified, is reacted with a carboxylic acid R⁸COOH having the meaning specified for R⁸ and/or a corresponding activated carboxylic acid derivative to the quinazolinone derivative of formula I wherein Q denotes —CR⁸═N—,

g) in the event of a group Q having the meaning —CO— (IIIg) an isobenzofurandione derivative of formula Ig.2

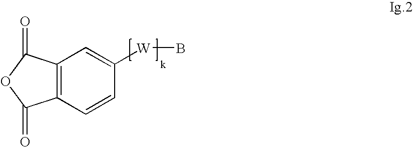

wherein W, B and k have the meanings specified, is reacted with an amine of formula Ig.1

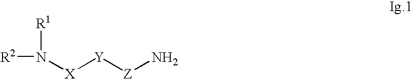

wherein R¹, R², X, Y and Z have the meanings specified, to form the compound of formula I wherein Q denotes —CO—.

This invention also includes the physiologically acceptable salts of the carboxamide compounds according to the invention as described above and hereinafter.

Also covered by this invention are compositions containing at least one according to the invention carboxamide compound and/or a salt according to the invention optionally together with one or more physiologically acceptable excipients.

Also covered by this invention are pharmaceutical compositions containing at least one carboxamide compound according to the invention and/or a salt according to the invention optionally together with one or more inert carriers and/or diluents.

The invention also relates to the use of at least one carboxamide compound according to the invention and/or a salt according to the invention for influencing the eating behaviour of a mammal.

The invention also relates to the use of at least one carboxamide compound according to the invention and/or a salt according to the invention for reducing the body weight and/or for preventing an increase in the body weight of a mammal.

The invention also relates to the use of at least one carboxamide compound according to the invention and/or a salt according to the invention for preparing a pharmaceutical composition with an MCH-receptor-antagonistic activity.

Moreover, the invention relates to the use of at least one carboxamide compound according to the invention and/or a salt according to the invention for preparing a pharmaceutical composition which is suitable for the prevention and/or treatment of symptoms and/or diseases which are caused by MCH or are otherwise causally connected with MCH.

The invention also relates to the use of at least one carboxamide compound according to the invention and/or a salt according to the invention for preparing a pharmaceutical composition which is suitable for the prevention and/or treatment of metabolic disorders and/or eating disorders, particularly obesity, bulimia, bulimia nervosa, cachexia, anorexia, anorexia nervosa and hyperphagia.

This invention also relates to the use of at least one carboxamide compound according to the invention and/or a salt according to the invention for preparing a pharmaceutical composition which is suitable for the prevention and/or treatment of diseases and/or disorders associated with obesity, particularly diabetes, especially type II diabetes, complications of diabetes including diabetic retinopathy, diabetic neuropathy, diabetic nephropathy, insulin resistance, pathological glucose tolerance, encephalorrhagia, cardiac insufficiency, cardiovascular diseases, particularly arteriosclerosis and high blood pressure, arthritis and gonitis.

Moreover, the invention relates to the use of at least one carboxamide compound according to the invention and/or a salt according to the invention for preparing a pharmaceutical composition which is suitable for the prevention and/or treatment of hyperlipidaemia, cellulitis, fat accumulation, malignant mastocytosis, systemic mastocytosis, emotional disorders, affective disorders, depression, anxiety, sleep disorders, reproductive disorders, sexual disorders, memory disorders, epilepsy, forms of dementia and hormonal disorders.

Another object of the invention is the use of at least one carboxamide compound according to the invention and/or a salt according to the invention for preparing a pharmaceutical composition which is suitable for the prevention and/or treatment of micturition disorders, such as for example urinary incontinence, hyperactive urinary bladder, urgency, nycturia and enuresis.

Furthermore the invention relates to processes for preparing a pharmaceutical composition according to the invention, characterised in that at least one carboxamide compound according to the invention and/or a salt according to the invention is incorporated in one or more inert carriers and/or diluents by a non-chemical method.

The invention further relates to a pharmaceutical composition containing a first active substance selected from the carboxamide compounds according to the invention and/or the corresponding salts, as well as a second active substance selected from the group consisting of active substances for the treatment of diabetes, active substances for the treatment of diabetic complications, active substances for the treatment of obesity, preferably other than MCH antagonists, active substances for the treatment of high blood pressure, active substances for the treatment of hyperlipidaemia, including arteriosclerosis, active substances for the treatment of arthritis, active substances for the treatment of anxiety states and active substances for the treatment of depression, optionally together with one or more inert carriers and/or diluents.

### DETAILED DESCRIPTION OF THE INVENTION

Unless otherwise specified the groups, residues, substituents and indices, particularly A, B, W, X, Y, Z, R¹ to R⁹, R¹¹ to R²², L¹, L², L³ and k, have one of the meanings given above or hereinafter.

A preferred embodiment of this invention comprises compounds of formula I wherein

R³ denotes H, C₁₋₆-alkyl, C₃₋₇-cycloalkyl, C₃₋₇-cycloalkyl-C₁₋₄-alkyl-, C₁₋₃-alkoxy-C₂₋₆-alkyl-, amino-C₂₋₆-alkyl-, C₁₋₃-alkyl-amino-C₂₋₆-alkyl- or di-(C₁₋₃-alkyl)-amino-C₂₋₆-alkyl-,

B has one of the meanings given for Cy, while the bond to the group W or optionally directly to the group A is formed via a C atom of the carbocyclic moiety or of the optionally fused-on phenyl or pyridine ring or via an N or C atom of the heterocyclic moiety,

while if k=0 the group B and the group A may be connected to one another via a common C atom forming a spirocyclic ring system or

via two common, adjacent atoms forming a fused, bicyclic ring system,

Cy denotes a carbo- or heterocyclic group selected from one of the following meanings

a saturated 3- to 7-membered carbocyclic group,

an unsaturated 5- to 7-membered carbocyclic group,

a phenyl group,

a saturated 4- to 7-membered or unsaturated 5- to 7-membered heterocyclic group with an N, O or S atom as heteroatom,

a saturated or unsaturated 5- to 7-membered heterocyclic group with two or more N atoms or with one or two N atoms and an O or S atom as heteroatoms,

an aromatic heterocyclic 5- or 6-membered group with one or more identical or different heteroatoms selected from N, O and/or S,

while the above mentioned 5-, 6- or 7-membered groups may be connected via two common, adjacent C atoms fused with a phenyl or pyridine ring, and

in the above mentioned 5-, 6- or 7-membered groups a —CH₂-group may be replaced by a —CO—, —C(═CH₂)—, —(SO)— or —(SO₂)— group, and

the above mentioned saturated 6- or 7-membered groups may also occur as bridged ring systems with an imino, N—(C₁₋₃-alkyl)-imino-, methylene-, C₁₋₃-alkyl-methylene- or di-(C₁₋₃-alkyl)-methylene-bridge, and

the above mentioned cyclic groups may be mono- or polysubstituted at one or more C atoms with R²⁰, or in the case of a phenyl group may also additionally be monosubstituted by nitro, and/or may be substituted at one or more N atoms with R²¹,

R¹⁵ denotes H, C₁₋₄-alkyl, C₃₋₇-cycloalkyl, C₃₋₇-cycloalkyl-C₁₋₃-alkyl-, phenyl or phenyl-C₁₋₃-alkyl-,

R¹⁷ has one of the meanings given for R¹⁶ or denotes phenyl, phenyl-C₁₋₃-alkyl-, dioxolan-2-yl, C₁₋₃-alkylcarbonyl-, hydroxycarbonyl-C₁₋₃-alkyl-, C₁₋₃-alkylcarbonylamino-C₂₋₃-alkyl-, C₁₋₃-alkylsulphonyl- or C₁₋₃-alkylsulphonylamino-C₂₋₃-alkyl-,

R²² denotes phenyl, phenyl-C₁₋₃-alkoxy-, C₁₋₃-alkoxy, C₁₋₃-alkylthio, carboxy, C₁₋₃-alkylcarbonyl, C₁₋₃-alkoxycarbonyl, aminocarbonyl, C₁₋₃-alkylaminocarbonyl, di-(C₁₋₃-alkyl)-aminocarbonyl, C₁₋₃-alkyl-sulphonyl, C₁₋₃-alkyl-sulphinyl, C₁₋₃-alkyl-sulphonylamino, amino, C₁₋₃-alkylamino-, di-(C₁₋₃-alkyl)-amino-, phenyl-C₁₋₃-alkylamino- or N—(C₁₋₃-alkyl)-phenyl-C₁₋₃-alkylamino-, acetylamino-, propionylamino-, phenylcarbonyl, phenylcarbonylamino-, phenylcarbonylmethylamino-, hydroxyalkylaminocarbonyl, (4-morpholinyl)carbonyl, (1-pyrrolidinyl)carbonyl, (1-piperidinyl)carbonyl, (hexahydro-1-azepinyl)carbonyl, (4-methyl-1-piperazinyl)carbonyl, methylenedioxy, aminocarbonyl-amino- or alkylaminocarbonylamino-

while in the groups and residues A, B, W, X, Y, Z, R¹ to R⁹ and R¹¹ to R²² in each case one or more C atoms may be mono- or polysubstituted by F and/or in each case one or two C atoms may be monosubstituted by Cl or Br independently of one another and

the H atom of any carboxy group present or an H atom bound to an N atom may be replaced in each case by a group which can be cleaved in vivo,

the tautomers, diastereomers, enantiomers, mixtures thereof and the salts thereof.

According to the first group of the preferred embodiments the group A and the group R³ are not directly connected to one another. Therefore the group A has one of the meanings given for Cy.

According to the second group of the preferred embodiments the group A and the group R³ are connected to one another in such a way that the group

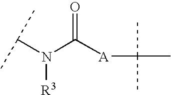

of formula I denotes a group of partial formula II

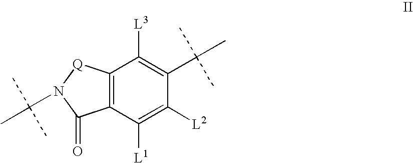

and

Q denotes a group selected from the partial formulae IIIa to IIIg

—CR⁶R⁷— IIIa,

—CR⁶═CR⁷— IIIb,

—N═CR⁸— IIIc,

—N═N— IIId,

—CO—NR⁹— IIIe,

—CR⁸═N— IIIf,

—CO— IIIg.

Preferred meanings for the group Q are selected from the partial formulae IIIb, IIId, IIIe, IIIf and IIIg, particularly IIId, IIIe, IIIf and IIIg.

Preferred meanings for the substituents R⁶, R⁷, R⁸ and R⁹ are independently of one another H and C₁₋₄-alkyl, particularly H, methyl or ethyl.

Preferably the substituents L¹, L², L³ independently of one another have one of the following meanings H, F, Cl, Br, CH₃, CHF₂, CF₃, C₂H₅, C₃H₇, CH(CH₃)₂, OCH₃, OCHF₂, OCF₃, OC₂H₅, OC₃H₇ and OCH(CH₃)₂.

Preferably only one of the substituents L¹, L², L³ has a meaning other than H, particularly one of the meanings mentioned above as being preferred. Particularly preferably all three substituents L¹, L², L³ represent H.

Preferably the groups R¹, R² independently of one another denote H, C₁₋₆-alkyl, C₃₋₇-cycloalkyl, C₃₋₇-cycloalkyl-C₁₋₃-alkyl-, ω-hydroxy-C₂₋₃-alkyl-, ω-(C₁₋₃-alkoxy)-C₂₋₃-alkyl-, C₁₋₄-alkoxy-carbonyl-C₁₋₃-alkyl-, amino-C₂₋₄-alkyl-, C₁₋₃-alkyl-amino-C₂₋₄-alkyl- or di-(C₁₋₃-alkyl)-amino-C₂₋₄-alkyl-, phenyl or phenyl-C₁₋₃-alkyl-, while in the above mentioned groups and residues one or more C atoms may be mono- or polysubstituted by F and/or one or two C atoms independently of one another may be monosubstituted by Cl or Br, and the phenyl group may be mono- or polysubstituted by the above defined group R¹² and/or may be monosubstituted by nitro.

Most preferably the groups R¹, R² independently of one another denote C₁₋₄-alkyl, C₃₋₇-cycloalkyl, C₃₋₇-cycloalkyl-C₁₋₃-alkyl-, ω-hydroxy-C₂₋₃-alkyl-, ω-(C₁₋₃-alkoxy)-C₂₋₃-alkyl-, C₁₋₄-alkoxy-carbonyl-C₁₋₃-alkyl-, while one of the groups R¹, R² may also denote H.

Preferably, also, R¹ and R² form an alkylene bridge in such a way that R¹R²N-denotes a group selected from azetidine, pyrrolidine, piperidine, azepan, 2,5-dihydro-1H-pyrrole, 1,2,3,6-tetrahydro-pyridine, 2,3,4,7-tetrahydro-1H-azepinyl, 2,3,6,7-tetrahydro-1H-azepine, piperazine, wherein the free imine function may be substituted by R¹³, morpholine and thiomorpholine, while according to the general definition of R¹ and R² one or more H atoms may be replaced by R¹⁴, and/or the above mentioned groups may be substituted by one or two identical or different carbo- or heterocyclic groups Cy in a manner specified according to the general definition of R¹ and R².

Particularly preferably, the group

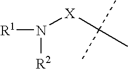

is defined according to one of the following partial formulae

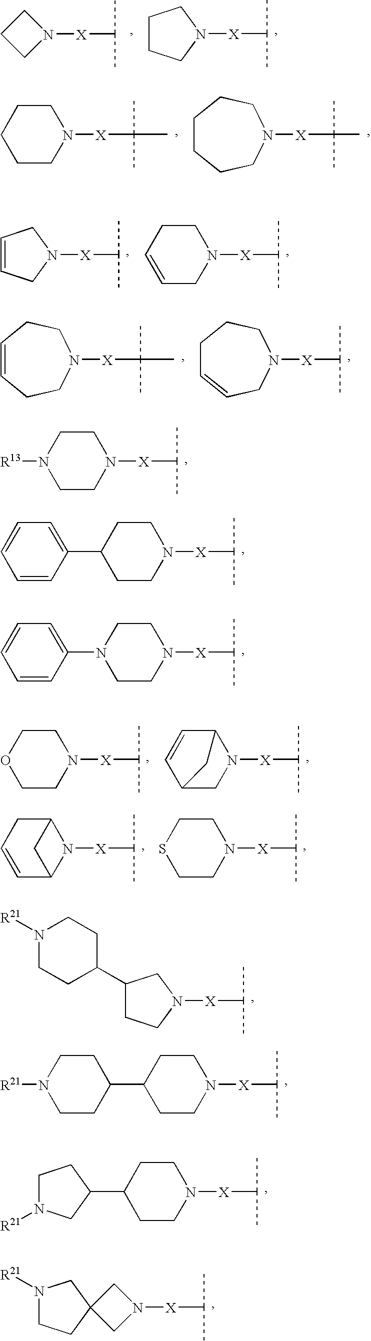

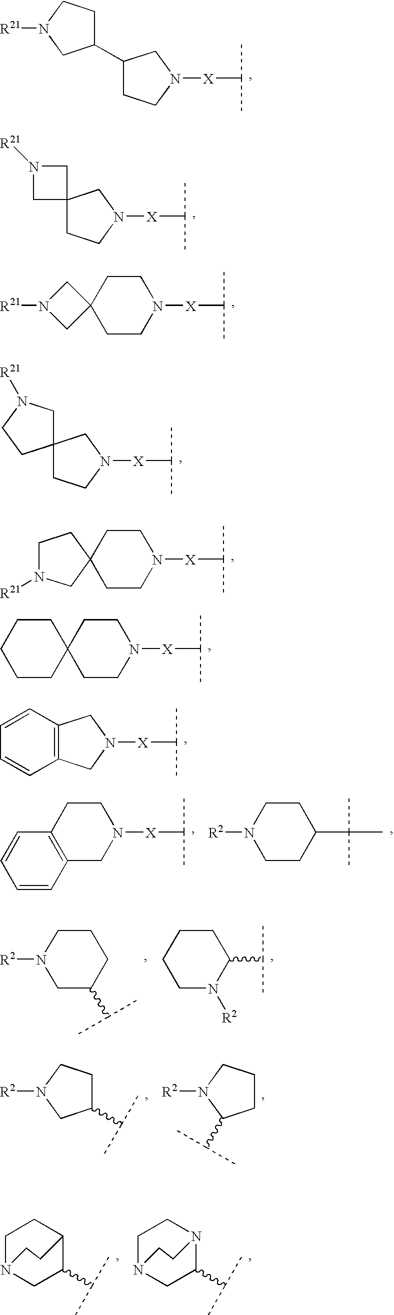

wherein one or more H atoms of the heterocycle formed by the group R¹R²N— may be replaced by R¹⁴ and the ring connected to the heterocycle formed by the group R¹R²N— may be mono- or polysubstituted at one or more C atoms by R²⁰, and in the case of a phenyl ring may also additionally be monosubstituted by nitro.

Most particularly preferred are the groups R¹R²N described above, wherein R¹ and R² form with the N atom of the group R¹R²N— a pyrrolidine, piperidine or 2,5-dihydro-1H-pyrrole ring, which may be substituted as specified.

Preferred meanings for the group R¹⁴ are C₁₋₄-alkyl, C₁₋₄-cycloalkyl, hydroxy, C₁₋₄-alkoxy, C₁₋₄-alkoxy-C₁₋₃-alkyl-, hydroxy-C₁₋₃-alkyl, C₁₋₄-alkyl-carbonyl, C₁₋₄-alkoxy-carbonyl, C₁₋₄-alkoxy-carbonyl-C₁₋₃-alkyl-, C₁₋₄-alkoxy-carbonylamino-, C₁₋₄-alkoxy-carbonylamino-C₁₋₃-alkyl-, amino, (C₁₋₄-alkyl)-amino-, di-(C₁₋₄-alkyl)-amino-, phenyl, phenyloxy, pyridinyl and pyridinyloxy.

A preferred piperidine group substituted by the group Cy has the structure

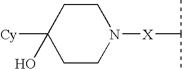

wherein Cy preferably denotes phenyl, which may be substituted as specified.

Preferably the alkylene bridge X has no, or at most one, —NR⁴— group. The position of the NR⁴ group within the alkylene bridge X is preferably selected so that together with the amino group NR¹R² or another adjacent amino group no aminal function is formed or two N atoms are adjacent to one another. Therefore, in the event that a —CH₂-group is replaced by —NR⁴—, the alkylene bridge preferably denotes C₂₋₇-alkylene-NR⁴—C₀₋₅-alkylene, while the bridge X has a maximum of 7 bridging C atoms in addition to the N atom and the C atoms may be substituted in the specified manner.

Preferably X denotes a single bond or an unbranched bridge selected from C₁₋₆-alkylene, C₂₋₆-alkenylene, C₂₋₆-alkynylene, C₁₋₆-alkylenoxy, carbonyl, carbonyl-C₁₋₆-alkylene or C₁₋₆-alkylene-amino-, wherein the amino group may be substituted by R⁴, while one or two C atoms may be substituted in the manner specified in the general definition of X and/or the alkylene bridge may be connected to R¹ in the manner specified.

Particularly preferably, X denotes a single bond, carbonyl or an alkylene bridge selected from methylene, 1,2-ethylene, 1,3-propylene and 1,4-butylene, wherein one or two C atoms may be substituted independently of one another with a hydroxy, ω-hydroxy-C₁₋₃-alkyl, ω-(C₁₋₃-alkoxy)-C₁₋₃-alkyl- and/or C₁₋₃-alkoxy group and/or in each case with one or two identical or different C₁₋₄-alkyl groups, and in each case one or more C atoms may be mono- or polysubstituted by F and/or in each case one or two C atoms may be monosubstituted by Cl or Br independently of one another.

If in group X one or two C atoms are substituted by a hydroxy and/or C₁₋₃-alkoxy group, the substituted C atom is preferably not directly adjacent to an amino group, particularly —NR¹R² or —NR⁴—.

Most preferably, the bridge X is a single bond, —CH₂— or —CH(CH₃)—.

In the event that a —CH₂-group is replaced by —NR⁵— in the bridge Z, the position of the NR⁵ group within the group Z is preferably selected so that together with the amino group —NR³— or another adjacent amino group no aminal function is formed or two N atoms are adjacent to one another.

Preferred meanings of the bridge Z are methylene, 1,2-ethylene, 1,3-propylene, 1,4-butylene, methyleneoxy, 1,2-ethyleneoxy, 1,3-propyleneoxy and 1,4-butyleneoxy, wherein one or two C atoms may be substituted independently of one another by a hydroxy, ω-hydroxy-C₁₋₃-alkyl, ω-(C₁₋₃-alkoxy)-C₁₋₃-alkyl- and/or C₁₋₃-alkoxy group and/or in each case by one or two identical or different C₁₋₄-alkyl groups, and in each case one or more C atoms may be mono- or polysubstituted by F and/or in each case one or two C atoms independently of one another may be monosubstituted by Cl or Br and R³ may be connected to Z so as to include the N atoms connected to R³, forming a heterocyclic group.

If in the group Z one or two C atoms are substituted by a hydroxy and/or C₁₋₃-alkoxy group, the substituted C atom is preferably not directly adjacent to an amino group, particularly —NR³— or —NR⁵—.

Particularly preferably, Z is selected from the group —CH₂—, —CH₂—CH₂—, —CH₂—CH(CH₃)—, —CH₂—C(CH₃)₂—, —CH(CH₃)—CH₂—, —C(CH₃)₂—CH₂— and —CH₂—O—, particularly —CH₂—CH₂— or —CH(CH₃)—CH₂—.

Moreover according to a particularly preferred definition Z is connected to R³ so that the group of partial formula

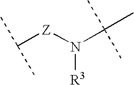

has a meaning selected from 1,3-pyrrolidinylene, 1,3-piperidinylene, 1,2,5,6-tetrahydropyridin-1,3-ylene and 3-hydroxy-1,3-piperidinylene.

Preferably the group R³ is selected from among methyl, ethyl, n-propyl, iso-propyl, 2-hydroxyethyl, 3-hydroxy-n-propyl or 2-hydroxy-1-methyl-ethyl, while in the groups specified one, two or three H atoms may be replaced by F, or R³ is selected from the group H, amino-C₂₋₃-alkyl-, C₁₋₃-alkyl-amino-C₂₋₃-alkyl- or di-(C₁₋₃-alkyl)-amino-C₂₋₃-alkyl-.

Particularly preferred meanings of the group R³ are H, methyl or ethyl, particularly H or methyl.

Preferred meanings of the groups R⁴ and/or R⁵ are H, C₁₋₄-alkyl, C₃₋₆-cycloalkyl and C₃₋₆-cycloalkyl-C₁₋₃-alkyl-, particularly H and C₁₋₄-alkyl.

Preferred meanings of the group R¹¹ are C₁₋₆-cycloalkyl, hydroxy, C₁₋₄-alkoxy, amino, C₁₋₄-alkyl-amino- and di-(C₁₋₄-alkyl)-amino-.

Preferred meanings of the group R²⁰ are halogen, hydroxy, cyano, C₁₋₄-alkyl, C₃₋₇-cycloalkyl and hydroxy-C₁₋₃-alkyl. Particularly preferably R²⁰ denotes F, Cl, Br, I, OH, cyano, methyl, difluoromethyl, trifluoromethyl, ethyl, n-propyl, iso-propyl, methoxy, difluoromethoxy, trifluoromethoxy, ethoxy, n-propoxy or iso-propoxy.

The group Y is preferably selected from among the bivalent cyclic groups 1,2-cyclopropylene, 1,3-cyclobutylene, 1,3-cyclopentylene, 1,3-cyclopentenylene, 1,3- and 1,4-cyclohexylene, 1,3-phenylene, 1,4-phenylene, 1,3- and 1,4-cyclohexenylene, 1,4-cycloheptylene, 1,4-cycloheptenylene, 1,3-pyrrolidinylene, 1,3-pyrrolinylene, 1,3-pyrrolylene, 1,4-piperidinylene, 1,4-tetrahydropyridinylene, 1,4-dihydropyridinylene, 2,4- and 2,5-pyridinylene or 1,4-piperazinylene, while the above mentioned 5-, 6- or 7-membered groups may be connected via two common, adjacent C atoms fused with a phenyl or pyridine ring, the above mentioned cyclic groups may be mono- or polysubstituted at one or more C atoms by R²⁰, in the case of a phenyl group they may also additionally be monosubstituted by nitro, and/or may be substituted at one or more N atoms with R²¹, and R¹ may be connected to Y and/or R³ may be connected to Y in the manner specified in the general definition.

Most particularly preferred meanings of the group Y are selected from the group of cyclic structures consisting of:

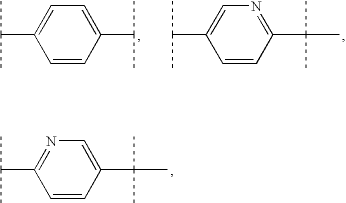

while the cyclic groups may be mono- or disubstituted, preferably monosubstituted, by R²⁰, preferably by halogen, CF₃, C₁₋₄-alkyl and/or C₁₋₄-alkoxy.

In addition, the group Y may also be linked to the group R¹ in such a way that the group of partial formula

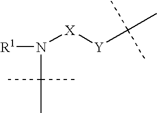

has a meaning selected from the following partial formulae

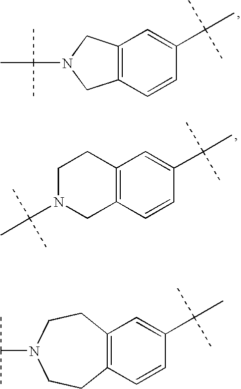

Preferred meanings for the group A are selected from among the bivalent cyclic groups 1,2-cyclopropylene, 1,3-cyclobutylene, 1,3-cyclopentylene, 1,3-cyclopentenylene, 1,3- and 1,4-cyclohexylene, 1,3- and 1,4-phenylene, 1,3- and 1,4-cyclohexenylene, 1,4-cycloheptylene, 1,4-cycloheptenylene, 1,3-pyrrolidinylene, 1,3-pyrrolinylene, 1,3-pyrrolylene, 1,4-piperidinylene, 1,4-tetrahydropyridinylene, 1,4-dihydropyridinylene, 2,4- and 2,5-pyridinylene, 1,4-piperazinylene, 7-aza-bicyclo[2.2.1]heptan-2,7-diyl and 8-aza-bicyclo[3.2.1]octan-3,8-diyl, while the above mentioned 5-, 6- or 7-membered groups may be connected via two common, adjacent C atoms fused with a phenyl or pyridine ring, and the above mentioned cyclic groups may be mono- or polysubstituted at one or more C atoms with R²⁰, in the case of a phenyl group they may also additionally be monosubstituted by nitro, and/or substituted by R²¹ at one or more N atoms.

Most particularly preferred meanings for the group A are selected from the group of cyclic structures consisting of:

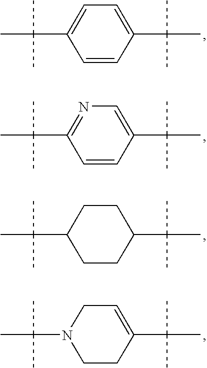

while the cyclic groups may be mono- or disubstituted, preferably monosubstituted, by R²⁰, preferably by halogen, CF₃, C₁₋₄-alkyl and/or C₁₋₄-alkoxy.

The bivalent cyclic groups specified for Y and/or A in each case include the mirror-symmetrical forms, i.e. the forms in which the link to the adjacent groups, to X and Z in the case of Y and also to CO and W in the case of A, is swapped over. Thus, for example, 1,4-cyclohexenylene denotes both

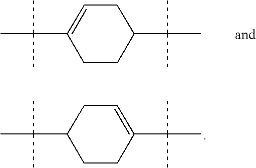

The bivalent cyclic groups given above for the groups Y and A include all the possible isomers. Some meanings mentioned above as being preferred will be explained more fully hereinafter:

The definition tetrahydropyridinylene comprises the meanings 1,2,3,4-tetrahydropyridin-1,4- and -3,6-ylene, 1,2,3,6-tetrahydropyridin-1,4, -2,5- and -3,6-ylene, 2,3,4,5-tetrahydropyridin-2,5- and -3,6-ylene. The preferred meaning is 1,2,3,6-tetrahydropyridin-1,4-ylene.

The definition dihydropyridinylene comprises the meanings 1,4- and 1,2-dihydropyridin-1,4-ylene as well as 1,2-, 1,4-, 1,6-, 2,3-, 2,5-, 3,4-, 4,5- and 5,6-dihydropyridin-2,5-ylene. The preferred meaning is 1,2-dihydropyridin-1,4-ylene.

Preferably the groups A and/or B are unsubstituted or mono- or disubstituted by R²⁰, most preferably unsubstituted or monosubstituted by R²⁰.

Preferred meanings for the group B according to a first embodiment are selected from the group C₁₋₆-alkyl, C₁₋₆-alkenyl, C₁₋₆-alkynyl, C₃₋₇-cycloalkyl-C₁₋₃-alkyl-, C₃₋₇-cycloalkenyl-C₁₋₃-alkyl-, C₃₋₇-cycloalkyl-C₁₋₃-alkenyl- or C₃₋₇-cycloalkyl-C₁₋₃-alkynyl-, wherein one or more C atoms may be mono- or polysubstituted by halogen and/or monosubstituted by hydroxy or cyano and/or cyclic groups may be mono- or polysubstituted by R²⁰, and

W denotes a single bond, —O—, a C₁₋₄-alkylene, C₂₋₄-alkenylene, C₂₋₄-alkynylene, C₁₋₄-alkyleneoxy, Oxy-C₁₋₄-alkylene, C₁₋₃-alkylene-oxy-C₁₋₃-alkylene, imino, N—(C₁₋₃-alkyl)-imino-, imino-C₁₋₄-alkylene-, N—(C₁₋₃-alkyl)-imino-C₁₋₄-alkylene-, C₁₋₄-alkylene-imino- or C₁₋₄-alkylene-N—(C₁₋₃-alkyl)-imino-group, while one or two C atoms independently of one another may be substituted by a hydroxy, ω-hydroxy-C₁₋₃-alkyl, ω-(C₁₋₃-alkoxy)-C₁₋₃-alkyl and/or C₁₋₃-alkoxy group and/or with one or two identical or different C₁₋₄-alkyl groups, and

k denotes 0 or 1, particularly 1 and

R²⁰ has one of the meanings given hereinbefore.

In the above mentioned preferred meanings for B, k preferably has the value 1 and W preferably denotes a single bond, imino or N—(C₁₋₃-alkyl)-imino, particularly a single bond.

Particularly preferably, the group B denotes C₃₋₆-alkynyl, particularly C₃₋₆-alk-1-ynyl, and/or the group W denotes a single bond, while k=1.

Preferred meanings for the group B according to a second embodiment are selected from among the cyclic groups cyclopropyl, cyclobutyl, cyclopentyl, cyclopentenyl, cyclohexyl, cyclohexanonyl, cyclohexenyl, phenyl, cycloheptyl, cycloheptenyl, aziridinyl, azetidinyl, pyrrolidinyl, pyrrolinyl, pyrrolyl, piperidinyl, tetrahydropyridinyl, dihydropyridinyl, pyridinyl, azepanyl, piperazinyl, 1H-pyrazolyl, imidazolyl, triazolyl, tetrazolyl, morpholinyl, thiomorpholinyl, indolyl, isoindolyl, quinolinyl, benzoimidazolyl, isoquinolinyl, furanyl and thienyl, while the bond to the group W or optionally directly to the group A is formed via a C atom of the carbocyclic moiety or of the optionally fused-on phenyl or pyridine ring or via an N or C atom of the heterocyclic moiety, or B together with the group W connected via a double bond is selected from the group cyclopentylidene-methyl, cyclohexylidene-methyl and cyclohexanon-4-ylidene-methyl, and the above mentioned cyclic groups may be mono- or polysubstituted at one or more C atoms with R²⁰, in the case of a phenyl group they may also additionally be monosubstituted by nitro, and/or may be substituted at one or more N atoms with R²¹.

Most particularly preferably the group B denotes phenyl, which is mono-, di- or trisubstituted, preferably mono- or disubstituted by R²⁰.

The definitions of B given above include all the possible isomers for the groups in question. Thus, in particular, the following isomers are included: cyclopenten-1-, 3- and 4-yl, cyclohexanon-4-yl, cyclohexen-1-, 3- and 4-yl, cyclohepten-1-, 3-, 4- and 5-yl, aziridin-1-yl, azetidin-1-yl, pyrrolidin-1-yl, pyrrolin-1-yl, pyrrol-1-yl, piperidin-1- and 4-yl, pyridin-2, -3- and -4-yl, azepan-1-yl, piperazin-1-yl, 4-methyl-piperazin-1-yl, morpholin-4-yl, thiomorpholin-4-yl, quinolin-2-, 3-, 4-, 5-, 6-, 7- and 8-yl, isoquinolin-1-, 3-, 4-, 5-, 6-, 7- and 8-yl, 1H-benzoimidazol-1-, 2-, 4-, 5-, 6- and 7-yl.

The definition pyrazole comprises the isomers 1H-, 3H- and 4H-pyrazole. Preferably pyrazolyl denotes 1H-pyrazol-1-yl.

The definition imidazole comprises the isomers 1H-, 2H- and 4H-imidazole. A preferred meaning of imidazolyl is 1H-imidazol-1-yl.

The definition tetrahydropyridine comprises the isomers 1,2,3,4-, 1,2,3,6- and 2,3,4,5-tetrahydropyridin. Preferably tetrahydropyridinyl denotes 1,2,3,4- and 1,2,3,6-tetrahydropyridin-1-yl.

The definition dihydropyridine comprises the isomers 1,2-, 1,4-, 2,3-, 2,5- and 4,5-dihydropyridine. Preferably dihydropyridinyl denotes 1,2- and 1,4-dihydropyridin-1-yl.

The definition triazole comprises the isomers 1H, 3H- and 4H-[1,2,4]-triazole as well as 1H, 2H- and 4H-[1,2,3]-triazole. The definition triazolyl therefore comprises 1H-[1,2,4]-triazol-1-, 3- and 5-yl, 3H-[1,2,4]-triazol-3- and 5-yl, 4H-[1,2,4]-triazol-3,4- and 5-yl, 1H-[1,2,3]-triazol-1,4- and 5-yl, 2H-[1,2,3]-triazol-2,4- and 5-yl and 4H-[1,2,3]-triazol-4- and 5-yl.

The term tetrazole comprises the isomers 1H-, 2H- and 5H-tetrazole. The definition tetrazolyl therefore comprises 1H-tetrazol-1- and 5-yl, 2H-tetrazol-2- and 5-yl as well as 5H-tetrazol-5-yl.

The definition indole comprises the isomers 1H- and 3H-indol. The term indolyl preferably denotes 1H-indol-1-yl.

The definition isoindole comprises the isomers 1H- and 2H-isoindole. The term isoindolyl preferably denotes 2H-isoindol-2-yl.

Generally, the bond to one of the above mentioned heterocyclic groups, particularly to a pyrazolyl, imidazolyl, tetrahydropyridinyl, dihydropyridinyl, triazolyl, tetrazolyl, indolyl or isoindolyl group, may be formed via a C atom or optionally an N atom of an imine function.

The group B is preferably unsubstituted, mono-, di- or trisubstituted by R²⁰. Particularly preferably B is mono- or disubstituted by R²⁰. In the event that B is a substituted six-membered ring, there is preferably a substituent in the para position to the bond of the group

A[W]ₖ.

The index k may assume the values 0 or 1. In the preferred case k=1 the bridge W has the meanings specified, preferably the meanings of a single bond, —CH₂— or —CH═. Preferred meanings of partial formula -A-W-B are selected from the structures mentioned in the following list, where V denotes a C or an N atom, preferably a C atom, and the cyclic groups mentioned may be mono- or polysubstituted at one or more C atoms with R²⁰ and in the case of phenyl or phenylene groups may also additionally be monosubstituted by nitro:

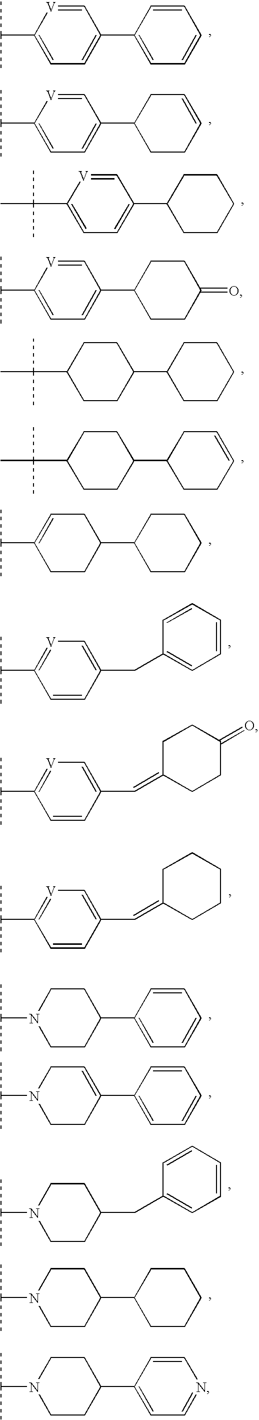

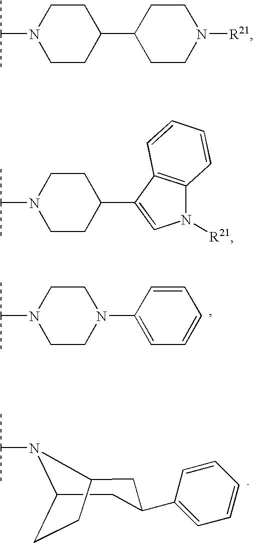

Most particularly preferred are the compounds of formula I, wherein k=1 and W denotes a single bond.

The index k may also assume the value 0. According to a first sub-variant the group A is connected to the group B via a common C atom forming a spirocyclic ring system, while the group A denotes a saturated 5- to 7-membered carbo- or heterocyclic group and the group B denotes a saturated 4- to 7-membered carbo- or heterocyclic group, and the heterocyclic groups in each case have an N, O or S atom, and a phenyl or pyridine ring may be fused to a 5- to 7-membered group B via two adjacent C atoms, and the above mentioned cyclic groups may be mono- or polysubstituted by R²⁰ at one or more C atoms, and in the case of a fused-on phenyl ring may also additionally be monosubstituted by nitro, and/or may be substituted by R²¹ at one or more N atoms.

Preferred meanings of partial formula -A-W-B according to this second sub-variant are selected from the structures listed in the following Table, while the cyclic groups listed may be mono- or polysubstituted by R²⁰ at one or more C atoms and in the case of the phenyl ring may also additionally be monosubstituted by nitro:

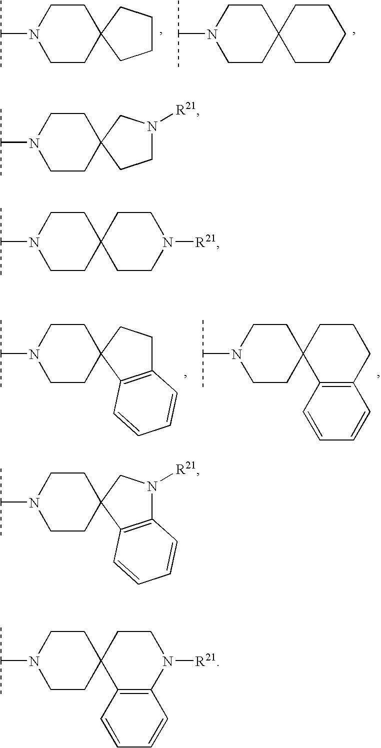

According to a second sub-variant, where k=0, the group B is linked to the group A via two common, adjacent atoms forming a fused, bicyclic saturated, unsaturated or aromatic, 8- to 12-membered ring system, which may contain one or more identical or different heteroatoms selected from N, O and/or S, and the bicyclic ring system may be mono- or polysubstituted at one or more C atoms with R²⁰, in the case of a fused-on phenyl ring it may also additionally be monosubstituted by nitro, and/or may be substituted by R²¹ at one or more N atoms.

Preferred meanings of partial formula -A-W-B according to this first sub-variant are selected from the structures listed in the following Table, while the cyclic groups listed may be mono- or polysubstituted by R²⁰ at one or more C atoms and in the case of the phenyl ring may also additionally be monosubstituted by nitro.

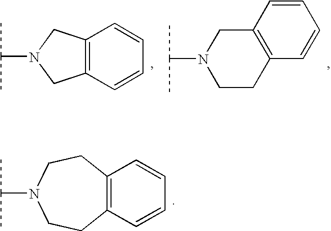

Preferred compounds according to the invention are those wherein one or more of the groups, residues, substituents and/or indices have one of the meanings mentioned above as being preferred.

Preferred meanings of the substituents R²⁰ are selected from among fluorine, chlorine, bromine, CF₃, C₁₋₄-alkyl and C₁₋₄-alkoxy.

Particularly preferred compounds according to the invention are those wherein

Y, A independently of one another are selected from among the bivalent cyclic groups 1,4-phenylene, 1,4-cyclohexylene, 1,4-cyclohexenylene, 1,4-piperidinylene, 1,2,3,6-tetrahydro-pyridin-1,4-ylene, 2,5-pyridinylene and 1,4-piperazinylene, while A may also be connected to R³ according to claim 3, and the above mentioned cyclic groups may be mono- or polysubstituted by R²⁰ at one or more C atoms, in the case of a phenyl group they may also additionally be monosubstituted by nitro, and/or may be substituted by R²¹ at one or more N atoms,

B denotes phenyl or cyclohexyl, while the above mentioned groups may be mono- or polysubstituted by R²⁰ and/or the phenyl ring may additionally be monosubstituted by nitro, while R²⁰ has the meanings given in claim 1, and

k has the value 1,

W is a single bond, —CH₂— or —CH═, and

Z denotes —CH₂—CH₂—, —CH₂—CH(CH₃)—, —CH₂—C(CH₃)₂—, —CH(CH₃)—CH₂—, —C(CH₃)₂—CH₂— or —CH₂—O— or

is linked to R³ in such a way that the group of partial formula

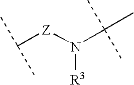

of formula I has a meaning selected from 1,3-pyrrolidinylene and 1,3-piperidinylene.

Particularly preferred compounds according to the invention are listed in the following group of formulae I.1 to I.14:

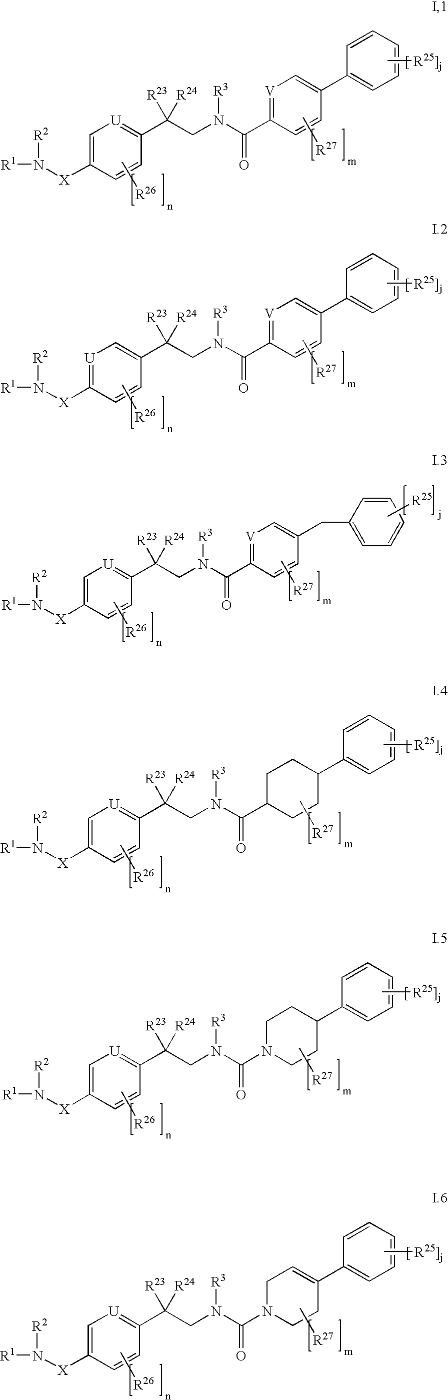

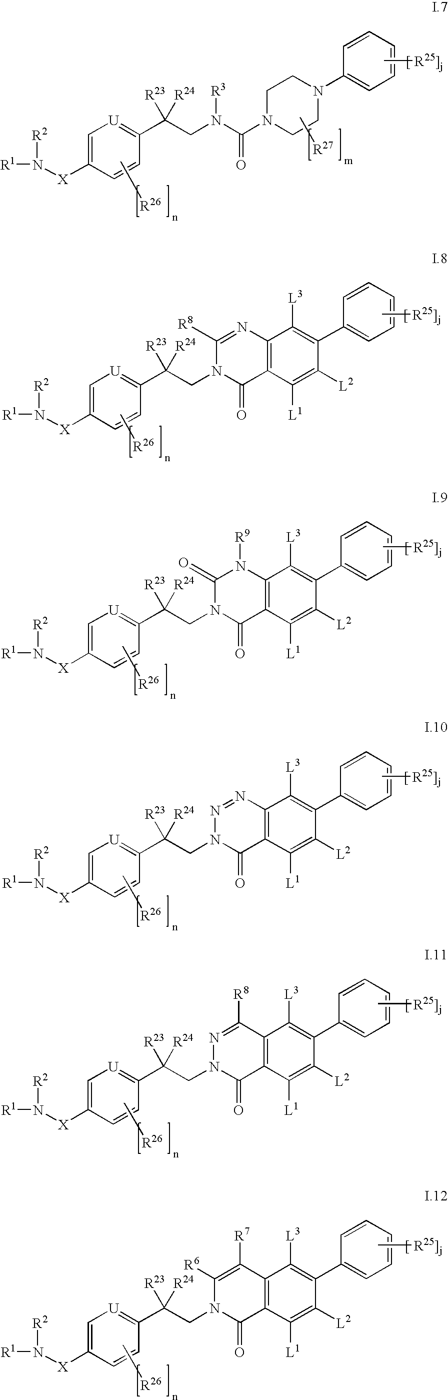

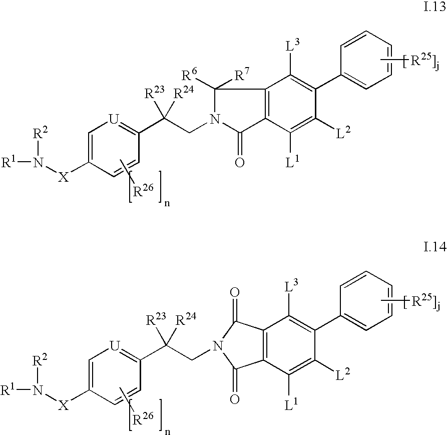

wherein

U, V independently of one another denote C or N,

R²³, R²⁴ independently of one another denote H, F, methyl, trifluoromethyl, ethyl, iso-propyl or n-propyl,

while in formulae I.1 to I.6 R²⁴ may be linked to R³ in such a way that the group of partial formula

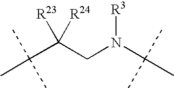

has a meaning selected from 1,3-pyrrolidinylene and 1,3-piperidinylene, and

R²⁵,

R²⁶, R²⁷ independently of one another have one of the meanings given for R²⁰ or in the case of a phenyl group also simply denote nitro, while residues R²⁵, R²⁶, R²⁷ occurring several times may have identical or different meanings, and

j is 0, 1, 2, 3 or 4 and

m, n independently of one another represent 0, 1 or 2.

Most particularly preferred are compounds according to the above formulae I.1, I.2, I.8, I.10 and I.12. In particular, especially preferred compounds may be described by the following formulae

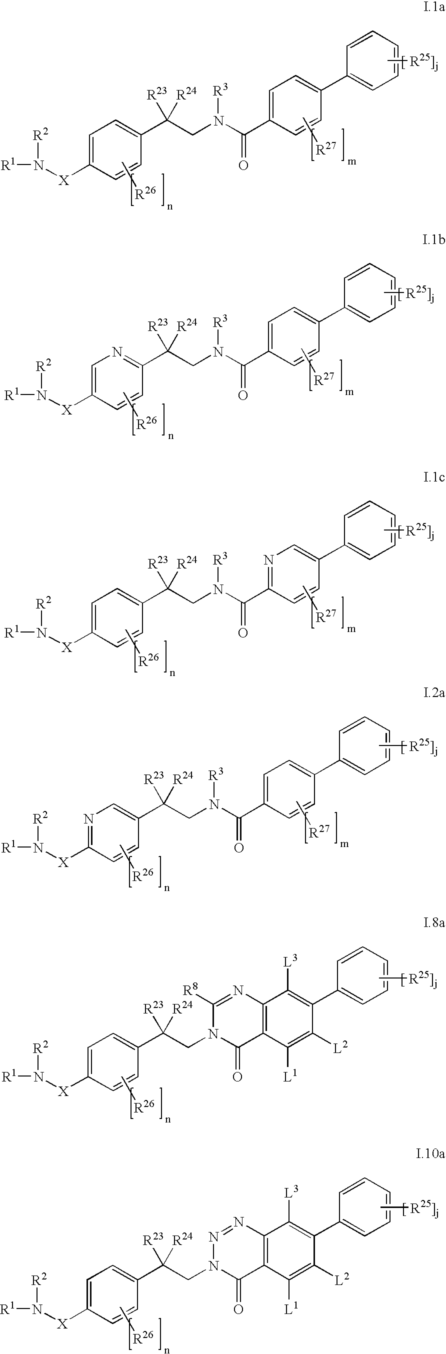

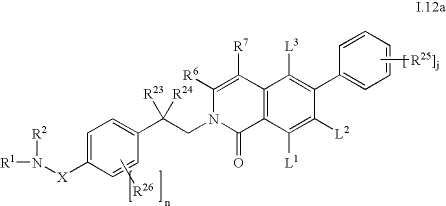

wherein the groups and substituents are defined as above and hereinafter.

Also preferred according to the invention are compounds of the following partial formula

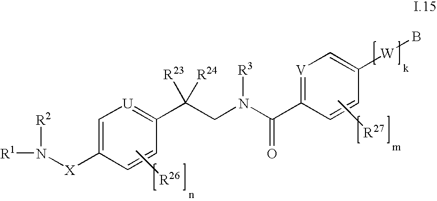

wherein

B is selected from among C₁₋₆-alkyl, C₁₋₆-alkenyl, C₁₋₆-alkynyl, C₃₋₇-cycloalkyl-C₁₋₃-alkyl, C₃₋₇-cycloalkenyl-C₁₋₃-alkyl, C₃₋₇-cycloalkyl-C₁₋₃-alkenyl or C₃₋₇-cycloalkyl-C₁₋₃-alkynyl, wherein one or more C atoms may be mono- or polysubstituted by halogen and/or monosubstituted by hydroxy or cyano and/or cyclic groups may be mono- or polysubstituted by R²⁰, and

W denotes a single bond, —O—, a C₁₋₄-alkylene, C₂₋₄-alkenylene, C₂₋₄-alkynylene, C₁₋₄-alkylenoxy, Oxy-C₁₋₄-alkylene, C₁₋₃-alkylene-oxy-C₁₋₃-alkylene, imino, N—(C₁₋₃-alkyl)-imino-, imino-C₁₋₄-alkylene-, N—(C₁₋₃-alkyl)-imino-C₁₋₄-alkylene-, C₁₋₄-alkylene-imino- or C₁₋₄-alkylene-N—(C₁₋₃-alkyl)-imino-group, while one or two C atoms independently of one another may be substituted with a hydroxy, ω-hydroxy-C₁₋₃-alkyl, ω-(C₁₋₃-alkoxy)-C₁₋₃-alkyl and/or C₁₋₃-alkoxy group and/or with one or two identical or different C₁₋₄-alkyl groups, and

k denotes 0 or 1.

Moreover, according to this embodiment, compounds are preferred wherein the group B denotes C₁₋₆-alkyl, C₁₋₆-alkynyl, C₃₋₇-cycloalkyl-C₁₋₃-alkyl- or C₃₋₇-cycloalkyl-C₁₋₃-alkynyl-, wherein one or more C atoms may be mono- or polysubstituted by halogen and/or monosubstituted by hydroxy or cyano and/or cyclic groups may be mono- or polysubstituted by R²⁰, and/or

W denotes a single bond, —O—, imino or N—(C₁₋₃-alkyl)-imino-, while one or two C atoms independently of one another may be substituted with a hydroxy, ω-hydroxy-C₁₋₃-alkyl, ω-(C₁₋₃-alkoxy)-C₁₋₃-alkyl and/or C₁₋₃-alkoxy group and/or with one or two identical or different C₁₋₄-alkyl groups and k=1.

Most particularly preferred meanings for the group -W-B according to this embodiment are selected from among C₁₋₈-alkyl, —C≡C—C₁₋₆-alkyl, —CH═CH—C₁₋₆-alkyl, —O—C₁₋₆-alkyl, —NH(C₁₋₆-alkyl) and —N(C₁₋₆-alkyl)(C₁₋₃-alkyl), particularly selected from among C₃₋₈-alkyl, —C≡C—C₃₋₆-alkyl, —CH═CH—C₃₋₆-alkyl, —O—C₃₋₆-alkyl, —NH(C₃₋₆-alkyl) and —N(C₃₋₆-alkyl)(C₁₋₃-alkyl).

Particularly preferred among the compounds according to the invention previously described as being preferred, particularly of partial formulae I.1 to I.15, are those wherein the groups R¹, R², R³, L¹, L², L³ and/or group X have one of the meanings mentioned as being preferred in each case.

In particular, especially preferred compounds according to the invention are those compounds wherein X is selected from —CH₂—, —CH(CH₃)— or —C(CH₃)₂—.

Also particularly preferred are those compounds of partial formulae I.1 to I.15 wherein

a) the group U denotes an N atom and the group V denotes a C atom, or

b) the group U denotes a C atom and the group V denotes an N atom, or

c) the two groups U and V each denote a C atom.

In particularly preferred compounds according to the invention the substituents R²⁵, R²⁶, R²⁷ independently of one another have a meaning selected from among F, Cl, Br, I, OH, cyano, methyl, difluoromethyl, trifluoromethyl, ethyl, n-propyl, iso-propyl, methoxy, difluoromethoxy, trifluoromethoxy, ethoxy, n-propoxy or iso-propoxy, and also, in the case of the substitution of a phenyl group, simply nitro, while R²⁵, R²⁶, R²⁷ occurring several times may have identical or different meanings, and j is 0, 1 or 2, and m, n independently of one another denote 0 or 1.

Preferred meanings of the groups R⁶, R⁷, R⁸ and/or R⁹ in the compounds described as preferred according to the invention are, independently of one another, H, methyl, trifluoromethyl, ethyl, iso-propyl or n-propyl, and also F in the case of R⁶ and R⁷.

Particularly preferred individual compounds are selected from the group

(1) 7-(4-chloro-phenyl)-3-[2-(4-pyrrolidin-1-ylmethyl-phenyl)-ethyl]-3H-quinazolin-4-one

(2) 3-[2-(4-pyrrolidin-1-ylmethyl-phenyl)-ethyl]-7-p-tolyl-3H -quinazolin-4-one

(3) 3-[2-(4-pyrrolidin-1-ylmethyl-phenyl)-ethyl]-7-(4-trifluormethyl-phenyl)-3H-quinazolin-4-one

(4) 7-(4-methoxy-phenyl)-3-[2-(4-pyrrolidin-1-ylmethyl-phenyl)-ethyl]-3H-quinazolin-4-one

(5) 7-(3,4-dichloro-phenyl)-3-[2-(4-pyrrolidin-1-ylmethyl-phenyl)-ethyl]-3H-quinazolin-4-one

(6) 7-(4-fluoro-phenyl)-3-[2-(4-pyrrolidin-1-ylmethyl-phenyl)-ethyl]-3H-quinazolin-4-one

(7) 7-(4-ethyl-phenyl)-3-[2-(4-pyrrolidin-1-ylmethyl-phenyl)-ethyl]-3H-quinazolin-4-one

(8) 2-methyl-3-[2-(4-pyrrolidin-1-ylmethyl-phenyl)-ethyl]-7-(4-trifluoromethyl-phenyl)-3H-quinazolin-4-one

(9) 2-methyl-3-[2-(4-pyrrolidin-1-ylmethyl-phenyl)-ethyl]-7-p-tolyl-3H-quinazolin-4-one

(10) 7-(4-chloro-phenyl)-2-methyl-3-[2-(4-pyrrolidin-1-ylmethyl-phenyl)-ethyl]-3H-quinazolin-4-one

(11) 7-(4-chloro-phenyl)-3-[2-(4-pyrrolidin-1-ylmethyl-phenyl)-ethyl]-1H-quinazolin-2,4-dione

(12) 7-(4-chloro-phenyl)-3-{2-[4-((S)-2-methoxymethyl-pyrrolidin-1-ylmethyl)-phenyl]-ethyl}-3H-quinazolin-4-one

(13) 7-(4-chloro-phenyl)-3-[2-(4-dimethylaminomethyl-phenyl)-ethyl]-3H-quinazolin-4-one

(14) 7-(4-chloro-phenyl)-3-[2-(4-piperidin-1-ylmethyl-phenyl)-ethyl]-3H-quinazolin-4-one

(15) 7-(4-chloro-phenyl)-3-[2-(4-morpholin-4-ylmethyl-phenyl)-ethyl]-3H-quinazolin-4-one

(16) 7-(4-chloro-phenyl)-3-[2-(4-pyrrolidin-1-ylmethyl-phenyl)-ethyl]-3H-benzo[d][1,2,3]triazin-4-one

(17) 5-(4-fluoro-phenyl)-2-[2-(4-pyrrolidin-1-ylmethyl-phenyl)-ethyl]-isoindol-1,3-dione

(18) 4′-chloro-biphenyl-4-carboxylic acid [2-(4-pyrrolidin-1-ylmethyl-phenyl)-ethyl]-amide

(19) 4′-chloro-biphenyl-4-carboxylic acid-[2-(4-diethylaminomethyl-phenyl)-ethyl]-amide

(20) 4′-chloro-biphenyl-4-carboxylic acid-[2-(4-piperidin-1-ylmethyl-phenyl)-ethyl]-amide

(21) 4′-methoxy-biphenyl-4-carboxylic acid-[2-(4-diethylaminomethyl-phenyl)-ethyl]-amide

(22) 4′-chloro-biphenyl-4-carboxylic acid-[2-(4-diethylaminomethyl-phenyl)-ethyl]-methyl-amide

(23) 4-(4-chloro-phenyl)-cyclohexanecarboxylic acid-[2-(4-pyrrolidin-1-ylmethyl-phenyl)-ethyl]-amide

(24) 4-methylphenyl-piperidine-1-carboxylic acid-[2-(4-pyrrolidin-1-ylmethyl-phenyl)-ethyl]-amide

(25) 4-(4-chloro-phenyl)-3,6-dihydro-2H-pyridine-1-carboxylic acid-[2-(4-pyrrolidin-1-ylmethyl-phenyl)-ethyl]-amide

(26) 4-(4-chloro-phenyl)-piperidine-1-carboxylic acid-[2-(4-pyrrolidin-1-ylmethyl-phenyl)-ethyl]-amide

(27) 4′-chloro-biphenyl-4-carboxylic acid-[2-(4-pyrrolidin-1-ylmethyl-phenyl)-propyl]-amide

(28) 4′-chloro-biphenyl-4-carboxylic acid-(4-pyrrolidin-1-ylmethyl-benzyloxy)-amide

(29) 4-cyclohexyl-N-[2-(4-pyrrolidin-1-ylmethyl-phenyl)-ethyl]-benzamide

(30) 4′-chloro-biphenyl-4-carboxylic acid-[2-(3-methoxy-4-pyrrolidin-1-ylmethyl-phenyl)-ethyl]-amide

(31) 7-(4-chloro-phenyl)-3-{2-[6-(4-methyl-piperazin-1-yl)-pyridin-3-yl]-ethyl}-3H-quinazolin-4-one

(32) 4′-chloro-biphenyl-4-carboxylic acid-{2-[6-(4-methyl-piperazin-1-yl)-pyridin-3-yl]-ethyl}-amide

(33) 7-(3-methoxy-phenyl)-3-[2-(4-pyrrolidin-1-ylmethyl-phenyl)-ethyl]-3H-quinazolin-4-one

(34) 4-(4-oxo-cyclohexyl)-N-[2-(4-pyrrolidin-1-ylmethyl-phenyl)-ethyl]-benzamide

(35) 4-cyclohexyl-1-cylohexylcarboxylic acid-[2-(4-pyrrolidin-1-ylmethyl-phenyl)-ethyl]-amide

(36) 4-benzyl-piperidine-1-carboxylic acid-[2-(4-pyrrolidin-1-ylmethyl-phenyl)-ethyl]-amide

(37) 4-cyclohexyl-piperidine-1-carboxylic acid-[2-(4-pyrrolidin-1-ylmethyl-phenyl)-ethyl]-amide

(38) 4-(4-chloro-phenyl)-piperazine-1-carboxylic acid-[2-(4-pyrrolidin-1-ylmethyl-phenyl)-ethyl]-amide

(39) 4-(4-fluoro-phenyl)-piperidine-1-carboxylic acid-[2-(4-pyrrolidin-1-ylmethyl-phenyl)-ethyl]-amide

(40) 4-(4-methoxy-phenyl)-piperazine-1-carboxylic acid-[2-(4-pyrrolidin-1-ylmethyl-phenyl)-ethyl]-amide

(41) 4-phenyl-piperidine-1-carboxylic acid-[2-(4-pyrrolidin-1-ylmethyl-phenyl)-ethyl]-amide

(42) (4′-chloro-biphenyl-4-yl)-[3-(4-pyrrolidin-1-ylmethyl-phenyl)-piperidin-1-yl]-methanone

(43) 4′-chloro-biphenyl-4-carboxylic acid-[2-methyl-2-(4-pyrrolidin-1-ylmethyl-phenyl)-propyl]-amide

(44) 4′-chloro-biphenyl-4-carboxylic acid-[2-(4-pyrrolidin-1-ylmethyl-cyclohexyl)-ethyl]-amide

(45) 4-benzyl-N-[2-(4-pyrrolidin-1-ylmethyl-phenyl)-ethyl]-benzamide

(46) 4-(4-oxo-cyclohexylidenemethyl)-N-[2-(4-pyrrolidin-1-ylmethyl-phenyl)-ethyl]-benzamide

(47) 4′-chloro-biphenyl-4-carboxylic acid-[2-(2-fluoro-4-pyrrolidin-1-ylmethyl-phenyl)-ethyl]-amide

(48) 5-(4-chloro-phenyl)-2-[2-(4-pyrrolidin-1-ylmethyl-phenyl)-ethyl]-2,3-dihydro-isoindol-1-one

(49) 4-piperidin-1-yl-N-[2-(4-pyrrolidin-1-ylmethyl-phenyl)-ethyl]-benzamide

(50) 7-(4-chloro-phenyl)-3-{2-[4-(4-hydroxy-4-phenyl-piperidin-1-ylmethyl)-phenyl]-ethyl}-3H-benzo[d][1,2,3]triazin-4-one

(51) 7-(4-chloro-phenyl)-3-{2-[4-(3-Aza-spiro[5.5]undec-3-ylmethyl)-phenyl]-ethyl}-3H-quinazolin-4-one

(52) 7-(4-chloro-phenyl)-3-{2-[4-(3-aza-spiro[5.5]undec-3-ylmethyl)-phenyl]-ethyl}-3H-benzo[d][1,2,3]triazin-4-one

(53) 7-(4-chloro-phenyl)-3-{2-[4-(4-hydroxy-4-phenyl-piperidin-1-ylmethyl)-phenyl]-ethyl}-3H-quinazolin-4-one

(54) 7-(4-chloro-phenyl)-3-(2-{4-[4-(pyridin-2-yloxy)-piperidin-1-ylmethyl]-phenyl}-ethyl)-3H-quinazolin-4-one

(55) 6-(4-chloro-phenyl)-2-[2-(4-pyrrolidin-1-ylmethyl-phenyl)-ethyl]-2H-isoquinolin-1-one

(56) 4′-chloro-biphenyl-4-carboxylic acid [2-(3-bromo-4-pyrrolidin-1-ylmethyl-phenyl)-ethyl]-amide

(57) 4′-chloro-biphenyl-4-carboxylic acid [2-(3-methyl-4-pyrrolidin-1-ylmethyl-phenyl)-ethyl]-amide

(58) 4′-chloro-biphenyl-4-carboxylic acid {2-[4-(1-ethyl-piperidin-2-yl)-phenyl]-ethyl}-amide

(59) 4′-chloro-biphenyl-4-carboxylic acid{2-[4-(4-acetyl-piperazin-1-ylmethyl)-phenyl]-ethyl}-amide

(60) 4′-chloro-biphenyl-4-carboxylic acid{2-[4-(2-aza-bicyclo[2.2.1]hept-5-en-2-ylmethyl)-phenyl]-ethyl}-amide

(61) 4′-chloro-biphenyl-4-carboxylic acid {2-[4-(1,3-dihydro-isoindol-2-ylmethyl)-phenyl]-ethyl}-amide

(62) 4′-chloro-biphenyl-4-carboxylic acid (2-{4-[(diisopropylamino)-methyl]-phenyl}-ethyl)-amide

(63) 4′-chloro-biphenyl-4-carboxylic acid {2-[3-bromo-4-(2,5-dihydro-pyrrol-1-ylmethyl)-phenyl]-ethyl}-amide

(64) 4′-chloro-biphenyl-4-carboxylic acid{2-[4-(2-dimethylaminomethyl-pyrrolidin-1-ylmethyl)-phenyl]-ethyl}-amide

(65) 4′-chloro-biphenyl-4-carboxylic acid{2-[4-(3-dimethylamino-pyrrolidin-1-ylmethyl)-phenyl]-ethyl}-amide

(66) 4′-chloro-biphenyl-4-carboxylic acid [2-(2-bromo-4-pyrrolidin-1-ylmethyl-phenyl)-ethyl]-amide

(67) 4-pent-1-ynyl-N-[2-(4-pyrrolidin-1-ylmethyl-phenyl)-ethyl]-benzamide

(68) 4′-chloro-biphenyl-4-carboxylic acid [2-(6-pyrrolidin-1-ylmethyl-pyridin-3-yl)-ethyl]-amide

(69) 4′-chloro-biphenyl-4-carboxylic acid [2-(1-pyrrolidin-1-yl-indan-5-yl)-ethyl]-amide

(70) 4′-chloro-biphenyl-4-carboxylic acid [2-(2-nitro-4-pyrrolidin-1-ylmethyl-phenyl)-ethyl]-amide

(71) 2′,4′-dichloro-biphenyl-4-carboxylic acid [2-(4-pyrrolidin-1-ylmethyl-phenyl)-ethyl]-amide

(72) 4′-chloro-biphenyl-4-carboxylic acid{2-[4-(3-amino-pyrrolidin-1-ylmethyl)-phenyl]-ethyl}-amide

(73) 4′-chloro-biphenyl-4-carboxylic acid{2-[4-(2-aminomethyl-pyrrolidin-1-ylmethyl)-phenyl]-ethyl}-amide

(74) 4′-chloro-biphenyl-4-carboxylic acid{2-[4-(2-methyl-2,6-diaza-spiro[3.4]oct-6-ylmethyl)-phenyl]-ethyl}-amide

(75) 4′-chloro-biphenyl-4-carboxylic acid [2-(5-pyrrolidin-1-ylmethyl-pyridin-2-yl)-ethyl]-amide

(76) 4′-chloro-biphenyl-4-carboxylic acid [2-(3-ethyl-4-pyrrolidin-1-ylmethyl-phenyl)-ethyl]-amide

(77) 4′-bromo-biphenyl-4-carboxylic acid {2-[4-(2,5-dihydro-pyrrol-1-ylmethyl)-phenyl]-ethyl}-amide

(78) 4-(5-chloro-thiophen-2-yl)-N-[2-(4-pyrrolidin-1-ylmethyl-phenyl)-ethyl]-benzamide

(79) 4′-chloro-biphenyl-4-carboxylic acid [2-(2-methyl-4-pyrrolidin-1-ylmethyl-phenyl)-ethyl]-amide

(80) 4′-bromo-3-fluoro-biphenyl-4-carboxylic acid {2-[3-bromo-4-(2,5-dihydro-pyrrol-1-ylmethyl)-phenyl]-ethyl}-amide

(81) 4′-chloro-2-fluoro-biphenyl-4-carboxylic acid [2-(4-pyrrolidin-1-ylmethyl-phenyl)-ethyl]-amide

(82) 4′-ethyl-biphenyl-4-carboxylic acid [2-(4-pyrrolidin-1-ylmethyl-phenyl)-ethyl]-amide

(83) tert.butyl [1-(4-{2-[(4′-chloro-biphenyl-4-carbonyl)-amino]-ethyl}-benzyl)-pyrrolidin-2-ylmethyl]-carbaminate

(84) 4′-chloro-biphenyl-4-carboxylic acid {2-[4-(2-methyl-piperidin-1-ylmethyl)-phenyl]-ethyl}-amide

(85) 4′-chloro-biphenyl-4-carboxylic acid {2-[4-(2-methyl-pyrrolidin-1-ylmethyl)-phenyl]-ethyl}-amide

(86) 4′-chloro-biphenyl-4-carboxylic acid (2-{4-[(cyclopropylmethyl-amino)-methyl]-phenyl}-ethyl)-amide

(87) 4′-chloro-biphenyl-4-carboxylic acid {2-[4-(3,4-dihydro-1H-isoquinolin-2-ylmethyl)-phenyl]-ethyl}-amide

(88) 4′-chloro-biphenyl-4-carboxylic acid [2-(4-{[(2-hydroxy-ethyl)-methyl-amino]-methyl}-phenyl)-ethyl]-amide

(89) tert.butyl [1-(4-{2-[(4′-chloro-biphenyl-4-carbonyl)-amino]-ethyl}-benzyl)-pyrrolidin-3-yl]-carbaminate

(90) 4′-chloro-biphenyl-4-carboxylic acid {2-[4-(2,6-dimethyl-piperidin-1-ylmethyl)-phenyl]-ethyl}-amide

(91) 4′-chloro-biphenyl-4-carboxylic acid [2-(4-azetidin-1-ylmethyl-phenyl)-ethyl]-amide

(92) 3,4′-dichloro-biphenyl-4-carboxylic acid [2-(4-pyrrolidin-1-ylmethyl-phenyl)-ethyl]-amide

(93) 4′-fluoro-biphenyl-4-carboxylic acid [2-(4-pyrrolidin-1-ylmethyl-phenyl)-ethyl]-amide

(94) 4′-chloro-3-fluoro-biphenyl-4-carboxylic acid [2-(4-pyrrolidin-1-ylmethyl-phenyl)-ethyl]-amide

(95) 2′-fluoro-4′-chloro-biphenyl-4-carboxylic acid [2-(4-pyrrolidin-1-ylmethyl-phenyl)-ethyl]-amide

(96) 5-(4-chloro-phenyl)-pyridine-2-carboxylic acid [2-(4-pyrrolidin-1-ylmethyl-phenyl)-ethyl]-amide

(97) 4′-chloro-biphenyl-4-carboxylic acid {2-[4-(2,5-dihydro-pyrrol-1-ylmethyl)-phenyl]-ethyl}-amide

(98) 4′-bromo-biphenyl-4-carboxylic acid [2-(4-pyrrolidin-1-ylmethyl-phenyl)-ethyl]-amide

(99) 4′-chloro-biphenyl-4-carboxylic acid {2-[4-(1-pyrrolidin-1-yl-ethyl)-phenyl]-ethyl}-amide

Most particularly preferred are the above mentioned individual compounds of formulae (1), (2), (3), (4), (5), (6), (7), (8), (9), (10), (11), (12), (13), (14), (15), (16), (17), (18), (19), (20), (21), (22), (23), (24), (25), (25), (26), (27), (28), (29), (30), (47) as well as (50) to (99).

Some expressions used hereinbefore and below to describe the compounds according to the invention will now be defined more fully.

The term halogen denotes an atom selected from among F, Cl, Br and I.

The term C₁₋ₙ-alkyl, where n has a value of 3 to 8, denotes a saturated, branched or unbranched hydrocarbon group with 1 to n C atoms. Examples of such groups include methyl, ethyl, n-propyl, iso-propyl, butyl, iso-butyl, sec-butyl, tert-butyl, n-pentyl, iso-pentyl, neo-pentyl, tert-pentyl, n-hexyl, iso-hexyl, etc.

The term C₁₋ₙ-alkylene, where n may have a value of 1 to 8, denotes a saturated, branched or unbranched hydrocarbon bridge with 1 to n C atoms. Examples of such groups include methylene (—CH₂—), ethylene (—CH₂—CH₂—), 1-methyl-ethylene (—CH(CH₃)—CH₂—), 1,1-dimethyl-ethylene (—C(CH₃)₂—CH₂—), n-prop-1,3-ylene (—CH₂—CH₂—CH₂—), 1-methylprop-1,3-ylene (—CH(CH₃)—CH₂—CH₂—), 2-methylprop-1,3-ylene (—CH₂—CH(CH₃)—CH₂—), etc., as well as the corresponding mirror-symmetrical forms.

The term C₂₋ₙ-alkenyl, where n has a value of 3 to 6, denotes a branched or unbranched hydrocarbon group with 2 to n C atoms and at least one C═C-double bond. Examples of such groups include vinyl, 1-propenyl, 2-propenyl, iso-propenyl, 1,3-butadienyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 2-hexenyl, 3-hexenyl, 2,4-hexadienyl, 5-hexenyl etc.

The term C₁₋ₙ-alkoxy denotes a —O—C₁₋ₙ-alkyl group, wherein C₁₋ₙ-alkyl is defined as above. Examples of such groups include methoxy, ethoxy, n-propoxy, iso-propoxy, n-butoxy, iso-butoxy, sec-butoxy, tert-butoxy, n-pentoxy, iso-pentoxy, neo-pentoxy, tert-pentoxy, n-hexoxy, iso-hexoxy etc.

The term C₁₋ₙ-alkylthio denotes an —S—C₁₋ₙ-alkyl group, wherein C₁₋ₙ-alkyl is defined as above. Examples of such groups include methylthio, ethylthio, n-propylthio, iso-propylthio, n-butylthio, iso-butylthio, sec-butylthio, tert-butylthio, n-pentylthio, iso-pentylthio, neo-pentylthio, tert-pentylthio, n-hexylthio, iso-hexylthio, etc.

The term C₁₋ₙ-alkylcarbonyl denotes a —C(═O)—C₁₋ₙ-alkyl group, wherein C₁₋ₙ-alkyl is defined as above. Examples of such groups include methylcarbonyl, ethylcarbonyl, n-propylcarbonyl, iso-propylcarbonyl, n-butylcarbonyl, iso-butylcarbonyl, sec-butylcarbonyl, tert-butylcarbonyl, n-pentylcarbonyl, iso-pentylcarbonyl, neo-pentylcarbonyl, tert-pentylcarbonyl, n-hexylcarbonyl, iso-hexylcarbonyl, etc.

The term C₃₋ₙ-cycloalkyl denotes a saturated mono-, bi-, tri- or spirocarbocyclic group with 3 to n C atoms. Examples of such groups include cyclopropyl, cyclobutyl, cyclopentyl, cyclohexyl, cycloheptyl, cyclooctyl, cyclononyl, cyclododecyl, bicyclo[3.2.1.]octyl, spiro[4.5]decyl, norpinyl, norbonyl, norcaryl, adamantyl, etc.

The term C₃₋ₙ-cycloalkylcarbonyl denotes a —C(═O)—C₃₋ₙ-cycloalkyl group, wherein C₃₋ₙ-cycloalkyl is defined as above.

The term aryl denotes a carbocyclic, aromatic ring system, such as for example phenyl, biphenyl, naphthyl, anthracenyl, phenanthrenyl, fluorenyl, indenyl, pentalenyl, azulenyl, biphenylenyl, etc.

The term heteroaryl used in this application denotes a heterocyclic, aromatic ring system which comprises in addition to at least one C atom one or more heteroatoms selected from N, O and/or S. Examples of such groups are furanyl, thiophenyl (thienyl), pyrrolyl, oxazolyl, thiazolyl, imidazolyl, isoxazolyl, isothiazolyl, 1,2,3-triazolyl, 1,3,5-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuranyl, benzothiophenyl (thianaphthenyl), indazolyl, benzimidazolyl, benzthiazolyl, benzisothiazolyl, benzoxazolyl, benzisoxazolyl, purinyl, quinazolinyl, quinozilinyl, quinolinyl, isoquinolinyl, quinoxalinyl, naphthyridinyl, pteridinyl, carbazolyl, azepinyl, diazepinyl, acridinyl, etc. The term heteroaryl also comprises the partially hydrogenated heterocyclic, aromatic ring systems, particularly those listed above. Examples of such partially hydrogenated ring systems are 2,3-dihydrobenzofuranyl, pyrolinyl, pyrazolinyl, indolinyl, oxazolidinyl, oxazolinyl, oxazepinyl, etc.

Terms such as aryl-C₁₋ₙ-alkyl, heteroaryl-C₁₋ₙ-alkyl, etc. refer to C₁₋ₙ-alkyl, as defined above, which is substituted with an aryl or heteroaryl group. Many of the terms given above may be used repeatedly in the definition of a formula or group and in each case have one of the meanings given above, independently of one another.

The term “unsaturated carbocyclic group” or “unsaturated heterocyclic group”, as used particularly in the definition of the group Cy, comprises in addition to the totally unsaturated groups, the corresponding, only partially unsaturated groups, particularly mono- and diunsaturated groups.

The term “optionally substituted” used in this application indicates that the group thus designated is either unsubstituted or mono- or polysubstituted by the substituents specified. If the group in question is polysubstituted, the substituents may be identical or different.

The residues and substituents described above may be mono- or polysubstituted by fluorine as described. Preferred fluorinated alkyl groups are fluoromethyl, difluoromethyl and trifluoromethyl. Preferred fluorinated alkoxy groups are fluoromethoxy, difluoromethoxy and trifluoromethoxy. Preferred fluorinated alkylsulphinyl and alkylsulphonyl groups are trifluoromethylsulphinyl and trifluoromethylsulphonyl.

The compounds of general formula I according to the invention may have acid groups, predominantly carboxyl groups, and/or basic groups such as e.g. amino functions. Compounds of general formula I may therefore be present as internal salts, as salts with pharmaceutically useable inorganic acids such as hydrochloric acid, sulphuric acid, phosphoric acid, sulphonic acid or organic acids (such as for example maleic acid, fumaric acid, citric acid, tartaric acid or acetic acid) or as salts with pharmaceutically useable bases such as alkali or alkaline earth metal hydroxides or carbonates, zinc or ammonium hydroxides or organic amines such as e.g. diethylamine, triethylamine, triethanolamine inter alia.

The compounds according to the invention may be obtained using methods of synthesis which are known in principle. Preferably the compounds are obtained by the method of preparation described above and explained more fully hereinafter.

The method of preparation according to the invention to obtain the first group of the preferred embodiments, i.e. those compounds in which the group A and the group R³ are not directly linked to one another, basically distinguishes between two cases.

The first case covers those compounds of formula I wherein the group A denotes a nitrogen-heterocyclic group connected via a nitrogen atom to the carboxamide group, which may comprise in addition to the nitrogen atom one or more heteroatoms selected from N, O and S. The reaction of the amine of formula I-1 with the secondary amine of formula I-2 is illustrated in the following general reaction plan:

Reaction Plan 1:

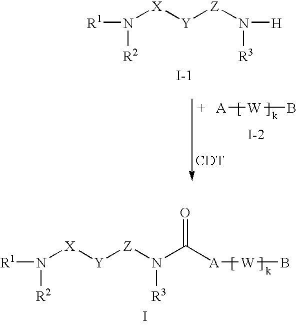

Preferably the amine compound of formula I-1 is first reacted with CDT (1,1′-carbonyldi-(1,2,4-triazole)) in a solvent or mixture of solvents and then the reaction mixture is further reacted with the amine compound of formula I-2, while the minimum of one base is added to the reaction mixture before and/or after the reaction of the amine compound with CDT. Advantageously the amine compound of formula I-1 is reacted with CDT in a temperature range of −20° C. to 20° C. and then this reaction mixture is reacted with the amine compound of formula I-2 in a temperature range of 40° C. to 100° C. in a molar ratio of the amine compound of formula I-1:amine compound of formula I-2:CDT:base of 1±0.25:1±0.25:1±0.25:3±1.5. Preferably nitrogen bases, particularly tert.amine, such as for example triethylamine, are used as bases.

The amine compound of formula I-2 may be a saturated N-heterocyclic compound, such as for example a piperazine derivative according to the following reaction plan 2.

Reaction Plan 2

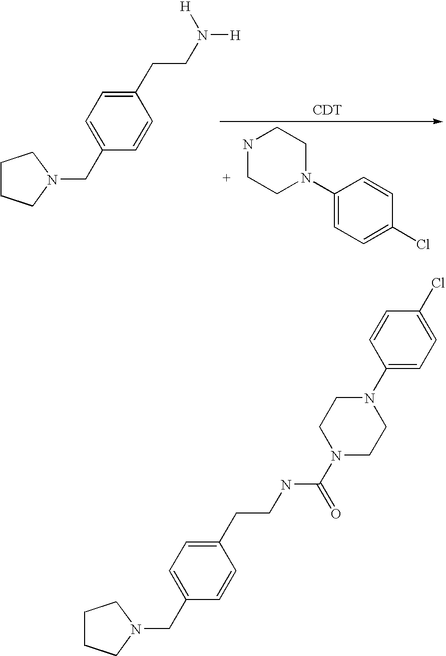

The second case of preparation processes covers the other compounds of formula I which are not covered by case 1, wherein the group A is not directly linked to R³. The reaction of the carboxylic acid compound of formula I-3 with TBTU (2-(1H-benzotriazol-1-yl)-1,1,3,3-tetramethyluronium-tetrafluoroborate) and the amine compound of formula I-1 in a solvent or mixture of solvents in the presence of at least one base is shown in reaction plan 3.

Reaction Plan 3:

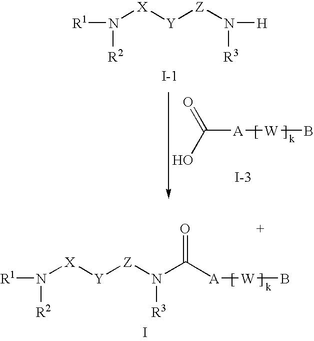

Preferably the carboxylic acid compound of formula I-3 is reacted with TBTU in a solvent or mixture of solvents and then the reaction mixture is further reacted with the amine compound of formula I-1, while the minimum of one base is added to the reaction mixture before and/or after the reaction of the carboxylic acid compound with TBTU. Instead of a carboxylic acid it is also possible to use the corresponding activated carboxylic acid derivatives, such as for example esters, ortho-esters, carboxylic acid chlorides or anhydrides. Preferably the base used is a nitrogen base, particularly a tert.-amine, such as for example triethylamine. Advantageously the carboxylic acid compound of formula I-3 is reacted with TBTU and then this reaction mixture is used with the amine compound of formula I-1 in a temperature range of 0° C. to 60° C. in a molar ratio of the carboxylic acid compound of formula I-3:amine compound of formula I-1:TBTU:base of 1±0.25:1±0.25:1±0.25:1 to 4.

The starting compound of formula I-3 may be obtained by methods known to the skilled man. Thus, biaryl compounds are obtained using Suzuki coupling, for example starting from p-bromoarylcarboxylic acid derivatives and arylboric acid derivatives in the presence of Pd[0] catalysts.

The method of preparation according to the invention for the second group of preferred embodiments, i.e. those compounds wherein the group A and the group R³ are joined together, distinguishes between seven cases, depending on the meanings IIIa to IIIg of the group Q.

According to the first case, in which Q denotes —CR⁶R⁷— (IIIa), an amine compound of formula Ia.1 is reacted with an o-bromomethyl-benzoic acid ester derivative of formula Ia.2, as shown in the following reaction plan 4, in which in the interests of clarity the substituents L¹, L², L³ on the phenyl ring have been omitted.

Reaction Plan 4:

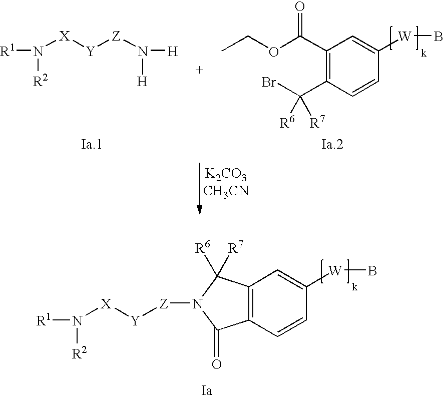

Preferably the o-bromomethyl-benzoic acid ester derivative of formula Ia.2 is reacted with the amine compound of formula Ia.1 in a solvent or mixture of solvents, while at least one base is added. Instead of an o-bromomethyl-benzoic acid ester derivative of formula Ia.2 other corresponding o-benzyl-benzoic acid ester derivatives (iodine or mesylate instead of bromine) may also be used.

Preferably potassium carbonate or caesium carbonate is used as base, but tert. amine bases such as triethylamine are also common. Advantageously the o-bromomethyl-benzoic acid ester derivative of formula Ia.2 is used in acetonitrile with the amine of formula Ia.1 and with potassium carbonate as base in a temperature range of 40-80° C. in a molar ratio of the o-bromomethyl-benzoic acid ester derivative of formula Ia.2:amine of formula Ia.1:potassium carbonate of 1±0.25:1±0.25:3±0.50.

According to the second case, in which Q denotes —CR⁶═R⁷— (IIIb), an isoquinolinone derivative of formula Ib.3 is reacted with an electrophilic compound of formula Ib.4 to form an isoquinoline derivative of formula Ib.5, which is further derivatised by known methods to obtain the compound of formula I. The isoquinolinone derivative of formula Ib.3 is obtainable from cinnamic acid derivatives of formula Ib.1 by reaction with (EtO)₂P(O)N₃. The synthesis of the base substance was described by M. Becker et al. in Bioorganic & Medicinal Chemistry Letters 9 (1999), 2753-2758. The reaction is illustrated in the following reaction plan 5, in which in the interests of clarity the substituents L¹, L², L³ on the phenyl ring have been omitted.

Reaction Plan 5:

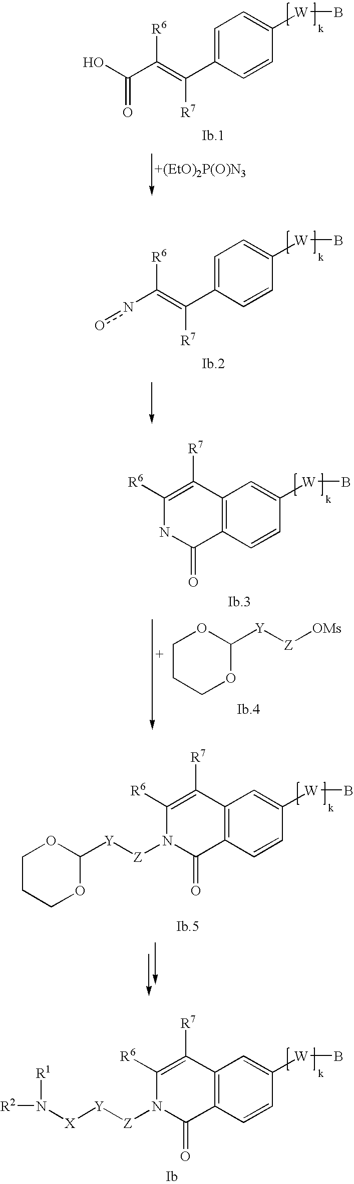

A compound of formula Ib.2 is advantageously obtained by the reaction sequence described hereinafter. The acrylic acid derivative Ib.1 is first reacted by the action of chlorinating agents such as thionyl chloride, phosphorus pentachloride or oxalyl chloride without or optionally in an inert solvent such as dichloromethane to obtain the acid chloride at temperatures between 0° C. and 80° C. This is converted by the action of sodium azide in a solvent or mixture of solvents into the acrylic acid azide derivative. The solvents used may be for example dioxane, tetrahydrofuran or water. Preferably the isocyanate derivative Ib.2 is synthesised directly by the action of phosphoric acid diphenylester azide on the acrylic acid derivative Ib.1 in the presence of a base in a solvent at temperatures between 0° C. and 150° C. Suitable solvents include for example toluene or dioxane. Tertiary amines such as for example triethylamine may be used as bases. The above reactions have reaction times of between one and twelve hours. Advantageously the reaction of the acrylic acid derivative Ib.1 with phosphoric acid diphenylester azide and triethylamine in a molar ratio of 1±0.25:1±0.25:1±0.25 takes place in toluene as solvent.

The isocyanate derivative Ib.2 is heated in a solvent optionally in the presence of a base such as for example tributylamine and forms the isoquinolone derivative of formula Ib.3. Preferably the reaction takes place in diphenylether in the region of the melting point. Heat sources which may be used are oil, metal baths or a microwave.

The reaction of the isoquinolone derivative of formula Ib.3 with the mesylate derivative of formula Ib.4 to form the isoquinolone derivative of formula Ib.5 is carried out in a solvent in the presence of a base at temperatures between 0° C. and 150° C. Advantageously the reaction of the isoquinolone derivative Ib.3 with the mesylate derivative of formula Ib.4 and sodium hydride in a molar ratio of 1±0.25:1±0.25:1±0.25 takes place in DMF as solvent.

The isoquinolone derivative of formula Ib.5 is first reacted in a solvent in the presence of an acid, in order to convert the acetal into the corresponding aldehyde. This is converted into a compound of formula Ib in the presence of a hybrid converter, an amine and an acid in a solvent. Examples of hybrid converters include for example sodium triacetoxyborohydride, sodium borohydride and sodium cyanoborohydride. Advantageously the reaction of the aldehydes, liberated from the isoquinolone derivative Ib.5, with an amine and sodium cyanoborohydride in a molar ratio of 1±0.25:1±0.25:0.8±0.25 takes place in methanol and acetic acid at temperatures of around 20° C.

The synthesis of isoquinolines of formula Ib, including the starting compounds and subsequent derivatisation to form the amine, will be illustrated by means of the following plan of synthesis of a specific compound, while the synthesis of the educt 1 can be inferred from the following Diagram 6, in order to prepare phthalazinones (Diagram 8).

Reaction Plan 6:

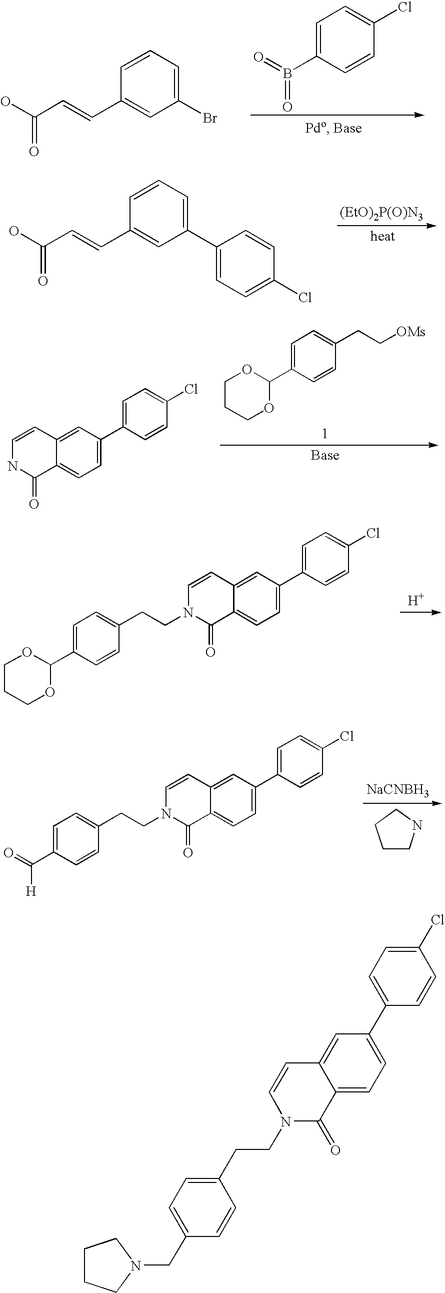

According to the third case in which Q denotes —N═CR⁸— (IIIc), a phthalazinone derivative of formula Ic.4 is reacted with an electrophilic compound of formula Ic.5 to form a phthalazinone derivative of formula Ic.6, which is further derivatised by known methods to form the compound of formula Ic. The phthalazinone derivative of formula Ic.4 for R⁸=hydrogen is obtainable starting from the phenyloxazole derivative of formula Ic.1 by acylation to form an o-oxazolyl-benzaldehyde derivative of formula Ic.2 and subsequent cyclisation to form a 3-hydroxy-3H-isobenzofuran-1-one derivative of formula Ic.3. The synthesis of the base substance was described by M. Napoletano et al., Bioorganic & Medicinal Chemistry Letters 12 (2002), 5-8. The reaction to form compounds of general formula Ic is illustrated in the following reaction plan 7, in which in the interests of clarity the substituents L¹, L², L³ on the phenyl ring have been omitted.

Reaction Plan 7:

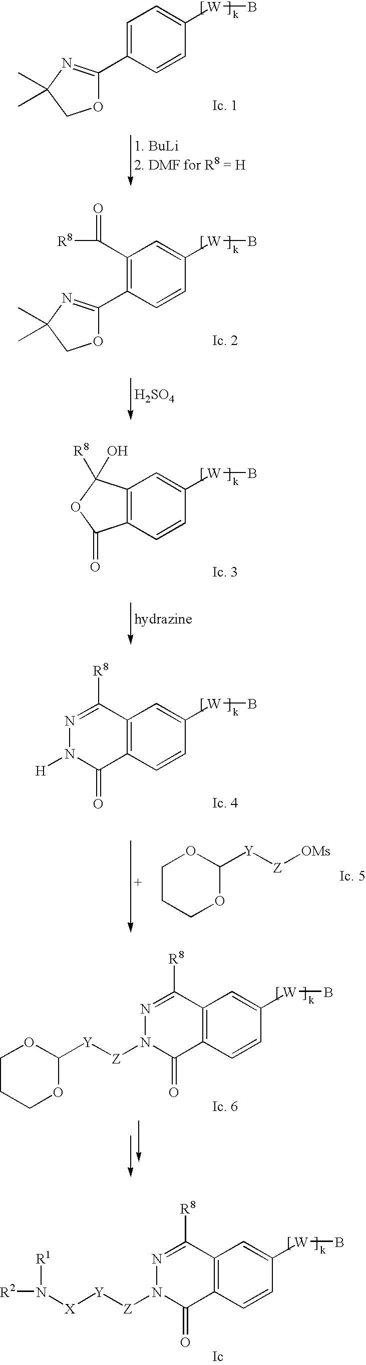

The above reaction sequence is described in more detail hereinafter: The oxazoline derivative Ic.1 is metallised using a suitable organometallic reagent and then reacted with a formaldehyde equivalent such as for example dimethylformamide or an orthoformate at temperatures between −70° C. and 20° C., preferably at temperatures between −20° C. and 0° C., to form a compound of formula Ic.2. Suitable solvents include for example dioxane, tetrahydrofuran or diethyl ether. By the action of aqueous sulphuric acid in a solvent such as for example ethanol at a temperature close to the boiling point of the solvent or mixture of solvents over a period of one to 24 hours, a compound of general formula Ic.3 may be obtained. The phthalazinone derivative of formula Ic.4 may be obtained by reacting a compound of formula Ic.3 with hydrazine in acetic acid and optionally in a solvent at temperatures in range between 20 and 120 degrees celsius. The synthesis to obtain the phthalazinone derivative of formula Ic is carried out analogously to the reactions as described for the synthesis of a compound of general formula Ib.

The synthesis of phthalazinone derivatives of formula Ic, particularly the starting compounds and the subsequent derivatisation, will now be illustrated with reference to a plan of synthesis 8 of a specific compound in which the abbreviations have the following meanings: LAH denotes lithium aluminium hydride, BuLi denotes n-butyllithium, DMF denotes dimethylformamide, MeOH is methanol and Ms-Cl is methanesulphonic acid chloride.

Reaction Plan 8

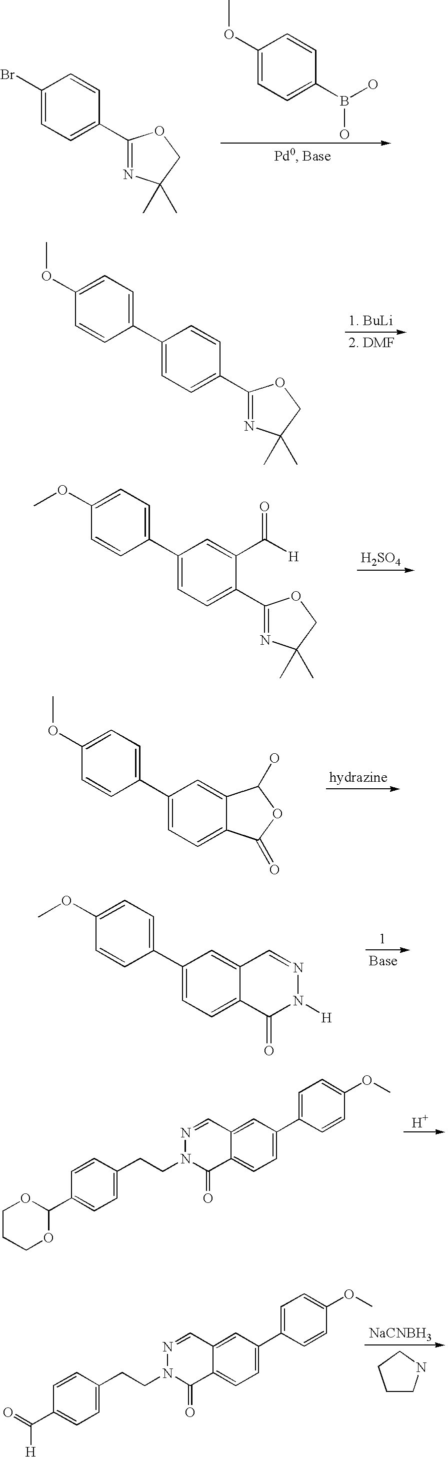

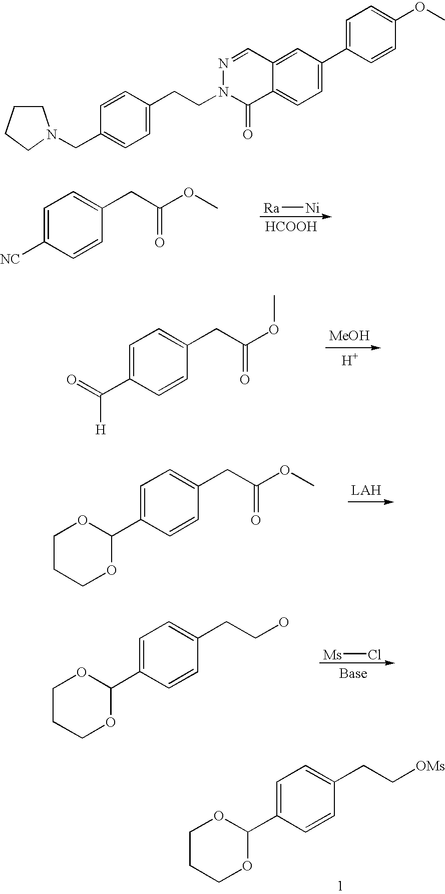

According to the fourth case, in which Q denotes —N═N— (IIId), an o-amino-benzamide derivative of formula Id.1 is reacted in the presence of a suitable nitrite compound and an acid via a diazonium intermediate to form a benzotriazinone derivative of formula Id. The reaction is illustrated in the following reaction plan 9, in which in the interests of clarity the substituents L¹, L², L³ on the phenyl ring have been omitted.

Reaction Plan 9:

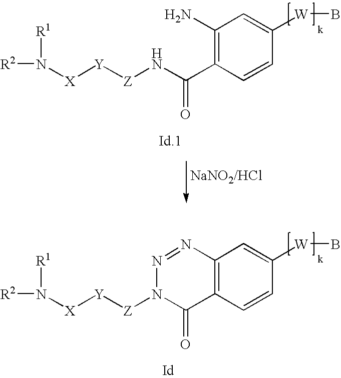

Preferably a compound of general formula Id.1 is reacted in a solvent such as for example methanol in the presence of an inorganic acid, for example hydrochloric acid, and a salt containing the nitrite ion at a temperature between −10° C. and 30° C. Advantageously the reaction of the amino compound Id.1 with sodium nitrite in a molar ratio of 1±0.25:1,5±0.25 takes place in methanol as solvent and in the presence of hydrochloric acid.

According to the fifth case, in which Q denotes —CO—NR⁹— (IIIe), an o-amino-benzamide derivative of formula Ie.1 is reacted in the presence of CDI to form a quinazolinedione derivative of formula Ie. CDI is added to the benzamide derivative of formula Ie.1 in a molar ratio of greater than or equal to 1 and the reaction is carried out at least partially in a temperature range of 35° C. to 100° C., preferably in the region of the boiling temperature of the reaction mixture. The reaction is illustrated in the following reaction plan 10, in which in the interests of clarity the substituents L¹, L², L³ on the phenyl ring have been omitted.

Reaction Plan 10:

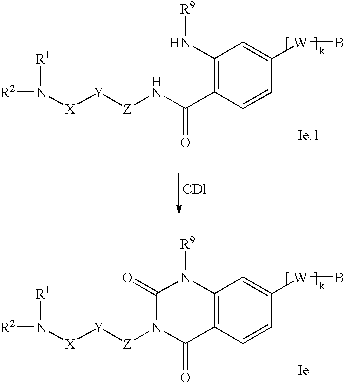

According to the sixth case, in which Q denotes —CR⁸═N— (IIIf), an o-amino-benzamide derivative of formula If.1 is reacted with a carboxylic acid R⁸COOH and/or a corresponding activated carboxylic acid derivative to form the quinazolinone derivative of formula If. Suitable activated carboxylic acid derivatives are for example esters, ortho-esters, carboxylic acid chlorides and anhydrides. The optionally activated carboxylic acid is added to the carboxamide compound of formula If.1 in a molar ratio of greater than or equal to 1 and the reaction is at least partially carried out in a temperature range of 35° C. to 100° C., preferably in the region of the boiling temperature of the reaction mixture. The reaction is illustrated in the following reaction plan 11, in which in the interests of clarity the substituents L¹, L², L³ on the phenyl ring have been omitted.

Reaction Plan 11:

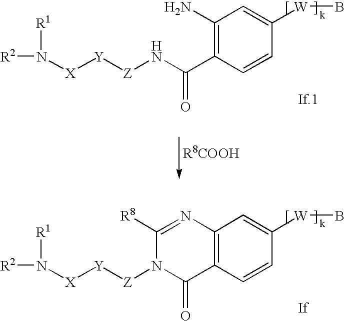

The synthesis of quinazolinone derivatives of formula If, particularly the starting compounds, will be illustrated with reference to a plan of synthesis 12 of a specific compound, in which the following abbreviations are used: CDI for carbonyldiimidazole, TBTU for 2-(1H-benzotriazol-1-yl)-1,1,3,3-tetramethyluronium-tetrafluoroborate and NEt₃ for triethylamine. First the synthesis plans for the two starting compounds 1 and 2 are shown.

Reaction Plan 12

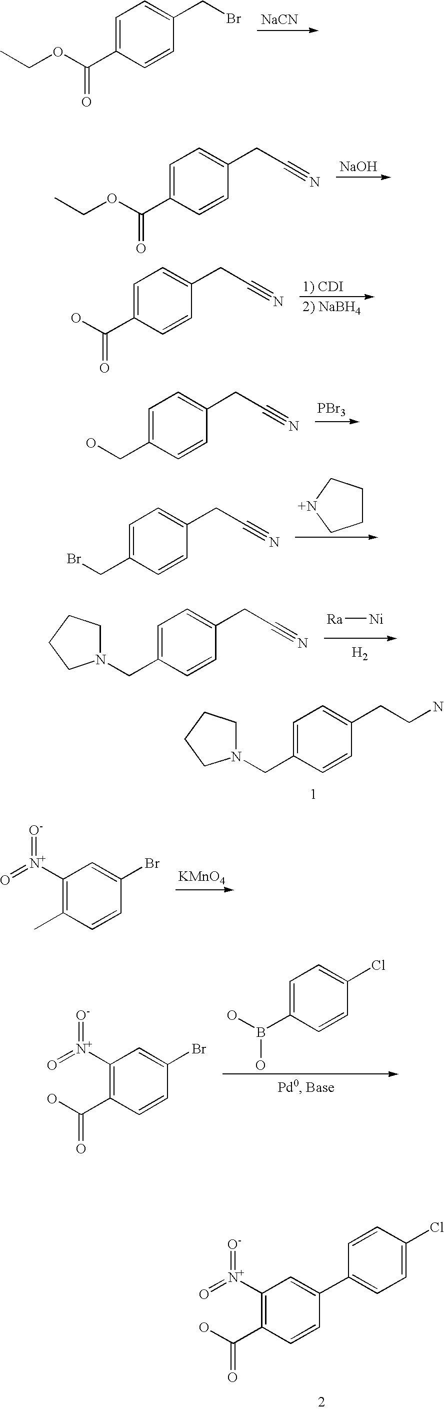

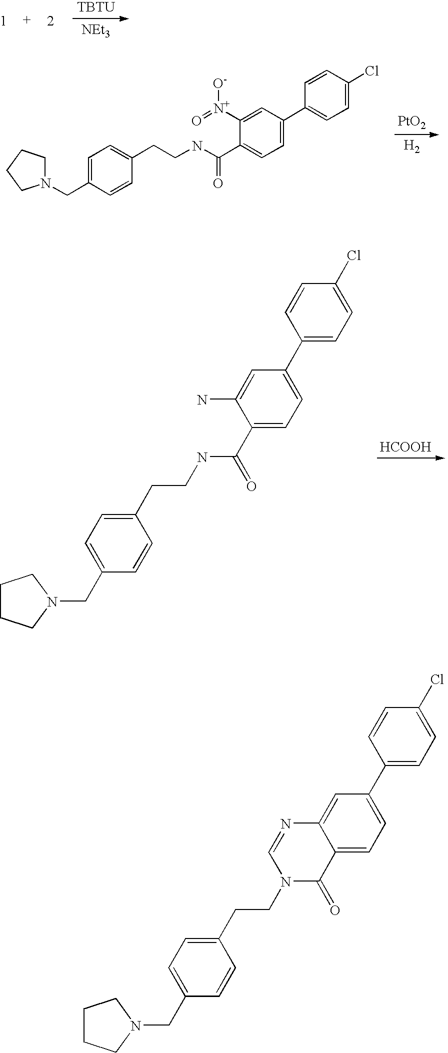

The starting compounds 1 and 2 are linked together via an amide link using TBTU. The nitro group in the ortho position to the amide bond obtained is reduced to form the amine in the presence of PtO₂. Cyclisation to form the quinazolinone is carried out using a carboxylic acid, in this case formic acid.

According to the seventh case in which Q denotes —CO— (IIIg), an isobenzofurandione derivative of formula Ig.2 is reacted with an amine compound of formula Ig.1 to form the isoindoldione derivative of formula Ig. The reaction is illustrated in the following reaction plan 13, in which in the interests of clarity the substituents L¹, L², L³ on the phenyl ring have been omitted.

Reaction Plan 13:

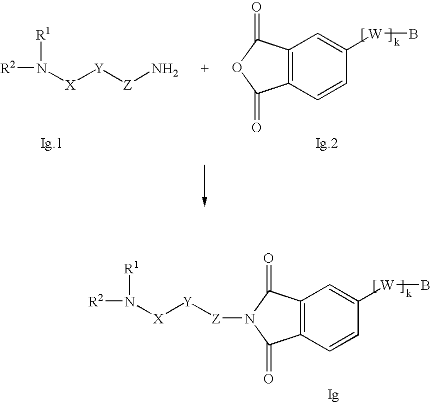

The isobenzofurandione derivative Ig.2 is reacted in a solvent such as for example acetic acid with an amine of general formula Ig.1 in a molar ratio of 1±0.25:1.5±0.25. The temperature during the reaction is preferably the boiling temperature of the solvent.

The isoindoldione derivative of formula Ig may however also be obtained according to the following synthesis plan 14. The synthesis of an individual compound as shown can readily be applied to other compounds of formula Ig, optionally modified, by anyone skilled in the art. First of all, the isoindoldione function is obtained from an isobenzofurandione derivative, binding an amine, and then a further aryl group is added by Suzuki coupling in the presence of Pd[0].

Reaction Plan 14

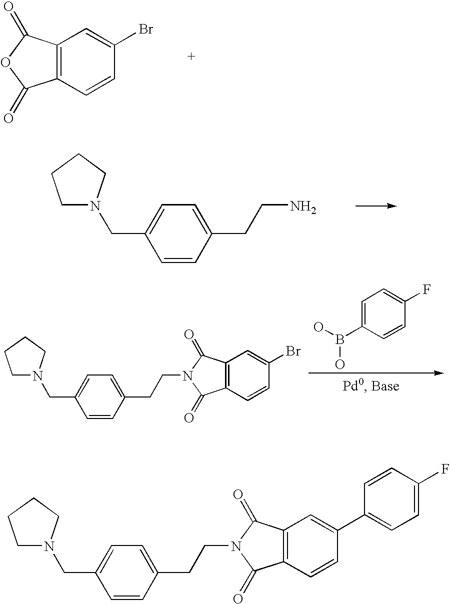

The possible methods described above for synthesising the compounds according to the invention may readily be modified and/or supplemented at least in their broad outline by the skilled man using known methods as described for example in Houben-Weyl, Methoden der organischen Chemie, with regard to the individual compounds which are to be synthesised.

In the reactions described above, any reactive groups present such as hydroxy, carboxy, amino or imino groups may be protected during the reaction by methods known from the literature by conventional protecting groups which are cleaved again after the reaction; the protecting groups conventionally used in peptide chemistry may be used, in particular. Information on this may be found in WO 98/11128 for example.

Stereoisomeric compounds of formula (I) may be separated in principle by conventional methods. The diastereomers may be separated on the basis of their different physico-chemical properties, e.g. by fractional crystallisation from suitable solvents, by high pressure liquid or column chromatography, using chiral or preferably non-chiral stationary phases.

As already mentioned, the compounds of formula (I) may be converted into the salts thereof, particularly for pharmaceutical use into the physiologically and pharmacologically acceptable salts thereof. These salts may be present on the one hand as physiologically and pharmacologically acceptable acid addition salts of the compounds of formula (I) with inorganic or organic acids. On the other hand, in the case of acidically bound hydrogen, the compound of formula (I) may also be converted by reaction with inorganic bases into physiologically and pharmacologically acceptable salts with alkali or alkaline earth metal cations as counter-ion. The acid addition salts may be prepared, for example, using hydrochloric acid, hydrobromic acid, sulphuric acid, phosphoric acid, methanesulphonic acid, acetic acid, fumaric acid, succinic acid, lactic acid, citric acid, tartaric acid or maleic acid. Moreover, mixtures of the above mentioned acids may be used. To prepare the alkali and alkaline earth metal salts of the compound of formula (I) with acidically bound hydrogen the alkali and alkaline earth metal hydroxides and hydrides are preferably used, while the hydroxides and hydrides of the alkali metals, particularly sodium and potassium are preferred and sodium and potassium hydroxide are most preferred.

The compounds according to the present invention, including the physiologically acceptable salts, are effective as antagonists of the MCH receptor, particularly the MCH-1 receptor, and exhibit good affinity in MCH receptor binding studies. Pharmacological test systems for MCH-antagonistic properties are described in the following experimental section.

As antagonists of the MCH receptor the compounds according to the invention are advantageously suitable as pharmaceutical active substances for the prevention and/or treatment of symptoms and/or diseases caused by MCH or causally connected with MCH in some other way. Generally the compounds according to the invention have low toxicity, they are well absorbed by oral route and have an intracerebral transitivity, particularly brain accessibility.

Therefore, MCH antagonists which contain at least one compound according to the invention, are particularly suitable in mammals, such as for example rats, mice, guinea pigs, hares, dogs, cats, sheep, horses, pigs, cattle, monkeys and also humans, for the treatment and/or prevention of symptoms and/or diseases which are caused by MCH or are otherwise causally connected with MCH.

Diseases caused by MCH or otherwise causally connected with MCH are particularly metabolic disorders, such as for example obesity, and eating disorders, such as for example bulimia, including bulimia nervosa. The indication obesity includes in particular exogenic obesity, hyperinsulinaemic obesity, hyperplasmic obesity, hyperphyseal adiposity, hypoplasmic obesity, hypothyroid obesity, hypothalamic obesity, symptomatic obesity, infantile obesity, upper body obesity, alimentary obesity, hypogonadal obesity, central obesity. This range of indications also includes cachexia, anorexia and hyperphagia. Compounds according to the invention may be particularly suitable for reducing hunger, reining in appetite, controlling eating behaviour and/or inducing a feeling of satiation.

In addition, the diseases caused by MCH or otherwise causally connected with MCH also include hyperlipidaemia, cellulitis, fatty accumulation, malignant mastocytosis, systemic mastocytosis, emotional disorders, affectivity disorders, depression, anxiety states, reproductive disorders, memory disorders, forms of dementia and hormonal disorders.

Compounds according to the invention are also suitable as active substances for the prevention and/or treatment of other illnesses and/or disorders, particularly those which accompany obesity, such as for example diabetes, diabetes mellitus, particularly type II diabetes, hyperglycaemia, particularly chronic hyperglycaemia, complications of diabetes including diabetic retinopathy, diabetic neuropathy, diabetic nephropathy, etc., insulin resistance, pathological glucose tolerance, cardiovascular diseases, particularly arteriosclerosis and high blood pressure, and gonitis.

MCH antagonists and formulations according to the invention may advantageously be used in combination with a dietary therapy, such as for example a dietary diabetes treatment, and exercise.

Another range of indications for which the compounds according to the invention are advantageously suitable is the prevention and/or treatment of micturition disorders, such as for example urinary incontinence, hyperactive bladder, nycturia, enuresis, while the hyperactive bladder and urinary incontinence may or may not be connected with benign prostatic hyperplasia.

The dosage required to achieve such an effect is conveniently, by intravenous or subcutaneous route, 0.001 to 30 mg/kg of body weight, preferably 0.01 to 5 mg/kg of body weight, and by oral or nasal route or by inhalation, 0.01 to 50 mg/kg of body weight, preferably 0.1 to 30 mg/kg of body weight, in each case 1 to 3× daily.

For this purpose, the compounds of formula I prepared according to the invention may be formulated, optionally together with other active substances as described hereinafter, together with one or more physiologically acceptable excipients, inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, to produce conventional galenic preparations such as plain or coated tablets, capsules, powders, granules, solutions, emulsions, syrups, aerosols for inhalation, ointments or suppositories.

In addition to pharmaceutical compositions the invention also includes compositions containing at least one carboxamide compound according to the invention and/or a salt according to the invention optionally together with one or more physiologically acceptable excipients. Such compositions may also be for example foodstuffs which may be solid or liquid, in which the compound according to the invention is incorporated.

For the above mentioned combinations it is possible to use as additional active substances particularly those which for example potentiate the therapeutic effect of an MCH antagonist according to the invention in terms of one of the indications mentioned above and/or which make it possible to reduce the dosage of an MCH antagonist according to the invention. Preferably one or more additional active substances are selected from among

active substances for the treatment of diabetes,

active substances for the treatment of diabetic complications,

active substances for the treatment of obesity, preferably other than MCH antagonists,

active substances for the treatment of high blood pressure,

active substances for the treatment of hyperlipidaemia, including arteriosclerosis,

active substances for the treatment of arthritis,

active substances for the treatment of anxiety states,

active substances for the treatment of depression.

The above mentioned categories of active substances will now be explained in more detail by means of examples.

Examples of active substances for the treatment of diabetes are insulin sensitisers, insulin secretion accelerators, biguanides, insulins, α-glucosidase inhibitors, β3 adreno-receptor agonists.

Insulin sensitisers include pioglitazone and its salts (preferably hydrochloride), troglitazone, rosiglitazone and its salts (preferably maleate), JTT-501, GI-262570, MCC-555, YM-440, DRF-2593, BM-13-1258, KRP-297, R-119702, GW-1929.

Insulin secretion accelerators include sulphonylureas, such as for example tolbutamide, chloropropamide, trazamide, acetohexamide, glydlopyramide and its ammonium salts, glibenclamide, gliclazide, glimepiride. Further examples of insulin secretion accelerators are repaglinide, nateglinide, mitiglinide (KAD-1229) and JTT-608.

Biguanides include metformin, buformin and phenformin.

Insulins include those obtained from animals, particularly cattle or pigs, semisynthetic human insulins which are synthesised enzymatically from insulin obtained from animals, human insulin obtained by genetic engineering, e.g. from Escherichi coli or yeasts. Moreover, the term insulin also includes insulin-zinc (containing 0.45 to 0.9 percent by weight of zinc) and protamine-insulin-zinc obtainable from zinc chloride, protamine sulphate and insulin. Insulation may also be obtained from insulin fragments or derivatives (for example INS-1, etc.).

Insulin may also include different kinds, e.g. with regard to the onset time and duration of effect (“ultra immediate action type”, “immediate action type”, “two phase type”, “intermediate type”, “prolonged action type”, etc.), which are selected depending on the pathological condition of the patient.

α-Glucosidase inhibitors include acarbose, voglibose, miglitol, emiglitate.

β₃ Adreno receptor agonists include AJ-9677, BMS-196085, SB-226552, AZ40140.

Active substances for the treatment of diabetes other than those mentioned above include ergoset, pramlintide, leptin, BAY-27-9955 as well as glycogen phosphorylase inhibitors, sorbitol dehydrogenase inhibitors, protein tyrosine phosphatase 1B inhibitors, dipeptidyl protease inhibitors, glipazide, glyburide.

Active substances for the treatment of diabetic complications include for example aldose reductase inhibitors, glycation inhibitors and protein kinase C inhibitors.

Aldose reductase inhibitors are for example tolrestat, epalrestat, imirestat, zenarestat, SNK-860, zopolrestat, ARI-50i, AS-3201.

An example of a glycation inhibitor is pimagedine.

Protein Kinase C inhibitors are for example NGF, LY-333531.

Active substances other than those mentioned above for the treatment of diabetic complications include alprostadil, thiapride hydrochloride, cilostazol, mexiletine hydrochloride, ethyl eicosapentate, memantine, pimagedine (ALT-711).

Active substances for the treatment of obesity, preferably other than MCH antagonists, include lipase inhibitors and anorectics.

A preferred example of a lipase inhibitor is orlistat.

Examples of preferred anorectics are phentermine, mazindol, dexfenfluramine, fluoxetine, sibutramine, baiamine, (S)-sibutramine, SR-141716, NGD-95-1.

Active substances other than those mentioned above for the treatment of obesity include lipstatin.

Moreover for the purposes of this application the active substance group of anti-obesity active substances also includes the anorectics, of which the β₃ agonists, thyromimetic active substances and NPY antagonists should be emphasised. The scope of the anti-obesity/anorectic active substances which are preferred here is indicated by the following additional list, by way of example: phenylpropanolamine, ephedrine, pseudoephedrine, phentermine, a cholecystokinin-A (hereinafter referred to as CCK-A) agonist, a monoamine reuptake inhibitor (such as for example sibutramine), a sympathomimetic active substance, a serotonergic active substance (such as for example dexfenfluramine or fenfluramine), a dopamine antagonist (such as for example bromocriptine), a melanocyte-stimulating hormone receptor agonist or mimetic, an analogue of melanocyte-stimulating hormone, a cannabinoid receptor antagonist, an MCH antagonist, the OB protein (hereinafter referred to as leptin), a leptin analogue, a leptin receptor agonist, a galanine antagonist, a GI lipase inhibitor or reducer (such as for example orlistat). Other anorectics include bombesin agonists, dehydroepiandrosterone or its analogues, glucocorticoid receptor agonists and antagonists, orexin receptor antagonists, urocortin binding protein antagonists, agonists of the Glucagon-like Peptide-1 receptor, such as for example exendin and ciliary neurotrophic factors, such as for example axokines.

Active substances for the treatment of high blood pressure include inhibitors of angiotensin converting enzyme, calcium antagonists, potassium channel openers and angiotensin II antagonists.

inhibitors of angiotensin converting enzyme include captopril, enalapril, alacepril, delapril (hydrochloride), lisinopril, imidapril, benazepril, cilazapril, temocapril, trandolapril, manidipine (hydrochloride).

Examples of calcium antagonists are nifedipine, amlodipine, efonidipine, nicardipine.

Potassium channel openers include levcromakalim, L-27152, AL0671, NIP-121.

Angiotensin II antagonists include telmisartan, losartan, candesartan cilexetil, valsartan, irbeartan, CS-866, E4177.

Active substances for the treatment of hyperlipidaemia, including arteriosclerosis, include HMG-CoA reductase inhibitors, fibrate compounds.

HMG-CoA reductase inhibitors include pravastatin, simvastatin, lovastatin, atorvastatin, fluvastatin, lipantil, cerivastatin, itavastatin, ZD-4522 and their salts.

Fibrate compounds include bezafibrate, clinofibrate, clofibrate and simfibrate.

Active substances for the treatment of arthritis include ibuprofen.

Active substances for the treatment of anxiety states include chlordiazepoxide, diazepam, oxozolam, medazepam, cloxazolam, bromazepam, lorazepam, alprazolam, fludiazepam.

Active substances for the treatment of depression include fluoxetine, fluvoxamine, imipramine, paroxetine, sertraline.

The dosage for these active substances is conveniently 1/5 of the lowest normal recommended dose up to 1/1 of the normal recommended dose.

In another embodiment the invention also relates to the use of at least one carboxamide compound according to the invention and/or a salt according to the invention for influencing the eating behaviour of a mammal. This use is particularly based on the fact that compounds according to the invention may be suitable for reducing hunger, restricting appetite, controlling eating behaviour and/or inducing a feeling of satiety. The eating behaviour is advantageously influenced so as to reduce food intake. Therefore, compounds according to the invention are advantageously used for reducing body weight. Another use according to the invention is the prevention of increases in body weight, for example in people who had previously taken steps to lose weight and are interested in maintaining their lower body weight. According to this embodiment it is preferably a non-therapeutic use. Such a non-therapeutic use might be a cosmetic use, for example to alter the external appearance, or an application to improve general health. The compounds according to the invention are preferably used non-therapeutically for mammals, particularly humans, not suffering from any diagnosed eating disorders, no diagnosed obesity, bulimia, diabetes and/or no diagnosed micturition disorders, particularly urinary incontinence. Preferably, the compounds according to the invention are suitable for non-therapeutic use in people whose BMI (body mass index), defined as their body weight in kilograms divided by their height (in meters) squared, is below a level of 30, particularly below 25.

The Examples that follow are intended to illustrate the invention:

Preliminary Remarks:

As a rule, melting points, ¹H-NMR and/or mass spectra have been obtained for the compounds prepared. Unless otherwise stated the R_{f} values were determined using ready-made silica gel 60 TLC plates F₂₅₄ (E. Merck, Darmstadt, Item no. 1.05714) without chamber saturation. The R_{f} values obtained under the heading Alox were determined using ready-made aluminium oxide 60 TLC plates F₂₅₄ (E. Merck, Darmstadt, Item no. 1.05713) without chamber saturation.

The HPLC data specified were measured under the parameters indicated below: Zorbax column (Agilent Technologies), SB (Stable Bond)-C18; 3.5 μm; 4.6×75 mm; column temperature: 30° C.; flow: 0.8 mL/min; injection volume: 5 μL; detection at 254 nm.

Method A: water:acetonitrile:formic acid 9:1:0.01 towards 1:9:0.01 over 9 min

Method B: water:acetonitrile:formic acid 9:1:0.01 towards 1:9:0.01 over 4 min, then 6 min 1:9:0.01

If there is no specific information as to the configuration, it is not clear whether there are pure enantiomers or whether partial or even total racemisation has taken place.

The following abbreviations are used above and hereinafter:

BOC-anhydride tert.-butyloxycarbonyl-anhydride

CDI carbonyldiimidazole

CDT 1,1′-carbonyldi-(1,2,4-triazole)

DMF dimethylformamide

ethyl acetate/EtOAc ethyl acetate

ether diethyl ether

HOBt 1-hydroxybenzotriazole-hydrate

Hünig base N,N-diisopropyl-ethylamine

conc. concentrated

Me methyl

MeOH methanol

RT room temperature (approx. 20° C.)

TBTU 2-(1H-benzotriazol-1-yl)-1,1,3,3-tetramethyluronium-tetrafluoroborate

THF tetrahydrofuran

eq. equivalent

calc. calculated

fnd. found

General Working Method I (TBTU Coupling):

Triethylamine (1.5 eq.) and TBTU (1.0 eq.) are added successively to a solution of carboxylic acid (1.0 eq.) in THF or DMF. Depending on the carboxylic acid the mixture is stirred for 10 min to 12 h between ambient temperature and 40° C. before the amine (1.0 eq.) is added. The reaction is stirred for 30 min to 2 h between ambient temperature and 40° C., before semisaturated NaHCO₃ solution is added. After extraction of the aqueous phase with a suitable solvent (e.g. ethyl acetate) the organic phase is dried over magnesium sulphate. The solvent is removed using the rotary evaporator; further purification is carried out by column chromatography or HPLC. The reaction may also be carried out in a Chemspeed automatic synthesiser.

General Working Method II (CDT Coupling):

CDT (1 eq.) is added to a solution of the primary amine (1.0 eq.) in DMF (1 mmol/mL) at 0° C. and the mixture is stirred at 0° C. for a further 30 min. The reaction is heated to 25° C. and triethylamine (3 eq.) is added. Then the secondary amine (1.0 eq.) in DMF (0.25 mmol/mL) is added and the reaction solution is heated to 60 to 80° C. for 30 min to 3 h. DMF is removed in vacuo and the residue is taken up with dichloromethane and 5%-Na₂CO₃ solution or with water and tert-butylmethyl ether. The organic phase is extracted with water and the solvent is removed using the rotary evaporator optionally after drying over magnesium sulphate; further purification is carried out by column chromatography or crystallisation. The reaction may also be carried out in a Chemspeed automatic synthesiser.

### EXAMPLE 1.1

### 7-(4-chloro-phenyl)-3-[2-(4-pyrrolidin-1-ylmethyl-phenyl)-ethyl]-3H-quinazolin-4-one

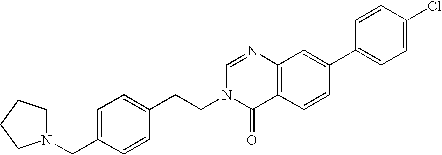

1.1.a. 4-Bromo-2-nitro-benzoic acid

To a reaction mixture of 82 g (0.379 mol) 4-bromo-2-nitro-toluene in 700 ml of pyridine and 500 ml of water are added batchwise 174.5 g (1.104 mol) of potassium permanganate within eight hours. The reaction mixture is stirred for 12 hours at 60° C. Then a further 20 g (0.092 mol) of 4-bromo-2-nitro-toluene, 50 ml of pyridine and 30 g (0.189 mol) of potassium permanganate are added one after another. The reaction mixture is stirred for 12 hours at 60° C., combined with 200 ml of ethanol and refluxed for 30 minutes. Then the reaction mixture is filtered hot and the filtrate is evaporated down in the rotary evaporator. The residue remaining is made alkaline with 10% sodium hydroxide solution and extracted with diethyl ether. The aqueous phase is separated off and acidified with dilute hydrochloric acid. The crystals formed are filtered off, washed with water, azeotropically dried with tetrahydrofuran and stirred with diisopropylether.

Yield: 37 g (32.8% of theory);

C₇H₄BrNO₄ (M=246.018);

calc.: molar peak (M+Na)⁺: 268/270 fnd.: molar peak (M+Na)⁺: 268/270;

R_{f} value: 0.46 (silica gel, dichloromethane/methanol/acetic acid 8:2:0.1).

1.1.b. 4′-Chloro-3-nitro-biphenyl-4-carboxylic acid

0.288 g (0.25 mmol) of tetrakis-(triphenylphosphine)-palladium, 1.25 g (7.99 mmol) of 4-chloro-phenyl-boric acid in 30 ml of methanol and 2.31 g (21.7 mmol) of sodium carbonate in 14 ml of water are added one after another to a solution of 1.92 g (7.81 mmol) of 4-bromo-2-nitro-benzoic acid in 30 ml dioxane. The reaction mixture is heated to 110° C. in a microwave at 300 Watt for one hour. Then the reaction mixture is evaporated down in the rotary evaporator, the residue is taken up in water and adjusted to pH 3 with 1 M hydrochloric acid. The aqueous solution is extracted with ethyl acetate. The organic phase is dried over sodium sulphate, the solvent is distilled off using the rotary evaporator and the residue is stirred with diisopropylether.

Yield: 2.04 g (93.9% of theory);

C₁₃H₈ClNO₄ (M=277.666);

calc.: molar peak (M−H)⁻: 276 fnd.: molar peak (M−H)⁻: 276;

R_{f} value: 0.5 (silica gel, dichloromethane/methanol/acetic acid 9:1:0.1).

1.1.c. Ethyl 4-cyanomethyl-benzoate

To a solution of 147.5 g (2.263 mol) of potassium cyanide in 250 ml of hot water is added dropwise a solution of 500 g (2.057 mol) of ethyl 4-bromomethyl-benzoate in 1000 ml of ethanol. The reaction mixture is refluxed for one hour and stirred for 12 hours at ambient temperature. A further 73.7 g (0.5 mol) of potassium cyanide are added and the mixture is refluxed for two hours. The solid in the reaction mixture is filtered off and the filtrate is filtered through a mixture of silica gel and activated charcoal. The filtrate obtained is evaporated down and the residue is poured onto 1000 ml of water. The aqueous solution is extracted with tert-butylmethylether and the organic phase is extracted three times with water. Then the organic phase is dried over magnesium sulphate and the solvent is distilled off using the rotary evaporator. The purification is carried out by column chromatography on silica gel (petroleum ether/ethyl acetate 8:2).

Yield: 164.46 g (42.2% of theory);

C₁₁H₁₁NO₂ (M=189.216);

calc.: molar peak (M+H)⁺: 190 fnd.: molar peak (M+H)⁺: 190;

R_{f} value: 0.3 (silica gel, petroleum ether/ethyl acetate 8:2).

1.1.d. 4-Cyanomethyl-benzoic acid

A solution of 10 g (53 mmol) of ethyl 4-cyanomethyl-benzoate and 2.02 ml of a 1 M sodium hydroxide solution in 100 ml of ethanol is refluxed for one hour. Then the reaction solution is evaporated down and the residue is combined with ice water. Concentrated hydrochloric acid is added dropwise to the reaction solution until no more precipitate is formed. The precipitate is filtered off, washed twice with water and dried.

Yield: 4.7 g (55% of theory);

C₉H₇NO₂ (M=161.162);

calc.: molar peak (M−H)⁻: 160 fnd.: molar peak (M−H)⁻: 160.

1.1.e. (4-hydroxymethyl-phenyl)-acetonitrile

5.17 g (32 mmol) of CDI are added to a solution of 4.7 g (29 mmol) of 4-cyanomethyl-benzoic acid in 250 ml of tetrahydrofuran and stirred until the development of gas has ended. This reaction mixture is added dropwise to a solution of 3.29 g (87 mmol) of sodium borohydride in 200 ml of water in such a way that the temperature does not exceed 30° C. It is stirred for two hours and the reaction mixture is adjusted to pH 3-4 with potassium hydrogen sulphate solution. Then it is extracted with ethyl acetate, the organic phase is dried over magnesium sulphate and the solvent is separated off using the rotary evaporator.

Yield: 2.6 g (60.9% of theory);

C₉H₉NO (M=147.178);

calc.: molar peak (M−H)⁻: 146;

fnd.: molar peak (M−H)⁻: 146.

1.1.f. (4-bromomethyl-phenyl)-acetonitrile

0.86 ml (9 mmol) of phosphorus tribromide are added dropwise at 0° C. to a solution of 2.6 g (17.66 mmol) of (4-hydroxymethyl-phenyl)-acetonitrile in 25 ml tert-butylmethylether. After the end of the reaction the reaction mixture is combined with water at ambient temperature, the organic phase is separated off and extracted successively with sodium hydrogen carbonate solution and water. The organic phase is dried over magnesium sulphate and the solvent is distilled off using the rotary evaporator.

Yield: 2.9 g (78.1% of theory);

C₉H₈BrN (M=210.075);

calc.: molar peak (M+H)⁺: 209/211 fnd.: molar peak (M+H)⁺: 209/211.

1.1.g. (4-Pyrrolidin-1-ylmethyl-phenyl)-acetonitrile

0.446 ml (5.44 mmol) of pyrrolidine and 1.366 g (9.882 mmol) of potassium carbonate are added to 20 ml of dimethylformamide. While stirring 1.038 g (4.941 mmol) of (4-bromomethyl-phenyl)-acetonitrile are added and the mixture is stirred for 12 hours at ambient temperature. The reaction mixture is evaporated down in the rotary evaporator and the residue is extracted with ethyl acetate and water. The organic phase is dried over magnesium sulphate and the solvent is eliminated using the rotary evaporator.

Yield: 0.732 g (74% of theory);

C₁₃H₁₆N₂ (M=200.286);

calc.: molar peak (M+H)⁺: 201 fnd.: molar peak (M+H)⁺: 201;

R_{f} value: 0.5 (silica gel, dichloromethane/methanol/ammonia 9:1:0.1).

1.1.h 2-(4-Pyrrolidin-1-ylmethyl-phenyl)-ethylamine

A reaction mixture of 0.73 g (3,66 mmol) of (4-pyrrolidin-1-ylmethyl-phenyl)-acetonitrile and 0.1 g of Raney nickel in 25 ml of methanolic ammonia solution is hydrogenated for 9 h at 50° C. and 3 bar hydrogen.

Yield: 0.72 g (96.4% of theory);

C₁₃H₂₀N₂ (M=204.31);

calc.: molar peak (M+H)⁺: 205 fnd.: molar peak (M+H)⁺: 205;

R_{f} value: 0.23 (silica gel, dichloromethane/methanol/ammonia 9:1:0.1).

1.1.i. 4′-chloro-3-nitro-biphenyl-4-carboxylic acid-[2-(4-pyrrolidin-1-ylmethyl-phenyl)-ethyl]-amide

A solution of 0.4 (1.44 mmol) of 4′-chloro-3-nitro-biphenyl-4-carboxylic acid, 0.29 g (1.44 mmol) of 2-(4-pyrrolidin-1-ylmethyl-phenyl)-ethylamine, 0.46 g (1.44 mmol) of TBTU, 0,19 g (1.44 mmol) of HOBT and 0.42 ml (3 mmol) of triethylamine in 30 ml of tetrahydrofuran is stirred for 14 hours at ambient temperature. The reaction mixture is evaporated down in the rotary evaporator, extracted with water and ethyl acetate and dried over magnesium sulphate. The purification is carried out by column chromatography on silica gel (eluant: dichloromethane/methanol/ammonia=90:10:1).

Yield: 0.47 g (70.3% of theory);

C₂₆H₂₆ClN₃O₃ (M=463.96);

calc.: molar peak (M+H)⁺: 464/466 fnd.: molar peak (M+H)⁺: 464/466;

R_{f} value: 0.36 (silica gel, dichloromethane/methanol/ammonia 9:1:0.1).

1.1.j. 4′-Chloro-3-amino-biphenyl-4-carboxylic acid-[2-(4-pyrrolidin-1-ylmethyl-phenyl)-ethyl]-amide

A reaction mixture of 0.47 g (1.01 mmol) of 4′-chloro-3-nitro-biphenyl-4-carboxylic acid-[2-(4-pyrrolidin-1-ylmethyl-phenyl)-ethyl]-amide and 0.1 g of Raney nickel in 50 ml of methanolic ammonia solution is hydrogenated for 24 hours at 20° C. and 3 bar hydrogen. The crude product is further reacted without purification.

Yield: 0.46 g crude;

C₂₆H₂₈ClN₃O (M=433.98);

calc.: molar peak (M+H)⁺: 434/436 fnd.: molar peak (M+H)⁺: 434/436;

R_{f} value: 0.34 (silica gel, dichloromethane/methanol/ammonia 9:1:0.1).

1.1.k. 7-(4-chloro-phenyl)-3-[2-(4-pyrrolidin-1-ylmethyl-phenyl)-ethyl]-3H-quinazolin-4-one

0.46 g (1.06 mmol) of 4′-chloro-3-amino-biphenyl-4-carboxylic acid-[2-(4-pyrrolidin-1-ylmethyl-phenyl)-ethyl]-amide and 5 ml of formic acid are stirred for 3 h at ambient temperature and 2 h at 100° C. The reaction mixture is combined with water, made alkaline with 6N sodium hydroxide solution and the precipitate is suction filtered. The precipitate is taken up in dichloromethane and dried over magnesium sulphate. The solvent is distilled off using the rotary evaporator and the residue is triturated with diisopropylether.

Yield: 0.3 g (64.6% of theory);

melting point: 178-179° C.;

C₂₇H₂₆ClN₃O (M=443.98);

calc.: molar peak (M+H)⁺: 444 fnd.: molar peak (M+H)⁺: 444;

R_{f} value: 0.35 (silica gel, dichloromethane/methanol/ammonia 9:1:0.1).

### EXAMPLE 1.2

### 3-[2-(4-Pyrrolidin-1-ylmethyl-phenyl)-ethyl]-7-p-tolyl-3H-quinazolin-4-one

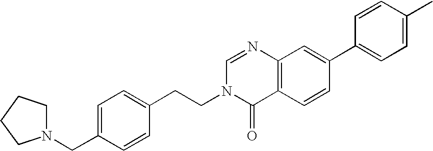

1.2.a. 4′-methyl-3-nitro-biphenyl-4-carboxylic acid

Prepared analogously to Example 1.1.b from 4-bromo-2-nitro-benzoic acid and 4-methyl-phenyl-boric acid.

Yield: 1.48 g (70.8% of theory);

C₁₄H₁₁NO₄ (M=257.24);

calc.: molar peak (M−H)⁻: 256 fnd.: molar peak (M−H)⁻: 256;

R_{f} value: 0.54 (silica gel, dichloromethane/methanol/acetic acid 9:1:0.1).

1.2.b. 4′-methyl-3-nitro-biphenyl-4-carboxylic acid-[2-(4-pyrrolidin-1-ylmethyl-phenyl)-ethyl]-amide

Prepared analogously to Example 1.1.i from 4′-methyl-3-nitro-biphenyl-4-carboxylic acid and 2-(4-pyrrolidin-1-ylmethyl-phenyl)-ethylamine.

Yield: 0,51 g (78.3% of theory);

C₂₇H₂₉N₃O₃ (M=443,55);

calc.: molar peak (M+H)⁺: 444 fnd.: molar peak (M+H)⁺: 444;

R_{f} value: 0.35 (silica gel, dichloromethane/methanol/ammonia 9:1:0.1).

1.2.c. 4′-methyl-3-amino-biphenyl-4-carboxylic acid-[2-(4-pyrrolidin-1-ylmethyl-phenyl)-ethyl]-amide

Prepared analogously to Example 1.1.j from 4′-methyl-3-nitro-biphenyl-4-carboxylic acid-[2-(4-pyrrolidin-1-ylmethyl-phenyl)-ethyl]-amide.

Yield: 0.2 g (69.2% of theory);

C₂₈H₃₁N₃O (M=413.56);

calc.: molar peak (M+H)⁺: 414 fnd.: molar peak (M+H)⁺: 414;

R_{f} value: 0.36 (silica gel, dichloromethane/methanol/ammonia 9:1:0.1).

### EXAMPLE 1.3

### 3-[2-(4-pyrrolidin-1-ylmethyl-phenyl)-ethyl]-7-(4-trifluormethyl-phenyl)-3H-quinazolin-4-one

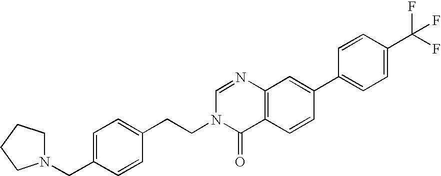

1.3.a. 4′-trifluoromethyl-3-nitro-biphenyl-4-carboxylic acid

Prepared analogously to Example 1.1.b from 4-bromo-2-nitro-benzoic acid and 4-trifluoromethyl-phenyl-boric acid.

Yield: 1.24 g (49% of theory);

C₁₄H₈F₃NO₄ (M=311.21);

calc.: molar peak (M−H)⁻: 310 fnd.: molar peak (M−H)⁻: 310;

R_{f} value: 0.3 (silica gel, dichloromethane/methanol/acetic acid 9:1:0.1).

1.3.b. 4′-trifluoromethyl-3-nitro-biphenyl-4-carboxylic acid-[2-(4-pyrrolidin-1-ylmethyl-phenyl)-ethyl]-amide

Prepared analogously to Example 1.1.i from 4′-trifluoromethyl-3-nitro-biphenyl-4-carboxylic acid and 2-(4-pyrrolidin-1-ylmethyl-phenyl)-ethylamine.

Yield: 0.36 g (49.3% of theory);

C₂₇H₂₆F₃N₃O₃ (M=497.52);

calc.: molar peak (M+H)⁺: 498 fnd.: molar peak (M+H)⁺: 498;

R_{f} value: 0.3 (silica gel, dichloromethane/methanol/ammonia 9:1:0.1).

1.3.c. 4′-trifluoromethyl-3-amino-biphenyl-4-carboxylic acid-[2-(4-pyrrolidin-1-ylmethyl-phenyl)-ethyl]-amide

A reaction mixture of 0.1 g (0.2 mmol) of 4′-trifluoromethyl-3-nitro-biphenyl-4-carboxylic acid-[2-(4-pyrrolidin-1-ylmethyl-phenyl)-ethyl]-amide and 0.08 g platinum oxide in 50 ml ethyl acetate is hydrogenated at 20° C. for 2.5 h. The catalyst is filtered off. The purification is carried out by column chromatography on silica gel (eluant: dichloromethane/methanol/ammonia=90:10:1).

Yield: 0.06 g (63.8% of theory);

C₂₇H₂₈N₃N₃O (M=467.53);

calc.: molar peak (M+H)⁺: 468 fnd.: molar peak (M+H)⁺: 468;

R_{f} value: 0.46 (silica gel, dichloromethane/methanol/ammonia 9:1:0.1).

### EXAMPLE 1.4

### 7-(4-Methoxy-phenyl)-3-[2-(4-pyrrolidin-1-ylmethyl-phenyl)-ethyl]-3H-quinazolin-4-one

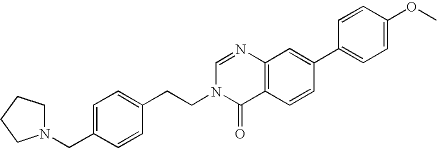

1.4.a. 4′-methoxy-3-nitro-biphenyl-4-carboxylic acid

Prepared analogously to Example 1.1.b from 4-bromo-2-nitro-benzoic acid and 4-methoxy-phenyl-boric acid.

Yield: 0.38 g (48.9% of theory);

C₁₄H₁₁NO₅ (M=273.24);

calc.: molar peak (M−H)⁻: 272 fnd.: molar peak (M−H)⁻: 272;

R_{f} value: 0.39 (silica gel, dichloromethane/methanol/acetic acid 9:1:0.1).

1.4.b. 4′-methoxy-3-nitro-biphenyl-4-carboxylic acid-[2-(4-pyrrolidin-1-ylmethyl-phenyl)-ethyl]-amide

Prepared analogously to Example 1.1.j from 4′-methoxy-3-nitro-biphenyl-4-carboxylic acid and 2-(4-pyrrolidin-1-ylmethyl-phenyl)-ethylamine.

Yield: 0.23 g (57% of theory);

C₂₇H₂₉N₃O₄ (M=459.55);

calc.: molar peak (M+H)⁺: 460 fnd.: molar peak (M+H)⁺: 460;

R_{f} value: 0.48 (silica gel, dichloromethane/methanol/ammonia 9:1:0.1).

1.4.c. 4′-methoxy-3-amino-biphenyl-4-carboxylic acid-[2-(4-pyrrolidin-1-ylmethyl-phenyl)-ethyl]-amide

Prepared analogously to Example 1.3.c from 4′-methoxy-3-nitro-biphenyl-4-carboxylic acid-[2-(4-pyrrolidin-1-ylmethyl-phenyl)-ethyl]-amide.

Yield: 0.09 g (42% of theory);

C₂₇H₃₁N₃O₂ (M=429.56);

calc.: molar peak (M+H)⁺: 430 fnd.: molar peak (M+H)⁺: 430;

R_{f} value: 0.44 (silica gel, dichloromethane/methanol/ammonia 9:1:0.1).

### EXAMPLE 1.5

### 7-(3,4-dichloro-phenyl)-3-[2-(4-pyrrolidin-1-ylmethyl-phenyl)-ethyl]-3H-quinazolin-4-one

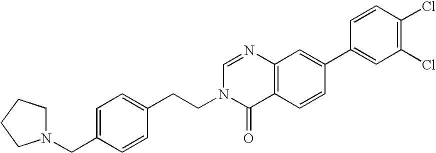

1.5.a. 3′,4′-dichloro-3-nitro-biphenyl-4-carboxylic acid

Prepared analogously to Example 1.1.b from 4-bromo-2-nitro-benzoic acid and 3,4-dichloro-phenyl-boric acid.

Yield: 0.72 g (28.4% of theory);

C₁₃H₇Cl₂NO₄ (M=312.11);

calc.: molar peak (M−H)⁻: 310/312/314 fnd.: molar peak (M−H)⁻: 310/312/314;

R_{f} value: 0.39 (silica gel, dichloromethane/methanol/acetic acid 9:1:0.1).

1.5.b. 3′,4′-dichloro-3-nitro-biphenyl-4-carboxylic acid-[2-(4-pyrrolidin-1-ylmethyl-phenyl)-ethyl]-amide

Prepared analogously to Example 1.1.i from 3′,4′-dichloro-3-nitro-biphenyl-4-carboxylic acid and 2-(4-pyrrolidin-1-ylmethyl-phenyl)-ethylamine.

Yield: 0.47 g (64.2% of theory);

C₂₆H₂₅Cl₂N₃O₃ (M=498.41);

calc.: molar peak (M+H)⁺: 498/500/502 fnd.: molar peak (M+H)⁺: 498/500/502;

R_{f} value: 0.24 (silica gel, dichloromethane/methanol/ammonia 9:1:0.1).

1.5.c. 3′,4′-dichloro-3-amino-biphenyl-4-carboxylic acid-[2-(4-pyrrolidin-1-ylmethyl-phenyl)-ethyl]-amide

Prepared analogously to Example 1.3.c from 3′,4′-dichloro-3-nitro-biphenyl-4-carboxylic acid-[2-(4-pyrrolidin-1-ylmethyl-phenyl)-ethyl]-amide.

Yield: 0.11 g (25% of theory);

C₂₆H₂₇Cl₂N₃O (M=468.43);

calc.: molar peak (M+H)⁺: 468/470/472 fnd.: molar peak (M+H)⁺: 468/470/472;

R_{f} value: 0.46 (silica gel, dichloromethane/methanol/ammonia 9:1:0.1).

### EXAMPLE 1.6

### 7-(3-methoxy-phenyl)-3-[2-(4-pyrrolidin-1-ylmethyl-phenyl)-ethyl]-3H-quinazolin-4-one

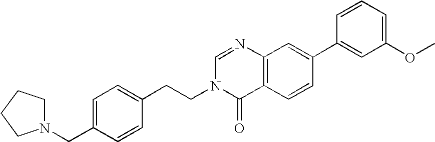

1.6.a. 3′-methoxy-3-nitro-biphenyl-4-carboxylic acid

Prepared analogously to Example 1.1.b from 4-bromo-2-nitro-benzoic acid and 3-methoxy-phenyl-boric acid.

Yield: 0.39 g (73.6% of theory);

C₁₄H₁₁NO₅ (M=273.24);

calc.: molar peak (M+H)⁺: 274 fnd.: molar peak (M+H)⁺: 274;

R_{f} value: 0.35 (silica gel, dichloromethane/methanol/acetic acid 9:1:0.1).

1.6.b. 3′-methoxy-3-nitro-biphenyl-4-carboxylic acid-[2-(4-pyrrolidin-1-ylmethyl-phenyl)-ethyl]-amide

Prepared analogously to Example 1.1.i from 3′-methoxy-3-nitro-biphenyl-4-carboxylic acid and 2-(4-pyrrolidin-1-ylmethyl-phenyl)-ethylamine.

Yield: 0.39 g (57% of theory);

C₂₇H₂₉N₃O₄ (M=459.55);

calc.: molar peak (M+H)⁺: 460 fnd.: molar peak (M+H)⁺: 460;

R_{f} value: 0.23 (silica gel, dichloromethane/methanol/ammonia 9:1:0.1).

1.6.c. 3′-methoxy-3-amino-biphenyl-4-carboxylic acid-[2-(4-pyrrolidin-1-ylmethyl-phenyl)-ethyl]-amide

Prepared analogously to Example 1.1.j from 3′-methoxy-3-nitro-biphenyl-4-carboxylic acid-[2-(4-pyrrolidin-1-ylmethyl-phenyl)-ethyl]-amide.

Yield: 0.11 g (30.6% of theory);

C₂₇H₃₁N₃O₂ (M=429.56);

calc.: molar peak (M+H)⁺: 430 fnd.: molar peak (M+H)⁺: 430;

R_{f} value: 0.36 (silica gel, dichloromethane/methanol/ammonia 9:1:0.1).

### EXAMPLE 1.7

### 7-(4-fluoro-phenyl)-3-[2-(4-pyrrolidin-1-ylmethyl-phenyl)-ethyl]-3H-quinazolin-4-one

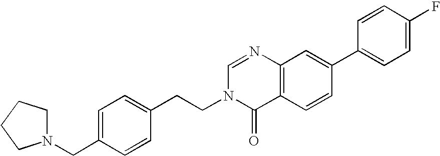

1.7.a. 4′-fluoro-3-nitro-biphenyl-4-carboxylic acid

Prepared analogously to Example 1.1.b from 4-bromo-2-nitro-benzoic acid and 4-fluoro-phenyl-boric acid.

Yield: 1.3 g (61.2% of theory);

C₁₃H₈FNO₄ (M=261.21);

calc.: molar peak (M−H)⁻: 260 fnd.: molar peak (M−H)⁻: 260;

R_{f} value: 0.34 (silica gel, dichloromethane/methanol/acetic acid 9:1:0.1).

1.7.b. 4′-fluoro-3-nitro-biphenyl-4-carboxylic acid-[2-(4-pyrrolidin-1-ylmethyl-phenyl)-ethyl]-amide

Prepared analogously to Example 1.1.i from 4′-fluoro-3-nitro-biphenyl-4-carboxylic acid and 2-(4-pyrrolidin-1-ylmethyl-phenyl)-ethylamine.

Yield: 0.38 g (57.8% of theory);

C₂₆H₂₆FN₃O₃ (M=447.51);

calc.: molar peak (M+H)⁺: 448 fnd.: molar peak (M+H)⁺: 448;

R_{f} value: 0.24 (silica gel, dichloromethane/methanol/ammonia 9:1:0.1).

1.7.c. 4′-fluoro-3-amino-biphenyl-4-carboxylic acid-[2-(4-pyrrolidin-1-ylmethyl-phenyl)-ethyl]-amide

Prepared analogously to Example 1.3.c from 4′-fluoro-3-nitro-biphenyl-4-carboxylic acid-[2-(4-pyrrolidin-1-ylmethyl-phenyl)-ethyl]-amide.

Yield: 0.06 g (32% of theory);

C₂₆H₂₈FN₃O (M=417.53);

calc.: molar peak (M+H)⁺: 418 fnd.: molar peak (M+H)⁺: 418;

R_{f} value: 0.63 (silica gel, dichloromethane/methanol/ammonia 9:1:0.1).

### EXAMPLE 1.8

### 7-(4-Ethyl-phenyl)-3-[2-(4-pyrrolidin-1-ylmethyl-phenyl)-ethyl]-3H-quinazolin-4-one

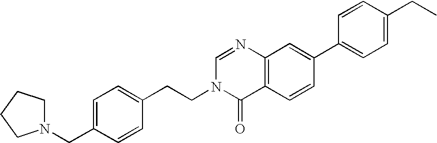

1.8.a. 4′-Vinyl-3-nitro-biphenyl-4-carboxylic acid

Prepared analogously to Example 1.1.b from 4-bromo-2-nitro-benzoic acid and 4-vinyl-phenyl-boric acid.

Yield: 0.58 g (53% of theory);

C₁₅H₁₁NO₄ (M=269.25);

calc.: molar peak (M−H)⁻: 268 fnd.: molar peak (M−H)⁻: 268;

R_{f} value: 0.39 (silica gel, dichloromethane/methanol/acetic acid 9:1:0.1).

1.8.b. 4′-Vinyl-3-nitro-biphenyl-4-carboxylic acid-[2-(4-pyrrolidin-1-ylmethyl-phenyl)-ethyl]-amide

Prepared analogously to Example 1.1.i from 4′-vinyl-3-nitro-biphenyl-4-carboxylic acid and 2-(4-pyrrolidin-1-ylmethyl-phenyl)-ethylamine.

Yield: 0.38 g (56.8% of theory);

C₂₈H₂₉N₃O₃ (M=455.56);

calc.: molar peak (M+H)⁺: 456 fnd.: molar peak (M+H)⁺: 456;

R_{f} value: 0.21 (silica gel, dichloromethane/methanol/ammonia 9:1:0.1).

1.8.c. 4′-ethyl-3-amino-biphenyl-4-carboxylic acid-[2-(4-pyrrolidin-1-ylmethyl-phenyl)-ethyl]-amide

Prepared analogously to Example 1.3.c from 4′-vinyl-3-nitro-biphenyl-4-carboxylic acid-[2-(4-pyrrolidin-1-ylmethyl-phenyl)-ethyl]-amide.

Yield: 0.15 g (63.9% of theory);

C₂₈H₃₃N₃O (M=427.59);

calc.: molar peak (M+H)⁺: 428 fnd.: molar peak (M+H)⁺: 428;

R_{f} value: 0.47 (silica gel, dichloromethane/methanol/ammonia 9:1:0.1).

The following compounds were prepared analogously to Example 1.1.k:

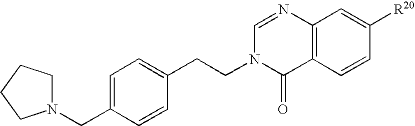

| | | | empirical | mass | mp | |
|---|---|---|---|---|---|---|
| Example | R²⁰ | educt | formula | spectrum | [° C.] | R_{f value} |
| 1.1 | 4-chloro-phenyl | 1.1.k | C₂₇H₂₆ClN₃O | 444 [M + H]⁺ | 178- | 0.35 |
| | | | | | 179 | (A) |
| 1.2 | 4-methyl-phenyl | 1.2.c | C₂₈H₂₉N₃O | 424 [M + H]⁺ | 157- | 0.36 |
| | | | | | 158 | (A) |
| 1.3 | 4-trifluoromethyl- | 1.3.c | C₂₈H₂₆F₃N₃O | 478 [M + H]⁺ | 179- | 0.4 |
| | phenyl | | | | 181 | (A) |
| 1.4 | 4-methoxy- | 1.4.c | C₂₈H₂₉N₃O₂ | 440 [M + H]⁺ | 143- | 0.37 |
| | phenyl | | | | 144 | (A) |
| 1.5 | 3,4-dichloro- | 1.5.c | C₂₇H₂₅Cl₂N₃O | 478/80/82 | 148- | 0.36 |
| | phenyl | | | [M + H]⁺ | 149 | (A) |
| 1.6 | 3-methoxy- | 1.6.c | C₂₈H₂₉N₃O₂ | 440 [M + H]⁺ | wax | 0.14 |
| | phenyl | | | | | (A) |
| 1.7 | 4-fluoro-phenyl | 1.7.c | C₂₇H₂₆FN₃O | 428 [M + H]⁺ | 160- | 0.45 |
| | | | | | 161 | (A) |
| 1.8 | 4-ethyl-phenyl | 1.8.c | C₂₉H₃₁N₃O | 438 [M + H]⁺ | 165- | 0.37 |
| | | | | | 166 | (A) |

R_{f} value: A = (silica gel, dichloromethane/methanol/ammonia 9:1:0.1)

### EXAMPLE 1.9

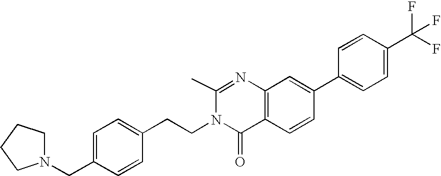

1.9.a 7-(4-trifluoromethyl-phenyl)-2-methyl-3-[2-(4-pyrrolidin-1-ylmethyl-phenyl)-ethyl]-3H-quinazolin-4-one

A solution of 0.07 g (0.15 mmol) of 4′-trifluoromethyl-3-amino-biphenyl-4-carboxylic acid-[2-(4-pyrrolidin-1-ylmethyl-phenyl)-ethyl]-amide (cf. Exam pie 1.3.c) in 4 ml acetic acid and 0.028 ml (0.3 mmol) of acetic anhydride is refluxed for 12 hours. The reaction solution is diluted with water, adjusted to pH 8 with dilute sodium hydroxide solution and extracted with dichloromethane. The organic phase is dried over magnesium sulphate. The purification is carried out by column chromatography on silica gel (eluant: dichloromethane/methanol/ammonia 90:10:1)

Yield: 0.008 g (11% of theory);

C₂₉H₂₈F₃N₃O (M=491.56);

calc.: molar peak (M+H)⁺: 492 fnd.: molar peak (M+H)⁺: 492;

R_{f} value: 0.36 (silica gel, dichloromethane/methanol/ammonia 9:1:0.1).

The following compounds were prepared analogously to Example 1.9.a:

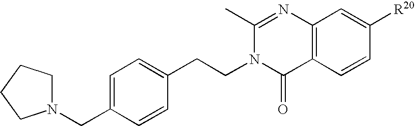

| | | | empirical | mass | mp | |
|---|---|---|---|---|---|---|
| Example | R²⁰ | educt | formula | spectrum | [° C.] | R_{f value} |
| 1.9 | 4-trifluoromethyl- | 1.3.c | C₂₉H₂₈F₃N₃O | 492 [M + H]⁺ | wax | 0.36 |
| | phenyl | | | | | (A) |
| 1.10 | 4-methyl-phenyl | 1.2.c | C₂₉H₃₁N₃O | 437 [M + H]⁺ | wax | 0.66 |
| | | | | | | (A) |
| 1.11 | 4-chloro-phenyl | 1.1.j | C₂₈H₂₈ClN₃O | 458/60 | 160- | 0.40 |
| | | | | [M + H]⁺ | 163 | (A) |

R_{f} value: A = (silica gel, dichloromethane/methanol/ammonia 9:1:0.1)

### EXAMPLE 1.10

### 2-methyl-3-[2-(4-pyrrolidin-1-ylmethyl-phenyl)-ethyl]-7-p-tolyl-3H-quinazolin-4-one

### EXAMPLE 1.11

### 7-(4-chloro-phenyl)-2-methyl-3-[2-(4-pyrrolidin-1-ylmethyl-phenyl)-ethyl]-3H-quinazolin-4-one

### EXAMPLE 1.12

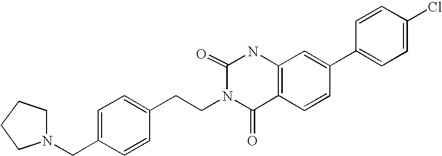

1.12.a 7-(4-chloro-phenyl)-3-[2-(4-pyrrolidin-1-ylmethyl-phenyl)-ethyl]-1H-quinazolin-2,4-dione

A reaction mixture of 0.3 g (0.69 mmol) of 4′-chloro-3-amino-biphenyl-4-carboxylic acid-[2-(4-pyrrolidin-1-ylmethyl-phenyl)-ethyl]-amide (cf. Example 1.1.j) and 0.1 g (0.65 mmol) of CDI in 50 ml of tetrahydrofuran is refluxed for 24 hours. Then a further 0.1 g CDI are added and the reaction mixture is refluxed for a further 24 hours. The reaction mixture is evaporated down in the rotary evaporator. The purification is carried out by column chromatography on silica gel (eluant: dichloromethane/methanol/ammonia 60:1:0.1)

Yield: 0.2 g (62.9% of theory);

melting point: 274-276° C.;

C₂₇H₂₆ClN₃O₂ (M=459.98);

calc.: molar peak (M+H)⁺: 460/462 fnd.: molar peak (M+H)⁺: 460/462;

R_{f} value: 0.1 (silica gel, dichloromethane/methanol/ammonia 50:1:0.1).

### EXAMPLE 1.13

### 7-(4-chloro-phenyl)-3-{2-[4-((S)-2-methoxymethyl-pyrrolidin-1-ylmethyl)-phenyl]-ethyl}-3H-quinazolin-4-one

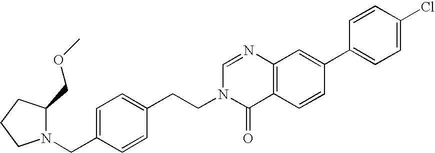

1.13.a [4-(2-(S)-methoxymethyl-pyrrolidin-1-ylmethyl)-phenyl]-acetonitrile

Prepared analogously to Example 1.1.g from 2-(S)-methoxymethyl-pyrrolidine and (4-bromomethyl-phenyl)-acetonitrile.

Yield: 0.9 g (51.6% of theory);

C₁₅H₂₀N₂O (M=244.33);

calc.: molar peak (M+H)⁺: 245 fnd.: molar peak (M+H)⁺: 245;

R_{f} value: 0.3 (silica gel, cyclohexane/ethyl acetate 1:1).

1.13.b 2-[4-(2-(S)-methoxymethyl-pyrrolidin-1-ylmethyl)-phenyl]-ethylamine

Prepared analogously to Example 1.1.h from [4-(2-(S)-methoxymethyl-pyrrolidin-1-ylmethyl)-phenyl]-acetonitrile

Yield: 0.5 g (54.7% of theory);

C₁₅H₂₄N₂O (M=248.37);

calc.: molar peak (M+H)⁺: 249 fnd.: molar peak (M+H)⁺: 249;

R_{f} value: 0.3 (silica gel, dichloromethane/ethanol/ammonia 20:1:0.1).

1.13.c 4′-chloro-3-nitro-biphenyl-4-carboxylic acid-{2-[4-(2-(S)-methoxymethyl-pyrrolidin-1-ylmethyl)-phenyl]-ethyl}-amide

Prepared analogously to Example 1.1.i from 4′-chloro-3-nitro-biphenyl-4-carboxylic acid and 2-[4-(2-(S)-methoxymethyl-pyrrolidin-1-ylmethyl)-phenyl]-ethylamine.

Yield: 0.5 g (54.7% of theory);

C₂₈H₃₀ClN₃O₄ (M=508.02);

calc.: molar peak (M+H)⁺: 508/510 fnd.: molar peak (M+H)⁺: 508/510;

R_{f} value: 0.6 (silica gel, dichloromethane/ethanol/ammonia 20:1:0.1).

1.13.d 4′-chloro-3-amino-biphenyl-4-carboxylic acid-{2-[4-(2-(S)-methoxymethyl-pyrrolidin-1-ylmethyl)-phenyl]-ethyl}-amide

Prepared analogously to Example 1.3.c from 4′-chloro-3-nitro-biphenyl-4-carboxylic acid-{2-[4-(2-(S)-methoxymethyl-pyrrolidin-1-ylmethyl)-phenyl]-ethyl}-amide.

Yield: 0.24 g (51% of theory);

C₂₈H₃₂ClN₃O₂ (M=478.03);

calc.: molar peak (M+H)⁺: 478/480 fnd.: molar peak (M+H)⁺: 478/480;

R_{f} value: 0.2 (silica gel, dichloromethane/methanol/ammonia 10:1:0.1).

### EXAMPLE 1.14

### 7-(4-chloro-phenyl)-3-[2-(4-dimethylaminomethyl-phenyl)-ethyl]-3H-quinazolin-4-one

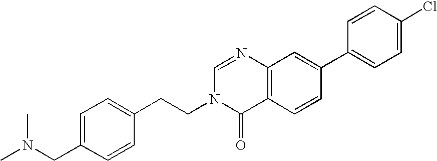

1.14.a (4-dimethylaminomethyl-phenyl)-acetonitrile

Prepared analogously to Example 1.1.g from dimethylamine and (4-bromomethyl-phenyl)-acetonitrile.

Yield: 1.0 g (30% of theory);

C₁₁H₁₄N₂ (M=174.24);

calc.: molar peak (M+H)⁺: 175 fnd.: molar peak (M+H)⁺: 175;

R_{f} value: 0.2 (silica gel, cyclohexane/ethyl acetate 1:1).

1.14.b 2-(4-dimethylaminomethyl-phenyl)-ethylamine

Prepared analogously to Example 1.1.h from (4-dimethylaminomethyl-phenyl)-acetonitrile

Yield: 1.0 g crude;

C₁₁H₁₈N₂ (M=178.28);

calc.: molar peak (M+H)⁺: 179 fnd.: molar peak (M+H)⁺: 179;

R_{f} value: 0.2 (silica gel, dichloromethane/ethanol/ammonia 20:1:0.1).

1.14.c 4′-chloro-3-nitro-biphenyl-4-carboxylic acid-[2-(4-dimethylaminomethyl-phenyl)-ethyl]-amide

Prepared analogously to Example 1.1.i from 4′-chloro-3-nitro-biphenyl-4-carboxylic acid and 2-(4-dimethylaminomethyl-phenyl)-ethylamine

Yield: 0.5 g (63.4% of theory);

C₂₄H₂₄ClN₃O₃ (M=437.93);

calc.: molar peak (M+H)⁺: 438/440 fnd.: molar peak (M+H)⁺: 438/440;

R_{f} value: 0.35 (silica gel, dichloromethane/ethanol/ammonia 20:1:0.1).

1.14.d 4′-chloro-3-amino-biphenyl-4-carboxylic acid-[2-(4-dimethylaminomethyl-phenyl)-ethyl]-amide

Prepared analogously to Example 1.3.c from 4′-chloro-3-nitro-biphenyl-4-carboxylic acid-[2-(4-dimethylaminomethyl-phenyl)-ethyl]-amide

Yield: 0.2 g (43% of theory);

C₂₄H₂₆ClN₃O (M=407.94);

calc.: molar peak (M+H)⁺: 408/410 fnd.: molar peak (M+H)⁺: 408/410;

R_{f} value: 0.2 (silica gel, dichloromethane/methanol/ammonia 20:1:0.1).

### EXAMPLE 1.15

### 7-(4-chloro-phenyl)-3-[2-(4-piperidin-1-ylmethyl-phenyl)-ethyl]-3H-quinazolin-4-one

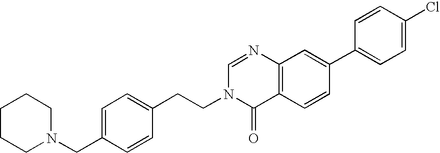

1.15.a (4-piperidin-1-ylmethyl-phenyl)-acetonitrile

Prepared analogously to Example 1.1.g from piperidine and (4-bromomethyl-phenyl)-acetonitrile.

Yield: 1.6 g (39% of theory);

C₁₄H₁₈N₂(M=214.31);

calc.: molar peak (M+H)⁺: 215 fnd.: molar peak (M+H)⁺: 215;

R_{f} value: 0.4 (silica gel, cyclohexane/ethyl acetate 1:1).

1.15.b 2-(4-piperidin-1-ylmethyl-phenyl)-ethylamine

Prepared analogously to Example 1.1.h from (4-piperidin-1-ylmethyl-phenyl)-acetonitrile

Yield: 1.4 g (85.9% of theory);

C₁₄H₂₂N₂ (M=218.34);

calc.: molar peak (M+H)⁺: 219 fnd.: molar peak (M+H)⁺: 219;

R_{f} value: 0.2 (silica gel, dichloromethane/ethanol/ammonia 20:1:0.1).

1.15.c 4′-chloro-3-nitro-biphenyl-4-carboxylic acid-[2-(4-piperidin-1-ylmethyl-phenyl)-ethyl]-amide

Prepared analogously to Example 1.1.i from 4′-chloro-3-nitro-biphenyl-4-carboxylic acid and 2-(4-piperidin-1-ylmethyl-phenyl)-ethylamine.

Yield: 0.07 g (40.7% of theory);

C₂₇H₂₈ClN₃O₃ (M=477.99);

calc.: molar peak (M+H)⁺: 478/480 fnd.: molar peak (M+H)⁺: 478/480;

R_{f} value: 0.5 (silica gel, dichloromethane/ethanol/ammonia 20:1:0.1).

1.15.d 4′-chloro-3-amino-biphenyl-4-carboxylic acid-[2-(4-piperidin-1-ylmethyl-phenyl)-ethyl]-amide

Prepared analogously to Example 1.3.c from 4′-chloro-3-nitro-biphenyl-4-carboxylic acid-[2-(4-piperidin-1-ylmethyl-phenyl)-ethyl]-amide

Yield: 0.05 g (76.4% of theory);

C₂₇H₃₀ClN₃O (M=448.01).

### EXAMPLE 1.16

### 7-(4-chloro-phenyl)-3-[2-(4-morpholin-4-ylmethyl-phenyl)-ethyl]-3H-quinazolin-4-one

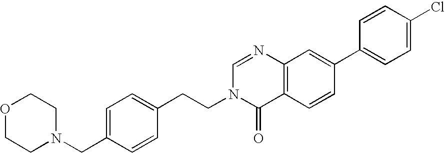

1.16.a (4-morpholin-4-ylmethyl-phenyl)-acetonitrile

Prepared analogously to Example 1.1.g from morpholine and (4-bromomethyl-phenyl)-acetonitrile.

Yield: 1.63 g (98.9% of theory);

C₁₃H₁₆N₂O (M=216.28);

calc.: molar peak (M+H)⁺: 217 fnd.: molar peak (M+H)⁺: 217;

R_{f} value: 0.33 (silica gel, cyclohexane/ethyl acetate 1:1).

1.16.b 2-(4-morpholin-1-ylmethyl-phenyl)-ethylamine

Prepared analogously to Example 1.1.h from (4-morpholin-1-ylmethyl-phenyl)-acetonitrile

Yield: 1.65 g (99.4% of theory);

C₁₃H₂₀N₂O (M=220.31);

calc.: molar peak (M+H)⁺: 221 fnd.: molar peak (M+H)⁺: 221;

R_{f} value: 0.54 (silica gel, dichloromethane/ethanol/ammonia 9:1:0.1).

1.16.c 4′-chloro-3-nitro-biphenyl-4-carboxylic acid-[2-(4-morpholin-1-ylmethyl-phenyl)-ethyl]-amide

Prepared analogously to Example 1.1.i from 4′-chloro-3-nitro-biphenyl-4-carboxylic acid and 2-(4-morpholin-1-ylmethyl-phenyl)-ethylamine.

Yield: 0.53 g (76.6% of theory);

C₂₆H₂₆ClN₃O₄ (M=479.97);

calc.: molar peak (M+H)⁺: 480/482 fnd.: molar peak (M+H)⁺: 480/482;

R_{f} value: 0.5 (silica gel, dichloromethane/ethanol/ammonia 90:1:0.1).

1.16.d 4′-chloro-3-amino-biphenyl-4-carboxylic acid-[2-(4-morpholin-1-ylmethyl-phenyl)-ethyl]-amide

Prepared analogously to Example 1.3.c from 4′-chloro-3-nitro-biphenyl-4-carboxylic acid-[2-(4-morpholin-1-ylmethyl-phenyl)-ethyl]-amide

Yield: 0.45 g (90.6% of theory);

C₂₆H₂₈ClN₃O₂ (M=449.98);

calc.: molar peak (M+H)⁺: 450/452 fnd.: molar peak (M+H)⁺: 450/452;

R_{f} value: 0.67 (silica gel, dichloromethane/ethanol/ammonia 90:1:0.1).

The following compounds were prepared analogously to Example 1.1.k:

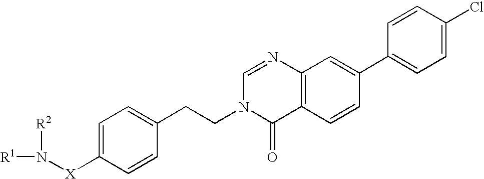

| | | | empirical | mass | mp | |
|---|---|---|---|---|---|---|
| Example | R¹R²N—X— | educt | formula | spectrum | ]° C.] | R_{f} value |
| 1.13 | 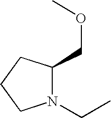 | 1.13.d | C₂₉H₃₀ClN₃O₂ | 488/490 [M + H]⁺ | 133-135 | 0.3 (C) |
| 1.14 | 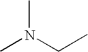 | 1.14.d | C₂₅H₂₄ClN_{3O} | 418/420 [M + H]⁺ | 183 | 0.66 (C) |
| 1.15 | 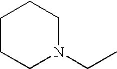 | 1.15.d | C₂₈H₂₈ClN_{3O} | 458 [M + H]⁺ | 169-170 | 0.4 (D) |
| 1.16 | 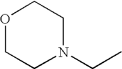 | 1.16.d | C₂₇H₂₆ClN₃O₂ | 460/462 [M + H]⁺ | 169-170 | 0.77 (A) |

R_{f} value:
A = (silica gel, dichloromethane/methanol/ammonia 9:1:0.1)
C = (silica gel, dichloromethane/methanol/ammonia 10:1:0.1)
D = (silica gel, dichloromethane/ethanol/ammonia 20:1:0.1)

### EXAMPLE 1.17

### 7-(4-chloro-phenyl)-3-{2-[6-(4-methyl-piperazin-1-yl)-pyrid in-3-yl]-ethyl}-3H-quinazolin-4-one

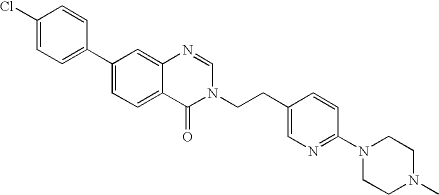

1.17.a (6-chloro-pyridin-3-yl)-acetonitrile

A solution of 7.5 g (41.66 mmol) of 2-chloro-5-chloromethyl-pyridine, dissolved in 100 ml of ethanol, is added dropwise to a solution of 6.91 g (41.66 mmol) of potassium iodide and 2.24 g (49.01 mmol) of sodium cyanide in 400 ml of an ethanol/water mixture (9:1). Then the reaction mixture is heated to 85° C. for five hours. The solvent is substantially distilled off in vacuo and the residue is extracted with water and ethyl acetate. The organic phase is washed with water three times and dried over sodium sulphate. The purification is carried out by column chromatography on silica gel (eluant: dichloromethane/ethanol).

Yield: 2.9 g (45.6% of theory);

C₇H₅ClN₂ (M=152.58);

calc.: molar peak (M+H)⁺: 151/153 fnd.: molar peak (M+H)⁺: 151/153.

1.17.b [6-(4-methyl-piperazin-1-yl)-pyridin-3-yl]-acetonitrile

A solution of 2.9 g (19 mmol) of (6-chloro-pyridin-3-yl)-acetonitrile, 5.27 ml (38 mmol) of triethylamine and 2.1 ml (19 mmol) of N-methylpiperazine in 50 ml of n-butanol is heated to 180° C. for two hours in the microwave. The solvent is distilled off in vacuo, the residue suspended in water and then extracted with ethyl acetate. The combined organic phases are extracted three times with water and dried over sodium sulphate. The purification is carried out by column chromatography on Alox (eluant: petroleum ether/ethyl acetate 1:1).

Yield: 1 g (24.6% of theory);

melting point: 58-59° C.;

C₁₂H₁₆N₄ (M=216.28);

calc.: molar peak (M+H)⁺: 217 fnd.: molar peak (M+H)⁺: 217;

R_{f} value: 0.35 (silica gel, dichloromethane/methanol/ammonia 9:1:0.1).

1.17.c 2-[6-(4-methyl-piperazin-1-yl)-pyridin-3-yl]-ethylamine

Prepared analogously to Example 1.1.i from [6-(4-methyl-piperazin-1-yl)-pyridin-3-yl]-acetonitrile.

Yield: 0.94 g (96% of theory);

C₁₂H₂₀N₄ (M=220.32);

calc.: molar peak (M+H)⁺: 221 fnd.: molar peak (M+H)⁺: 221.

1.17.d 4′-chloro-3-nitro-biphenyl-4-carboxylic acid-{2-[6-(4-methyl-piperazin-1-yl)-pyridin-3-yl]-ethyl}-amide

Prepared analogously to Example 1.1.j from 4′-chloro-3-nitro-biphenyl-4-carboxylic acid and 2-[6-(4-methyl-piperazin-1-yl)-pyridin-3-yl]-ethylamine.

Yield: 0.48 g (36.7% of theory);

melting point: 158-159° C.;

C₂₅H₂₆ClN₅O₃ (M=479.97);

calc.: molar peak (M+H)⁺: 480/482 fnd.: molar peak (M+H)⁺: 480/482.

1.17.e 4′-chloro-3-amino-biphenyl-4-carboxylic acid-{2-[6-(4-methyl-piperazin-1-yl)-pyridin-3-yl]-ethyl}-amide

Prepared analogously to Example 1.1.i from 4′-chloro-3-nitro-biphenyl-4-carboxylic acid-{2-[6-(4-methyl-piperazin-1-yl)-pyridin-3-yl]-ethyl}-amide.

Yield: 0.12 g (64% of theory);

melting point: 198-199° C.;

C₂₅H₂₈ClN₅O (M=449.98);

calc.: molar peak (M+H)⁺: 450/452 fnd.: molar peak (M+H)⁺: 450/452.

1.17.f 7-(4-chloro-phenyl)-3-{2-[6-(4-methyl-piperazin-1-yl)-pyridin-3-yl]-ethyl}-3H-quinazolin-4-one

Prepared analogously to Example 1.1.l from 4′-chloro-3-amino-biphenyl-4-carboxylic acid-{2-[6-(4-methyl-piperazin-1-yl)-pyridin-3-yl]-ethyl}-amide and formic acid.

Yield: 0.06 g (53.5% of theory);

melting point: 263-264° C.;

C₂₆H₂₆ClN₅O (M=459.98);

calc.: molar peak (M+H)⁺: 460/462 fnd.: molar peak (M+H)⁺: 4460/462.

### EXAMPLE 1.18

### 7-(4-chloro-phenyl)-3-[2-(4-pyrrolidin-1-ylmethyl-phenyl)-ethyl]-3H-benzo[d][1,2,3]triazin-4-one

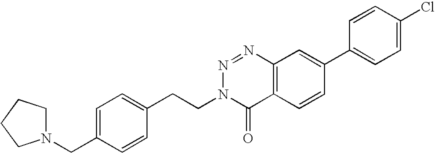

1.18.a 7-(4-chloro-phenyl)-3-{2-[4-(1-pyrrolidin-1-yl-ethyl)-phenyl]-ethyl}-3H-benzo[d][1,2,3]triazin-4-one

A solution of 0.09 g (0.93 mmol) of sodium nitrite in 2 ml of water is slowly added dropwise to a solution of 0.27 g (0.62 mmol) of 4′-chloro-3-amino-biphenyl-4-carboxylic acid-[2-(4-pyrrolidin-1-ylmethyl-phenyl)-ethyl]-amide (cf. Example 1.1.j) in 10 ml of methanol and 1N hydrochloric acid at a temperature between 0° C. and 5° C. Then the reaction mixture is stirred for three hours at ambient temperature, then diluted with 30 ml of water and made alkaline with ammonia solution. The aqueous solution is extracted with ethyl acetate. The combined organic phases are washed with water three times, dried over sodium sulphate and filtered through activated charcoal. The solvent is removed and the residue washed with diisopropylether.

Yield: 0.09 g (32.5% of theory);

melting point: 151-152° C.;

C₂₆H₂₅ClN₄O (M=444.96);

calc.: molar peak (M+H)⁺: 445/447 fnd.: molar peak (M+H)⁺: 445/447;

R_{f} value: 0.35 (silica gel, dichloromethane/ethanol=10:1).

### EXAMPLE 1.19

### 7-(4-chloro-phenyl)-3-(4-pyrrolidin-1-ylmethyl-benzyl)-3H-benzo[d][1,2,3]triazin-4-one

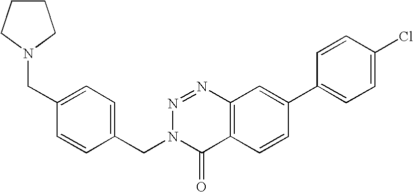

1.19.a 4-(1-pyrrolidin-1-yl-ethyl)-benzonitrile

Prepared analogously to Example 1.1.g from piperidine and 4-bromomethyl-benzonitrile

Yield: 2.4 g (85.9% of theory);

C₁₂H₁₄N₂ (M=186.25);

calc.: molar peak (M+H)⁺: 187 fnd.: molar peak (M+H)⁺: 187;

R_{f} value: 0.63 (silica gel, dichloromethane/methanol/ammonia=8:2:1).

1.19.b 4-(1-pyrrolidin-1-yl-ethyl)-benzylamine

Prepared analogously to Example 1.1.h from 4-(1-pyrrolidin-1-yl-ethyl)-benzonitrile

Yield: 2.42 g (98.7% of theory);

C₁₂H₁₈N₂ (M=190.29);

calc.: molar peak (M+H)⁺: 191 fnd.: molar peak (M+H)⁺: 191;

R_{f} value: 0.26 (silica gel, dichloromethane/methanol/ammonia=90:10:1).

1.19.c 4′-chloro-3-nitro-biphenyl-4-carboxylic acid-4-(1-pyrrolidin-1-yl-ethyl)-benzylamide

Prepared analogously to Example 1.1.i from 4′-chloro-3-nitro-biphenyl-4-carboxylic acid and 2-(4-4-(1-pyrrolidin-1-yl-ethyl)-benzylamine.

Yield: 0.28 g (28.8% of theory);

C₂₅H₂₄ClN₃O₃ (M=449.94);

calc.: molar peak (M+H)⁺: 450/452 fnd.: molar peak (M+H)⁺: 450/452.

1.19.d. 3-amino-4′-chloro-biphenyl-4-carboxylic acid-4-(1-pyrrolidin-1-yl-ethyl)-benzylamide

Prepared analogously to Example 1.3.c from 4′-chloro-3-nitro-biphenyl-4-carboxylic acid-4-(1-pyrrolidin-1-yl-ethyl)-benzylamide.

Yield: 0.19 g (72.7% of theory);

C₂₅H₂₆ClN₃O (M=419.95);

calc.: molar peak (M+H)⁺: 420/422 fnd.: molar peak (M+H)⁺: 420/422.

1.19.e 7-(4-chloro-phenyl)-3-[4-(1-pyrrolidin-1-yl-ethyl)-benzyl]-3H-benzo[d][1,2,3]triazin-4-one

Prepared analogously to Example 1.18.a from 3-amino-4′-chloro-biphenyl-4-carboxylic acid-4-(1-pyrrolidin-1-yl-ethyl)-benzylamide.

Yield: 0.045 g (31.4% of theory);

melting point: 147-148° C.;

C₂₅H₂₃ClN₄O (M=430.94);

calc.: molar peak (M+H)⁺: 431/433 fnd.: molar peak (M+H)⁺: 431/433;

R_{f} value: 0.3 (silica gel, dichloromethane/ethanol=10:1).

### EXAMPLE 1.20

### 5-(4-fluoro-phenyl)-2-[2-(4-pyrrolidin-1-ylmethyl-phenyl)-ethyl]-isoindol-1,3-dione

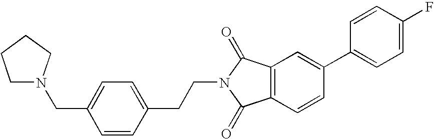

1.20.a 5-bromo-2-{2-[4-(1-pyrrolidin-1-yl-ethyl)-phenyl]-ethyl}-isoindol-1,3-dione

A solution of 0.8 g (3.52 mmol) of 5-bromo-isobenzofuran-1,3-dione and 0.72 g (3.52 mmol) of 2-(4-pyrrolidin-1-ylmethyl-phenyl)-ethylamine (cf. Example 1.1.h) in 10 ml acetic acid is heated to 110° C. for four hours. Then the reaction mixture is poured into water, made alkaline with 2N sodium hydroxide solution and the precipitate is filtered off. The precipitate is washed several times with water and dried.

Yield: 0.5 g (34.3% of theory);

C₂₁H₂₁BrN₂O₂ (M=413.31);

calc.: molar peak (M+H)⁺: 413/415 fnd.: molar peak (M+H)⁺: 413/415.

1.20.b. 5-(4-fluoro-phenyl)-2-{2-[4-(1-pyrrolidin-1-yl-ethyl)-phenyl]-ethyl}-isoindol-1,3-dione

Prepared analogously to Example 1.1.b from 5-bromo-2-{2-[4-(1-pyrrolidin-1-yl-ethyl)-phenyl]-ethyl}-isoindol-1,3-dione and 4-fluoro-phenylboric acid.

Yield: 0.01 g (4.8% of theory);

C₂₇H₂₅FN₂O₂ (M=428.51);

calc.: molar peak (M+H)⁺: 429 fnd.: molar peak (M+H)⁺: 429.

### EXAMPLE 1.21

### 7-(4-chloro-phenyl)-3-{2-[4-(4-phenyl-piperidin-1-ylmethyl)-phenyl]-ethyl}-3H-quinazolin-4-one

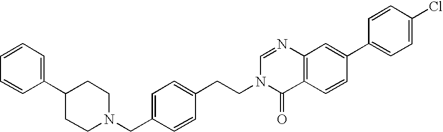

1.21.a [4-(4-phenyl-piperidin-1-ylmethyl)-phenyl]-acetonitrile

Prepared analogously to Example 1.1.g from 4-phenylpiperidine and (4-bromomethyl-phenyl)-acetonitrile.

Yield: 3.8 g (98% of theory);

C₂₀H₂₂N₂ (M=290.41);

calc.: molar peak (M+H)⁺: 291 fnd.: molar peak (M+H)⁺: 291;

R_{f} value: 0.5 (silica gel, cyclohexane/ethyl acetate 1:1).

1.21.b 2-[4-(4-phenyl-piperidin-1-ylmethyl)-phenyl]-ethylamine

Prepared analogously to Example 1.1.h from [4-(4-phenyl-piperidin-1-ylmethyl)-phenyl]-acetonitrile.

Yield: 3.6 g crude;

C₂₀H₂₆N₂ (M=294.44);

calc.: molar peak (M+H)⁺: 295 fnd.: molar peak (M+H)⁺: 295;

R_{f} value: 0.49 (silica gel, dichloromethane/ethanol 20:1).

1.21.c 4′-chloro-3-nitro-biphenyl-4-carboxylic acid-{2-[4-(4-phenyl-piperidin-1-ylmethyl)-phenyl]-ethyl}-amide

Prepared analogously to Example 1.1.i from 4′-chloro-3-nitro-biphenyl-4-carboxylic acid and 2-[4-(4-phenyl-piperidin-1-ylmethyl)-phenyl]-ethylamine

Yield: 1.33 g (70.7% of theory);

C₃₃H₃₂ClN₃O₃ (M=554.09);

calc.: molar peak (M+H)⁺: 554/556 fnd.: molar peak (M+H)⁺: 554/556;

R_{f} value: 0.58 (silica gel, dichloromethane/ethanol/ammonia 10:1:0.1).

1.21.d 4′-chloro-3-amino-biphenyl-4-carboxylic acid-{2-[4-(4-phenyl-piperidin-1-ylmethyl)-phenyl]-ethyl}-amide

Prepared analogously to Example 1.3.c from 4′-chloro-3-nitro-biphenyl-4-carboxylic acid-{2-[4-(4-phenyl-piperidin-1-ylmethyl)-phenyl]-ethyl}-amide

Yield: 0.82 g (65.2% of theory);

C₃₃H₃₄ClN₃O (M=524.11);

calc.: molar peak (M+H)⁺: 524/526/528 fnd.: molar peak (M+H)⁺: 524/526/528;

R_{f} value: 0.65 (silica gel, dichloromethane/methanol 10:1).

### EXAMPLE 1.22

### 7-(4-chloro-phenyl)-3-{2-[4-(4-phenyl-piperazin-1-ylmethyl)-phenyl]-ethyl}-3H-quinazolin-4-one

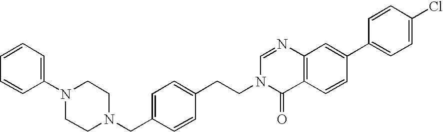

1.22.a [4-(4-phenyl-piperazin-1-ylmethyl)-phenyl]-acetonitrile

Prepared analogously to Example 1.1.g from 4-phenylpiperazine and (4-bromomethyl-phenyl)-acetonitrile.

Yield: 3.7 g (97% of theory);

C₁₉H₂₁N₃ (M=291.39);

calc.: molar peak (M+H)⁺: 292 fnd.: molar peak (M+H)⁺: 292;

R_{f} value: 0.6 (silica gel, cyclohexane/ethyl acetate 1:1).

1.22.b 2-[4-(4-phenyl-piperazin-1-ylmethyl)-phenyl]-ethylamine

Prepared analogously to Example 1.1.h from [4-(4-phenyl-piperazin-1-ylmethyl)-phenyl]-acetonitrile

Yield: 1.1 g (28.6% of theory);

C₁₉H₂₅N₃ (M=295.43);

calc.: molar peak (M+H)⁺: 296 fnd.: molar peak (M+H)⁺: 296.

1.22.c 4′-chloro-3-nitro-biphenyl-4-carboxylic acid-{2-[4-(4-phenyl-piperazin-1-ylmethyl)-phenyl]-ethyl}-amide

Prepared analogously to Example 1.1.i from 4′-chloro-3-nitro-biphenyl-4-carboxylic acid and 2-[4-(4-phenyl-piperazin-1-ylmethyl)-phenyl]-ethylamine

Yield: 0.32 g (18.2% of theory);

C₃₂H₃₁ClN₄O₃ (M=555.08);

calc.: molar peak (M+H)⁺: 555/557 fnd.: molar peak (M+H)⁺: 555/557.

1.22.d 4′-chloro-3-amino-biphenyl-4-carboxylic acid-{2-[4-(4-phenyl-piperazin 1-ylmethyl)-phenyl]-ethyl}-amide

Prepared analogously to Example 1.3.c from 4′-chloro-3-nitro-biphenyl-4-carboxylic acid-{2-[4-(4-phenyl-piperazin-1-ylmethyl)-phenyl]-ethyl}-amide

Yield: 0.11 g (38.8% of theory);

C₃₂H₃₃ClN₄O (M=525.09);

calc.: molar peak (M+H)⁺: 525/527 fnd.: molar peak (M+H)⁺: 525/527.

### EXAMPLE 1.23

### 7-(4-chloro-phenyl)-3-{2-[4-(4-hydroxy-4-phenyl-piperidin-1-ylmethyl)-phenyl]-ethyl}-3H-quinazolin-4-one

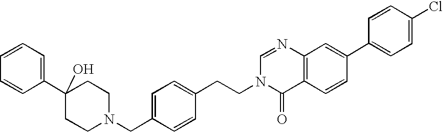

1.23.a [4-(4-hydroxy-4-phenyl-piperidin-1-ylmethyl)-phenyl]-acetonitrile

Prepared analogously to Example 1.1.g from 4-hydroxy-4-phenylpiperidine and (4-bromomethyl-phenyl)-acetonitrile.

Yield: 3.8 g (98% of theory);

C₂₀H₂₂N₂O (M=306.41);

calc.: molar peak (M+H)⁺: 307 fnd.: molar peak (M+H)⁺: 307;

R_{f} value: 0.1 (silica gel, cyclohexane/ethyl acetate 1:1).

1.23.b 2-[4-(4-hydroxy-4-phenyl-piperidin-1-ylmethyl)-phenyl]-ethylamine

Prepared analogously to Example 1.1.h from [4-(4-hydroxy-4-phenyl-piperidin-1-ylmethyl)-phenyl]-acetonitrile

Yield: 3.36 g (92.1% of theory);

C₂₀H₂₆N₂O (M=310.44);

calc.: molar peak (M+H)⁺: 311 fnd.: molar peak (M+H)⁺: 311;

R_{f} value: 0.1 (silica gel, dichloromethane/methanol/acetic acid 9:1:0.1).

1.23.c 4′-chloro-3-nitro-biphenyl-4-carboxylic acid-{2-[4-(4-hydroxy-4-phenyl-piperidin-1-ylmethyl)-phenyl]-ethyl}-amide

Prepared analogously to Example 1.1.i from 4′-chloro-3-nitro-biphenyl-4-carboxylic acid and 2-[4-(4-hydroxy-4-phenyl-piperidin-1-ylmethyl)-phenyl]-ethylamine

Yield: 1.2 g (65.3% of theory);

C₃₃H₃₂ClN₃O₄ (M=570.09);

calc.: molar peak (M+H)⁺: 570/572 fnd.: molar peak (M+H)⁺: 570/572;

R_{f} value: 0.35 (silica gel, dichloromethane/methanol/ammonia 10:1:0.1).

1.23.d 4′-chloro-3-amino-biphenyl-4-carboxylic acid-{2-[4-(4-hydroxy-4-phenyl-piperidin-1-ylmethyl)-phenyl]-ethyl}-amide

Prepared analogously to Example 1.3.c from 4′-chloro-3-nitro-biphenyl-4-carboxylic acid-{2-[4-(4-hydroxy-4-phenyl-piperidin-1-ylmethyl)-phenyl]-ethyl}-amide

Yield: 1.04 g (91.5% of theory);

C₃₃H₃₄ClN₃O₂ (M=540.11);

melting point: 175-180° C.;

calc.: molar peak (M+H)⁺: 540/542/544 fnd.: molar peak (M+H)⁺: 540/542/544;

R_{f} value: 0.34 (silica gel, dichloromethane/methanol/ammonia 10:1:0.1).

1.23.e. 7-(4-chloro-phenyl)-3-{2-[4-(4-hydroxy-4-phenyl-piperidin-1-ylmethyl)-phenyl]-ethyl}-3H-quinazolin-4-one

Prepared analogously to Example 1.1.k. from 4′-chloro-3-amino-biphenyl-4-carboxylic acid-{2-[4-(4-hydroxy-4-phenyl-piperidin-1-ylmethyl)-phenyl]-ethyl}-amide.

Yield: 0.025 g (8.2% of theory);

melting point: 204-205° C.;

C₃₄H₃₂ClN₃O₂ (M=550.10);

calc.: molar peak (M+H)⁺: 550/552 fnd.: molar peak (M+H)⁺: 550/552;

R_{f} value: 0.46 (silica gel, dichloromethane/ethanol/ammonia 10:1:0.1).

### EXAMPLE 1.24

### 7-(4-chloro-phenyl)-3-{2-[4-(4-phenyl-3.6-dihydro-2H-piperidin-1-ylmethyl)-phenyl]-ethyl}-3H-quinazolin-4-one

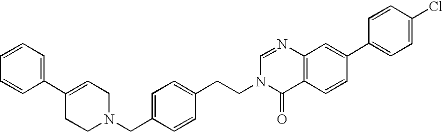

1.24.a. 7-(4-chloro-phenyl)-3-{2-[4-(4-phenyl-3.6-dihydro-2H-piperidin-1-ylmethyl)-phenyl]-ethyl}-3H-quinazolin-4-one

Prepared analogously to Example 1.1.k. from 4′-chloro-3-amino-biphenyl-4-carboxylic acid-{2-[4-(4-hydroxy-4-phenyl-piperidin-1-ylmethyl)-phenyl]-ethyl}-amide as by-product in Example 123.e.

Yield: 0.08 g (27.1% of theory);

melting point: 166-167° C.;

C₃₄H₃₀ClN₃O (M=532.09);

calc.: molar peak (M+H)⁺: 532/534 fnd.: molar peak (M+H)⁺: 532/534;

R_{f} value: 0.57 (silica gel, dichloromethane/ethanol/ammonia 10:1).

### EXAMPLE 1.25

### 7-(4-chloro-phenyl)-3-{2-[4-(3-Aza-spiro[5.5]undec-3-ylmethyl)-phenyl]-ethyl}-3H-quinazolin-4-one

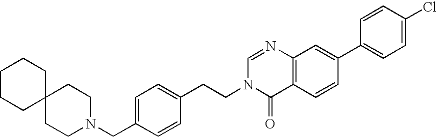

1.25.a [4-(3-Aza-spiro[5.5]undec-3-ylmethyl)-phenyl]-acetonitrile

Prepared analogously to Example 1.1.g from 3-aza-spiro[5.5]undecane and (4-bromomethyl-phenyl)-acetonitrile.

Yield: 3.38 g (98% of theory);

C₁₉H₂₆N₂ (M=282.43);

calc.: molar peak (M+H)⁺: 283 fnd.: molar peak (M+H)⁺: 283;

R_{f} value: 0.56 (silica gel, cyclohexane/ethyl acetate 1:1).

1.25.b 2-[4-(3-Aza-spiro[5.5]undec-3-ylmethyl)-phenyl]-ethylamine

Prepared analogously to Example 1.1.h from [4-(3-aza-spiro[5.5]undec-3-ylmethyl)-phenyl]-acetonitrile

Yield: 3.33 g (96.6% of theory);

C₁₉H₃₀N₂ (M=286.46);

calc.: molar peak (M+H)⁺: 287 fnd.: molar peak (M+H)⁺: 287;

R_{f} value: 0.18 (silica gel, dichloromethane/ethanol 20:1).

1.25.c 4′-chloro-3-nitro-biphenyl-4-carboxylic acid-{2-[4-(3-aza-spiro[5.5]undec-3-ylmethyl)-phenyl]-ethyl}-amide

Prepared analogously to Example 1.1.i from 4′-chloro-3-nitro-biphenyl-4-carboxylic acid and 2-[4-(3-aza-spiro[5.5]undec-3-ylmethyl)-phenyl]-ethylamine

Yield: 1 g (52.5% of theory);

C₃₂H₃₆ClN₃O₃ (M=546.11);

calc.: molar peak (M+H)⁺: 546/548 fnd.: molar peak (M+H)⁺: 546/548;

R_{f} value: 0.3 (silica gel, dichloromethane/ethanol 20:1).

1.25.d 4′-chloro-3-amino-biphenyl-4-carboxylic acid-{2-[4-(3-aza-spiro[5.5]undec-3-ylmethyl)-phenyl]-ethyl}-amide

Prepared analogously to Example 1.3.c from 4′-chloro-3-nitro-biphenyl-4-carboxylic acid-{2-[4-(3-aza-spiro[5.5]undec-3-ylmethyl)-phenyl]-ethyl}-amide

Yield: 0.8 g (84.7% of theory);

C₃₂H₃₈ClN₃O (M=516.13);

calc.: molar peak (M+H)⁺: 516/518 fnd.: molar peak (M+H)⁺: 516/518;

R_{f} value: 0.38 (silica gel, dichloromethane/methanol 10:1).

### EXAMPLE 1.26

### 7-(4-chloro-phenyl)-3-(2-{4-[4-(pyridin-2-yloxy)-piperidin-1-ylmethyl]-phenyl}-ethyl)-3H-quinazolin-4-one

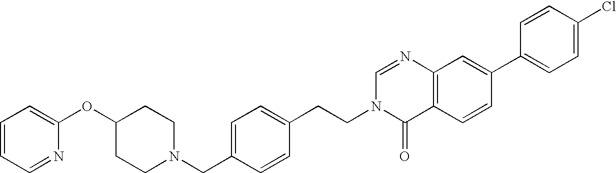

1.26.a {4-[4-(pyridin-2-yloxy)-piperidin-1-ylmethyl]-phenyl}-acetonitrile

Prepared analogously to Example 1.1.g from 2-(piperidin-4-yloxy)-pyridine and (4-bromomethyl-phenyl)-acetonitrile.

Yield: 0.91 g (49.8% of theory);

C₁₉H₂₁N₃O (M=307.39);

calc.: molar peak (M+H)⁺: 308 fnd.: molar peak (M+H)⁺: 308;

R_{f} value: 0.49 (silica gel, dichloromethane/methanol/ammonia 9:1:0.1).

1.26.b 2-{4-[4-(pyridin-2-yloxy)-piperidin-1-ylmethyl]-phenyl}-ethylamine

Prepared analogously to Example 1.1.h from {4-[4-(pyridin-2-yloxy)-piperidin-1-ylmethyl]-phenyl}-acetonitrile

Yield: 0.92 g (99.8% of theory);

C₁₉H₂₅N₃O (M=311.43);

calc.: molar peak (M+H)⁺: 312 fnd.: molar peak (M+H)⁺: 312;

R_{f} value: 0.16 (silica gel, dichloromethane/methanol/ammonia 9:1:0.1).

1.26.c 4′-chloro-3-nitro-biphenyl-4-carboxylic acid-(2-{4-[4-(pyridin-2-yloxy)-piperidin-1-ylmethyl]-phenyl}-ethyl)-amide

Prepared analogously to Example 1.1.i from 4′-chloro-3-nitro-biphenyl-4-carboxylic acid and 2-{4-[4-(pyridin-2-yloxy)-piperidin-1-ylmethyl]-phenyl}-ethylamine

Yield: 0.8 g (97.2% of theory);

C₃₂H₃₁ClN₄O₄ (M=571.08);

calc.: molar peak (M+H)⁺: 571/573 fnd.: molar peak (M+H)⁺: 571/573;

R_{f} value: 0.52 (silica gel, dichloromethane/methanol/ammonia 9:1:0.1).

1.26.d 4′-chloro-3-amino-biphenyl-4-carboxylic acid-(2-{4-[4-(pyridin-2-yloxy)-piperidin-1-ylmethyl]-phenyl}-ethyl)-amide

Prepared analogously to Example 1.3.c from 4′-chloro-3-nitro-biphenyl-4-carboxylic acid-(2-{4-[4-(pyridin-2-yloxy)-piperidin-1-ylmethyl]-phenyl}-ethyl)-amide

Yield: 0.38 g (50% of theory);

C₃₂H₃₃ClN₄O₂ (M=541.09);

calc.: molar peak (M+H)⁺: 541/543 fnd.: molar peak (M+H)⁺: 541/543;

R_{f} value: 0.5 (silica gel, dichloromethane/methanol/ammonia 9:1:0.1).

### EXAMPLE 1.27

### 7-(4-chloro-phenyl)-3-(2-{4-[4-(pyridin-2-ylamino)-piperidin-1-ylmethyl]-phenyl}-ethyl)-3H-quinazolin-4-one

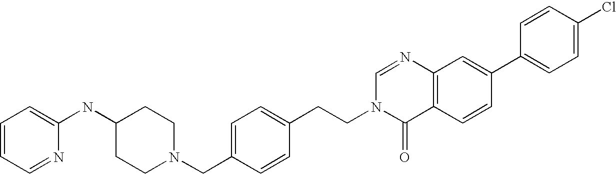

1.27.a {4-[4-(pyridin-2-ylamino)-piperidin-1-ylmethyl]-phenyl}-acetonitrile

Prepared analogously to Example 1.1.g from 2-(piperidin-4-ylamino)-pyridine and (4-bromomethyl-phenyl)-acetonitrile.

Yield: 1.57 g (86.1% of theory);

C₁₉H₂₂N₄ (M=306.41);

calc.: molar peak (M+H)⁺: 307 fnd.: molar peak (M+H)⁺: 307;

R_{f} value: 0.43 (silica gel, dichloromethane/methanol/ammonia 9:1:0.1).

1.27.b 2-{4-[4-(pyridin-2-ylamino)-piperidin-1-ylmethyl]-phenyl}-ethylamine

Prepared analogously to Example 1.1.h from {4-[4-(pyridin-2-ylamino)-piperidin-1-ylmethyl]-phenyl}-acetonitrile

Yield: 1.62 g (99.8% of theory);

C₁₉H₂₆N₄ (M=310.44);

calc.: molar peak (M+H)⁺: 311 fnd.: molar peak (M+H)⁺: 311;

R_{f} value: 0.1 (silica gel, dichloromethane/methanol/ammonia 9:1:0.1).

1.27.c 4′-chloro-3-nitro-biphenyl-4-carboxylic acid-(2-{4-[4-(pyridin-2-ylamino)-piperidin-1-ylmethyl]-phenyl}-ethyl)-amide

Prepared analogously to Example 1.1.i from 4′-chloro-3-nitro-biphenyl-4-carboxylic acid and 2-{4-[4-(pyridin-2-ylamino)-piperidin-1-ylmethyl]-phenyl}-ethylamine

Yield: 0.36 g (43.8% of theory);

C₃₂H₃₂ClN₅O₃ (M=570.09);

calc.: molar peak (M+H)⁺: 570/572 fnd.: molar peak (M+H)⁺: 570/572;

R_{f} value: 0.28 (silica gel, dichloromethane/methanol/ammonia 9:1:0.1).

1.27.d 4′-chloro-3-amino-biphenyl-4-carboxylic acid-(2-{4-[4-(pyridin-2-ylamino)-piperidin-1-ylmethyl]-phenyl}-ethyl)-amide

Prepared analogously to Example 1.3.c from 4′-chloro-3-nitro-biphenyl-4-carboxylic acid-(2-{4-[4-(pyridin-2-ylamino)-piperidin-1-ylmethyl]-phenyl}-ethyl)-amide

Yield: 0.29 g (85.7% of theory);

C₃₂H₃₄ClN₅O (M=540.11);

calc.: molar peak (M+H)⁺: 540/542 fnd.: molar peak (M+H)⁺: 540/542;

R_{f} value: 0.27 (silica gel, dichloromethane/methanol/ammonia 9:1:0.1).

The following compounds were prepared analogously to Example 1.1.k:

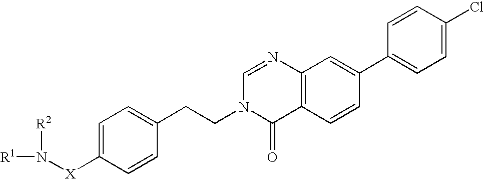

| | | | empirical | mass | mp | |
|---|---|---|---|---|---|---|
| Example | R₁R₂—N—X— | educt | formula | spectrum | [° C.] | R_{f} value |
| 1.21 | 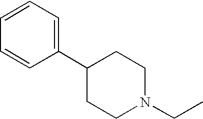 | 1.21.d | C₃₄H₃₂ClN₃O | 534/536 [M + H]³⁰ | 178-179 | 0.72 (E) |
| 1.22 | 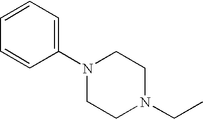 | 1.22.d | C₃₃H₃₁ClN₄O | 535/537 [M + H]³⁰ | 199-200 | |
| 1.23 | 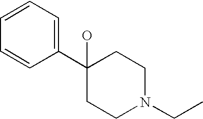 | 1.23.d | C₃₄H₃₂ClN₃O₂ | 550/552 [M + H]³⁰ | 204-205 | 0.46 (F) |
| 1.24 | 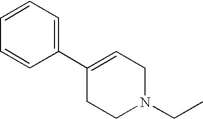 | 1.23.d | C₃₄H₃₀ClN₃O | 532/534 [M + H]³⁰ | 166-167 | 0.57 (E) |
| 1.25 | 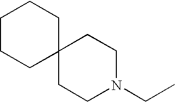 | 1.25.d | C₃₃H₃₆ClN₃O | 526/528 [M + H]³⁰ | 184-185 | 0.62 (E) |
| 1.26 | 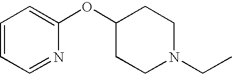 | 1.26.d | C₃₃H₃₁ClN₄O₂ | 551/553 [M + H]³⁰ | 154-158 | 0.46 (A) |
| 1.27 | 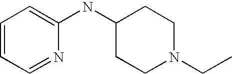 | 1.27.d | C₃₃H₃₂ClN₅O | 550/552 [M + H]³⁰ | 164-166 | 0.45 (A) |

R_{f} value:
A = (silica gel, dichloromethane/methanol/ammonia 9:1:0.1)
E = (silica gel, dichloromethane/ethanol 10:1)
F = (silica gel, dichloromethane/ethanol/ammonia 10:1:0.1)

### EXAMPLE 1.28

### 7-(4-chloro-phenyl)-3-{(2-[4-(4-phenyl-piperidin-1-ylmethyl)-phenyl]-ethyl}-3H-benzo[d][1,2,3]triazin-4-one

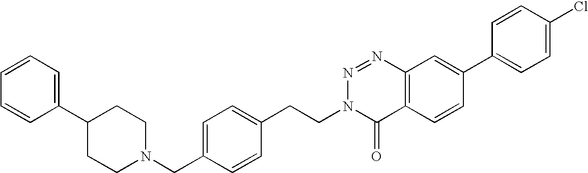

1.28.a 7-(4-chloro-phenyl)-3-{2-[4-(4-phenyl-piperidin-1-ylmethyl)-phenyl]-ethyl}-3H-benzo[d][1,2,3]triazin-4-one

Prepared analogously to Example 1.18.a from 4′-chloro-3-amino-biphenyl-4-carboxylic acid-{2-[4-(4-phenyl-piperidin-1-ylmethyl)-phenyl]-ethyl}-amide.

Yield: 0.13 g (50.9% of theory);

melting point: 183-184° C.;

C₃₃H₃₁ClN₄O (M=535.09);

calc.: molar peak (M+H)⁺: 535/537 fnd.: molar peak (M+H)⁺: 535/537;

R_{f} value: 0.66 (silica gel, dichloromethane/ethanol 10:1).

### EXAMPLE 1.29

### 7-(4-chloro-phenyl)-3-{2-[4-(4-hydroxy-4-phenyl-piperidin-1-ylmethyl)-phenyl]-ethyl}-3H-benzo[d][1,2,3]triazin-4-one

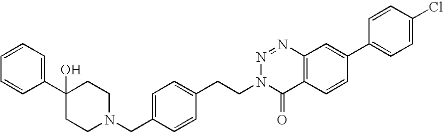

1.29.a 7-(4-chloro-phenyl)-3-{2-[4-(4-hydroxy-4-phenyl-piperidin-1-ylmethyl)-phenyl]-ethyl}-3H-benzo[d][1,2,3]triazin-4-one

Prepared analogously to Example 1.18.a from 4′-chloro-3-amino-biphenyl-4-carboxylic acid-{2-[4-(4-hydroxy-4-phenyl-piperidin-1-ylmethyl)-phenyl]-ethyl}-amide.

Yield: 0.21 g (68.7% of theory);

melting point: 265-266° C.;

C₃₃H₃₁ClN₄O₂ (M=551.09);

calc.: molar peak (M+H)⁺: 551/553 fnd.: molar peak (M+H)⁺: 551/553;

R_{f} value: 0.53 (silica gel, dichloromethane/ethanol 10:1).

### EXAMPLE 1.30

### 7-(4-chloro-phenyl)-3-{2-[4-(3-aza-spiro[5.5] undec-3-ylmethyl)-phenyl]-ethyl}-3H-benzo[d][1,2,3]triazin-4-one

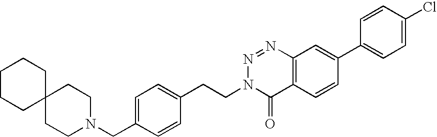

1.30.a 7-(4-chloro-phenyl)-3-{2-[4-(3-aza-spiro[5.5]undec-3-ylmethyl)-phenyl]-ethyl}-3H-benzo[d][1,2,3]triazin-4-one

Prepared analogously to Example 1.18.a from 4′-chloro-3-amino-biphenyl-4-carboxylic acid-{2-[4-(3-aza-spiro[5.5]undec-3-ylmethyl)-phenyl]-ethyl}-amide.

Yield: 0.14 g (54.9% of theory);

melting point: 165-166° C.;

C₃₂H₃₅ClN₄O (M=527.11);

calc.: molar peak (M+H)⁺: 527 fnd.: molar peak (M+H)⁺: 527;

R_{f} value: 0.56 (silica gel, dichloromethane/ethanol 10:1).

### EXAMPLE 1.31

### 6-(4-chloro-phenyl)-2-[2-(4-pyrrolidin-1-ylmethyl-phenyl)-ethyl]-2H-isoquinolin-1-one

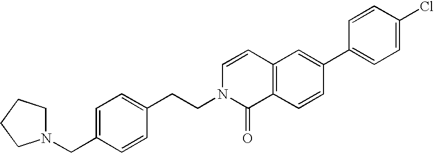

1.31.a. 2-[2-(4-bromo-phenyl)-ethoxy]-tetrahydro-pyran

0.025 g of p-toluenesulphonic acid and 2.575 ml (28.22 mmol) of dihydropyran are added successively to a solution of 4.83 g (24.02 mmol) of 2-(4-bromo-phenyl)-ethanol in 12 ml dichloromethane at 0° C. Then the reaction mixture is stirred for three hours at ambient temperature. The reaction mixture is extracted with sodium hydrogen carbonate solution and the organic phase is dried over sodium sulphate. The purification is carried out by column chromatography on Alox (eluant: cyclohexane/ethyl acetate=8:2).

Yield: 37 g (32.8% of theory);

C₁₃H₁₇BrO₂ (M=285.18);

calc.: molar peak (M)⁺: 284/286 fnd.: molar peak (M)⁺: 284/286.

1.31.b 4-[2-(tetrahydro-pyran-2-yloxy)-ethyl]-benzaldehyde

11.5 ml (18.41 mmol) of a 1.6 M n-butyllithium solution are added dropwise to a solution of 5 g (17.53 mmol) of 2-[2-(4-bromo-phenyl)-ethoxy]-tetrahydro-pyran in 80 ml of tetrahydrofuran at −70° C. and stirred for one hour at this temperature. Then 2.8 ml (36.46 mmol) of dimethylformamide are added dropwise and the reaction mixture is stirred for another two hours at −70° C. The reaction mixture is combined with ammonium chloride solution and extracted with ethyl acetate. The combined organic phases are extracted three times with saturated sodium chloride solution and dried over sodium sulphate. The purification is carried out by column chromatography on silica gel (eluant: cyclohexane/ethyl acetate=6:4).

Yield: 2.8 g (68.2% of theory);

C₁₄H₁₈O₃ (M=234.29);

calc.: molar peak (M+H)⁺: 235 fnd.: molar peak (M+H)⁺: 235;

R_{f} value: 0.57 (silica gel, petroleum ether/ethyl acetate 3:1).

1.31.c 4-(2-hydroxy-ethyl)-benzaldehyde

A solution of 2.8 g (11.95 mmol) of 4-[2-(tetrahydro-pyran-2-yloxy)-ethyl]-benzaldehyde in a mixture of 48 ml 1M hydrochloric acid and 60 ml acetone is stirred for five hours at 5° C. The reaction mixture is combined with 140 ml saturated sodium hydrogen carbonate solution and extracted with ethyl acetate. The combined organic phases are extracted three times with water and dried over sodium sulphate. The purification is carried out by column chromatography on silica gel (eluant: cyclohexane/ethyl acetate=1:1).

Yield: 1.3 g (72.4% of theory);

C₉H₁₀O₂ (M=150.17);

calc.: molar peak (M+H)⁺: 151 fnd.: molar peak (M+H)⁺: 151;

Rf value: 0.52 (silica gel, petroleum ether/ethyl acetate 1:1).

1.31.d 2-(4-[1.3]dioxane-2-yl-phenyl)-ethanol

A suspension of 9.4 g (62.59 mmol) of 4-(2-hydroxy-ethyl)-benzaldehyde, 15.83 ml (219.07 mmol) of 1,3-propanediol, 0.3 g p-toluenesulphonic acid and 150 ml of toluene is refluxed for three hours. The reaction mixture is extracted three times with saturated sodium hydrogen carbonate solution and the organic phase is dried over sodium sulphate.

Yield: 8 g (61.4% of theory);

C₁₂H₁₆O₃ (M=208.26);

calc.: molar peak (M+H)⁺: 209 fnd.: molar peak (M+H)⁺: 209.

1.31.e methanesulphonic acid-2-(4-[1.3]dioxan-2-yl-phenyl)-ethyl ester

8 g (38.41 mmol) of 2-(4-[1.3]dioxan-2-yl-phenyl)-ethanol and 10.65 ml (42.25 mmol) of triethylamine are dissolved in 300 ml dichloromethane and at 0° C. combined with 3.27 ml methanesulphonic acid chloride, dissolved in 50 ml dichloromethane. The reaction mixture is stirred for one hour at ambient temperature, extracted three times with water and the organic phase is dried over sodium sulphate. The purification is carried out by column chromatography on silica gel (eluant: petroleum ether/ethyl acetate=1:1).

Yield: 7.7 g (70% of theory);

C₁₃H₁₈O₅S (M=286.34);

calc.: molar peak (M+H)⁺: 287 fnd.: molar peak (M+H)⁺: 287;

R_{f} value: 0.49 (silica gel, petroleum ether/ethyl acetate 1:1).

1.31.f (E)-3-(3-bromo-phenyl)-acryloylazide

To a solution of 25 g (111.1 mmol) of (E)-3-(3-bromo-phenyl)-acrylic acid and 15.26 ml (110.10 mmol) of triethylamine in 800 ml acetone are added dropwise at 0° C. 11.5 ml (121.11 mmol) of ethyl chloroformate. After one hour 11.45 g (176.16 mmol) of sodium azide, dissolved in 88 ml of distilled water, are also added dropwise at 0° C. The reaction mixture is allowed to warm up to ambient temperature and then poured onto 1.3 l of ice water. The precipitate formed is filtered off, washed with water and dried at 30° C. in the circulating air dryer.

Yield: 21.1 g (76.1% of theory);

C₉H₆BrN₃O (M=252.07);

calc.: molar peak (M+H)⁺: 256/258 fnd.: molar peak (M+H)⁺: 256/258;

R_{f} value: 0.85 (silica gel, petroleum ether/ethyl acetate 1:1).

1.31.g 6-bromo-2H-isoquinolin-1-one

150 g biphenylether and 7.08 ml (29.75 mmol) of tributylamine are heated to 100° C. At this temperature 5 g (19.83 mmol) of (E)-3-(3-bromo-phenyl)-acryloylazide are added and then heated to 195-205° C. for two hours. Then the reaction mixture is left to cool and poured into cooled n-hexane. The precipitate is filtered off and washed with a mixture of cooled n-hexane and diethyl ether. Then the solid is dried in the circulating air dryer at 50° C. The solid is stirred with a mixture of diisopropylether and ethyl acetate and the drying process is repeated.

Yield: 0.6 g (13.5% of theory).

C₉H₆BrN₃O (M=224.05);

calc.: molar peak (M+H)⁺: 224/226 fnd.: molar peak (M+H)⁺: 224/226.

1.31.h 6-(4-chloro-phenyl)-2H-isoquinolin-1-one

A reaction mixture of 0.57 g (2.54 mmol) of 6-bromo-2H-isoquinolin-1-one, 0.398 g (2.54 mmol) of 4-chlorophenylboric acid, 2.6 ml of a 2M sodium carbonate solution in 20 ml dioxane and 5 ml of methanol is heated to 110° C. for two hours in the microwave. Then the reaction mixture is poured into water, the precipitate filtered off and dried in the circulating air dryer at 40° C.

Yield: 0.42 g (64.6% of theory);

C₁₅H₁₀ClNO (M=255.70);

calc.: molar peak (M+H)⁺: 256/258 fnd.: molar peak (M+H)⁺: 256/258.

R_{f} value: 0.6 (silica gel, dichloromethane/ethanol 10:1).

1.31.i 2-[2-(4-formyl-phenyl)-ethyl]-6-(4-chloro-phenyl)-2H-isoquinolin-1-one

A solution of 0.41 g (1.6 mmol) of 6-(4-chloro-phenyl)-2H-isoquinolin-1-one in 10 ml of dimethylformamide is combined with 0.18 g (1.6 mmol) of potassium tert.butoxide and stirred for 30 minutes at 50° C. Then 0.46 g (1.6 mmol) of methanesulphonic acid-2-(4-[1.3]dioxan-2-yl-phenyl)-ethyl ester is added. The reaction mixture is heated in the microwave for five hours at 180° C. and then poured onto a 10% citric acid solution. It is extracted with ethyl acetate. The organic phase is extracted three times with water and dried over sodium sulphate. The purification is carried out by column chromatography on silica gel (eluant: petroleum ether/ethyl acetate=3:1 to 1:1).

Yield: 0.15 g (24.1% of theory);

C₂₄H₁₈ClNO₂ (M=387.87);

calc.: molar peak (M+H)⁺: 388/390 fnd.: molar peak (M+H)⁺: 388/390;

R_{f} value: 0.7 (silica gel, petroleum ether/ethyl acetate 1:1).

1.31.j 6-(4-chloro-phenyl)-2-[2-(4-pyrrolidin-1-ylmethyl-phenyl)-ethyl]-2H-isoquinolin-1-one

0.14 g (0.36 mmol) of 2-[2-(4-formyl-phenyl)-ethyl]-6-(4-chloro-phenyl)-2H-isoquinolin-1-one and 0.03 ml (0.36 mmol) of pyrrolidine are dissolved in 40 ml dichloromethane. The pH is adjusted to three with glacial acetic acid. Then 0.076 g (0.36 mmol) of sodium triacetoxyborohydride are added and the mixture is stirred for 48 hours at ambient temperature. Then the reaction mixture is extracted with 2M sodium carbonate solution and dried over sodium sulphate. The purification is carried out by column chromatography on silica gel (eluant: dichloromethane/ethanol 10:1 to 1:1).

Yield: 0.04 g (25% of theory);

melting point: 136-137° C.;

C₂₈H₂₇ClN₂O (M=442.99);

calc.: molar peak (M+H)⁺: 443 fnd.: molar peak (M+H)⁺: 443;

R_{f} value: 0.5 (silica gel, dichloromethane/methanol 10:1).

The following compounds are prepared analogously to Examples 1.1 to 1.31:

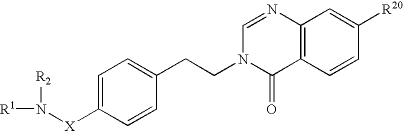

| Example | R¹R²N—X— | R²⁰ |
|---|---|---|
| 1.32 | 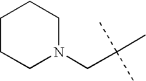 | 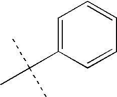 |
| 1.33 | 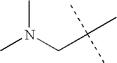 | 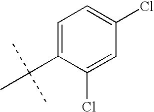 |
| 1.34 | 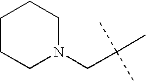 | 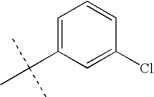 |
| 1.35 | 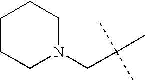 | 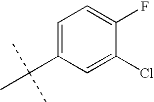 |
| 1.36 | 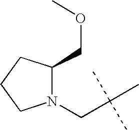 | 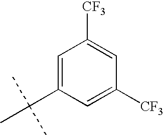 |
| 1.37 | 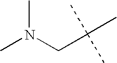 | 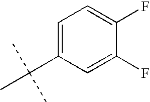 |
| 1.38 | 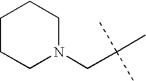 | 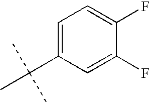 |
| 1.39 | 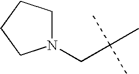 | 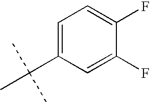 |
| 1.40 | 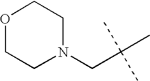 | 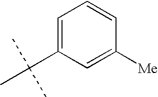 |
| 1.41 | 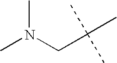 | 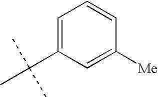 |
| 1.42 | 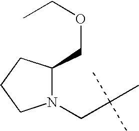 | 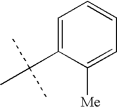 |
| 1.43 | 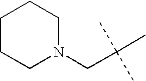 | 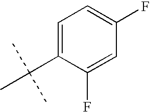 |
| 1.44 | 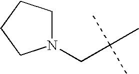 | 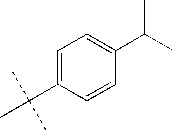 |
| 1.45 | 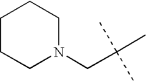 | 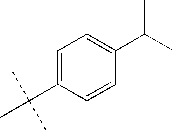 |
| 1.46 | 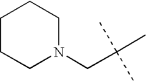 | 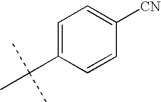 |
| 1.47 | 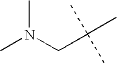 | 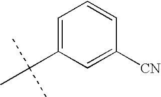 |
| 1.48 | 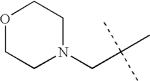 | 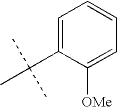 |
| 1.49 | 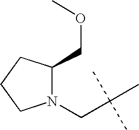 | 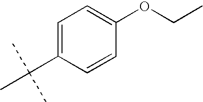 |
| 1.50 | 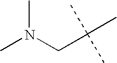 | 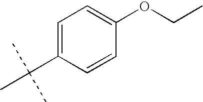 |
| 1.51 | 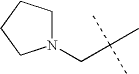 | 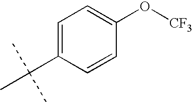 |
| 1.52 | 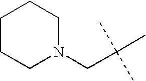 | 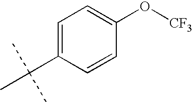 |
| 1.53 | 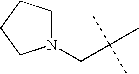 | 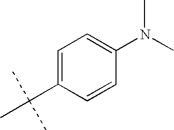 |
| 1.54 | 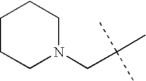 | 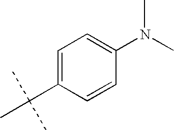 |
| 1.55 | 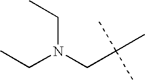 | 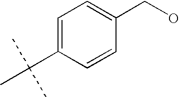 |
| 1.56 | 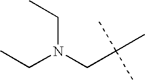 | 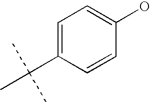 |
| 1.57 | 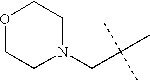 | 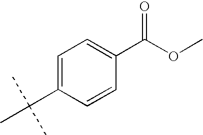 |
| 1.58 | 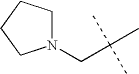 | 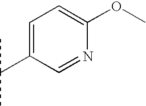 |
| 1.59 | 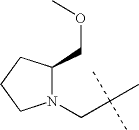 | 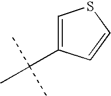 |
| 1.60 | 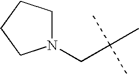 | 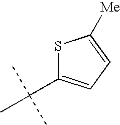 |
| 1.61 | 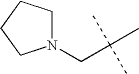 | 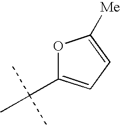 |
| 1.62 | 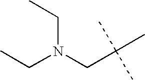 | 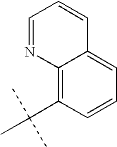 |

### EXAMPLE 2.1

### 4′-chloro-biphenyl-4-carboxylic acid [2-(4-pyrrolidin-1-ylmethyl-phenyl)-ethyl]-amide

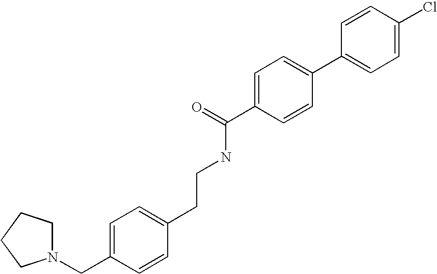

2.1.a 4′-chloro-biphenyl-4-carboxylic acid

5.83 g (29.0 mmol) of 4-bromo-benzoic acid is dissolved in 50 mL dioxane and 29 mL 2M sodium carbonate solution. 4.5 g (29.0 mmol) of 4-chlorophenylboric acid and 1.68 g (1.45 mmol) of tetrakis-(triphenylphosphine)-palladium are added successively and the reaction is refluxed for 6 h. The hot reaction solution is suction filtered through a glass fibre filter. The filtrate is extracted with ethyl acetate. The aqueous phase is acidified with citric acid and stirred for one hour at 0° C. The precipitate formed is filtered off, washed with water and dried in vacuo.

Yield: 5.1 g (75.6% of theory);

C₁₃H₉ClO₂ (M=232.668);

calc.: molar peak (M−H)⁻: 231/233 fnd.: molar peak (M−H)⁻: 231/233.

2.1.b. 4′-chloro-biphenyl-4-carboxylic acid [2-(4-pyrrolidin-1-ylmethyl-phenyl)-ethyl]-amide

471 mg (1.47 mmol) of TBTU and 0.26 mL (1.47 mmol) of Hünig base are added to a suspension of 251 mg (1.08 mmol) of 4′-chloro-biphenyl-4-carboxylic acid in 5 mL THF at ambient temperature. The reaction mixture is stirred for 10 min and then 200 mg (0.98 mmol) of 2-(4-pyrrolidin-1-ylmethyl-phenyl)-ethylamine (cf. Example 1.1.h) are added. The mixture is stirred overnight. The reaction solution is combined with saturated NaHCO₃ solution, the aqueous phase is extracted with ethyl acetate and the organic phase is dried over magnesium sulphate. The solvent is distilled off using the rotary evaporator and the residue is stirred with tert-butylmethylether while heating. The solid formed is filtered off, washed with a little tert-butylmethylether and dried in the air.

Yield: 210 mg (51.2% of theory);

C₂₆H₂₇ClN₂O (M=418.971);

calc.: molar peak (M+H)⁺: 419/421 fnd.: molar peak (M+H)⁺: 419/421;

R_{f} value: 0.57 (silica gel, dichloromethane/methanol/acetic acid 9:1:0.1).

### EXAMPLE 2.2

### 4′-chloro-biphenyl-4-carboxylic acid-[2-(4-diethylaminomethyl-phenyl)-ethyl]-amide

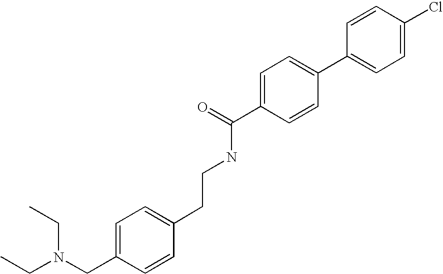

2.2.a (4-diethylaminomethyl-phenyl)-acetonitrile

0.88 mL (8.38 mmol) of diethylamine is dissolved in 30 mL acetone and 2.1 g (15.2 mmol) of potassium carbonate and 1.6 g (7.62 mmol) of (4-bromomethyl-phenyl)-acetonitrile are successively added (cf. 1.1.f). The reaction mixture is stirred for 2 h at ambient temperature, filtered through a glass frit and swashed with ethyl acetate. The filtrate is evaporated down in the rotary evaporator, extracted with water and ethyl acetate. The organic phase is dried over magnesium sulphate and the solvent is removed using the rotary evaporator. Further purification is carried out by column chromatography on silica gel (eluant: dichloromethane/methanol 9:1).

Yield: 900 mg (58.4% of theory);

C₁₃H₁₈N₂ (M=202.30);

calc.: molar peak (M+H)⁺: 203 fnd.: molar peak (M+H)⁺: 203;

R_{f} value: 0.65 (silica gel, dichloromethane/methanol 9:1).

2.2.b. 2-(4-diethylaminomethyl-phenyl)-ethylamine

A solution of 900 mg (4.45 mmol) of (4-diethylaminomethyl-phenyl)-acetonitrile in 20 mL methanolic ammonia solution is combined with 100 mg of Raney nickel and shaken at 50° C. and 5 bar in the autoclave. After the catalyst has been removed by suction filtering the solvent is removed using the rotary evaporator.

Yield: 900 mg (98.0% of theory);

C₁₃H₂₂N₂ (M=206.334);

calc.: molar peak (M+H)⁺: 207 fnd.: molar peak (M+H)⁺: 207;

R_{f} value: 0.12 (silica gel, dichloromethane/methanol/NH₃ 9:1:0.1).

2.2.c. 4′-chloro-biphenyl-4-carboxylic acid-[2-(4-diethylaminomethyl-phenyl)-ethyl]-amide

Prepared analogously to Example 2.1.b from 4′-chloro-biphenyl-4-carboxylic acid (248 mg, 1.07 mmol) and 2-(4-diethylaminomethyl-phenyl)-ethylamine (200 mg, 0.97 mmol).

Yield: 280 mg (68.6% of theory);

C₂₆H₂₉ClN₂O (M=420.987);

calc.: molar peak (M+H)⁺: 421/423 fnd.: molar peak (M+H)⁺: 421/423;

R_{f} value: 0.49 (silica gel, dichloromethane/methanol/NH₃ 9:1:0.1).

### EXAMPLE 2.3

### 4′-chloro-biphenyl-4-carboxylic acid-[2-(4-piperidin-1-ylmethyl-phenyl)-ethyl]-amide

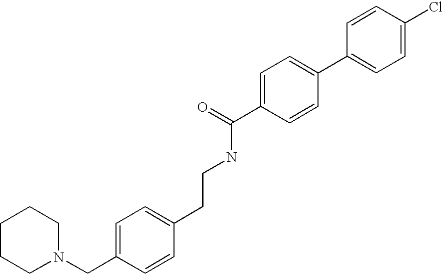

2.3.a. 4′-chloro-biphenyl-4-carboxylic acid-[2-(4-piperidin-1-ylmethyl-phenyl)-ethyl]-amide

Prepared analogously to Example 2.1.b from 4′-chloro-biphenyl-4-carboxylic acid (234 mg, 1.01 mmol) and 2-(4-piperidin-1-ylmethyl-phenyl)-ethylamine (cf. 1.15.b, 200 mg, 0.92 mmol).

Yield: 260 mg (65.6% of theory);

C₂₇H₂₉ClN₂O (M=432.998);

calc.: molar peak (M+H)⁺: 433/435 fnd.: molar peak (M+H)⁺: 433/435;

R_{f} value: 0.57 (silica gel, dichloromethane/methanol/NH₃ 9:1:0.1).

### EXAMPLE 2.4

4′-methoxy-biphenyl-4-carboxylic acid-[2-(4-diethylaminomethyl-phenyl)-ethyl]-amide

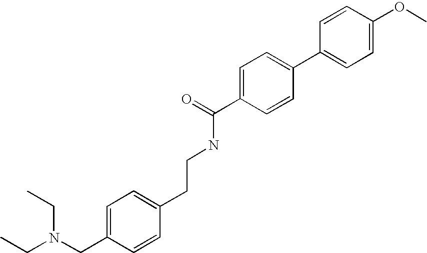

2.4.a 1-(4′-methoxy-biphenyl-4-yl)-ethanone

4-methoxybiphenyl is added to a solution of 11.3 g (85.0 mmol) of aluminium chloride in 100 mL of carbon disulphide. The mixture is heated to 40° C. and then very slowly 6.07 ml (81.4 mmol) of acetyl chloride are added. The reaction is refluxed for one hour. After cooling the reaction solution is added to 100 g of ice and 25 mL conc. hydrochloric acid. After extraction with dichloromethane the organic phase is dried over magnesium sulphate. The solvent is eliminated using the rotary evaporator and the residue is recrystallised from isopropanol.

Yield: 8.8 g (48.0% of theory);

C₁₅H₁₄O₂ (M=226.278);

calc.: molar peak (M+H)⁺: 227 fnd.: molar peak (M+H)⁺: 227.

2.4.b 4′-methoxy-biphenyl-4-carboxylic acid

6.0 mL (117 mmol) of bromine is slowly added dropwise to a solution of 15.6 g (390.9 mmol) of NaOH in 70 mL water at 0° C. Then 8.8 g (39.1 mmol) of 1-(4′-methoxy-biphenyl-4-yl)-ethanone in 50 mL dioxane is slowly added. After three hours the solid formed is filtered off, taken up in dichloromethane and filtered again. The filtrate is freed from solvent using the rotary evaporator.

Yield: 9.0 g (100.0% of theory);

C₁₅H₁₄O₂ (M=228.250);

calc.: molar peak (M−H)⁻: 227 fnd.: molar peak (M−H)⁻: 227.

2.4.c 4′-methoxy-biphenyl-4-carboxylic acid chloride

A solution of 3.0 g (0.013 mol) of 4′-methoxy-biphenyl-4-carboxylic acid in 47.4 mL (0.65 mol) of thionyl chloride is stirred at 50° C. for three hours. After removal of thionyl chloride using the rotary evaporator the product is obtained as a yellowish solid, which is stored in the refrigerator.

Yield: 3.2 g (99.8% of theory);

C₁₅H₁₄O₂ (M=246.696);

calc.: molar peak (M+H)⁺: 246/248 fnd.: molar peak (M+H)⁺: 246/248.

2.4.d 4′-methoxy-biphenyl-4-carboxylic acid-[2-(4-diethylaminomethyl-phenyl)-ethyl]-amide

287 mg (1.16 mmol) of acid chloride is added at 0° C. to a solution of 200 mg (0.97 mmol) of 2-(4-diethylaminomethyl-phenyl)-ethylamine and 0.25 mL (1.45 mmol) of Hünig base in 5 mL dichloromethane. The reaction is stirred overnight and then combined with semisaturated NaHCO₃ solution. The aqueous phase is washed with dichloromethane and the combined organic phase is dried over magnesium sulphate. After elimination of the solvent using the rotary evaporator the residue is triturated with tert-butylmethylether and the solid formed is suction filtered.

Yield: 90 mg (22.3% of theory);

C₂₇H₃₂N₂O₂ (M=416.568);

calc.: molar peak (M+H)⁺: 417 fnd.: molar peak (M+H)⁺: 417.

R_{f} value: 0.46 (silica gel, ethyl acetate/methanol/NH₃ 9:1:0.1).

### EXAMPLE 2.5

### 4′-chloro-biphenyl-4-carboxylic acid-[2-(4-diethylaminomethyl-phenyl)-ethyl]-methyl-amide

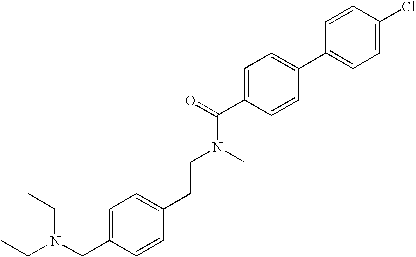

2.5.a Tert-butyl [2-(4-diethylaminomethyl-phenyl)-ethyl]-carbaminate

815 mg (3.73 mmol) of BOC-anhydride is added to a solution of 700 mg (3.93 mmol) of 2-(4-diethylaminomethyl-phenyl)-ethylamine in 5.0 mL dichloromethane and 0.52 mL (3.73 mmol) of triethylamine and stirred overnight at ambient temperature. The mixture is combined with saturated NaHCO₃ solution. The aqueous phase is washed with dichloromethane and the organic phase is dried over magnesium sulphate. After elimination of the solvent using the rotary evaporator the residue is purified by column chromatography on silica gel (eluant: dichloromethane/methanol/NH₃=9:1:0.1).

Yield: 600 mg (57.7% of theory);

C₁₈H₃₀N₂O₂ (M=306.452);

calc.: molar peak (M+H)⁺: 307 fnd.: molar peak (M+H)⁺: 307.

2.5.b [2-(4-diethylaminomethyl-phenyl)-ethyl]-methyl-amine

600 mg (1.96 mmol) of tert-butyl [2-(4-diethylaminomethyl-phenyl)-ethyl]-carbaminate in THF is slowly added dropwise to a suspension of 250 mg (6.59 mmol) of lithium aluminium hydride in 10 mL tetrahydrofuran. The reaction is stirred overnight and heated to 50° C. for a further hour. Working up is carried out by the successive addition of 0.25 mL water, 0.25 mL 15% NaOH solution and 0.75 mL water. After filtration the organic phase is dried over magnesium sulphate and the solvent is eliminated using the rotary evaporator.

Yield: 350 mg (81.1% of theory);

C₁₄H₂₄N₂ (M=220.361);

calc.: molar peak (M+H)⁺: 221 fnd.: molar peak (M+H)⁺: 221.

2.5.c 4′-chloro-biphenyl-4-carboxylic acid-[2-(4-diethylaminomethyl-phenyl)-ethyl]-methyl-amide

Prepared analogously to Example 2.1.b from 4′-chloro-biphenyl-4-carboxylic acid (222 mg, 0.95 mmol) and [2-(4-diethylaminomethyl-phenyl)-ethyl]-methyl-amine (175 mg, 0.79 mmol).

Yield: 60 mg (17.4% of theory);

C₂₇H₃₁ClN₂O (M=435.014);

calc.: molar peak (M+H)⁺: 435/437 fnd.: molar peak (M+H)⁺: 435/437;

R_{f} value: 0.39 (silica gel, ethyl acetate/methanol/NH₃ 9:1:0.1).

### EXAMPLE 2.6

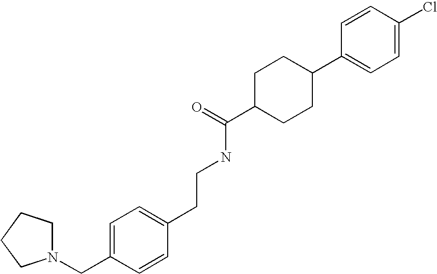

2.6.a. 4-(4-chloro-phenyl)-cyclohexanecarboxylic acid-[2-(4-pyrrolidin-1-ylmethyl-phenyl)-ethyl]-amide

Prepared according to general working method I from 4-(4-chloro-phenyl)-cyclohexanecarboxylic acid (239 mg, 1.0 mmol) and 2-(4-pyrrolidin-1-ylmethyl-phenyl)-ethylamine (204 mg, 1.0 mmol).

Yield: 65 mg (15.3% of theory);

C₂₆H₃₃ClN₂O (M=425.019);

calc.: molar peak (M+H)⁺: 425/427 fnd.: molar peak (M+H)⁺: 425/427;

R_{f} value: 0.3 (silica gel, ethyl acetate/methanol/NH₃ 9:1:0.1).

### EXAMPLE 2.7

### 4-piperidin-1-yl-N-[2-(4-pyrrolidin-1-ylmethyl-phenyl)-ethyl]-benzamide

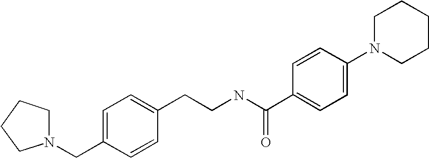

2.7.a ethyl 4-piperidin-1-yl-benzoate

0.41 mL piperidine is added to a suspension of 0.5 mL (4.13 mmol) of ethyl 4-fluoro-benzoate and 571 mg (4.13 mmol) of potassium carbonate in 20 mL DMSO. The reaction mixture is stirred overnight at 70° C., a further 1 mL (2.44 mmol) of piperidine is added and stirring is continued for a further 6 h at 70° C. After filtration water is added, the mixture is extracted with ethyl acetate, the organic phase is separated off and the solvent eliminated using the rotary evaporator. The product is further reacted without purification.

Yield: 706 mg (73.2% of theory);

C₁₄H₁₉NO₂ (M=233.313);

calc.: molar peak (M+H)⁺: 234 fnd.: molar peak (M+H)⁺: 234;

Retention time HPLC: 6.2 min (method A).

2.7.b 4-piperidin-1-yl-benzoic acid

0.78 mL (0.74 mmol) of 2N NaOH are added to a solution of 350 mg (1.50 mmol) of ethyl 4-piperidin-1-yl-benzoate in 10 mL ethanol. The reaction solution is stirred for 2 h at 60° C. and then the pH is adjusted to 6-7 with 1N HCl. The precipitate formed is dried overnight after filtration under high vacuum.

Yield: 158 mg (51.3% of theory);

C₁₂H₁₅NO₂ (M=205.259);

calc.: molar peak (M+H)⁺: 206 fnd.: molar peak (M+H)⁺: 206;

Retention time HPLC: 6.2 min (method A).

2.7.c 4-piperidin-1-yl-N-[2-(4-pyrrolidin-1-ylmethyl-phenyl)-ethyl]-benzamide

Prepared according to general working method I from 2-(4-pyrrolidin-1-ylmethyl-phenyl)-ethylamine (157 mg, 0.77 mmol) and 4-piperidin-1-yl-benzoic acid (158 mg, 0.77 mmol).

Yield: 102 mg (33.8% of theory);

C₂₅H₃₃N₃O (M=391.561);

calc.: molar peak (M+H)⁺: 392 fnd.: molar peak (M+H)⁺: 392;

Retention time HPLC: 4.4 min (method A).

### EXAMPLE 2.8

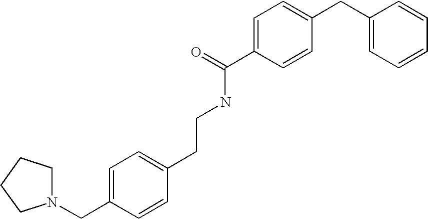

2.8.a 4-benzyl-N-[2-(4-pyrrolidin-1-ylmethyl-phenyl)-ethyl]-benzamide

Prepared according to general working method I described hereinbefore from diphenylmethane-4-carboxylic acid (104 mg, 0.49 mmol) and 2-(4-pyrrolidin-1-ylmethyl-phenyl)-ethylamine (100 mg, 0.49 mmol).

Yield: 66 mg (33.9% of theory);

C₂₇H₃₀N₂O (M=398.553);

calc.: molar peak (M+H)⁺: 399 fnd.: molar peak (M+H)⁺: 399;

R_{f} value: 0.46 (silica gel, ethyl acetate/methanol/NH₃ 9:1:0.1).

### EXAMPLE 2.9

### 4-(4-oxo-cyclohexylidenemethyl)-N-[2-(4-pyrrolidin-1-ylmethyl-phenyl)-ethyl]-benzamide

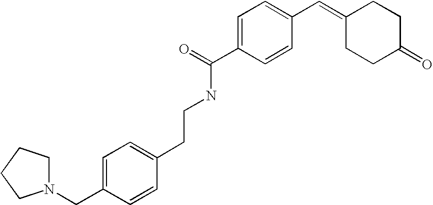

2.9.a ethyl 4-(1,4-dioxa-spiro[4.5]dec-8-ylidenemethyl)-benzoate

350 mL (0.56 mol, 1.6 M in hexane) of n-BuLi solution is added dropwise at −20° C. to a solution of 90.0 mL (0.63 mol) of diisopropylamine in 100 mL THF and the reaction solution is stirred for 30 min at −20° C. 112 g (0.37 mol) of ethyl 4-(diethoxy-phosphorylmethyl)-benzoate in 100 mL THF are slowly added dropwise. The reaction solution is stirred for 1 h at −20° C. and then 58 g (0.37 mol) of 1,4-dioxa-spiro[4.5]decan-8-one in 200 mL THF are added dropwise. The reaction solution is stirred for 30 min at −12° C. and then heated to ambient temperature over 2 h. Water is added, the aqueous phase is extracted with ether, ethyl acetate and dichloromethane. The organic phase is filtered through silica gel. After elimination of the solvent using the rotary evaporator the residue is purified by chromatography (silica gel, petroleum ether/ethyl acetate 9:1).

Yield: 80 g (72.0% of theory).

2.9.b 4-(1,4-dioxa-spiro[4.5]dec-8-ylidenemethyl)-benzoic acid

20 g NaOH in 130 mL water are added to a solution of 35 g (0.12 mol) of ethyl 4-(1,4-dioxa-spiro[4.5]dec-8-ylidenemethyl)-benzoate in 150 mL ethanol and the mixture is refluxed for 2 h. The reaction solution is added to 400 g of ice and 60 mL conc. hydrochloric acid, the aqueous phase is extracted with ethyl acetate and the solvent is eliminated using the rotary evaporator.

Yield: 32 g (91.4% of theory).

melting point: 164-165° C.

2.9.c 4-(4-oxo-cyclohexylidenemethyl)-N-[2-(4-pyrrolidin-1-ylmethyl-phenyl)-ethyl]-benzamide

Prepared according to general working method I from 4-(1,4-dioxa-spiro[4.5]dec-8-ylidenmethyl)-benzoic acid (134 mg, 0.49 mmol) and 2-(4-pyrrolidin-1-ylmethyl-phenyl)-ethylamine (100 mg, 0.49 mmol).

Yield: 57 mg (28.0% of theory);

C₂₇H₃₂N₂O₂ (M=416.568);

calc.: molar peak (M+H)⁺: 417 fnd.: molar peak (M+H)⁺: 417;

R_{f} value: 0.36 (silica gel, ethyl acetate/methanol/NH₃ 9:1:0.1).

### EXAMPLE 2.10

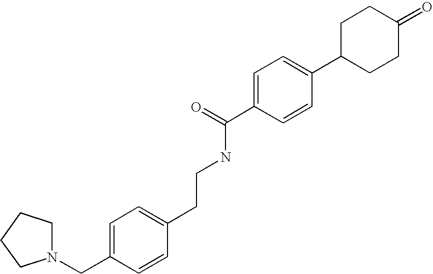

2.10.a 4-(4-oxo-cyclohexyl)-N-[2-(4-pyrrolidin-1-ylmethyl-phenyl)-ethyl]-benzamide

Prepared according to general working method I from 4-(4-oxo-cyclohexyl)-benzoic acid (128 mg, 0.49 mmol) and 2-(4-pyrrolidin-1-ylmethyl-phenyl)-ethylamine (100 mg, 0.49 mmol).

Yield: 26 mg (13.1% of theory);

C₂₆H₃₂N₂O₂ (M=404.557);

calc.: molar peak (M+H)⁺: 405 fnd.: molar peak (M+H)⁺: 405;

R_{f} value: 0.31 (silica gel, ethyl acetate/methanol/NH₃ 9:1:0.1).

### EXAMPLE 2.11

### 4-cyclohexyl-1-cyclohexylcarboxylic acid-[2-(4-pyrrolidin-1-ylmethyl-phenyl)-ethyl]-amide

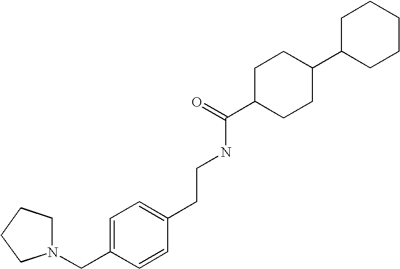

2.11.a 4-cyclohexyl-1-cyclohexylcarboxylic acid

0.44 mL conc. hydrochloric acid and 100 mg platinum oxide are added to a solution of 500 mg (2.10 mmol) of 4-(4-chlorophenyl)-cyclohexanecarboxylic acid in 10 mL methanol. The reaction mixture is stirred at 50° C. and 5 bar hydrogen for 3 h. After separation of the catalyst the solvent is eliminated using the rotary evaporator.

Yield: 440 mg (99.9% of theory);

C₁₃H₂₂O₂ (M=210.319);

calc.: molar peak (M−H)⁻: 209 fnd.: molar peak (M−H)⁻: 209.

2.11.b 4-cyclohexyl-1-cyclohexylcarboxylic acid-[2-(4-pyrrolidin-1-ylmethyl-phenyl)-ethyl]-amide

Prepared according to general working method I from bicyclohexyl-4-carboxylic acid (103 mg, 0.49 mmol) and 2-(4-pyrrolidin-1-ylmethyl-phenyl)-ethylamine (100 mg, 0.49 mmol).

Yield: 2.0 mg (1.0% of theory);

C₂₆H₄₀N₂O (M=396.622);

calc.: molar peak (M+H)⁺: 397 fnd.: molar peak (M+H)⁺: 397;

R_{f} value: 0.46 (silica gel, ethyl acetate/methanol/NH₃ 9:1:0.1).

### EXAMPLE 2.12

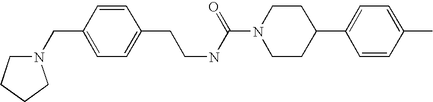

2.12.a 4-methylphenyl-piperidine-1-carboxylic acid-[2-(4-pyrrolidin-1-ylmethyl-phenyl)-ethyl]-amide

Prepared according to general working method II described hereinbefore from 4-methylphenyl-piperidine (175 mg, 1.0 mmol) and 2-(4-pyrrolidin-1-ylmethyl-phenyl)-ethylamine (204 mg, 1.0 mmol).

Yield: 90.0 mg (22.2% of theory);

C₂₆H₃₅N₃O (M=405.558);

calc.: molar peak (M+H)⁺: 406 fnd.: molar peak (M+H)⁺: 406;

R_{f} value: 0.30 (silica gel, ethyl acetate/methanol/NH₃ 9:1:0.1).

### EXAMPLE 2.13

### 4-(4-chloro-phenyl)-3.6-dihydro-2H-pyridine-1-carboxylic acid-[2-(4-pyrrolidin-1-ylmethyl-phenyl)-ethyl]-amide

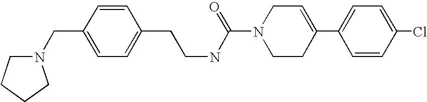

2.13.a 4-(4-chloro-phenyl)-1,2,3,6-tetrahydro-pyridine

4-chloro-methylstyrene is added dropwise at 60° C. to 100 mL (1.2 mol) of formalin solution (37% in water) and 32.1 g (0.6 mol) of ammonium chloride. The reaction mixture is stirred for 3 h at 60° C. and then cooled to ambient temperature. 100 mL methanol are added and the mixture is stirred overnight. After evaporation of the solvent using the rotary evaporator the residue is combined with 150 mL conc. hydrochloric acid and stirred for 4 h at 100° C. After cooling to ambient temperature it is added to ice and made alkaline with NaOH chips. After repeated extraction with ether the organic phase is dried over sodium sulphate. After elimination of the solvent using the rotary evaporator the residue is purified by column chromatography on silica gel (eluant: ethyl acetate:methanol:NH₃ 9:1:0.1).

Yield: 17.0 g (29.3% of theory);

C₁₁H₁₂ClN (M=193.678);

calc.: molar peak (M+H)⁺: 194 fnd.: molar peak (M+H)⁺: 194;

R_{f} value: 0.26 (silica gel, ethyl acetate/methanol/NH₃ 6:4:0.4).

2.13.b 4-(4-chloro-phenyl)-3.6-dihydro-2H-pyridine-1-carboxylic acid-[2-(4-pyrrolidin-1-ylmethyl-phenyl)-ethyl]-amide

Prepared according to general working method II from 4-(4-chloro-phenyl)-1,2,3,6-tetrahydro-pyridine (193 mg, 1.0 mmol) and 2-(4-pyrrolidin-1-ylmethyl-phenyl)-ethylamine (204 mg, 1.0 mmol).

Yield: 40.0 mg (9.4% of theory);

C₂₅H₃₀ClN₃O (M=423.990);

calc.: molar peak (M+H)⁺: 424/426 fnd.: molar peak (M+H)⁺: 424/426;

R_{f} value: 0.30 (silica gel, ethyl acetate/methanol/NH₃ 9:1:0.1).

### EXAMPLE 2.14

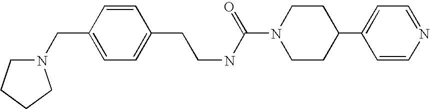

2.14.a 3,4,5,6-tetrahydro-2H-[4.4′]bipyridinyl-1-carboxylic acid-[2-(4-pyrrolidin-1-ylmethyl-phenyl)-ethyl]-amide

Prepared according to general working method II from 1,2,3,4,5,6-hexahydro-[4.4′]bipyridinyl (81 mg, 0.50 mmol) and 2-(4-pyrrolidin-1-ylmethyl-phenyl)-ethylamine (102 mg, 0.50 mmol).

Yield: 43.8 mg (22.3% of theory);

C₂₄H₃₂N₄O (M=392.549);

calc.: molar peak (M+H)⁺: 393 fnd.: molar peak (M+H)⁺: 393;

R_{f} value: 0.14 (silica gel, ethyl acetate/methanol/NH₃ 9:1:0.1).

### EXAMPLE 2.15

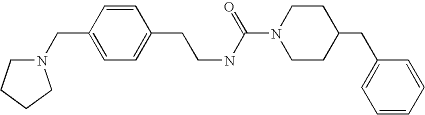

2.15.a 4-benzyl-piperidine-1-carboxylic acid-[2-(4-pyrrolidin-1-ylmethyl-phenyl)-ethyl]-amide

Prepared according to general working method II from 4-benzyl-piperidine (87.7 mg, 0.50 mmol) and 2-(4-pyrrolidin-1-ylmethyl-phenyl)-ethylamine (102 mg, 0.50 mmol).

Yield: 33.5 mg (16.5% of theory);

C₂₆H₃₅N₃O (M=405.6);

calc.: molar peak (M+H)⁺: 406 fnd.: molar peak (M+H)⁺: 406;

R_{f} value: 0.36 (silica gel, ethyl acetate/methanol/NH₃ 9:1:0.1).

### EXAMPLE 2.16

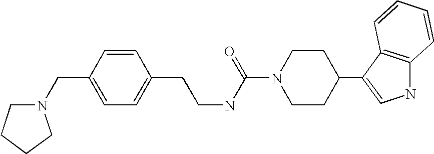

2.16.a 4-(1H-indol-3-yl)-piperidine-1-carboxylic acid-[2-(4-pyrrolidin-1-ylmethyl-phenyl)-ethyl]-amide

Prepared according to general working method II from 3-piperidin-4-yl-1H-indole (100 mg, 0.50 mmol) and 2-(4-pyrrolidin-1-ylmethyl-phenyl)-ethylamine (102 mg, 0.50 mmol).

Yield: 56.5 mg (26.2% of theory);

C₂₇H₃₄N₄O (M=430.6);

calc.: molar peak (M+H)⁺: 431 fnd.: molar peak (M+H)⁺: 431;

R_{f} value: 0.36 (silica gel, ethyl acetate/methanol/NH₃ 9:1:0.1).

### EXAMPLE 2.17

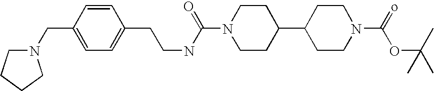

2.17.a tert-butyl 1′-[2-(4-pyrrolidin-1-ylmethyl-phenyl)-ethylcarbamoyl]-[4.4′]bipiperidinyl-1-carboxylate

Prepared according to general working method II from tert-butyl [4.4′]-bipiperidinyl-1-carboxylate (134 mg, 0.50 mmol) and 2-(4-pyrrolidin-1-ylmethyl-phenyl)-ethylamine (102 mg, 0.50 mmol).

Yield: 51.0 mg (20.5% of theory);

C₂₉H₄₆N₄O₃ (M=498.7);

calc.: molar peak (M+H)⁺: 499 fnd.: molar peak (M+H)⁺: 499;

R_{f} value: 0.40 (silica gel, ethyl acetate/methanol/NH₃ 9:1:0.1).

### EXAMPLE 2.18

### 4-cyclohexyl-piperidine-1-carboxylic acid-[2-(4-pyrrolidin-1-ylmethyl-phenyl)-ethyl]-amide

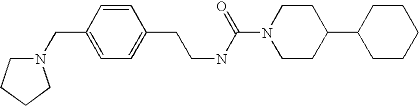

2.18.a 4-cyclohexyl-piperidine

To a solution of 1.0 g (6.4 mmol) of 4-phenylpyridin in 20 mL methanol are added 1.35 mL conc. hydrochloric acid and 200 mg platinum oxide. The reaction mixture is stirred at 50° C. and 3 bar hydrogen for 2.5 h. After separation of the catalyst the solvent is eliminated using the rotary evaporator, while the product is precipitated as the hydrochloride.

Yield: 1.2 (91.4% of theory);

C₁₁H₂₁N*HCl (M=203.758);

calc.: molar peak (M+H)⁺: 168 fnd.: molar peak (M+H)⁺: 168.

2.18.b 4-cyclohexyl-piperidine-1-carboxylic acid-[2-(4-pyrrolidin-1-ylmethyl-phenyl)-ethyl]-amide

Prepared according to general working method II from 4-cyclohexyl-piperidine (83.7 mg, 0.50 mmol) and 2-(4-pyrrolidin-1-ylmethyl-phenyl)-ethylamine (102 mg, 0.50 mmol).

Yield: 38.0 mg (19.1% of theory);

C₂₅H₃₉N₃O (M=397.6);

calc.: molar peak (M+H)⁺: 398 fnd.: molar peak (M+H)⁺: 398;

R_{f} value: 0.54 (silica gel, ethyl acetate/methanol/NH₃ 9:1:0.1).

### EXAMPLE 2.19

### 4-(4-chloro-phenyl)-piperidine-1-carboxylic acid-[2-(4-pyrrolidin-1-ylmethyl-phenyl)-ethyl]-amide

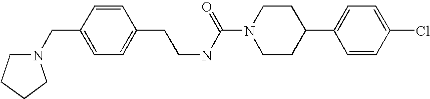

2.19.a 4-(4-chloro-phenyl)-piperidine

To a solution of 5.0 g (21.7 mmol) of 4-(4-chloro-phenyl)-1,2,3,6-tetrahydro-pyridine (cf. 2.13.a) in 20 mL methanol are added 500 mg Pd/C. The reaction mixture is stirred for 7 h at ambient temperature and 10 psi hydrogen. After separation of the catalyst the solvent is eliminated using the rotary evaporator. Further purification is carried out by column chromatography on silica gel (eluant: dichloromethane/methanol/ammonia=5:4.9:0.1).

Yield: 3.2 (75.3% of theory);

C₁₁H₁₄ClN (M=195.694);

calc.: molar peak (M+H)⁺: 196/198 fnd.: molar peak (M+H)⁺: 196/198.

R_{f} value: 0.37 (silica gel, dichloromethane/methanol/NH₃ 5:4.9:0.1).

2.19.b 4-(4-chloro-phenyl)-piperidine-1-carboxylic acid-[2-(4-pyrrolidin-1-ylmethyl-phenyl)-ethyl]-amide

Prepared according to general working method II from 4-(4-chloro-phenyl)-piperidine (97.9 mg, 0.50 mmol) and 2-(4-pyrrolidin-1-ylmethyl-phenyl)-ethylamine (102 mg, 0.50 mmol).

Yield: 9.0 mg (4.2% of theory);

C₂₅H₃₂ClN₃O (M=426.0);

calc.: molar peak (M+H)⁺: 426/428 fnd.: molar peak (M+H)⁺: 426/428;

R_{f} value: 0.49 (silica gel, ethyl acetate/methanol/NH₃ 9:1:0.1).

### EXAMPLE 2.20

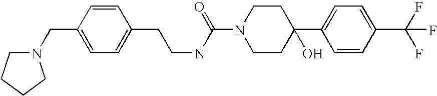

2.20.a 4-hydroxy-4-(4-trifluoromethyl-phenyl)-piperidine-1-carboxylic acid-[2-(4-pyrrolidin-1-ylmethyl-phenyl)-ethyl]-amide

Prepared according to general working method II from 4-hydroxy-4-(4-trifluoromethyl-phenyl)-piperidine (123 mg, 0.50 mmol) and 2-(4-pyrrolidin-1-ylmethyl-phenyl)-ethylamine (102 mg, 0.50 mmol).

Yield: 35.0 mg (14.7% of theory);

C₂₆H₃₂F₃N₃O₂ (M=475.6);

calc.: molar peak (M+H)⁺: 476 fnd.: molar peak (M+H)⁺: 476;

R_{f} value: 0.45 (silica gel, ethyl acetate/methanol/NH₃ 9:1:0.1).

### EXAMPLE 2.21

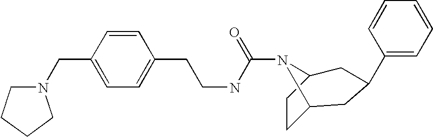

2.21.a 3-phenyl-8-aza-bicyclo[3.2.1]octane-8-carboxylic acid-[2-(4-pyrrolidin-1-ylmethyl-phenyl)-ethyl]-amide

Prepared according to general working method II from 3-phenyl-8-aza-bicyclo[3.2.1]octane (93.7 mg, 0.50 mmol) and 2-(4-pyrrolidin-1-ylmethyl-phenyl)-ethylamine (102 mg, 0.50 mmol).

Yield: 26.0 mg (12.5% of theory);

C₂₇H₃₅N₃O (M=417.6);

calc.: molar peak (M+H)⁺: 418 fnd.: molar peak (M+H)⁺: 418;

R_{f} value: 0.51 (silica gel, ethyl acetate/methanol/NH₃ 9:1:0.1).

### EXAMPLE 2.22

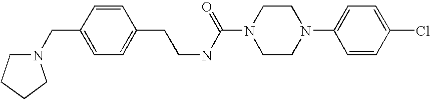

2.22.a 4-(4-chloro-phenyl)-piperazine-1-carboxylic acid-[2-(4-pyrrolidin-1-ylmethyl-phenyl)-ethyl]-amide

Prepared according to general working method II from 4-(4-chloro-phenyl)-piperazine (117 mg, 0.50 mmol) and 2-(4-pyrrolidin-1-ylmethyl-phenyl)-ethylamine (102 mg, 0.50 mmol).

Yield: 13.0 mg (6.1% of theory);

C₂₄H₃₁ClN₄O (M=427.0);

calc.: molar peak (M+H)⁺: 427/429 fnd.: molar peak (M+H)⁺: 427/429;

R_{f} value: 0.42 (silica gel, ethyl acetate/methanol/NH₃ 9:1:0.1).

### EXAMPLE 2.23

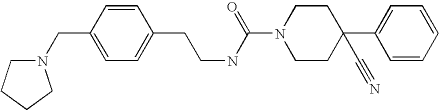

2.23.a 4-cyano-4-phenyl-piperidine-1-carboxylic acid-[2-(4-pyrrolidin-1-ylmethyl-phenyl)-ethyl]-amide

Prepared according to general working method II from 4-cyano-4-phenyl-piperidine (111 mg, 0.50 mmol) and 2-(4-pyrrolidin-1-ylmethyl-phenyl)-ethylamine (102 mg, 0.50 mmol).

Yield: 27.0 mg (13.0% of theory);

C₂₆H₃₂N₄O (M=416.6);

calc.: molar peak (M+H)⁺: 417 fnd.: molar peak (M+H)⁺: 417;

R_{f} value: 0.46 (silica gel, ethyl acetate/methanol/NH₃ 9:1:0.1).

### EXAMPLE 2.24

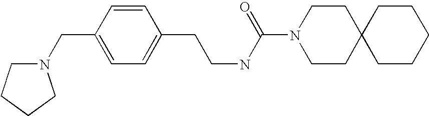

2.24.a 3-Aza-spiro[5.5]undecane-3-carboxylic acid-[2-(4-pyrrolidin-1-ylmethyl-phenyl)-ethyl]-amide

Prepared according to general working method II from 3-aza-spiro[5.5]undecane (76.7 mg, 0.50 mmol) and 2-(4-pyrrolidin-1-ylmethyl-phenyl)-ethylamine (102 mg, 0.50 mmol).

Yield: 24.0 mg (12.5% of theory);

C₂₄H₃₇N₃O (M=383.6);

calc.: molar peak (M+H)⁺: 384 fnd.: molar peak (M+H)⁺: 384;

R_{f} value: 0.49 (silica gel, ethyl acetate/methanol/NH₃ 9:1:0.1).

### EXAMPLE 2.25

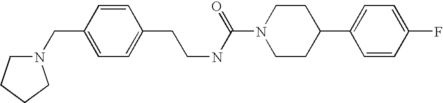

2.25.a 4-(4-fluoro-phenyl)-piperidine-1-carboxylic acid-[2-(4-pyrrolidin-1-ylmethyl-phenyl)-ethyl]-amide

Prepared according to general working method II from 4-(4-fluoro-phenyl)-piperidine (108 mg, 0.50 mmol) and 2-(4-pyrrolidin-1-ylmethyl-phenyl)-ethylamine (102 mg, 0.50 mmol).

Yield: 32.0 mg (15.6% of theory);

C₂₅H₃₂FN₃O (M=409.6);

calc.: molar peak (M+H)⁺: 410 fnd.: molar peak (M+H)⁺: 410;

R_{f} value: 0.50 (silica gel, ethyl acetate/methanol/NH₃ 9:1:0.1).

### EXAMPLE 2.26

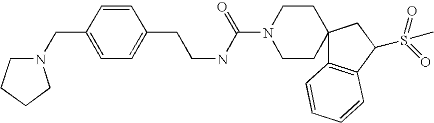

2.26.a 1.2-dihydro-1-(methylsulphonyl)-spiro[3H-indole-3,4′-piperidine]-1-carboxylic acid-[2-(4-pyrrolidin-1-ylmethyl-phenyl)-ethyl]-amide

Prepared according to general working method II from 1,2-dihydro-1-(methylsulphonyl)-spiro[3H-indole-3,4′-piperidine] (133.2 mg, 0.50 mmol) and 2-(4-pyrrolidin-1-ylmethyl-phenyl)-ethylamine (102 mg, 0.50 mmol).

Yield: 28.0 mg (11.3% of theory);

C₂₇H₃₆N₄O₃S (M=496.7);

calc.: molar peak (M+H)⁺: 497 fnd.: molar peak (M+H)⁺: 497;

R_{f} value: 0.42 (silica gel, ethyl acetate/methanol/NH₃ 9:1:0.1).

### EXAMPLE 2.27

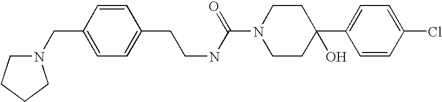

2.27.a 4-(4-chloro-phenyl)-4-hydroxy-piperidine-1-carboxylic acid-[2-(4-pyrrolidin-1-ylmethyl-phenyl)-ethyl]-amide

Prepared according to general working method II from 4-(4-chloro-phenyl)-4-hydroxy-piperidine (106 mg, 0.50 mmol) and 2-(4-pyrrolidin-1-ylmethyl-phenyl)-ethylamine (102 mg, 0.50 mmol).

Yield: 32.0 mg (14.5% of theory);

C₂₅H₃₂ClN₃O₂ (M=442.0);

calc.: molar peak (M+H)⁺: 442/444 fnd.: molar peak (M+H)⁺: 442/444;

R_{f} value: 0.44 (silica gel, ethyl acetate/methanol/NH₃ 9:1:0.1).

### EXAMPLE 2.28

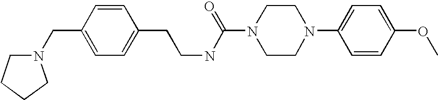

2.28.a 4-(4-methoxy-phenyl)-piperazine-1-carboxylic acid-[2-(4-pyrrolidin-1-ylmethyl-phenyl)-ethyl]-amide

Prepared according to general working method II from 4-(4-methoxy-phenyl)-piperazine (133 mg, 0.50 mmol) and 2-(4-pyrrolidin-1-ylmethyl-phenyl)-ethylamine (102 mg, 0.50 mmol).

Yield: 35.0 mg (16.6% of theory);

C₂₅H₃₄N₄O₂ (M=422.6);

calc.: molar peak (M+H)⁺: 423 fnd.: molar peak (M+H)⁺: 423;

R_{f} value: 0.47 (silica gel, ethyl acetate/methanol/NH₃ 9:1:0.1).

### EXAMPLE 2.29

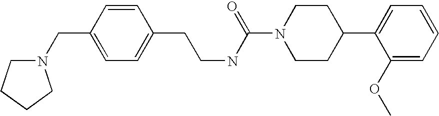

2.29. 4-(2-methoxy-phenyl)-piperidine-1-carboxylic acid-[2-(4-pyrrolidin-1-ylmethyl-phenyl)-ethyl]-amide

Prepared according to general working method II from 4-(2-methoxy-phenyl)-piperidine (114 mg, 0.50 mmol) and 2-(4-pyrrolidin-1-ylmethyl-phenyl)-ethylamine (102 mg, 0.50 mmol).

Yield: 20.0 mg (9.5% of theory);

C₂₆H₃₅N₃O₂ (M=421.6);

calc.: molar peak (M+H)⁺: 422 fnd.: molar peak (M+H)⁺: 422;

R_{f} value: 0.55 (silica gel, ethyl acetate/methanol/NH₃ 9:1:0.1).

### EXAMPLE 2.30

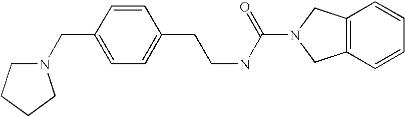

2.30.a 1,3-dihydro-isoindole-2-carboxylic acid-[2-(4-pyrrolidin-1-ylmethyl-phenyl)-ethyl]-amide

Prepared according to general working method II from 1,3-dihydro-isoindole (77.8 mg, 0.50 mmol) and 2-(4-pyrrolidin-1-ylmethyl-phenyl)-ethylamine (102 mg, 0.50 mmol).

Yield: 13.0 mg (7.4% of theory);

C₂₂H₂₇N₃O (M=349.48);

calc.: molar peak (M+H)⁺: 350 fnd.: molar peak (M+H)⁺: 350;

R_{f} value: 0.30 (silica gel, dichloromethane/methanol/NH₃ 9:1:0.1).

### EXAMPLE 2.31

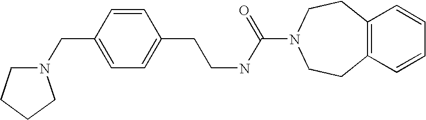

2.31.a 1,2,4,5-tetrahydro-benzo[d]azepine-3-carboxylic acid-[2-(4-pyrrolidin-1-ylmethyl-phenyl)-ethyl]-amide

Prepared according to general working method II from 1,2,4,5-tetrahydro-benzo[d]azepine (73.6 mg, 0.50 mmol) and 2-(4-pyrrolidin-1-ylmethyl-phenyl)-ethylamine (102 mg, 0.50 mmol).

Yield: 12.0 mg (6.4% of theory);

C₂₄H₃₁N₃O (M=377.534);

calc.: molar peak (M+H)⁺: 378 fnd.: molar peak (M+H)⁺: 378;

R_{f} value: 0.33 (silica gel, dichloromethane/methanol/NH₃ 9:1:0.1).

### EXAMPLE 2.32

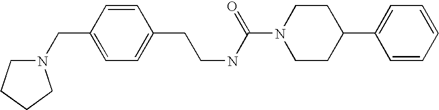

2.32.a 4-phenyl-piperidine-1-carboxylic acid-[2-(4-pyrrolidin-1-ylmethyl-phenyl)-ethyl]-amide

Prepared according to general working method II from 4-phenyl-piperidine (80.6 mg, 0.50 mmol) and 2-(4-pyrrolidin-1-ylmethyl-phenyl)-ethylamine (102 mg, 0.50 mmol).

Yield: 24.0 mg (12.3% of theory);

C₂₅H₃₃N₃O (M=391.561);

calc.: molar peak (M+H)⁺: 392 fnd.: molar peak (M+H)⁺: 392;

R_{f} value: 0.35 (silica gel, dichloromethane/methanol/NH₃ 9:1:0.1).

### EXAMPLE 2.33

### 4-(4-dimethylaminomethyl-phenyl)-piperidine-1-carboxylic acid-[2-(4-pyrrolidin-1-ylmethyl-phenyl)-ethyl]-amide

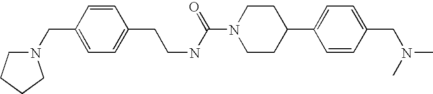

2.33.a tert.butyl 4-(4-dimethylaminomethyl-phenyl)-4-hydroxy-piperidine-1-carboxylate

236 mL (0.38 mol, 1.6M in hexane) n-BuLi is added dropwise over 35 min at −65° C. to a solution of 81 g (0.38 mol) of 4-bromodimethylbenzylamine in 450 mL THF. 75 g (0.38 mol) of tert.butyl 4-oxo-piperidine-1-carboxylate in 150 mL THF are added dropwise over 60 min, so that the temperature does not exceed −60° C. The reaction solution is stirred for 2 h at −65° C. and for a further 17 h at ambient temperature. The reaction mixture is combined with 300 mL ether, cooled to 5° C. and the precipitate formed is suction filtered. The precipitate is combined with 200 mL water and 700 mL ether and stirred for 10 min. The organic phase is dried over magnesium sulphate and the solvent eliminated using the rotary evaporator. The product obtained is dried in vacuo.

Yield: 45 g (35.7% of theory).

2.33.b dimethyl-[4-(1,2,3,6-tetrahydro-pyridin-4-yl)-benzyl]-amine

70 mL trifluoroacetic acid is added dropwise to a solution of 45 g (0.14 mol) of tert.butyl 4-(4-dimethylaminomethyl-phenyl)-4-hydroxy-piperidine-1-carboxylate in 140 mL dichloromethane at −10° C. The solution is stirred for 1.5 h at ambient temperature, cooled to −10° C. and 30 mL conc. sulphuric acid are added. After half an hour a further 10 mL sulphuric acid are added. After 1 h the solvent is eliminated using the rotary evaporator and added to 300 g of ice. The pH is adjusted to 14 with 6 N NaOH solution. The aqueous phase is saturated with potassium carbonate and extracted twice with ether. The combined organic phases are concentrated to dryness using the rotary evaporator.

Yield: 25.2 g (86.9%).

2.33.c dimethyl-(4-piperidin-4-yl-benzyl)-amine

6 g Pd/BaSO₄ are added to a solution of 16 g (74 mmol) of dimethyl-[4-(1,2,3,6-tetrahydro-pyridin-4-yl)-benzyl]-amine in 200 mL methanol. The solution is stirred for 1 h at ambient temperature in a hydrogen atmosphere, the catalyst is filtered off and the solvent eliminated using the rotary evaporator. The residue is dissolved in methanol, methanolic hydrochloric acid is added and then ether is added until the mixture becomes cloudy. After storage at −20° C. the hydrochloride obtained is suction filtered.

Yield: 16 g (84.9%).

2.33.d 4-(4-dimethylaminomethyl-phenyl)-piperidine-1-carboxylic acid-[2-(4-pyrrolidin-1-ylmethyl-phenyl)-ethyl]-amide

Prepared according to general working method II from 4-(4-dimethylaminomethyl-phenyl)-piperidine (127 mg, 0.50 mmol) and 2-(4-pyrrolidin-1-ylmethyl-phenyl)-ethylamine (102 mg, 0.50 mmol).

Yield: 37.0 mg (16.5% of theory);

C₂₈H₄₀N₄O (M=448.657);

calc.: molar peak (M+H)⁺: 449 fnd.: molar peak (M+H)⁺: 449;

R_{f} value: 0.37 (silica gel, dichloromethane/methanol/NH₃ 9:1:0.1).

### EXAMPLE 2.34

4′-chloro-biphenyl-4-yl)-[3-(4-pyrrolidin-1-ylmethyl-phenyl)-piperidin-1-yl]-methanone

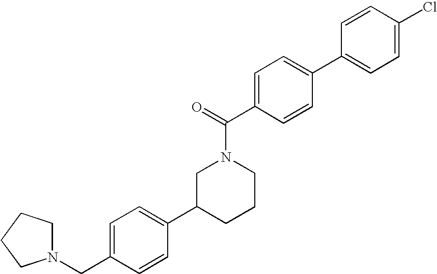

2.34.a 1-(4-Bromo-benzyl)-pyrrolidine

20.0 g (0.080 mol) of 4-bromobenzylbromide in THF is slowly added dropwise to a solution of 13.1 ml (0.16 mmol) of pyrrolidine and 200 mL tetrahydrofuran, so that the temperature does not exceed 20° C. The reaction solution is stirred overnight and after mixing with ice acidified with concentrated hydrochloric acid. After extraction with ether the aqueous phase is made alkaline with sodium hydroxide solution and saturated with potassium carbonate. After extraction with ether the organic phase is dried over magnesium sulphate and the solvent is eliminated using the rotary evaporator.

Yield: 18.1 g (94.2% of theory);

C₁₁H₁₄BrN (M=240.145);

calc.: molar peak (M+H)⁺: 240/242 fnd.: molar peak (M+H)⁺: 240/242;

R_{f} value: 0.19 (silica gel, petroleum ether/ethyl acetate 8:2).

2.34.b 3-(4-pyrrolidin-1-ylmethyl-phenyl)-pyridine

1.11 g (4.64 mmol) of 1-(4-bromo-benzyl)-pyrrolidine is dissolved in 10 mL dioxane and 5 mL 2M sodium carbonate solution. 570 mg (4.64 mmol) of pyridine-3-boric acid and 270 mg (0.23 mmol) of tetrakis-(triphenylphosphine)-palladium are added successively and the reaction is refluxed for 6 h. The reaction solution is suction filtered through a glass fibre filter. The filtrate is extracted several times with ethyl acetate. The organic phase is dried over magnesium sulphate and the solvent is eliminated using the rotary evaporator. Further purification is carried out by column chromatography on silica gel (eluant: ethyl acetate/methanol/NH₃=8:2:0.1).

Yield: 500 mg (45.2% of theory);

C₁₆H₁₈N₂ (M=238.335);

calc.: molar peak (M+H)⁺: 239 fnd.: molar peak (M+H)⁺: 239.

2.34.c 3-(4-pyrrolidin-1-ylmethyl-phenyl)-piperidine

4 mL 1M hydrochloric acid and 200 mg platinum oxide are added to a solution of 500 mg (2.10 mmol) of 3-(4-pyrrolidin-1-ylmethyl-phenyl)-pyridine in 10 mL ethanol. The reaction mixture is stirred at ambient temperature and 3 bar hydrogen for 4.5 h. After separation of the catalyst the solvent is eliminated using the rotary evaporator, while the product is precipitated as the hydrochloride.

Yield: 600 mg (100% of theory);

C₁₆H₂₄N₂*HCl (M=280.844);

calc.: molar peak (M+H)⁺: 245 fnd.: molar peak (M+H)⁺: 245.

2.34.d (4′-chloro-biphenyl-4-yl)-[3-(4-pyrrolidin-1-ylmethyl-phenyl)-piperidin-1-yl]-methanone

Prepared according to general working method I from 4′-chloro-biphenyl-4-carboxylic acid (183 mg, 0.78 mmol) and 3-(4-pyrrolidin-1-ylmethyl-phenyl)-piperidine (200 mg, 0.71 mmol).

Yield: 20.0 mg (6.1% of theory);

C₂₉H₃₁ClN₂O (M=459.036);

calc.: molar peak (M+H)⁺: 459/461 fnd.: molar peak (M+H)⁺: 459/461;

R_{f} value: 0.58 (silica gel, ethyl acetate/methanol/NH₃ 9:1:0.1).

### EXAMPLE 2.35

### 4′-chloro-biphenyl-4-carboxylic acid-[2-methyl-2-(4-pyrrolidin-1-ylmethyl-phenyl)-propyl]-amide

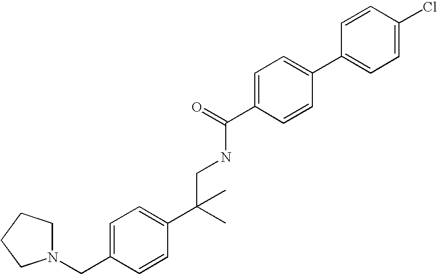

2.35.a 2-methyl-2-(4-pyrrolidin-1-ylmethyl-phenyl)-propionitrile

3.4 g (30 mmol) of potassium-tert-butoxide are added to a solution of 2.0 g (10.0 mmol) of (4-pyrrolidin-1-ylmethyl-phenyl)-acetonitrile (cf. 1.1.g) in 50 mL tetrahydrofuran at ambient temperature. The reaction solution is briefly stirred, combined with 1.9 mL (30 mmol) of methyl iodide, stirred for a further 2 h at ambient temperature and then evaporated to dryness using the rotary evaporator. The residue is distributed between water and ethyl acetate, the organic phase is washed with water and dried over magnesium sulphate. The solvent is removed using the rotary evaporator and the crude product is further reacted without purification.

Yield: 1.4 g (61.3% of theory);

C₁₅H₂₀N₂ (M=228.340);

calc.: molar peak (M+H)⁺: 229 fnd.: molar peak (M+H)⁺: 229;

R_{f} value: 0.40 (silica gel, ethyl acetate/methanol/NH₃ 9:1:0.1).

2.35. b 2-methyl-2-(4-pyrrolidin-1-ylmethyl-phenyl)-propylamine

150 mg of Raney nickel are added to a solution of 1.4 g (6.13 mmol) of 2-methyl-2-(4-pyrrolidin-1-ylmethyl-phenyl)-propionitrile in 20 mL methanolic ammonia solution. The reaction mixture is stirred overnight at 50° C. under 5 bar hydrogen atmosphere. After the catalyst has been filtered off the solvent is eliminated using the rotary evaporator.

Yield: 1.4 g (98.3% of theory);

C₁₅H₂₄N₂ (M=232.372);

calc.: molar peak (M+H)⁺: 233 fnd.: molar peak (M+H)⁺: 233;

R_{f} value: 0.30 (silica gel, ethyl acetate/methanol/NH₃ 9:1:0.1).

2.35.c. 4′-chloro-biphenyl-4-carboxylic acid-[2-methyl-2-(4-pyrrolidin-1-ylmethyl-phenyl)-propyl]-amide

Prepared according to general working method I from 4′-chloro-biphenyl-4-carboxylic acid (233 mg, 1.0 mmol) and 2-methyl-2-(4-pyrrolidin-1-ylmethyl-phenyl)-propylamine (232 mg, 1.0 mmol).

Yield: 400 mg (89.5% of theory);

C₂₈H₃₁ClN₂O (M=447.025);

calc.: molar peak (M+H)⁺: 447/449 fnd.: molar peak (M+H)⁺: 447/449;

R_{f} value: 0.35 (silica gel, ethyl acetate/methanol/NH₃ 9:1:0.1).

### EXAMPLE 2.36

### 4′-chloro-biphenyl-4-carboxylic acid-[2-(4-pyrrolidin-1-ylmethyl-phenyl)-propyl]-amide

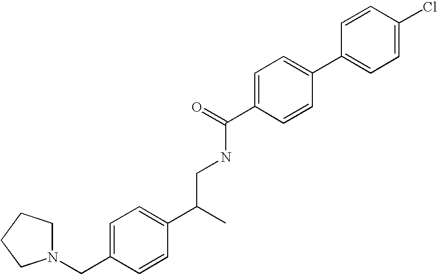

2.36.a 2-(4-pyrrolidin-1-ylmethyl-phenyl)-propionitrile

1.12 g (10 mmol) of potassium-tert-butoxide are added to a solution of 2.0 g (10.0 mmol) of (4-pyrrolidin-1-ylmethyl-phenyl)-acetonitrile (cf. 1.1.g) in 50 mL tetrahydrofuran at ambient temperature. The reaction solution is stirred for 30 min and then combined with 0.63 mL (10 mmol) of methyl iodide. The reaction is stirred for 1 h at 50° C. and then concentrated to dryness using the rotary evaporator. The residue is distributed between water and ethyl acetate, the organic phase is washed twice with water and dried over magnesium sulphate. The solvent is removed using the rotary evaporator and the crude product, which contains approx. 20% of the dimethylated compound, is further reacted without purification.

Yield: 0.5 g (23.3% of theory);

C₁₄H₁₈N₂ (M=214.313);

calc.: molar peak (M+H)⁺: 215 fnd.: molar peak (M+H)⁺: 215;

R_{f} value: 0.40 (silica gel, ethyl acetate/methanol/NH₃ 9:1:0.1).

2.36.b 2-(4-pyrrolidin-1-ylmethyl-phenyl)-propylamine

100 mg of Raney nickel are added to a solution of 400 mg (1.87 mmol) of 2-(4-pyrrolidin-1-ylmethyl-phenyl)-propionitrile in 20 mL methanolic ammonia solution. The reaction mixture is stirred overnight at 50° C. and 5 bar hydrogen atmosphere. After the catalyst has been filtered off the solvent is eliminated using the rotary evaporator. The amine, which contains approx. 20% of dimethylated compound, is further reacted without any more purification.

Yield: 0.4 g (98.6% of theory);

C₁₅H₂₂N₂ (M=218.345);

calc.: molar peak (M+H)⁺: 219 fnd.: molar peak (M+H)⁺: 219;

R_{f} value: 0.30 (silica gel, ethyl acetate/methanol/NH₃ 9:1:0.1).

2.36.c 4′-chloro-biphenyl-4-carboxylic acid-[2-(4-pyrrolidin-1-ylmethyl-phenyl)-propyl]-amide

Prepared according to general working method I from 4′-chloro-biphenyl-4-carboxylic acid (233 mg, 1.0 mmol) and 2-(4-pyrrolidin-1-ylmethyl-phenyl)-propylamine (218 mg, 1.0 mmol).

Yield: 10 mg (2.3% of theory);

C₂₈H₃₁ClN₂O (M=447.025);

calc.: molar peak (M+H)⁺: 447/449 fnd.: molar peak (M+H)⁺: 447/449;

R_{f} value: 0.35 (silica gel, ethyl acetate/methanol/NH₃ 9:1:0.1).

### EXAMPLE 2.37

### 4′-Chloro-biphenyl-4-carboxylic acid-(4-pyrrolidin-1-ylmethyl-benzyloxy)-amide

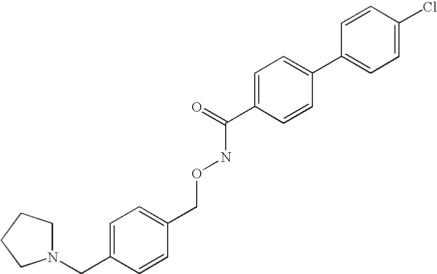

2.37.a 2-(4-pyrrolidin-1-ylmethyl-benzyloxy)-isoindol-1,3-dione

A mixture of 8.2 g (50 mmol) of N-hydroxy-phthalimide and 8.7 mL (50 mmol) of Hünig base in 125 mL acetonitrile is added at ambient temperature to a solution of 13.2 g (50 mmol) of α,α′-dibromo-p-xylene in 125 mL acetonitrile. The reaction solution is stirred for 10 min, then 4.1 mL (50 mmol) of pyrrolidine are added and stirring is continued for one hour. After filtration the mother liquor is evaporated to dryness using the rotary evaporator. The residue is purified by chromatography on silica gel (eluant: ethyl acetate/methanol/ammonia). The substance was further reacted immediately after purification.

Yield: 1.0 g (5.9% of theory);

R_{f} value: 0.60 (Alox, ethyl acetate/petroleum ether 1:1).

2.37.b O-(4-pyrrolidin-1-ylmethyl-benzyl)-hydroxylamine

50 mL 40% methylamine solution in water are added to a solution of 1.0 g (2.97 mmol) of 2-(4-pyrrolidin-1-ylmethyl-benzyloxy)-isoindol-1,3-dione in 50 mL toluene and the mixture is stirred for 2.5 days at ambient temperature. After separation of the organic phase the aqueous phase is extracted twice with tert-butylmethylether. The combined organic phases are washed with water and dried over magnesium sulphate. The solvent is eliminated using the rotary evaporator and the resulting product is further reacted without purification.

Yield: 260 mg (42.4% of theory);

C₁₂H₁₈N₂O (M=206.290);

calc.: molar peak (M+H)⁺: 207 fnd.: molar peak (M+H)⁺: 207.

2.37.c 4′-chloro-biphenyl-4-carboxylic acid-(4-pyrrolidin-1-ylmethyl-benzyloxy)-amide

Prepared according to general working method I from 4′-chloro-biphenyl-4-carboxylic acid (116 mg, 0.5 mmol) and O-(4-pyrrolidin-1-ylmethyl-benzyl)-hydroxylamine (103 mg, 0.5 mmol).

Yield: 10.0 mg (4.8% of theory);

C₂₀H₂₅ClN₂O₂ (M=420.943);

calc.: molar peak (M+H)⁺: 421/423 fnd.: molar peak (M+H)⁺: 421/423;

R_{f} value: 0.38 (silica gel, ethyl acetate/methanol/NH₃ 9:1:0.1).

### EXAMPLE 2.38

### 4′-chloro-biphenyl-4-carboxylic acid-[1,1-dimethyl-2-(4-pyrrolidin-1-ylmethyl-phenyl)-ethyl]-amide

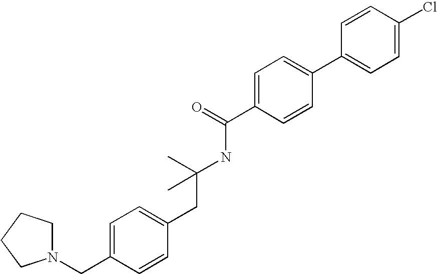

2.38.a ethyl (4-pyrrolidin-1-ylmethyl-phenyl)-acetate

3.0 g (15 mmol) of (4-pyrrolidin-1-ylmethyl-phenyl)-acetonitrile (cf. 1.1.g) is dissolved in ethanolic hydrochloric acid (saturated) and refluxed for 4 hours. The solvent is eliminated using the rotary evaporator and the residue is taken up with dilute NaHCO₃ solution and tert-butylmethylether. The organic phase is dried with sodium sulphate, suction filtered through activated charcoal and then the solvent is eliminated using the rotary evaporator.

Yield: 3.4 g (91.6% of theory);

C₁₅H₂₁NO₂ (M=247.340);

calc.: molar peak (M+H)⁺: 248 fnd.: molar peak (M+H)⁺: 248;

R_{f} value: 0.25 (silica gel, ethyl acetate/methanol/NH₃ 9:1:0.1).

2.38.b 2-methyl-1-(4-pyrrolidin-1-ylmethyl-phenyl)-propan-2-ol

3.4 g (13.8 mmol) of ethyl (4-pyrrolidin-1-ylmethyl-phenyl)-acetate in 20 mL tetrahydrofuran is added dropwise to 13.3 mL (40 mmol) of a 3.0 M methylmagnesium chloride solution in tetrahydrofuran at ambient temperature. The temperature rises to 40° C. The reaction mixture is stirred for one hour and then poured onto 100 mL ammonium chloride solution. The aqueous phase is extracted several times with dichloromethane. The combined organic phases are washed with saturated saline solution and dried over magnesium sulphate. The solvent is eliminated using the rotary evaporator and the residue is purified by column chromatography on Alox (activity 2-3) (eluant: cyclohexane: ethyl acetate 4:1).

Yield: 800 mg (24.9% of theory);

C₁₅H₂₃NO (M=233.357);

calc.: molar peak (M+H)⁺: 234 fnd.: molar peak (M+H)⁺: 234;

R_{f} value: 0.50 (Alox, petroleum ether/ethyl acetate 6:4).

2.38.c N-[1,1-dimethyl-2-(4-pyrrolidin-1-ylmethyl-phenyl)-ethyl]-formamide

A mixture of 2 mL sulphuric acid and 1 ml glacial acetic acid is added dropwise to a solution of 250 mg (5.0 mmol) of sodium cyanide in 2 mL glacial acetic acid, so that the temperature of the reaction mixture does not exceed 20° C. Then 800 mg (3.43 mmol) of 2-methyl-1-(4-pyrrolidin-1-ylmethyl-phenyl)-propan-2-ol in 2 mL glacial acetic acid are added dropwise. The temperature is kept below 20° C. The reaction solution is stirred for one hour at ambient temperature and then poured onto ice and neutralised with sodium carbonate solution. The aqueous phase is extracted with ether and the organic phase is dried over magnesium sulphate. The solvent is eliminated using the rotary evaporator and the product is further reacted without purification.

Yield: 520 mg (58.2% of theory);

C₁₆H₂₄N₂O (M=260.382);

calc.: molar peak (M+H)⁺: 261 fnd.: molar peak (M+H)⁺: 261.

2.38.d. 1,1-dimethyl-2-(4-pyrrolidin-1-ylmethyl-phenyl)-ethylamine

25 mL conc. hydrochloric acid are added to a solution of 520 mg (2 mmol) of N-[1,1-dimethyl-2-(4-pyrrolidin-1-ylmethyl-phenyl)-ethyl]-formamide in 10 mL ethanol and the mixture is refluxed overnight. After cooling the reaction solution is made alkaline with 25% aqueous sodium hydroxide solution and the aqueous phase is extracted several times with tert-butylmethylether. The combined organic phases are washed with water, dried over magnesium sulphate and filtered through activated charcoal. The solvent is eliminated using the rotary evaporator.

Yield: 380 mg (81.8% of theory);

C₁₅H₂₄N₂ (M=232.372);

calc.: molar peak (M+H)⁺: 233 fnd.: molar peak (M+H)⁺: 233;

R_{f} value: 0.10 (silica gel, ethyl acetate/methanol/NH₃ 9:1:0.1).

2.38.e 4′-chloro-biphenyl-4-carboxylic acid-[1,1-dimethyl-2-(4-pyrrolidin-1-ylmethyl-phenyl)-ethyl]-amide

Prepared according to general working method I from 4′-chloro-biphenyl-4-carboxylic acid (116 mg, 0.5 mmol) and 1,1-dimethyl-2-(4-pyrrolidin-1-ylmethyl-phenyl)-ethylamine (116 mg, 0.5 mmol).

Yield: 73.0 mg (32.7% of theory);

C₂₈H₃₁ClN₂O₂ (M=447.025);

calc.: molar peak (M+H)⁺: 447/449 fnd.: molar peak (M+H)⁺: 447/449;

R_{f} value: 0.48 (silica gel, ethyl acetate/methanol/NH₃ 9:1:0.1).

### EXAMPLE 2.39

### 4′-chloro-biphenyl-4-carboxylic acid-4-(2-pyrrolidin-1-yl-ethyl)-benzylamide

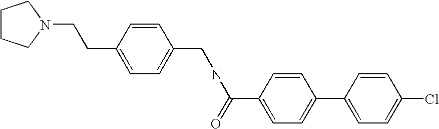

2.39.a 4-(2-pyrrolidin-1-yl-ethyl)-benzonitrile

91 mg (0.56 mmol) of potassium iodide, 453 mg (3.28 mmol) of potassium carbonate and 0.33 mL (2.74 mmol) of 1,4-dibromobutane are added successively to a solution of 500 mg (2.74 mmol) of 4-(2-amino-ethyl)-benzonitrile in 50 mL acetonitrile. The reaction is stirred for 6 h at 78° C. Another 0.08 mL (0.66 mmol) of 1,4-dibromobutane are added and the reaction is stirred overnight at 78° C. After filtration the filtrate is evaporated to dryness. The further purification is carried out by column chromatography on silica gel (dichloromethane/methanol 8:2).

Yield: 183.0 mg (33.4% of theory);

C₁₃H₁₆N₂ (M=200.286);

calc.: molar peak (M+H)⁺: 201 fnd.: molar peak (M+H)⁺: 201.

2.39.b 4-(2-pyrrolidin-1-yl-ethyl)-benzylamine

75 mg of Raney nickel is added to a solution of 183 mg (0.91 mmol) of 4-(2-pyrrolidin-1-yl-ethyl)-benzonitrile in 20 mL ethanolic ammonia solution. The reaction solution is stirred overnight at 50° C. and 3 bar hydrogen. Another 75 mg of Raney nickel are added and the mixture is stirred for a further 6 h at 50° C. and 3 bar hydrogen.

The catalyst is filtered off and the solvent is eliminated using the rotary evaporator. The crude product may be used without further purification.

Yield: 114.0 mg (61.0% of theory);

C₁₃H₂₀N₂ (M=204.318);

calc.: molar peak (M+H)⁺: 205 fnd.: molar peak (M+H)⁺: 205.

2.39.c 4′-chloro-biphenyl-4-carboxylic acid-4-(2-pyrrolidin-1-yl-ethyl)-benzylamide

Prepared according to general working method I from 4′-chloro-biphenyl-4-carboxylic acid (130 mg, 0.56 mmol) and 4-(2-pyrrolidin-1-yl-ethyl)-benzylamine (114 mg, 0.56 mmol).

Yield: 75.0 mg (32.1% of theory);

C₂₆H₂₇ClN₂O (M=418.971);

calc.: molar peak (M+H)⁺: 419/421 fnd.: molar peak (M+H)⁺: 419/421;

R_{f} value: 0.38 (silica gel, ethyl acetate/methanol/NH₃ 9:1:0.1).

### EXAMPLE 2.40

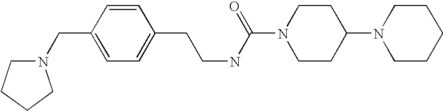

2.40.a [1,4′]bipiperidinyl-1′-carboxylic acid-[2-(4-pyrrolidin-1-ylmethyl-phenyl)-ethyl]-amide

Prepared according to general working method II from 4-piperidinopiperidine (84.1 mg, 0.50 mmol) and 2-(4-pyrrolidin-1-ylmethyl-phenyl)-ethylamine (102 mg, 0.50 mmol).

Yield: 3.0 mg (1.5% of theory);

C₂₄H₃₈N₄O (M=398.597);

calc.: molar peak (0.5M+H)⁺: 200 fnd.: molar peak (0.5M+H)⁺: 200;

Retention time HPLC: 1.59 min (method A).

### EXAMPLE 2.41

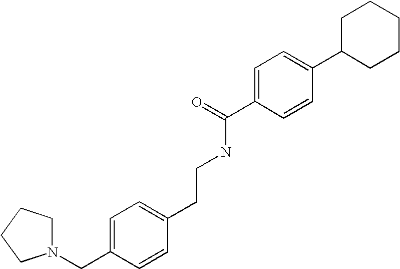

2.41.a 4-cyclohexyl-N-[2-(4-pyrrolidin-1-ylmethyl-phenyl)-ethyl]-benzamide

Prepared according to general working method I from 4-cyclohexylbenzoic acid (102 mg, 0.50 mmol) and 4-(2-pyrrolidin-1-yl-ethyl)-benzylamine (102 mg, 0.50 mmol).

Yield: 2.0 mg (1.0% of theory);

C₂₆H₃₄N₂O (M=390.574);

calc.: molar peak (M+H)⁺: 391 fnd.: molar peak (M+H)⁺: 391;

R_{f} value: 0.38 (silica gel, ethyl acetate/methanol/NH₃ 9:1:0.1).

### EXAMPLE 2.42

### 4′-chloro-biphenyl-4-carboxylic acid-[2-(4-pyrrolidin-1-ylmethyl-cyclohexyl)-ethyl]-amide

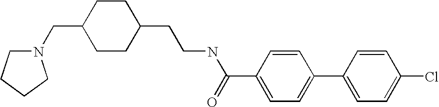

2.42.a 2-(4-pyrrolidin-1-ylmethyl-cyclohexyl)-ethylamine

1.52 mL conc. hydrochloric acid and 300 mg platinum oxide are added to a solution of 500 mg (2.45 mmol) of 2-(4-pyrrolidin-1-ylmethyl-phenyl)-ethylamine (cf. Example 1.1.h) in 10 mL methanol. The reaction mixture is stirred at 50° C. and 5 bar hydrogen for 50 h. After separation of the catalyst the solvent is eliminated using the rotary evaporator. The further purification is carried out by column chromatography on silica gel (dichloromethane/methanol/ammonia 8:2:0.2).

Yield: 130 mg (25.3% of theory);

C₁₃H₂₆N₂ (M=210.366);

calc.: molar peak (M+H)⁺: 211 fnd.: molar peak (M+H)⁺: 211;

R_{f} value: 0.14 (silica gel, dichloromethane/methanol/NH₃ 8:2:0.2).

2.42.b 4′-chloro-biphenyl-4-carboxylic acid-[2-(4-pyrrolidin-1-ylmethyl-cyclohexyl)-ethyl]-amide

Prepared according to general working method I from 4′-chloro-biphenyl-4-carboxylic acid (116 mg, 0.50 mmol) and 2-(4-pyrrolidin-1-ylmethyl-cyclohexyl)-ethylamine (105 mg, 0.50 mmol).

Yield: 53.0 mg (24.9% of theory);

C₂₆H₃₃ClN₂O (M=425.019);

calc.: molar peak (M+H)⁺: 425/427 fnd.: molar peak (M+H)⁺: 425/427;

R_{f} value: 0.16 (silica gel, ethyl acetate/methanol/NH₃ 9:1:0.1).

### EXAMPLE 2.43

### 4′-chloro-biphenyl-4-carboxylic acid-[2-(3-methoxy-4-pyrrolidin-1-ylmethyl-phenyl)-ethyl]-amide

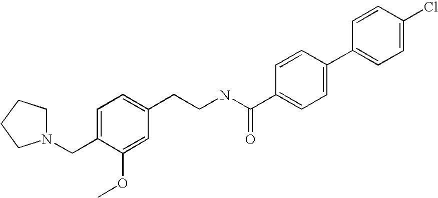

2.43.a 4-cyanomethyl-2-methoxy-benzoic acid

Prepared analogously to Example 1.1.d from methyl 4-cyanomethyl-2-methoxy-benzoate

Yield: 6.5 g (69.8% of theory);

C₁₀H₉NO₃ (M=191.18);

calc.: molar peak (M+H)⁺: 192 fnd.: molar peak (M+H)⁺: 192;

R_{f} value: 0.64 (silica gel, dichloromethane/ethanol 10:1).

2.43.b (4-hydroxymethyl-3-methoxy-phenyl)-acetonitrile

Prepared analogously to Example 1.1.e from 4-cyanomethyl-2-methoxy-benzoic acid.

Yield: 4.81 g (81% of theory);

C₁₀H₁₁NO₂ (M=177.20);

calc.: molar peak (M)⁺: 177 fnd.: molar peak (M)⁺: 177.

2.43.c (4-bromomethyl-3-methoxy-phenyl)-acetonitrile

Prepared analogously to Example 1.1.f from (4-hydroxymethyl-3-methoxy-phenyl)-acetonitrile

Yield: 4.2 g (64.6% of theory);

C₁₀H₁₀BrNO (M=240.10);

calc.: molar peak (M)⁺: 239/241 fnd.: molar peak (M)⁺: 239/241;

R_{f} value: 0.84 (silica gel, dichloromethane/ethanol 50:1).

2.43.d (3-methoxy-4-pyrrolidin-1-ylmethyl-phenyl)-acetonitrile

Prepared analogously to Example 1.1.g from (4-bromomethyl-3-methoxy-phenyl)-acetonitrile and piperidine.

Yield: 0.95 g (24.2% of theory);

C₁₄H₁₈N₂O (M=230.31);

calc.: molar peak (M+H)⁺: 231 fnd.: molar peak (M+H)⁺: 231.

2.43.e (3-methoxy-4-pyrrolidin-1-ylmethyl-phenyl)-ethylamine

Prepared analogously to Example 1.1.h from (3-methoxy-4-pyrrolidin-1-ylmethyl-phenyl)-acetonitrile. The crude product is further reacted immediately without purification.

2.43.f 4′-chloro-biphenyl-4-carboxylic acid-[2-(3-methoxy-4-pyrrolidin-1-ylmethyl-phenyl)-ethyl]-amide

Prepared according to general working method I from 2-(3-methoxy-4-pyrrolidin-1-ylmethyl-phenyl)-ethylamine and 4′-chloro-biphenyl-4-carboxylic acid.

Yield: 0.5 g (86.2% of theory);

melting point: 162-163° C.;

C₂₇H₂₉ClN₂O₂ (M=448.99);

calc.: molar peak (M+H)⁺: 449/451 fnd.: molar peak (M+H)⁺: 449/451;

R_{f} value: 0.85 (silica gel, dichloromethane/ethanol/ammonia 5:1:0.1).

### EXAMPLE 2.44

### 4′-chloro-biphenyl-4-carboxylic acid-[2-(2-fluoro-4-pyrrolidin-1-ylmethyl-phenyl)-ethyl]-amide

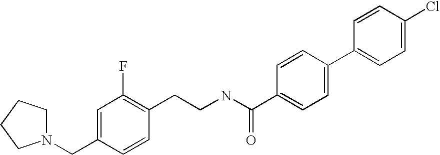

2.44.a (E)-3-(4-cyano-2-fluoro-phenyl)-acrylic acid 2.75 g (10 mmol) of palladium acetate and 7.0 g (25 mmol) of tri-o-tolylphosphane are added to a solution of 20.0 g (100 mmol) of 4-bromo-3-fluoro-benzonitrile in 200 mL DMF. Then 50 mL triethylamine and 30 mL (30 mmol) of ethyl acrylate are added. The reaction mixture is stirred for 3 h at 100° C., after cooling diluted with 400 mL dichloromethane and washed twice with water. The solvent is eliminated using the rotary evaporator and the residue is taken up in 250 mL methanol with heating. Insoluble constituents are removed by suction filtering through kieselguhr and the filtrate is evaporated down by half in the rotary evaporator. After filtering again, it is combined with 150 mL THF, 100 mL MeOH and 43 mL 2N NaOH and stirred for 2 h at ambient temperature. The solvent is eliminated using the rotary evaporator and the residue is combined with 100 mL water. After extraction with ether the aqueous phase is acidified with conc. hydrochloric acid. The crystals precipitated are dissolved in 300 ml of warm ethyl acetate, the aqueous phase is separated off. The ethyl acetate is distilled off and the crystals obtained are suspended in ether and suction filtered.

Yield: 11.5 g (60.2% of theory);

melting point: 214-218° C.

2.44.b 3-(4-cyano-2-fluoro-phenyl)-propionic acid

A solution of 11.5 g (60 mmol) of (E)-3-(4-cyano-2-fluoro-phenyl)-acrylic acid in 200 mL water is combined with 4.0 g 5% Pd/C and 24.4 g potassium carbonate. The mixture is shaken for 6 h at ambient temperature and normal hydrogen pressure in the autoclave. After suction filtering of the catalyst the mother liquor is acidified with conc. hydrochloric acid. The precipitated crystals are dissolved in 250 ml warm ethyl acetate and dried and the ethyl acetate is distilled off. The crystals obtained are stirred with ether/hexane and suction filtered.

Yield: 900 mg (98.0% of theory);

melting point: 102-106° C.

2.44.c tert.butyl [2-(4-cyano-2-fluoro-phenyl)-ethyl]-carbaminate

1.25 mL triethylamine and 0.61 mL (2.8 mmol) of diphenylphosphorylazide are added to a solution of 500 mg (2.6 mmol) of 3-(4-cyano-2-fluoro-phenyl)-propionic acid in 5 mL tert-butanol. The reaction mixture is refluxed overnight and then the solvent is eliminated using the rotary evaporator. The purification is carried out by column chromatography on silica gel (dichloromethane/methanol 9:1).

Yield: 138 mg (20.2% of theory);

C₁₄H₁₇FN₂O₂ (M=264.302);

calc.: molar peak (M+H)⁺: 265 fnd.: molar peak (M+H)⁺: 265.

2.44.d tert.butyl [2-(4-aminomethyl-2-fluoro-phenyl)-ethyl]-carbaminate

A solution of 138 mg (0.52 mmol) of tert.butyl [2-(4-cyano-2-fluoro-phenyl)-ethyl]-carbaminate in 15 mL ethanolic ammonia solution is combined with 75 mg of Raney nickel and the mixture is shaken overnight at 50° C. and 3 bar hydrogen in the autoclave. After the catalyst has been suction filtered the solvent is eliminated using the rotary evaporator.

Yield: 137 mg (97.8% of theory);

C₁₄H₂₁FN₂O₂ (M=268.334);

calc.: molar peak (M+H)⁺: 269 fnd.: molar peak (M+H)⁺: 269.

2.44.e tert.butyl [2-(2-fluoro-4-pyrrolidin-1-ylmethyl-phenyl)-ethyl]-carbaminate

To a solution of 300 mg (1.12 mmol) of tert.butyl [2-(4-aminomethyl-2-fluoro-phenyl)-ethyl]-carbaminate in 15 mL acetonitrile are added successively 42 mg (0.25 mmol) of potassium iodide, 180 mg (1.30 mmol) of potassium carbonate and 0.13 mL (1.11 mmol) of 1,4-dibromobutane. The reaction is stirred for 6 h at 78° C. Another 0.08 mL (0.66 mmol) of 1,4-dibromobutane are added and the reaction is stirred overnight at 78° C. The solvent is eliminated using the rotary evaporator and the product further reacted without purification.

Yield: 320 mg (88.8% of theory);

C₁₈H₂₇FN₂O₂ (M=322.426);

calc.: molar peak (M+H)⁺: 323 fnd.: molar peak (M+H)⁺: 323.

2.44.f 2-(2-fluoro-4-pyrrolidin-1-ylmethyl-phenyl)-ethylamine

To a solution of 232 mg (0.72 mmol) of tert.butyl [2-(2-fluoro-4-pyrrolidin-1-ylmethyl-phenyl)-ethyl]-carbaminate in 5 mL dichloromethane is added 1.5 mL trifluoroacetic acid. The reaction mixture is stirred for 2 h at ambient temperature. The solvent is eliminated using the rotary evaporator and the crude product is further reacted without purification.

Yield: 160 mg (100% of theory);

C₁₃H₁₉FN₂ (M=222.308);

calc.: molar peak (M+H)⁺: 223 fnd.: molar peak (M+H)⁺: 223.

2.44.g 4′-chloro-biphenyl-4-carboxylic acid-[2-(2-fluoro-4-pyrrolidin-1-ylmethyl-phenyl)-ethyl]-amide

Prepared according to general working method I from 2-(2-fluoro-4-pyrrolidin-1-ylmethyl-phenyl)-ethylamine (160 mg, 0.72 mmol) and 4′-chloro-biphenyl-4-carboxylic acid (168 mg, 0.72 mmol).

Yield: 49 mg (15.6% of theory);

C₂₆H₂₆ClFN₂O (M=436.961);

calc.: molar peak (M+H)⁺: 437/439 fnd.: molar peak (M+H)⁺: 437/439;

Retention time HPLC: 6.6 min (method A).

### EXAMPLE 2.45

### 4-pyridin-4-yl-N-[2-(4-pyrrolidin-1-ylmethyl-phenyl)-ethyl]-benzamide

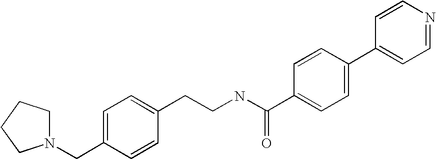

2.45a.methyl 4-pyridin-4-yl-benzoate

3.0 g (15 mmol) of 4-bromo-pyridine is dissolved in 50 mL dioxane and 15 mL 2M sodium carbonate solution. 2.7 g (15 mmol) of 4-methoxycarbonylphenyl-boric acid and 1.73 g (2 mmol) of tetrakis-(triphenylphosphine)-palladium are added successively and the reaction is refluxed for 6 h. The hot reaction solution is suction filtered through a glass fibre filter. The solvent is eliminated using the rotary evaporator and the purification is carried out by column chromatography on silica gel (dichloromethane/methanol 9:1).

Yield: 845 mg (26.4% of theory);

C₁₃H₁₁NO₂ (M=213.238);

calc.: molar peak (M+H)⁺: 214 fnd.: molar peak (M+H)⁺: 214;

Retention time HPLC: 4.1 min (method A).

2.45b. 4-pyridin-4-yl-benzoic acid

0.37 mL (0.74 mmol) of 2N NaOH are added to a solution of 150 mg (0.70 mmol) of methyl 4-pyridin-4-yl-benzoate in 10 mL ethanol. The reaction solution is stirred for 2 h at 60° C. and then the pH is adjusted to 6-7 with 1N HCl. After filtration the precipitate formed is dried overnight under high vacuum.

Yield: 84 mg (60.0% of theory);

C₁₂H₉NO₂ (M=199.211);

calc.: molar peak (M+H)⁺: 200 fnd.: molar peak (M+H)⁺: 200;

Retention time HPLC: 2.5 min (method A).

2.45c. 4-pyridin-4-yl-N-[2-(4-pyrrolidin-1-ylmethyl-phenyl)-ethyl]-benzamide

Prepared according to general working method I from 2-(4-pyrrolidin-1-ylmethyl-phenyl)-ethylamine (86 mg, 0.42 mmol) and 4-pyridin-4-yl-benzoic acid (84 mg, 0.42 mmol).

Yield: 65 mg (40.0% of theory);

C₂₅H₂₇N₃O (M=385.513);

calc.: molar peak (M+H)⁺: 386 fnd.: molar peak (M+H)⁺: 386;

Retention time HPLC: 4.7 min (Stable Bond C18; 3.5 μm; water:acetonitrile:formic acid 91:9:0.01).

### EXAMPLE 2.46

### 5-(4-chloro-phenyl)-2-[2-(4-pyrrolidin-1-ylmethyl-phenyl)-ethyl]-2.3-dihydro-isoindol-1-one

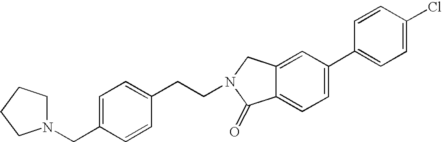

2.46.a ethyl 4-bromo-2-methyl-benzoate

A solution of 5.0 g (23.3 mmol) of 4-bromo-2-methyl-benzoic acid in 50 mL ethanolic hydrochloric acid is stirred for 8 h at 45° C. The reaction solution is cooled to ambient temperature overnight and then the solvent is eliminated using the rotary evaporator. The residue is taken up in ether, filtered and the solvent is eliminated using the rotary evaporator. The residue is further reacted without purification.

R_{f} value: 0.88 (silica gel, dichloromethane/ethanol 95:5).

2.46.b ethyl 4′-chloro-3-methyl-biphenyl-4-carboxylate

1.66 g (6.83 mmol) of ethyl 4-bromo-2-methyl-benzoate is dissolved in 70 mL dioxane and 7 mL 2M sodium carbonate solution. 1.07 g (6.83 mmol) of 4-chloro-phenyl-boric acid and 0.40 g (0.34 mmol) of tetrakis-(triphenylphosphine)-palladium are added successively, the reaction is refluxed for 6 h and stirred for a further 60 h at ambient temperature. The hot reaction solution is suction filtered through a glass fibre filter. The solvent is eliminated using the rotary evaporator. The residue is combined with water and the aqueous phase extracted with ethyl acetate. The organic phase is dried over magnesium sulphate and the solvent is eliminated using the rotary evaporator. The purification is carried out by column chromatography on silica gel (petroleum ether/ethyl acetate 8:2).

Yield: 1.3 g (69.3% of theory);

C₁₆H₁₅ClO₂ (M=274.750);

calc.: molar peak (M+H)⁺: 275/277 fnd.: molar peak (M+H)⁺: 275/277;

R_{f} value: 0.67 (silica gel, petroleum ether/ethyl acetate 8:2).

2.46.c ethyl 3-bromomethyl-4′-chloro-biphenyl-4-carboxylate

78 mg (0.47 mmol) of 2,2′-azobis(isobutyronitrile) are added to a solution of 1.3 g (4.73 mmol) of ethyl 4′-chloro-3-methyl-biphenyl-4-carboxylate and 0.84 g (4.73 mmol) of N-bromosuccinimide in 10 mL carbon tetrachloride. The reaction mixture is refluxed overnight. After filtration the solvent is evaporated down in the rotary evaporator. The purification is carried out by column chromatography on silica gel (petroleum ether/ethyl acetate 8:2).

Yield: 1.6 g (62.1% of theory);

C₁₆H₁₄BrClO₂ (M=353.646);

calc.: molar peak (M+H)⁺: 353/355/357 fnd.: molar peak (M+H)⁺: 353/355/357;

R_{f} value: 0.57 (silica gel, petroleum ether/ethyl acetate 8:2).

2.46.d 5-(4-chloro-phenyl)-2-[2-(4-pyrrolidin-1-ylmethyl-phenyl)-ethyl]-2,3-dihydro-isoindol-1-one

375 mg (1.47 mmol) of 2-(4-pyrrolidin-1-ylmethyl-phenyl)-ethylamine is slowly added dropwise at ambient temperature to a suspension of 800 mg (1.47 mmol) of ethyl 3-bromomethyl-4′-chloro-biphenyl-4-carboxylate and 508 mg (3.68 mmol) of potassium carbonate in 7.5 mL acetonitrile. The reaction mixture is refluxed for 5 hours. After elimination of the solvent using the rotary evaporator the residue is taken up in water and ethyl acetate. The aqueous phase is extracted with ethyl acetate and the combined organic phases are dried over magnesium sulphate. After elimination of the solvent using the rotary evaporator the residue is dissolved in DMF and purified by HPLC chromatography (Stable Bond C18; 3.5 μm; water:acetonitrile:formic acid 9:1:0.01 towards 1:9:0.01 over 9 min).

Yield: 82 mg (12.9% of theory);

C₂₇H₂₇ClN₂O₂ (M=430.982);

calc.: molar peak (M+H)⁺: 431/433 fnd.: molar peak (M+H)⁺: 431/433;

Retention time HPLC: 6.13 min (method A).

### EXAMPLE 2.47

### 4-piperidin-1-ylmethyl-piperidine-1-carboxylic acid-[2-(4-pyrrolidin-1-ylmethyl-phenyl)-ethyl]-amide

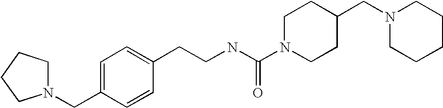

2.47.a 4-piperidin-1-ylmethyl-pyridine

242 mL piperidine (2.44 mol) are added dropwise to a solution of 100 g (0.61 mol) of 4-chloromethyl-pyridine in 600 mL dry methanol and the reaction mixture is stirred for one hour at 50° C. The solvent is eliminated using the rotary evaporator. The residue is made alkaline with 40% sodium hydroxide solution and the aqueous phase extracted with ether. The organic phase is dried over sodium sulphate and after filtration through activated charcoal the solvent is eliminated using the rotary evaporator. The crude product is further reacted without purification.

Yield: 106 g (98% of theory).

2.47.b 4-piperidin-1-ylmethyl-piperidine

A solution of 106 g (0.6 mol) of 4-piperidin-1-ylmethyl-pyridine in 1.0 L glacial acetic acid is combined with 7 g platinum dioxide and shaken in the autoclave at ambient temperature and 3 bar hydrogen. After the catalyst has been suction filtered the solvent is eliminated using the rotary evaporator. The crude product is further reacted without purification.

Yield: 48 g (43.9% of theory).

2.47.c 4-piperidin-1-ylmethyl-piperidine-1-carboxylic acid-[2-(4-pyrrolidin-1-ylmethyl-phenyl)-ethyl]-amide

Prepared according to general working method II from 4-piperidin-1-ylmethyl-piperidine (182 mg, 1.00 mmol) and 2-(4-pyrrolidin-1-ylmethyl-phenyl)-ethylamine (204 mg, 1.00 mmol).

Yield: 160.0 mg (38.8% of theory);

C₂₅H₄₀N₄O (M=412.624);

calc.: molar peak (M+H)⁺: 413 fnd.: molar peak (M+H)⁺: 413;

Retention time HPLC: 1.75 min (Stable Bond C18; 3.5 μm;

water:acetonitrile:formic acid 9:1:0.01 towards 4:6:0.01 over 8 min).

### EXAMPLE 2.48

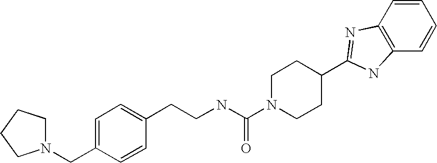

2.48.a 4-(1H-benzoimidazol-2-yl)-piperidine-1-carboxylic acid-[2-(4-pyrrolidin-1-ylmethyl-phenyl)-ethyl]-amide

Prepared according to general working method II from 2-piperidin-4-yl-1H-benzoimidazole (164 mg, 1.00 mmol) and 2-(4-pyrrolidin-1-ylmethyl-phenyl)-ethylamine (204 mg, 1.00 mmol).

Yield: 80.0 mg (18.5% of theory);

C₂₆H₃₃N₅O (M=431.586);

calc.: molar peak (M+H)⁺: 432 fnd.: molar peak (M+H)⁺: 432;

Retention time HPLC: 2.80 min (Stable Bond C18; 3.5 μm;

water:acetonitrile:formic acid 9:1:0.01 towards 4:6:0.01 over 8 min).

### EXAMPLE 2.49

### 4-(1-methyl-piperidin-4-yl)-N-[2-(4-pyrrolidin-1-ylmethyl-phenyl)-ethyl]-benzamide

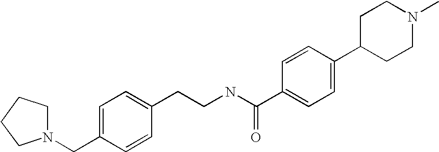

2.49.a methyl 4-piperidin-4-yl-benzoate

4.0 mL 1M hydrochloric acid and 200 mg platinum oxide are added to a solution of 695 mg (3.26 mmol) of methyl 4-pyridin-4-yl-benzoate (cf. Example 2.45.a) in 10 mL ethanol. The reaction mixture is stirred for 2 h at ambient temperature and 3 bar hydrogen. After another 300 mg platinum oxide and 6.0 mL 1M hydrochloric acid have been added the mixture is stirred for a further 16 h at ambient temperature and 3 bar hydrogen. After separation of the catalyst the solvent is eliminated using the rotary evaporator. The crude product is further reacted without purification.

Yield: 589 mg (82.4% of theory);

C₁₃H₁₇NO₂ (M=219.286);

calc.: molar peak (M+H)⁺: 220 fnd.: molar peak (M+H)⁺: 220;

Retention time HPLC: 3.5 min (method A).

2.49.b methyl 4-(1-methyl-piperidin-4-yl)-benzoate

48 mg (2.00 mmol) of sodium hydride is added batchwise to a solution of 429 mg (1.96 mmol) of methyl 4-piperidin-4-yl-benzoate in 10 mL DMF under a nitrogen atmosphere at 0° C. The reaction mixture is stirred for 1 h at ambient temperature. 0.13 mL (2.10 mmol) of methyl iodide is added dropwise and the solution is stirred for two hours at ambient temperature. The reaction solution is combined with water, the aqueous phase is extracted with ethyl acetate, the combined organic phases are dried over magnesium sulphate and the solvent is eliminated using the rotary evaporator. The purification is carried out by column chromatography (silica gel; dichloromethane/methanol 8:2).

Yield: 70 mg (15.3% of theory);

C₁₄H₁₉NO₂ (M=233.313);

calc.: molar peak (M+H)⁺: 234 fnd.: molar peak (M+H)⁺: 234;

Retention time HPLC: 2.7 min (method A).

2.49.c 4-(1-methyl-piperidin-4-yl)-benzoic acid

0.37 mL (0.74 mmol) of 2N NaOH are added to a solution of 70 mg (0.30 mmol) of methyl 4-(1-methyl-piperidin-4-yl)-benzoate in 10 mL ethanol. The reaction solution is stirred for 2 h at 60° C. and then adjusted to pH 6-7 using 1N HCl. After filtration the precipitate formed is dried overnight under high vacuum.

Yield: 50 mg (76.0% of theory);

C₁₃H₁₇NO₂ (M=219.286);

calc.: molar peak (M+H)⁺: 220 fnd.: molar peak (M+H)⁺: 220;

Retention time HPLC: 1.5 min (method A).

2.49.d 4-(1-methyl-piperidin-4-yl)-N-[2-(4-pyrrolidin-1-ylmethyl-phenyl)-ethyl]-benzamide

Prepared according to general working method I from 2-(4-pyrrolidin-1-ylmethyl-phenyl)-ethylamine (47 mg, 0.23 mmol) and 4-(1-methyl-piperidin-4-yl)-benzoic acid (50 mg, 0.23 mmol).

Yield: 22 mg (23.8% of theory);

C₂₆H₃₅N₃O (M=405.588);

calc.: molar peak (M+H)⁺: 406 fnd.: molar peak (M+H)⁺: 406;

Retention time HPLC: 2.4 min (method A).

### EXAMPLE 2.50

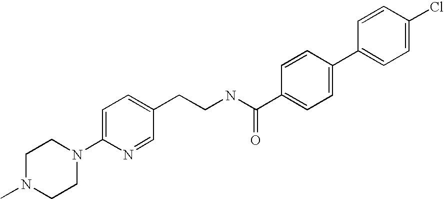

1.21.a 4′-chloro-biphenyl-4-carboxylic acid-{2-[6-(4-methyl-piperazin-1-yl)-pyridin-3-yl]-ethyl}-amide

Prepared analogously to Example 1.1.i from 2-[6-(4-methyl-piperazin-1-yl)-pyridin-3-yl]-ethylamine and 4′-chloro-biphenyl-4-carboxylic acid.

Yield: 0.94 g (96% of theory);

melting point: 211-213° C.;

C₂₅H₂₇ClN₄O (M=434.97);

calc.: molar peak (M+H)⁺: 435/437 fnd.: molar peak (M+H)⁺: 435/437.

### EXAMPLE 2.51

### 4′-chloro-biphenyl-4-carboxylic acid-{2-[4-(4-methyl-piperazine-1-carbonyl)-phenyl]-ethyl}-amide

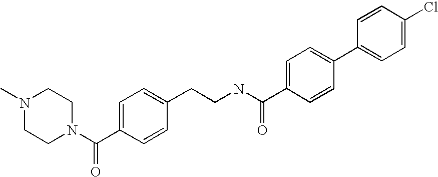

2.51.a [4-(4-methyl-piperazine-1-carbonyl)-phenyl]-acetonitrile

A solution of 2 g (12.41 mmol) of 4-cyanomethyl-benzoic acid, 1.25 g (12.5 mmol) of N-methylpiperazine, 4.01 g (12.5 mmol) of TBTU and 3.48 ml (25 mmol) of triethylamine in 40 ml DMF is stirred for 12 hours at ambient temperature. Then the reaction mixture is evaporated down to some extent and combined with water. This mixture is extracted with ethyl acetate and the solvent is distilled off using the rotary evaporator. The aqueous phase is also evaporated down and the organic phase is combined with the residue. The purification is carried out by column chromatography on silica gel (eluant: dichloromethane/ethanol/ammonia 30:1:0.1).

Yield: 2.6 g (86% of theory);

C₁₄H₁₇N₃O (M=243.31);

calc.: molar peak (M+H)⁺: 244 fnd.: molar peak (M+H)⁺: 244;

R_{f} value: 0.35 (silica gel, dichloromethane/ethanol/ammonia 20:1:0.1).

2.51.b [4-(2-amino-ethyl)-phenyl]-(4-methyl-piperazin-1-yl)-methanone

Prepared analogously to Example 1.1.i from [4-(4-methyl-piperazine-1-carbonyl)-phenyl]-acetonitrile.

Yield: 2.9 g (90% of theory);

C₁₄H₂₁N₃O×HCl (M=283.80);

R_{f} value: 0.25 (silica gel, dichloromethane/ethanol/ammonia 10:1:0.1).

2.51.c 4′-chloro-biphenyl-4-carboxylic acid-{2-[4-(4-methyl-piperazine-1-carbonyl)-phenyl]-ethyl}-amide

Prepared according to general working method I from [4-(2-amino-ethyl)-phenyl]-(4-methyl-piperazin-1-yl)-methanone and 4′-chloro-biphenyl-4-carboxylic acid.

Yield: 0.18 g (48.4% of theory);

melting point: 217-218° C.;

C₂₇H₂₈ClN₃O₂ (M=461.99);

calc.: molar peak (M+H)⁺: 462/464 fnd.: molar peak (M+H)⁺: 462/464;

R_{f} value: 0.25 (silica gel, dichloromethane/methanol/ammonia 10:1:0.1).

### EXAMPLE 2.52

### 4′-bromo-biphenyl-4-carboxylic acid [2-(4-pyrrolidin-1-ylmethyl-phenyl)-ethyl]-amide

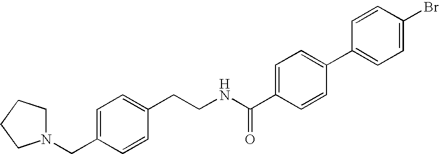

2.52a. methyl 4′-bromo-biphenyl-4-carboxylate

0.54 g (2.5 mmol) of methyl 4-bromo-benzoate is dissolved in 10 mL dioxane and 2.5 mL 2M-sodium carbonate solution. 0.6 g (3 mmol) of 4-bromophenyl-boric acid and 0.12 g (0.1 mmol) of tetrakis-(triphenylphosphine)-palladium are added successively and the reaction is refluxed for 5 hours. The reaction mixture is combined with water and EtOAc, filtered and the phases are separated. The aqueous phase is extracted with EtOAc and the combined organic phases are dried over MgSO₄. After elimination of the drying agent and solvent the residue is triturated with acetonitrile, suction filtered and dried in the air.

Yield: 100 mg (13.7% of theory);

C₁₄H₁₁BrO₂ (M=291.15);

calc.: molar peak (M+H)⁺: 291/293 fnd.: molar peak (M+H)⁺: 291/293;

R_{f} value: 0.68 (silica gel, petroleum ether/EtOAc 8:2).

2.52b. 4′-bromo-biphenyl-4-carboxylic acid

A solution of 100 mg (0.34 mmol) of methyl 4′-bromo-biphenyl-4-carboxylate in 3 mL THF is combined with 3 mL of a 1M NaOH solution in water and refluxed for 3 h. The reaction mixture is evaporated down in vacuo, the aqueous residue acidified with 1 M HCl, the product precipitated is filtered off and dried in the air.

Yield: 60 mg (63.1% of theory);

C₁₃H₉BrO₂ (M=277.19);

calc.: molar peak (M−H)⁻: 275/277 fnd.: molar peak (M−H)⁻: 275/277;

Retention time HPLC: 8.48 min (method A).

2.52.c 4′-bromo-biphenyl-4-carboxylic acid [2-(4-pyrrolidin-1-ylmethyl-phenyl)-ethyl]-amide

Prepared according to general working method I from 45 mg (0.22 mmol) of 2-(4-pyrrolidin-1-ylmethyl-phenyl)-ethylamine and 60 mg (0.22 mmol) of 4′-bromo-biphenyl-4-carboxylic acid.

Yield: 28 mg (27.5% of theory);

C₂₆H₂₇BrN₂O (M=463.42);

calc.: molar peak (M+H)⁺: 463/465 fnd.: molar peak (M+H)⁺: 463/465;

Retention time HPLC: 6.46 min (method A).

### EXAMPLE 2.53

### 4′-ethyl-biphenyl-4-carboxylic acid [2-(4-pyrrolidin-1-ylmethyl-phenyl)-ethyl]-amide

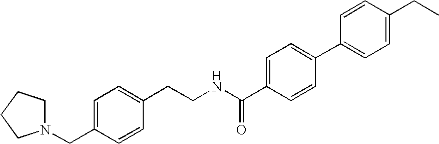

Prepared according to general working method I from 102 mg (0.5 mmol) of 2-(4-pyrrolidin-1-ylmethyl-phenyl)-ethylamine and 113 mg (0.5 mmol) of 4′-ethyl-biphenyl-4-carboxylic acid (Lancaster).

Yield: 65 mg (31.5% of theory);

C₂₈H₃₂N₂O (M=412.58);

calc.: molar peak (M+H)⁺: 463 fnd.: molar peak (M+H)⁺: 463;

Retention time HPLC: 6.64 min (method A).

### EXAMPLE 2.54

### biphenyl-4-carboxylic acid [2-(4-pyrrolidin-1-ylmethyl-phenyl)-ethyl]-amide

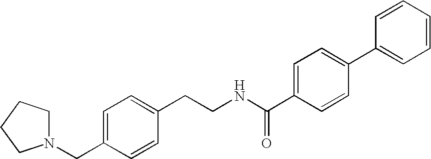

Prepared according to general working method I from 102 mg (0.5 mmol) of 2-(4-pyrrolidin-1-ylmethyl-phenyl)-ethylamine and 99 mg (0.5 mmol) of biphenyl-4-carboxylic acid.

Yield: 46 mg (23.9% of theory);

C₂₆H₂₈N₂O (M=384.53);

calc.: molar peak (M+H)⁺: 385 fnd.: molar peak (M+H)⁺: 385;

Retention time HPLC: 5.70 min (method A).

### EXAMPLE 2.55

### 4′-fluoro-biphenyl-4-carboxylic acid [2-(4-pyrrolidin-1-ylmethyl-phenyl)-ethyl]-amide

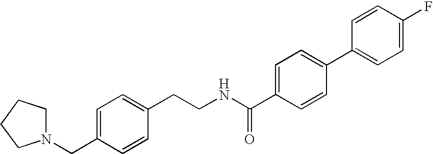

2.55a. 4′-fluoro-biphenyl-4-carboxylic acid

14.27 g (71 mmol) of 4-bromo-benzoic acid are dissolved in 120 mL dioxane and 70 mL 2M Na₂CO₃ solution. 10 g (71 mmol) of 4-fluorophenyl-boric acid and 4.1 g (4 mmol) of tetrakis-(triphenylphosphine)-palladium are added successively and the reaction is refluxed for 6 h. The catalyst is suction filtered and washed with hot water. The reaction mixture is combined with EtOAc, the phases are separated and the aqueous phase is acidified with citric acid. The precipitate formed is suction filtered, washed with water and dried at 45° C. in vacuo.

Yield: 4.9 g (31.9% of theory);

C₁₃H₉FO₂ (M=216.21);

calc.: molar peak (M−H)⁻: 215 fnd.: molar peak (M−H)⁻: 215.

2.55b. 4′-fluoro-biphenyl-4-carboxylic acid [2-(4-pyrrolidin-1-ylmethyl-phenyl)-ethyl]-amide

Prepared according to general working method I from 102 mg (0.5 mmol) of 2-(4-pyrrolidin-1-ylmethyl-phenyl)-ethylamine and 108 mg (0.5 mmol) of 4′-fluoro-biphenyl-4-carboxylic acid.

Yield: 12 mg (6.0% of theory);

C₂₆H₂₇FN₂O (M=402.52);

calc.: molar peak (M+H)⁺: 403 fnd.: molar peak (M+H)⁺: 403;

Retention time HPLC: 5.83 min (method A).

### EXAMPLE 2.56

### 4′-hydroxy-3′-nitro-biphenyl-4-carboxylic acid [2-(4-pyrrolidin-1-ylmethyl-phenyl)-ethyl]-amide

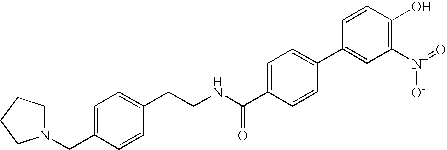

Prepared according to general working method I from 102 mg (0.5 mmol) of 2-(4-pyrrolidin-1-ylmethyl-phenyl)-ethylamine and 130 mg (0.5 mmol) of 4′-fluoro-3′-nitro biphenyl-4-carboxylic acid.

Yield: 9 mg (4.0% of theory);

C₂₆H₂₇N₃O₄ (M=445.52);

calc.: molar peak (M+H)⁺: 446 fnd.: molar peak (M+H)⁺: 446;

Retention time HPLC: 5.83 min (method A).

### EXAMPLE 2.57

### 3′-chloro-biphenyl-4-carboxylic acid [2-(4-pyrrolidin-1-ylmethyl-phenyl)-ethyl]-amide

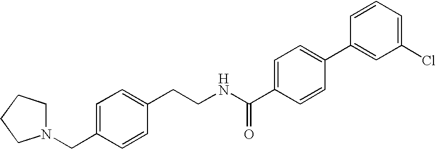

2.57a. 3′-chloro-biphenyl-4-carboxylic acid

Prepared analogously to Example 2.55a from 9.64 g (47.96 mmol) of 4-bromo-benzoic acid and 7.5 g (47.96 mmol) of 3-chlorophenyl-boric acid.

Yield: 6.2 g (55.6% of theory);

C₁₃H₉ClO₂ (M=232.67);

calc.: molar peak (M−H)⁻: 231/233 fnd.: molar peak (M−H)⁻: 231/233.

2.57b. 3′-chloro-biphenyl-4-carboxylic acid [2-(4-pyrrolidin-1-ylmethyl-phenyl)-ethyl]-amide

Prepared according to general working method I from 102 mg (0.5 mmol) of 2-(4-pyrrolidin-1-ylmethyl-phenyl)-ethylamine and 116 mg (0.5 mmol) of 3′-chloro-biphenyl-4-carboxylic acid.

Yield: 63 mg (30.1% of theory);

C₂₆H₂₇ClN₂O (M=418.97);

calc.: molar peak (M+H)⁺: 419/421 fnd.: molar peak (M+H)⁺: 419/421;

Retention time HPLC: 6.20 min (method A).

### EXAMPLE 2.58

### 3′.4′-dichloro-biphenyl-4-carboxylic acid [2-(4-pyrrolidin-1-ylmethyl-phenyl)-ethyl]-amide

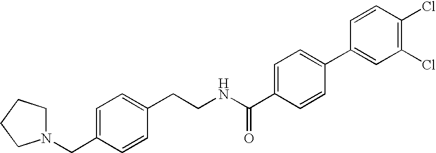

2.58a. 3′.4′-dichloro-biphenyl-4-carboxylic acid

Prepared analogously to Example 2.55a from 5.27 g (26.20 mmol) of 4-bromo-benzoic acid and 5.0 g (26.20 mmol) of 3′.4′-dichloro-phenylboric acid.

Yield: 4.05 g (57.9% of theory);

C₁₃H₈Cl₂O₂ (M=267.11);

calc.: molar peak (M−H)⁻: 265/267/269 fnd.: molar peak (M−H)⁻: 265/267/269.

2.58b. 3′.4′-dichloro-biphenyl-4-carboxylic acid [2-(4-pyrrolidin-1-ylmethyl-phenyl)-ethyl]-amide

Prepared according to general working method I from 102 mg (0.5 mmol) of 2-(4-pyrrolidin-1-ylmethyl-phenyl)-ethylamine and 134 mg (0.5 mmol) of 3′.4′-dichloro-biphenyl-4-carboxylic acid.

Yield: 45 mg (19.8% of theory);

C₂₆H₂₆Cl₂N₂O (M=453.42);

calc.: molar peak (M+H)⁺: 453/455/457 fnd.: molar peak (M+H)⁺: 453/455/457;

Retention time HPLC: 6.45 min (method A).

### EXAMPLE 2.59

### 2′,4′-dichloro-biphenyl-4-carboxylic acid [2-(4-pyrrolidin-1-ylmethyl-phenyl)-ethyl]-amide

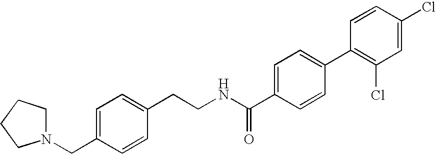

2.59a. 2′,4′-dichloro-biphenyl-4-carboxylic acid

Prepared analogously to Example 2.55a from 5.23 g (26.0 mmol) of 4-bromo-benzoic acid and 10.0 g (52.0 mmol) of 2,4-dichlorophenyl-boric acid, refluxing the reaction mixture for 48 h.

Yield: 1.5 g (21.6% of theory);

C₁₃H₈Cl₂O₂ (M=267.11);

calc.: molar peak (M−H)−: 265/267/269 fnd.: molar peak (M−H)−: 265/267/269.

2.59b. 2′,4′-dichloro-biphenyl-4-carboxylic acid [2-(4-pyrrolidin-1-ylmethyl-phenyl)-ethyl]-amide

Prepared according to general working method I from 102 mg (0.5 mmol) of 2-(4-pyrrolidin-1-ylmethyl-phenyl)-ethylamine and 134 mg (0.5 mmol) of 2′,4′-dichloro-biphenyl-4-carboxylic acid.

Yield: 72 mg (31.8% of theory);

C₂₆H₂₆Cl₂N₂O (M=453.42);

calc.: molar peak (M+H)⁺: 453/455/457 fnd.: molar peak (M+H)⁺: 453/455/457;

Retention time HPLC: 6.84 min (method A).

### EXAMPLE 2.60

### 2′-fluoro-4′-chloro-biphenyl-4-carboxylic acid [2-(4-pyrrolidin-1-ylmethyl-phenyl)-ethyl]-amide

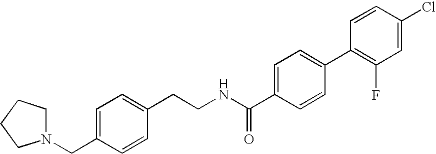

2.60a. 2′-fluoro-4′-chloro-biphenyl-4-carboxylic acid

Prepared analogously to Example 2.55a from 0.52 g (2.5 mmol) of 1-bromo-4-chloro-2-fluorobenzene and 0.5 g (3.0 mmol) of 4-carboxyphenyl-boric acid.

Yield: 0.5 g (79.8% of theory);

C₁₃H₈ClFO₂ (M=250.66);

calc.: molar peak (M−H)⁻: 249/251 fnd.: molar peak (M−H)⁻: 249/251;

Retention time HPLC: 8.39 min (method A).

2.60b. 2′-fluoro-4′-chloro-biphenyl-4-carboxylic acid [2-(4-pyrrolidin-1-ylmethyl-phenyl)-ethyl]-amide

Prepared according to general working method I from 102 mg (0.5 mmol) of 2-(4-pyrrolidin-1-ylmethyl-phenyl)-ethylamine and 125 mg (0.5 mmol) of 2′-fluoro-4′-chloro-biphenyl-4-carboxylic acid.

Yield: 36 mg (16.5% of theory);

C₂₆H₂₆ClFN₂O (M=436.96);

calc.: molar peak (M+H)⁺: 437/439 fnd.: molar peak (M+H)⁺: 437/439;

Retention time HPLC: 6.32 min (method A).

### EXAMPLE 2.61

### 3,4′-dichloro-biphenyl-4-carboxylic acid [2-(4-pyrrolidin-1-ylmethyl-phenyl)-ethyl]-amide

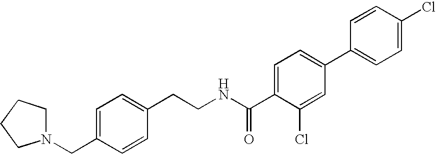

2.61 a. 3,4′-dichloro-biphenyl-4-carboxylic acid

Prepared analogously to Example 2.55a from 0.59 g (2.5 mmol) of 4-bromo-2-chloro-benzoic acid and 0.47 g (3.0 mmol) of 4-chlorophenyl-boric acid.

Yield: 0.55 g (82.4% of theory);

C₁₃H₈Cl₂O₂ (M=267.11);

calc.: molar peak (M−H)⁻: 265/267/269 fnd.: molar peak (M−H)⁻: 265/267/269;

Retention time HPLC: 8.83 min (method A).

2.61 b. 3,4′-dichloro-biphenyl-4-carboxylic acid [2-(4-pyrrolidin-1-ylmethyl-phenyl)-ethyl]-amide

Prepared according to general working method I from 102 mg (0.5 mmol) of 2-(4-pyrrolidin-1-ylmethyl-phenyl)-ethylamine and 134 mg (0.5 mmol) of 3,4′-dichloro-biphenyl-4-carboxylic acid.

Yield: 24 mg (10.6% of theory);

C₂₆H₂₆Cl₂N₂O (M=453.42);

calc.: molar peak (M+H)⁺: 453/455/457 fnd.: molar peak (M+H)⁺: 453/455/457;

Retention time HPLC: 6.41 min (method A).

### EXAMPLE 2.62

4′-chloro-3-fluoro-biphenyl-4-carboxylic acid [2-(4-pyrrolidin-1-ylmethyl-phenyl)-ethyl]-amide

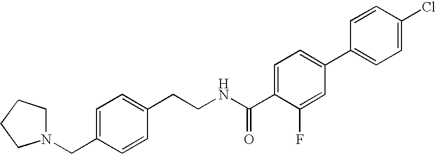

2.62a. 4′-chloro-3-fluoro-biphenyl-4-carboxylic acid

Prepared analogously to Example 2.55a from 0.55 g (2.5 mmol) of 4-bromo-2-fluoro-benzoic acid and 0.47 g (3.0 mmol) of 4-chlorophenyl-boric acid.

Yield: 0.60 g (95.7% of theory);

C₁₃H₈ClFO₂ (M=250.66);

calc.: molar peak (M−H)⁻: 249/251 fnd.: molar peak (M−H)⁻: 249/251;

Retention time HPLC: 8.22 min (method A).

2.62b. 4′-chloro-3-fluoro-biphenyl-4-carboxylic acid [2-(4-pyrrolidin-1-ylmethyl-phenyl)-ethyl]-amide

Prepared according to general working method I from 102 mg (0.5 mmol) of 2-(4-pyrrolidin-1-ylmethyl-phenyl)-ethylamine and 125 mg (0.5 mmol) of 4′-chloro-3-fluoro-biphenyl-4-carboxylic acid.

Yield: 37 mg (16.9% of theory);

C₂₆H₂₆ClFN₂O (M=436.96);

calc.: molar peak (M+H)⁺: 437/439 fnd.: molar peak (M+H)⁺: 437/439;

Retention time HPLC: 6.45 min (method A).

### EXAMPLE 2.63

### 4′-chloro-2-fluoro-biphenyl-4-carboxylic acid [2-(4-pyrrolidin-1-ylmethyl-phenyl)-ethyl]-amide

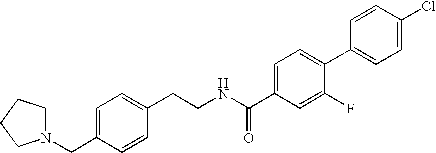

2.63a. 4′-chloro-2-fluoro-biphenyl-4-carboxylic acid

Prepared analogously to Example 2.55a from 0.66 g (3.0 mmol) of 4-bromo-3-fluoro-benzoic acid and 0.47 g (3.0 mmol) of 4-chlorophenyl-boric acid.

Yield: 0.60 g (79.8% of theory);

C₁₃H₈ClFO₂ (M=250.66);

calc.: molar peak (M−H)⁻: 249/251 fnd.: molar peak (M−H)⁻: 249/251;

Retention time HPLC: 8.50 min (method A).

2.63b. 4′-chloro-2-fluoro-biphenyl-4-carboxylic acid [2-(4-pyrrolidin-1-ylmethyl-phenyl)-ethyl]-amide

Prepared according to general working method I from 163 mg (0.8 mmol) of 2-(4-pyrrolidin-1-ylmethyl-phenyl)-ethylamine and 201 mg (0.8 mmol) of 4′-chloro-2-fluoro-biphenyl-4-carboxylic acid.

Yield: 74 mg (21.2% of theory);

C₂₆H₂₆ClFN₂O (M=436.96);

calc.: molar peak (M+H)⁺: 437/439 fnd.: molar peak (M+H)⁺: 437/439;

Retention time HPLC: 6.61 min (method A).

### EXAMPLE 2.64

### 3-nitro-biphenyl-4-carboxylic acid [2-(4-pyrrolidin-1-ylmethyl-phenyl)-ethyl]-amide

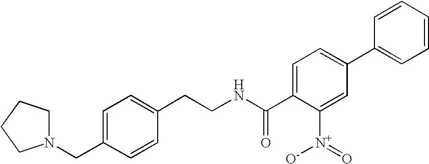

2.64a. 3-nitro-biphenyl-4-carboxylic acid

150 mg (0.13 mmol) of tetrakis-(triphenylphosphine)-palladium are added to a solution of 1.0 g (4.07 mmol) of 4-bromo-2-nitro-benzoic acid in 20 mL toluene and stirred for 10 min at RT. Then a solution of 0.5 g (4.10 mmol) of phenylboric acid in 10 mL MeOH and a solution of 1.0 g Na₂CO₃ in 10 mL water are added. The reaction mixture is refluxed for 5 h and stirred at RT over the weekend. The solvents are eliminated in vacuo, the residue is combined with water, acidified with conc. HCl, extracted with EtOAc, the organic phase is dried over Na₂SO₄ and then the solvent is removed.

Yield: 0.87 g (87.5% of theory);

R_{f} value: 0.40 (silica gel, dichloromethane/ethanol 3:1).

2.64b. 3-nitro-biphenyl-4-carboxylic acid [2-(4-pyrrolidin-1-ylmethyl-phenyl)-ethyl]-amide

Prepared according to general working method I from 102 mg (0.5 mmol) of 2-(4-pyrrolidin-1-ylmethyl-phenyl)-ethylamine and 122 mg (0.5 mmol) of 3-nitro-biphenyl-4-carboxylic acid.

Yield: 100 mg (46.6% of theory);

C₂₆H₂₇N₃O₃ (M=429.52);

calc.: molar peak (M+H)⁺: 430 fnd.: molar peak (M+H)⁺: 430;

Retention time HPLC: 5.83 min (method A).

### EXAMPLE 2.65

### 5-(4-chloro-phenyl)-pyridine-2-carboxylic acid [2-(4-pyrrolidin-1-ylmethyl-phenyl)-ethyl]-amide

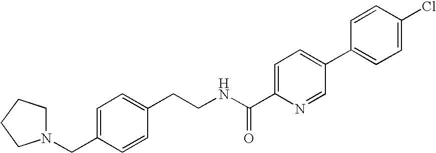

2.65a. 5-(4-chloro-phenyl)-pyridine-2-carboxylic acid

Prepared analogously to Example 2.55a from 0.51 g (2.5 mmol) of 5-bromo-pyridine-2-carboxylic acid and 0.47 g (3.0 mmol) of 4-chlorophenyl-boric acid.

Yield: 0.23 g (39.4% of theory);

C₁₂H₈ClNO₂ (M=233.66);

calc.: molar peak (M−H)⁻: 232/234 fnd.: molar peak (M−H)⁻: 232/234;

Retention time HPLC: 5.89 min (method A).

2.65b. 5-(4-chloro-phenyl)-pyridine-2-carboxylic acid [2-(4-pyrrolidin-1-ylmethyl-phenyl)-ethyl]-amide

Prepared according to general working method I from 102 mg (0.5 mmol) of 2-(4-pyrrolidin-1-ylmethyl-phenyl)-ethylamine and 116 mg (0.5 mmol) of 5-(4-chloro-phenyl)-pyridine-2-carboxylic acid.

Yield: 7 mg (3.3% of theory);

C₂₅H₂₆ClN₃O (M=419.96);

calc.: molar peak (M+H)⁺: 420/422 fnd.: molar peak (M+H)⁺: 420/422;

Retention time HPLC: 6.40 min (method A).

### EXAMPLE 2.66

### N-[2-(4-pyrrolidin-1-ylmethyl-phenyl)-ethyl]-4-thiophen-3-yl-benzamide

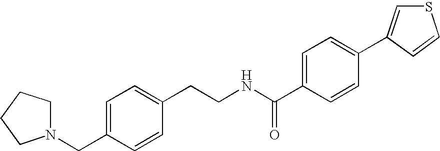

2.66a. 4-thiophen-3-yl-benzoate ethyl

Prepared analogously to Example 2.46b from 414 mg (1.5 mmol) of ethyl 4-iodo-benzoate and 230 mg (1.8 mmol) of thiophene-3-boric acid.

Yield: 348 mg (100% of theory);

C₁₃H₁₂O₂S (M=232.30);

calc.: molar peak (M+H)⁺: 233 fnd.: molar peak (M+H)⁺: 233;

Retention time HPLC: 6.20 min (method B).

2.66b. 4-thiophen-3-yl-benzoic acid

Prepared analogously to Example 2.7b from 280 mg (1.5 mmol) of ethyl 4-thiophen-3-yl-benzoate.

Yield: 146 mg (59.3% of theory);

C₁₁H₈O₂S (M=204.25);

calc.: molar peak (M−H)⁻: 203 fnd.: molar peak (M−H)⁻: 203;

Retention time HPLC: 7.60 min (method A).

2.66c. N-[2-(4-pyrrolidin-1-ylmethyl-phenyl)-ethyl]-4-thiophen-3-yl-benzamide

Prepared according to general working method I from 102 mg (0.5 mmol) of 2-(4-pyrrolidin-1-ylmethyl-phenyl)-ethylamine and 102 mg (0.5 mmol) of 4-thiophen-3-yl-benzoic acid.

Yield: 103 mg (53.0% of theory);

C₂₄H₂₆N₂OS (M=390.55);

calc.: molar peak (M+H)⁺: 391 fnd.: molar peak (M+H)⁺: 391;

Retention time HPLC: 6.10 min (method A).

### EXAMPLE 2.67

### N-[2-(4-pyrrolidin-1-ylmethyl-phenyl)-ethyl]-4-thiophen-2-yl-benzamide

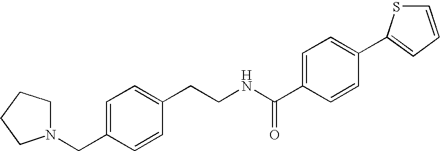

2.67a. ethyl 4-thiophen-2-yl-benzoate

Prepared analogously to Example 2.46b from 414 mg (1.5 mmol) of ethyl 4-iodo-benzoate and 230 mg (1.8 mmol) of thiophene-2-boric acid.

Yield: 348 mg (100% of theory);

C₁₃H₁₂O₂S (M=232.30);

calc.: molar peak (M+H)⁺: 233 fnd.: molar peak (M+H)⁺: 233;

Retention time HPLC: 6.29 min (method B).

2.67b. 4-thiophen-2-yl-benzoic acid

Prepared analogously to Example 2.7b from 280 mg (1.5 mmol) of ethyl 4-thiophen-2-yl-benzoate.

Yield: 126 mg (51.2% of theory);

C₁₁H₈O₂S (M=204.25);

calc.: molar peak (M−H)⁻: 203 fnd.: molar peak (M−H)⁻: 203;

Retention time HPLC: 7.60 min (method A).

2.67c. N-[2-(4-pyrrolidin-1-ylmethyl-phenyl)-ethyl]-4-thiophen-2-yl-benzamide

Prepared according to general working method I from 102 mg (0.5 mmol) of 2-(4-pyrrolidin-1-ylmethyl-phenyl)-ethylamine and 102 mg (0.5 mmol) of 4-thiophen-2-yl-benzoic acid.

Yield: 112 mg (57.5% of theory);

C₂₄H₂₆N₂OS (M=390.55);

calc.: molar peak (M+H)⁺: 391 fnd.: molar peak (M+H)⁺: 391;

Retention time HPLC: 6.05 min (method A).

### EXAMPLE 2.68

### 4-(5-chloro-thiophen-2-yl)-N-[2-(4-pyrrolidin-1-ylmethyl-phenyl)-ethyl]-benzamide

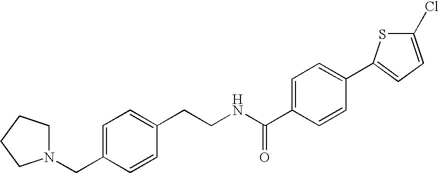

2.68a. 4-(5-chloro-thiophen-2-yl)-benzoic acid

Prepared analogously to Example 2.55a from 300 mg (1.52 mmol) of 2-bromo-5-chlorothiophene and 277 mg (1.67 mmol) of 4-carboxyphenyl-boric acid, using KHSO₄ solution to acidify the worked up reaction mixture.

Yield: 76 mg (21.0% of theory);

C₁₁H₇ClO₂S (M=238.69);

calc.: molar peak (M−H)⁻: 237/239 fnd.: molar peak (M−H)⁻: 237/239;

Retention time HPLC: 8.75 min (method A).

2.68b. 4-(5-chloro-thiophen-2-yl)-N-[2-(4-pyrrolidin-1-ylmethyl-phenyl)-ethyl]-benzamide

Prepared according to general working method I from 61 mg (0.3 mmol) of 2-(4-pyrrolidin-1-ylmethyl-phenyl)-ethylamine and 71 mg (0.3 mmol) of 4-(5-chloro-thiophen-2-yl)-benzoic acid.

Yield: 29 mg (22.9% of theory);

C₂₄H₂₅ClN₂OS (M=425.0);

calc.: molar peak (M+H)⁺: 425/427 fnd.: molar peak (M+H)⁺: 425/427;

Retention time HPLC: 6.65 min (method A).

### EXAMPLE 2.69

### 4-furan-2-yl-N-[2-(4-pyrrolidin-1-ylmethyl-phenyl)-ethyl]-benzamide

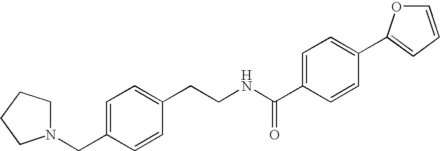

2.69a. 4-furan-2-yl-benzoic acid

Prepared analogously to Example 2.68a from 302 mg (1.5 mmol) of 4-bromo-benzoic acid and 201 mg (1.8 mmol) of furan-2-boric acid.

Yield: 166 mg (58.8% of theory);

C₁₁H₈O₃ (M=188.19);

calc.: molar peak (M−H)⁻: 187 fnd.: molar peak (M−H)⁻: 187;

Retention time HPLC: 6.82 min (method A).

2.69b. 4-furan-2-yl-N-[2-(4-pyrrolidin-1-ylmethyl-phenyl)-ethyl]-benzamide

Prepared according to general working method I from 102 mg (0.5 mmol) of 2-(4-pyrrolidin-1-ylmethyl-phenyl)-ethylamine and 94 mg (0.5 mmol) of 4-furan-2-yl-benzoic acid.

Yield: 91 mg (48.4% of theory);

C₂₄H₂₆N₂O₂ (M=374.49);

calc.: molar peak (M+H)⁺: 375 fnd.: molar peak (M+H)⁺: 375;

Retention time HPLC: 6.48 min (method A).

### EXAMPLE 2.70

### 4-(5-methyl-pyridin-2-yl)-N-[2-(4-pyrrolidin-1-ylmethyl-phenyl)-ethyl]-benzamide

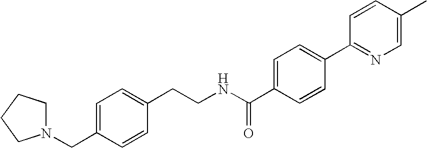

2.70a. 4-(5-methyl-pyridin-2-yl)-benzoic acid

Prepared analogously to Example 2.55a from 430 mg (2.50 mmol) of 2-bromo-5-methylpyridine and 498 mg (3.00 mmol) of 4-carboxyphenyl-boric acid.

Yield: 300 mg (56.3% of theory);

C₁₃H₁₁NO₂ (M=213.24);

calc.: molar peak (M+H)⁺: 214 fnd.: molar peak (M+H)⁺: 214;

Retention time HPLC: 4.55 min (method A).

2.70b. 4-(5-methyl-pyridin-2-yl)-N-[2-(4-pyrrolidin-1-ylmethyl-phenyl)-ethyl]-benzamide

Prepared according to general working method I from 102 mg (0.5 mmol) of 2-(4-pyrrolidin-1-ylmethyl-phenyl)-ethylamine and 107 mg (0.5 mmol) of 4-(5-methyl-pyridin-2-yl)-benzoic acid.

Yield: 53 mg (26.5% of theory);

C₂₆H₂₉N₃O (M=399.54);

calc.: molar peak (M+H)⁺: 400 fnd.: molar peak (M+H)⁺: 400;

Retention time HPLC: 3.98 min (method A).

### EXAMPLE 2.71

### 4-(6-methyl-pyridin-3-yl)-N-[2-(4-pyrrolidin-1-ylmethyl-phenyl)-ethyl]-benzamide

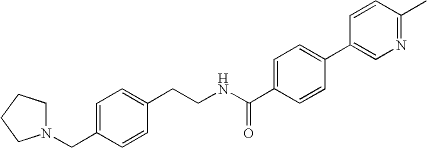

2.71 a. 4-(6-methyl-pyridin-3-yl)-benzoic acid

Prepared analogously to Example 2.55a from 430 mg (2.50 mmol) of 5-bromo-2-methylpyridine and 498 mg (3.00 mmol) of 4-carboxyphenyl-boric acid.

Yield: 300 mg (56.3% of theory);

C₁₃H₁₁NO₂ (M=213.24);

calc.: molar peak (M+H)⁺: 214 fnd.: molar peak (M+H)⁺: 214;

Retention time HPLC: 2.66 min (method A).

2.71 b. 4-(6-methyl-pyridin-3-yl)-N-[2-(4-pyrrolidin-1-ylmethyl-phenyl)-ethyl]-benzamide

Prepared according to general working method I from 102 mg (0.5 mmol) of 2-(4-pyrrolidin-1-ylmethyl-phenyl)-ethylamine and 107 mg (0.5 mmol) of 4-(6-methyl-pyridin-3-yl)-benzoic acid.

Yield: 48 mg (24.0% of theory);

C₂₆H₂₉N₃O (M=399.54);

calc.: molar peak (M+H)⁺: 400 fnd.: molar peak (M+H)⁺: 400;

Retention time HPLC: 3.06 min (method A).

### EXAMPLE 2.72

### 4-(4-Chloro-phenyl)-thiophene-2-carboxylic acid [2-(4-pyrrolidin-1-ylmethyl-phenyl)-ethyl]-amide

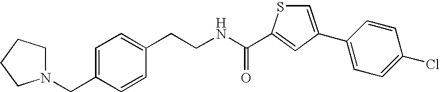

2.72a. methyl 4-(4-chloro-phenyl)-thiophene-2-carboxylate

420 mg (1.25 mmol) of methyl 4-bromo-thiophene-2-carboxylate are dissolved in 10 mL dioxane and 5 mL 2M Na₂CO₃ solution. 196 mg (0.06 mmol) of 4-chloro-phenyl-boric acid and 72 mg (0.06 mmol) of tetrakis-(triphenylphosphine)-palladium are added successively, the reaction is refluxed for 6 h and stirred for a further 60 h at RT. After being heated again, the hot reaction solution is suction filtered through a glass fibre filter, washed with dioxane, combined with semisaturated NaHCO₃ solution and extracted with EtOAc. The combined organic phases are dried over MgSO₄. After elimination of the drying agent and solvent the residue is purified by column chromatography on silica gel (petroleum ether/ethyl acetate 9:1).

Yield: 150 mg (47.3% of theory);

C₁₂H₉ClO₂S (M=252.72);

calc.: molar peak (M+H)⁺: 253/255 fnd.: molar peak (M+H)⁺: 253/255;

Retention time HPLC: 6.21 min (method B).

2.72b. 4-(4-chloro-phenyl)-thiophene-2-carboxylic acid

2 mL 1M NaOH solution are added to a solution of 150 mg methyl 4-(4-chloro-phenyl)-thiophene-2-carboxylate in 10 mL EtOH and the reaction solution is stirred at RT over the weekend. The solvent is evaporated down in vacuo, the residue combined with 2 mL 1N hydrochloric acid and cooled to 0° C. The precipitated product is suction filtered, washed with water and dried at 50° C.

Yield: 140 mg (98.7% of theory);

C₁₁H₇ClO₂S (M=238.69);

calc.: molar peak (M+H)⁺: 239/241 fnd.: molar peak (M+H)⁺: 239/241;

Retention time HPLC: 8.31 min (method A).

2.72c. 4-(4-chloro-phenyl)-thiophene-2-carboxylic acid [2-(4-pyrrolidin-1-ylmethyl-phenyl)-ethyl]-amide

Prepared according to general working method I from 144 mg (0.70 mmol) of 2-(4-pyrrolidin-1-ylmethyl-phenyl)-ethylamine and 140 mg (0.59 mmol) of 4-(4-chloro-phenyl)-thiophene-2-carboxylic acid.

Yield: 78 mg (31.3% of theory);

C₂₆H₂₉N₃O (M=425.00);

calc.: molar peak (M+H)⁺: 425/427 fnd.: molar peak (M+H)⁺: 425/427;

Retention time HPLC: 3.90 min (method A).

### EXAMPLE 2.73

### 4-(5-acetyl-thiophen-2-yl)-N-[2-(4-pyrrolidin-1-ylmethyl-phenyl)-ethyl]-benzamide

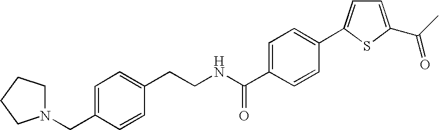

2.73a. 4-iodo-N-[2-(4-pyrrolidin-1-ylmethyl-phenyl)-ethyl]-benzamide

Prepared according to general working method I from 2.04 g (10.0 mmol) of 2-(4-pyrrolidin-1-ylmethyl-phenyl)-ethylamine and 2.48 g (10.0 mmol) of 4-iodo-benzoic acid.

Yield: 1.91 g (44.0% of theory);

C₂₀H₂₃IN₂O (M=434.32);

calc.: molar peak (M+H)⁺: 435 fnd.: molar peak (M+H)⁺: 435;

Retention time HPLC: 5.40 min (method A).

2.73b. 4-(5-acetyl-thiophen-2-yl)-N-[2-(4-pyrrolidin-1-ylmethyl-phenyl)-ethyl]-benzamide

Prepared analogously to Example 2.46b from 250 mg (0.58 mmol) of 4-iodo-N-[2-(4-pyrrolidin-1-ylmethyl-phenyl)-ethyl]-benzamide and 118 mg (0.69 mmol) of 5-acetyl-2-thiophene-boric acid, refluxing the reaction mixture for 15 h.

Yield: 50 mg (20.2% of theory);

C₂₆H₂₈N₂O₂S (M=432.59);

calc.: molar peak (M+H)⁺: 433 fnd.: molar peak (M+H)⁺: 433;

Retention time HPLC: 3.91 min (method B).

### EXAMPLE 2.74

### 4-(5-formyl-thiophen-2-yl)-N-[2-(4-pyrrolidin-1-ylmethyl-phenyl)-ethyl]-benzamide

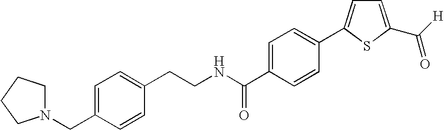

Prepared analogously to Example 2.46b from 250 mg (0.58 mmol) of 4-iodo-N-[2-(4-pyrrolidin-1-ylmethyl-phenyl)-ethyl]-benzamide and 107 mg (0.69 mmol) of 5-formyl-2-thiophene-boric acid, by refluxing the reaction mixture for 15 h.

Yield: 22 mg (9.1% of theory);

C₂₅H₂₆N₂O₂S (M=418.56);

calc.: molar peak (M+H)⁺: 419 fnd.: molar peak (M+H)⁺: 419;

Retention time HPLC: 3.82 min (method B).

### EXAMPLE 2.75

### 4′-chloro-biphenyl-4-carboxylic acid [2-(4-aminomethyl-phenyl)-ethyl]-amide

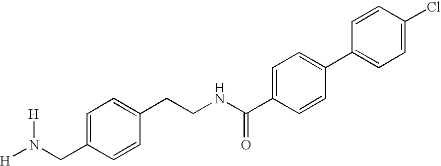

2.75a. ethyl 4-{2-[(4′-chloro-biphenyl-4-carbonyl)-amino]-ethyl}-benzoate

20 mL thionyl chloride and 1 mL DMF are added dropwise to 9.31 g (40 mmol) of 4′-chloro-biphenyl-4-carboxylic acid. The reaction mixture is heated to 60° C. for 2 h. Then the excess thionyl chloride is eliminated in vacuo at 50° C. and the residue is taken up in 200 mL CH₂Cl₂. This solution is added dropwise to 9.19 g (40 mmol) of ethyl 4-(2-amino-ethyl)-benzoate, used as the hydrochloride, in 100 mL of 10% aqueous Na₂CO₃ solution and the reaction mixture is stirred for a further hour at RT. After the addition of water and CH₂Cl₂ the organic phase is separated off, the aqueous phase is extracted with CH₂Cl₂, the combined organic phases are washed with semisaturated NaHCO₃ solution and water and dried over MgSO₄. After elimination of the drying agent the solution is filtered through activated charcoal, evaporated down in vacuo and the residue recrystallised from tert-butylmethylether.

Yield: 11.93 g (73.1% of theory);

C₂₄H₂₂ClNO₃ (M=407.90);

calc.: molar peak (M+H)⁺: 408 fnd.: molar peak (M+H)⁺: 408;

Retention time HPLC: 9.8 min (method A).

2.75b. 4-{2-[(4′-chloro-biphenyl-4-carbonyl)-amino]-ethyl}-benzoic acid

50 mL 2M NaOH solution are added to a solution of 11.93 g (29.25 mmol) of ethyl 4-{2-[(4′-chloro-biphenyl-4-carbonyl)-amino]-ethyl}-benzoate in 150 mL EtOH and stirred for 2 h at RT. The reaction solution is adjusted to pH 6-7 with 1N HCl solution, the precipitated product is filtered off and dried in the vacuum oven.

Yield: 10.74 g (96.7% of theory);

C₂₂H₁₈ClNO₃ (M=379.85);

calc.: molar peak (M+H)⁺: 380/382 fnd.: molar peak (M+H)⁺: 380/382;

Retention time HPLC: 8.0 min (method A).

2.75c. 4′-chloro-biphenyl-4-carboxylic acid [2-(4-hydroxymethyl-phenyl)-ethyl]-amide

4.82 g (29.69 mmol) of CDI are added to a solution of 10.74 g (28.28 mmol) of 4-{2-[(4′-chloro-biphenyl-4-carbonyl)-amino]-ethyl}-benzoic acid in 150 mL dry THF and the reaction mixture is heated to 50° C. for 2 h. This solution is added to a suspension of 2.14 g (56.56 mmol) of NaBH₄ in 5 mL water and stirred vigorously for a further hour at RT. Using 1N HCl the pH of the solution is adjusted to 6, it is then combined with EtOAc and filtered. The filtrate is washed with semisaturated NaHCO₃ solution and water and dried over MgSO₄. As the residue still contains unreacted 4-{2-[(4′-chloro-biphenyl-4-carbonyl)-amino]-ethyl}-benzoic acid after elimination of the drying agent and solvent the above reduction step is repeated. The product obtained is dried at 40° C.

Yield: 9.3 g (89.9% of theory);

C₂₂H₂₀ClNO₂ (M=365.86);

calc.: molar peak (M+H)⁺: 366/368 fnd.: molar peak (M+H)⁺: 366/368;

Retention time HPLC: 8.11 min (method A).

2.75d. 4′-chloro-biphenyl-4-carboxylic acid [2-(4-bromomethyl-phenyl)-ethyl]-amide

1.22 ml PBr₃ are added dropwise to a solution of 7.9 g (21.59 mmol) of 4′-chloro-biphenyl-4-carboxylic acid [2-(4-hydroxymethyl-phenyl)-ethyl]-amide in 300 mL CH₂Cl₂. The reaction mixture is stirred overnight at RT. The precipitate formed is suction filtered and the filtrate evaporated down. The residue is triturated with a little acetonitrile and CH₂Cl₂, suction filtered, combined with the precipitate obtained at first and dried in the air.

Yield: 8.6 g (92.9% of theory);

C₂₂H₁₉BrClNO (M=428.76);

calc.: molar peak (M+H)⁺: 428/430/432 fnd.: molar peak (M+H)⁺: 428/430/432;

R_{f} value: 0.40 (silica gel, CH₂Cl₂).

2.75e. 4′-chloro-biphenyl-4-carboxylic acid [2-(4-aminomethyl-phenyl)-ethyl]-amide

3 mL of a 0.5 M NH₃ solution in dioxane are added to a solution of 150 mg (0.35 mmol) of 4′-chloro-biphenyl-4-carboxylic acid [2-(4-bromomethyl-phenyl)-ethyl]-amide in 10 mL acetonitrile and stirred for 3 days at RT. The reaction mixture is evaporated down and the residue purified by column chromatography (silica gel, CH₂Cl₂/MeOH/NH₃ 9:1:0.1).

Yield: 8 mg (6.3% of theory);

C₂₂H₂₁ClN₂O (M=364.88);

calc.: molar peak (M+H)⁺: 365/367 fnd.: molar peak (M+H)⁺: 365/367;

Retention time HPLC: 5.97 min (method A).

### EXAMPLE 2.76

### 4′-chloro-biphenyl-4-carboxylic acid (2-{4-[(diisopropylamino)-methyl]-phenyl}-ethyl)-amide

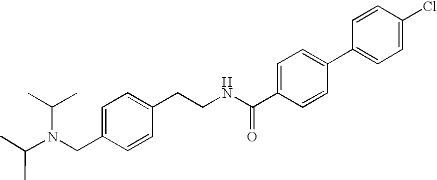

47 μL (0.33 mmol) of diisopropylamine are added to a suspension of 129 mg (0.3 mmol) of 4′-chloro-biphenyl-4-carboxylic acid [2-(4-bromomethyl-phenyl)-ethyl]-amide and 55 mg (0.4 mmol) of K₂CO₃ in 20 mL acetonitrile and the reaction mixture is stirred overnight at RT. It is diluted with CH₂Cl₂, filtered to remove insoluble inorganic salts and the filtrate is evaporated down. The residue is triturated with acetonitrile, suction filtered and dried in the air.

Yield: 75 mg (55.7% of theory);

C₂₈H₃₃ClN₂O (M=449.04);

calc.: molar peak (M+H)⁺: 449/451 fnd.: molar peak (M+H)⁺: 449/451;

R_{f} value: 0.35 (silica gel, CH₂Cl₂/MeOH/NH₃ 95:5:0.5).

### EXAMPLE 2.77

### 4′-chloro-biphenyl-4-carboxylic acid {2-[4-(3-oxo-piperazin-1-ylmethyl)-phenyl]-ethyl}-amide

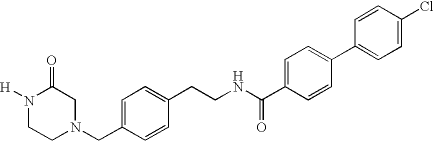

Prepared analogously to Example 2.76 from 129 mg (0.3 mmol) of 4′-chloro-biphenyl-4-carboxylic acid [2-(4-bromomethyl-phenyl)-ethyl]-amide and 33 mg (0.33 mmol) of piperazine-2-one.

Yield: 23 mg (17.1% of theory);

C₂₆H₂₆ClN₃O₂ (M=447.97);

calc.: molar peak (M+H)⁺: 448/450 fnd.: molar peak (M+H)⁺: 448/450;

R_{f} value: 0.10 (silica gel, CH₂Cl₂/MeOH/NH₃ 95:5:0.5).

### EXAMPLE 2.78

### Ethyl [(4-{2-[(4′-chloro-biphenyl-4-carbonyl)-amino]-ethyl}-benzyl)-methyl-amino]-acetate

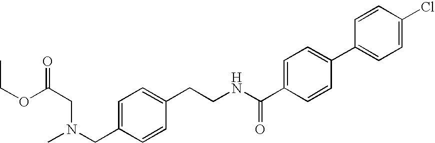

Prepared analogously to Example 2.76 from 257 mg (0.6 mmol) of 4′-chloro-biphenyl-4-carboxylic acid [2-(4-bromomethyl-phenyl)-ethyl]-amide, 193 mg K₂CO₃ and 101 mg (0.66 mmol) of ethyl methylamino-acetate (used as the hydrochloride).

Yield: 152 mg (54.5% of theory);

C₂₇H₂₉ClN₂O₃ (M=465.0);

calc.: molar peak (M+H)⁺: 465/467 fnd.: molar peak (M+H)⁺: 465/467;

R_{f} value: 0.40 (silica gel, CH₂Cl₂/MeOH/NH₃ 95:5:0.5).

### EXAMPLE 2.79

### [(4-{2-[(4′-chloro-biphenyl-4-carbonyl)-amino]-ethyl}-benzyl)-methyl-amino]-acetic acid

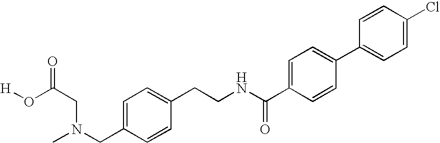

0.3 mL 1M NaOH solution are added to a solution of 80 mg (0.17 mmol) of ethyl [(4-{2-[(4′-chloro-biphenyl-4-carbonyl)-amino]-ethyl}-benzyl)-methyl-amino]-acetate in 3 mL EtOH and refluxed for 1 h. The solvent is evaporated down in vacuo and the residue combined with water and 0.3 mL 1 M HCl. The precipitate is suction filtered and dried at 40° C.

Yield: 76 mg (100% of theory);

C₂₅H₂₅ClN₂O₃ (M=436.94);

calc.: molar peak (M+H)⁺: 437/439 fnd.: molar peak (M+H)⁺: 437/439;

Retention time HPLC: 6.35 min (method A).

### EXAMPLE 2.80

### 4′-chloro-biphenyl-4-carboxylic acid{2-[4-(4-acetyl-piperazin-1-ylmethyl)-phenyl]-ethyl}-amide

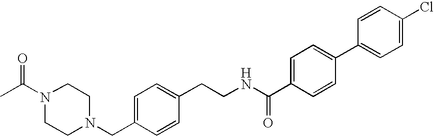

Prepared analogously to Example 2.76 from 129 mg (0.3 mmol) of 4′-chloro-biphenyl-4-carboxylic acid [2-(4-bromomethyl-phenyl)-ethyl]-amide and 42 mg (0.33 mmol) of 1-piperazin-1-yl-ethanone.

Yield: 60 mg (42.0% of theory);

C₂₈H₃₀ClN₃O₂ (M=476.02);

calc.: molar peak (M+H)⁺: 476/478 fnd.: molar peak (M+H)⁺: 476/478;

R_{f} value: 0.15 (silica gel, CH₂Cl₂/MeOH/NH₃ 95:5:0.5).

### EXAMPLE 2.81

### 4′-Chloro-biphenyl-4-carboxylic acid{2-[4-(2-aza-bicyclo[2.2.1]hept-5-en-2-ylmethyl)-phenyl]-ethyl}-amide

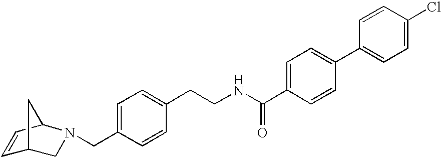

Prepared analogously to Example 2.76 from 129 mg (0.3 mmol) of 4′-chloro-biphenyl-4-carboxylic acid [2-(4-bromomethyl-phenyl)-ethyl]-amide and 31 mg (0.33 mmol) of 2-aza-bicyclo[2.2.1]hept-5-ene.

Yield: 100 mg (75.2% of theory);

C₂₈H₂₇ClN₂O (M=442.99);

calc.: molar peak (M+H)⁺: 443/445 fnd.: molar peak (M+H)⁺: 443/445;

R_{f} value: 0.08 (silica gel, CH₂Cl₂/MeOH/NH₃ 95:5:0.5).

### EXAMPLE 2.82

### 4′-chloro-biphenyl-4-carboxylic acid-{2-[4-(1,3-dihydro-isoindol-2-ylmethyl)-phenyl]-ethyl}-amide

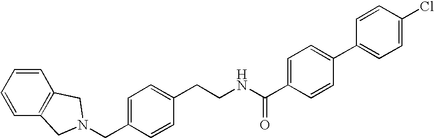

Prepared analogously to Example 2.76 from 129 mg (0.3 mmol) of 4′-chloro-biphenyl-4-carboxylic acid [2-(4-bromomethyl-phenyl)-ethyl]-amide, 97 mg K₂CO₃ and 51 mg (0.33 mmol) of 2,3-dihydro-1H-isoindole (used as the hydrochloride).

Yield: 80 mg (57.1% of theory);

C₃₀H₂₇ClN₂O (M=467.02);

calc.: molar peak (M+H)⁺: 467/469 fnd.: molar peak (M+H)⁺: 467/469;

R_{f} value: 0.40 (silica gel, CH₂Cl₂/MeOH/NH₃ 95:5:0.5).

### EXAMPLE 2.83

### 4′-chloro-biphenyl-4-carboxylic acid-{2-[4-(7-methyl-2.7-diaza-spiro[4.4]non-2-ylmethyl)-phenyl]-ethyl}-amide

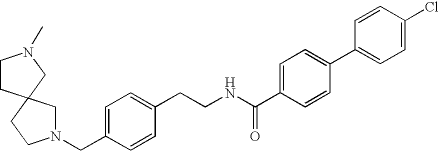

Prepared analogously to Example 2.76 from 129 mg (0.3 mmol) of 4′-chloro-biphenyl-4-carboxylic acid [2-(4-bromomethyl-phenyl)-ethyl]-amide and 46 mg (0.33 mmol) of 2-methyl-2.7-diaza-spiro[4.4]nonane.

Yield: 42 mg (28.7% of theory);

C₃₀H₃₄ClN₃O (M=488.08);

calc.: molar peak (M+H)⁺: 488/490 fnd.: molar peak (M+H)⁺: 488/490;

R_{f} value: 0.05 (silica gel, CH₂Cl₂/MeOH/NH₃ 95:5:0.5).

### EXAMPLE 2.84

### 4′-chloro-biphenyl-4-carboxylic acid-{2-[4-(3-diethylamino-azetidin-1-ylmethyl)-phenyl]-ethyl}-amide

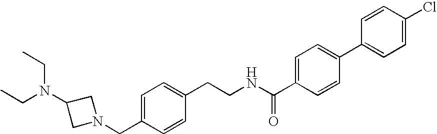

Prepared analogously to Example 2.76 from 129 mg (0.3 mmol) of 4′-chloro-biphenyl-4-carboxylic acid [2-(4-bromomethyl-phenyl)-ethyl]-amide, 138 mg K₂CO₃ and 66 mg (0.33 mmol) of azetidin-3-yl-diethyl-amine (used as bis-hydrochloride); the product is purified by column chromatography.

Yield: 15 mg (10.5% of theory);

C₂₉H₃₄ClN₃O (M=476.07);

calc.: molar peak (M+H)⁺: 476/478 fnd.: molar peak (M+H)⁺: 476/478;

R_{f} value: 0.10 (silica gel, CH₂Cl₂/MeOH/NH₃ 95:5:0.1).

### EXAMPLE 2.85

### Ethyl (S)-1-(4-{2-[(4′-chloro-biphenyl-4-carbonyl)-amino]-ethyl}-benzyl)-pyrrolidine-2-carboxylate

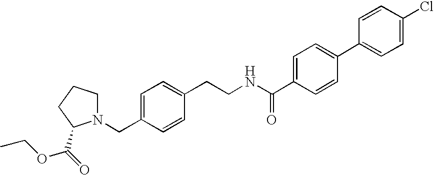

Prepared analogously to Example 2.76 from 257 mg (0.6 mmol) of 4′-chloro-biphenyl-4-carboxylic acid [2-(4-bromomethyl-phenyl)-ethyl]-amide, 193 mg K₂CO₃ and 119 mg (0.66 mmol) of ethyl (S)-pyrrolidine-2-carboxylate (used as the hydrochloride); the product is purified by column chromatography.

Yield: 160 mg (54.3% of theory);

C₂₉H₃₁ClN₂O₃ (M=491.04);

calc.: molar peak (M+H)⁺: 491/493 fnd.: molar peak (M+H)⁺: 491/493;

R_{f} value: 0.60 (silica gel, CH₂Cl₂/MeOH/NH₃ 95:5:0.5).

### EXAMPLE 2.86

### (S)-1-(4-{2-[(4′-chloro-biphenyl-4-carbonyl)-amino]-ethyl}-benzyl)-pyrrolidine-2-carboxylic acid

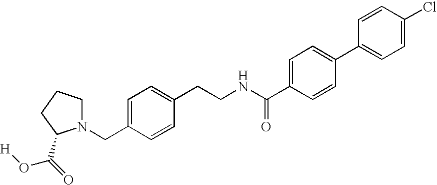

Prepared analogously to Example 2.79 from 130 mg (0.27 mmol) of ethyl (S)-1-(4-{2-[(4′-chloro-biphenyl-4-carbonyl)-amino]-ethyl}-benzyl)-pyrrolidine-2-carboxylate.

Yield: 120 mg (97.8% of theory);

C₂₇H₂₇ClN₂O₃ (M=462.98);

calc.: molar peak (M+H)⁺: 463/465 fnd.: molar peak (M+H)⁺: 463/465;

Retention time HPLC: 6.20 min (method A).

### EXAMPLE 2.87

### Tert.butyl [1-(4-{2-[(4′-chloro-biphenyl-4-carbonyl)-amino]-ethyl}-benzyl)-pyrrolidin-3-yl]-carbaminate

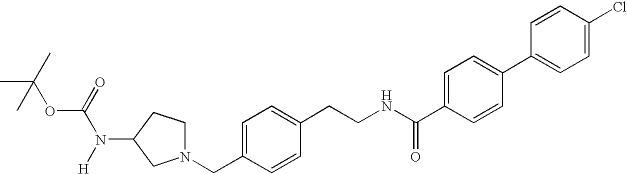

Prepared analogously to Example 2.76 from 429 mg (1.0 mmol) of 4′-chloro-biphenyl-4-carboxylic acid [2-(4-bromomethyl-phenyl)-ethyl]-amide and 205 mg (1.10 mmol) of tert.butyl pyrrolidin-3-yl-carbaminate.

Yield: 500 mg (93.6% of theory);

C₃₁H₃₆ClN₃O₃ (M=534.10);

calc.: molar peak (M+H)⁺: 534/536 fnd.: molar peak (M+H)⁺: 534/536;

R_{f} value: 0.33 (silica gel, CH₂Cl₂/MeOH/NH₃ 95:5:0.5).

### EXAMPLE 2.88

### 4′-chloro-biphenyl-4-carboxylic acid{2-[4-(3-amino-pyrrolidin-1-ylmethyl)-phenyl]-ethyl}-amide

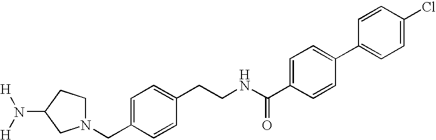

1 mL trifluoroacetic acid are added to a solution of 500 mg (0.94 mmol) of tert.butyl [1-(4-{2-[(4′-chloro-biphenyl-4-carbonyl)-amino]-ethyl}-benzyl)-pyrrolidin-3-yl]-carbaminate in 15 mL CH₂Cl₂ and the reaction mixture is stirred overnight. This is then evaporated down, the residue taken up in a little CH₂Cl₂ and combined with semisaturated NaHCO₃ solution. The precipitated product is suction filtered, triturated with acetonitrile and dried at 40° C.

Yield: 240 mg (59.1% of theory);

C₂₆H₂₈ClN₃O (M=433.99);

calc.: molar peak (M+H)⁺: 434/436 fnd.: molar peak (M+H)⁺: 434/436;

R_{f} value: 0.22 (silica gel, CH₂Cl₂/MeOH/NH₃ 9:1:0.1).

### EXAMPLE 2.89

### 4′-chloro-biphenyl-4-carboxylic acid{2-[4-(3-dimethylamino-pyrrolidin-1-ylmethyl)-phenyl]-ethyl}-amide

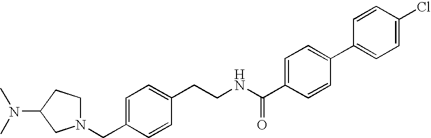

0.12 mL 37% aqueous formaldehyde solution, 28 mg (0.45 mmol) of NaBH₃CN and one drop of glacial acetic acid are added to a solution of 60 mg (0.14 mmol) of 4′-chloro-biphenyl-4-carboxylic acid{2-[4-(3-amino-pyrrolidin-1-ylmethyl)-phenyl]-ethyl}-amide in 5 mL acetonitrile. The reaction mixture is stirred overnight at RT and then combined with dilute NaOH solution and EtOAc. The phases are separated, the organic phase is dried over MgSO₄ and then freed from drying agent and solvent. The residue is purified by column chromatography.

Yield: 10 mg (15.7% of theory);

C₂₈H₃₂ClN₃O (M=462.04);

calc.: molar peak (M+H)⁺: 462/464 fnd.: molar peak (M+H)⁺: 462/464;

Retention time HPLC: 5.16 min (method A).

### EXAMPLE 2.90

### tert.butyl [1-(4-{2-[(4′-chloro-biphenyl-4-carbonyl)-amino]-ethyl}-benzyl)-pyrrolidin-2-ylmethyl]-carbaminate

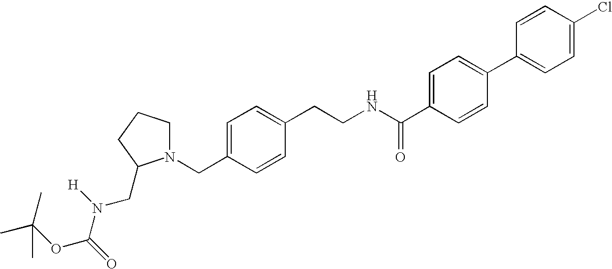

Prepared analogously to Example 2.76 from 230 mg (0.54 mmol) of 4′-chloro-biphenyl-4-carboxylic acid [2-(4-bromomethyl-phenyl)-ethyl]-amide and 116 mg (1.10 mmol) of tert.butyl pyrrolidin-2-ylmethyl-carbaminate.

Yield: 230 mg (78.3% of theory);

C₃₂H₃₈ClN₃O₃ (M=548.13);

calc.: molar peak (M+H)⁺: 548/550 fnd.: molar peak (M+H)⁺: 548/550;

R_{f} value: 0.35 (silica gel, CH₂Cl₂/MeOH/NH₃ 95:5:0.5).

### EXAMPLE 2.91

### 4′-chloro-biphenyl-4-carboxylic acid{2-[4-(2-aminomethyl-pyrrolidin-1-ylmethyl)-phenyl]-ethyl}-amide

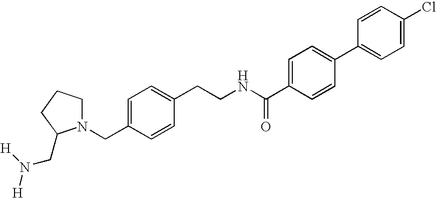

Prepared analogously to Example 2.88 from 230 mg (0.42 mmol) of tert.butyl [1-(4-{2-[(4′-chloro-biphenyl-4-carbonyl)-amino]-ethyl}-benzyl)-pyrrolidin-2-ylmethyl]-carbaminate

Yield: 188 mg (10% of theory);

C₂₇H₃₀ClN₃O (M=448.01);

calc.: molar peak (M+H)⁺: 448/450 fnd.: molar peak (M+H)⁺: 448/450;

R_{f} value: 0.35 (silica gel, CH₂Cl₂/MeOH/NH₃ 9:1:0.1).

### EXAMPLE 2.92

### 4′-chloro-biphenyl-4-carboxylic acid{2-[4-(2-dimethylaminomethyl-pyrrolidin-1-ylmethyl)-phenyl]-ethyl}-amide

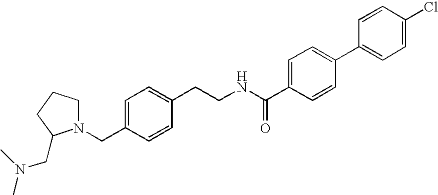

Prepared analogously to Example 2.89 from 40 mg (0.09 mmol) of 4′-chloro-biphenyl-4-carboxylic acid{2-[4-(2-aminomethyl-pyrrolidin-1-ylmethyl)-phenyl]-ethyl}-amide, 0.08 mL 37% aqueous formaldehyde solution and 19 mg (0.30 mmol) of NaBH₃CN.

Yield: 10 mg (23.6% of theory);

C₂₉H₃₄ClN₃O (M=476.07);

calc.: molar peak (M+H)⁺: 476/478 fnd.: molar peak (M+H)⁺: 476/478;

R_{f} value: 0.12 (silica gel, CH₂Cl₂/MeOH/NH₃ 9:1:0.1).

### EXAMPLE 2.93

### 4′-chloro-biphenyl-4-carboxylic acid{2-[4-(2-methyl-2,6-diaza-spiro[3.4]oct-6-ylmethyl)-phenyl]-ethyl}-amide

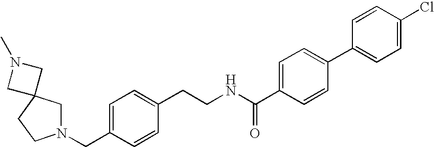

Prepared analogously to Example 2.76 from 250 mg (0.58 mmol) of 4′-chloro-biphenyl-4-carboxylic acid [2-(4-bromomethyl-phenyl)-ethyl]-amide, 97 mg K₂CO₃ and 81 mg (0.64 mmol) of 2-methyl-2,6-diaza-spiro[3.4]octane; the product is purified by HPLC.

Yield: 20 mg (7.2% of theory);

C₂₉H₃₂ClN₃O (M=474.05);

calc.: molar peak (M+H)⁺: 474/476 fnd.: molar peak (M+H)⁺: 474/476;

R_{f} value: 0.20 (silica gel, CH₂Cl₂/MeOH/NH₃ 9:1:0.1).

### EXAMPLE 2.94

### 3-[(4-{2-[(4′-chloro-biphenyl-4-carbonyl)-amino]-ethyl}-benzyl)-ethyl-amino]-propionic acid

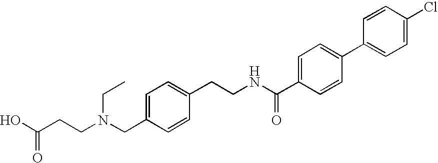

A suspension of 257 mg (0.6 mmol) of 4′-chloro-biphenyl-4-carboxylic acid [2-(4-bromomethyl-phenyl)-ethyl]-amide, 166 mg (1.2 mmol) of K₂CO₃ and 138 mg 3-ethylamino-propionic acid (0.9 mmol, used as the hydrochloride) in 20 mL acetonitrile is stirred for 3 days at RT. 5 mL of DMF are added and the mixture is heated to 50° C. for 3 h. The reaction mixture is filtered, the filtrate evaporated down and the residue is purified by HPLC.

Yield: 50 mg (17.9% of theory);

C₂₇H₂₉ClN₂O₃ (M=465.0);

calc.: molar peak (M+H)⁺: 465/467 fnd.: molar peak (M+H)⁺: 465/467;

Retention time HPLC: 5.85 min (method A).

### EXAMPLE 2.95

### methyl (S)-1-(4-{2-[(4′-chloro-biphenyl-4-carbonyl)-amino]-ethyl}-benzyl)-pyrrolidine-2-carboxylate

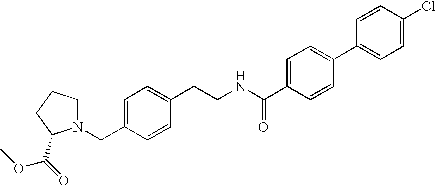

2.95a. ethyl 4-{2-[(4′-chloro-biphenyl-4-carbonyl)-amino]-ethyl}-benzoate

Prepared according to general working method I from 10.0 g (42.98 mmol) of 4′-chloro-biphenyl-4-carboxylic acid and 9.87 g (42.98 mmol) of ethyl 4-(2-amino-ethyl)-benzoate.

Yield: 10.64 g (60.7% of theory);

C₂₄H₂₂ClNO₃ (M=407.90);

calc.: molar peak (M+H)⁺: 408/410 fnd.: molar peak (M+H)⁺: 408/410;

R_{f} value: 0.87 (silica gel, CH₂Cl₂/MeOH 95:5).

2.95b. 4-{2-[(4′-chloro-biphenyl-4-carbonyl)-amino]-ethyl}-benzoic acid

14 mL 2 M NaOH solution are added to a solution of 10.64 g (26.08 mmol) of ethyl 4-{2-[(4′-chloro-biphenyl-4-carbonyl)-amino]-ethyl}-benzoate in 100 mL EtOH and the reaction mixture is heated to 60° C. overnight. Then a further 30 mL of NaOH solution are added and the mixture is kept at this temperature for a further 3 h. The reaction mixture is adjusted to pH 6-7 with 1M-HCl solution, the precipitated product is filtered off and dried in vacuo.

Yield: 7.65 g (77.2% of theory);

C₂₂H₁₈ClNO₃ (M=379.85);

calc.: molar peak (M+H)⁺: 380/382 fnd.: molar peak (M+H)⁺: 380/382;

Retention time HPLC: 8.1 min (method A).

2.95c. 4′-chloro-biphenyl-4-carboxylic acid [2-(4-hydroxymethyl-phenyl)-ethyl]-amide

3.24 g (20 mmol) of CDI are added to a solution of 7.2 g (18.97 mmol) of 4-{2-[(4′-chloro-biphenyl-4-carbonyl)-amino]-ethyl}-benzoic acid in 150 mL dry THF and the reaction mixture is heated to 50° C. for 2 h. This solution is added to a suspension of 1.44 g (38 mmol) of NaBH₄ in 5 mL water and stirred for a further hour. The reaction mixture is adjusted to pH 6-7 with 1M HCl solution and exhaustively extracted with EtOAc. The organic phase is washed with NaHCO₃ solution and with water and dried over MgSO₄. After elimination of the drying agent and solvent the residue is purified by chromatography (silica gel, CH₂Cl₂/MeOH 9:1). As there is still educt in the product, the procedure described above is repeated with 50% of the reagents used.

Yield: 2.85 g (41.0% of theory);

C₂₂H₂₂ClNO₂ (M=365.86);

calc.: molar peak (M+H)⁺: 366/368 fnd.: molar peak (M+H)⁺: 366/368;

Retention time HPLC: 8.0 min (method A).

2.95d. 4-{2-[(4′-chloro-biphenyl-4-carbonyl)-amino]-ethyl}-benzyl methanesulphonate

1.25 mL (9 mmol) of triethylamine are added to a solution of 1.0 g (2.73 mmol) of 4′-chloro-biphenyl-4-carboxylic acid [2-(4-hydroxymethyl-phenyl)-ethyl]-amide in 100 mL dry THF and the mixture is cooled to −20° C. Then 0.64 mL (8.2 mmol) of methanesulphonic acid chloride are added dropwise and the mixture is stirred for a further 2 h at this temperature. 5% NaHCO₃ solution is added and the mixture is extracted exhaustively with EtOAc. The organic phase is dried over Na₂SO₄, the drying agent and solvent removed and the residue dried at 30° C. in vacuo.

Yield: 1.21 g (99.7% of theory);

C₂₃H₂₂ClNO₄S (M=443.95);

calc.: molar peak (M+H)⁺: 444/446 fnd.: molar peak (M+H)⁺: 444/446;

Retention time HPLC: 8.8 min (method A).

2.95e. methyl (S)-1-(4-{2-[(4′-chloro-biphenyl-4-carbonyl)-amino]-ethyl}-benzyl)-pyrrolidine-2-carboxylate

Under an N₂ atmosphere a solution of 50 mg (0.3 mmol) of methyl (2S)-pyrrolidine-2-carboxylate (used as the hydrochloride) and 0.7 mL (0.5 mmol) of triethylamine in 4 mL DMF is stirred for 20 min at RT. Then 111 mg (0.25 mmol) of 4-{2-[(4′-chloro-biphenyl-4-carbonyl)-amino]-ethyl}-benzyl methanesulphonate are added and the mixture is heated to 60° C. for 2 h. The reaction mixture is evaporated down in vacuo and the residue purified by HPLC.

Yield: 4 mg (3.4% of theory);

C₂₈H₂₉ClN₂O₃ (M=477.01);

calc.: molar peak (M+H)⁺: 477/479 fnd.: molar peak (M+H)⁺: 477/479;

Retention time HPLC: 6.51 min (method A).

### EXAMPLE 2.96

### 4′-chloro-biphenyl-4-carboxylic acid {2-[4-(2-methyl-piperidin-1-ylmethyl)-phenyl]-ethyl}-amide

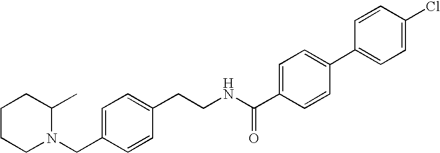

Prepared analogously to Example 2.95e from 111 mg (0.42 mmol) of 4-{2-[(4′-chloro-biphenyl-4-carbonyl)-amino]-ethyl}-benzyl methanesulphonate and 35 μL (0.3 mmol) of 2-methylpiperidine without using triethylamine.

Yield: 7 mg (6.3% of theory);

C₂₈H₃₁ClN₂O (M=447.03);

calc.: molar peak (M+H)⁺: 447/449 fnd.: molar peak (M+H)⁺: 447/449;

Retention time HPLC: 6.4 min (method A).

### EXAMPLE 2.97

### 4′-chloro-biphenyl-4-carboxylic acid {2-[4-(2-methyl-pyrrolidin-1-ylmethyl)-phenyl]-ethyl}-amide

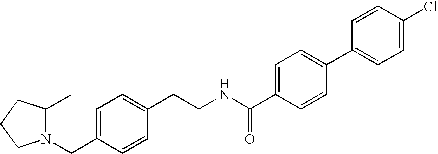

Prepared analogously to Example 2.95e from 111 mg (0.42 mmol) of 4-{2-[(4′-chloro-biphenyl-4-carbonyl)-amino]-ethyl}-benzyl methanesulphonate and 32 μL (0.3 mmol) of 2-methyl-pyrrolidine without using triethylamine.

Yield: 2 mg (1.8% of theory);

C₂₇H₂₉ClN₂O (M=433.0);

calc.: molar peak (M+H)⁺: 433/435 fnd.: molar peak (M+H)⁺: 433/435;

Retention time HPLC: 6.3 min (method A).

### EXAMPLE 2.98

### 4′-chloro-biphenyl-4-carboxylic acid (2-{4-[(cyclopropylmethyl-amino)-methyl]-phenyl}-ethyl)-amide

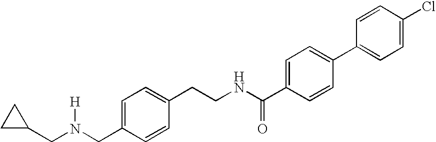

Prepared analogously to Example 2.95e from 111 mg (0.42 mmol) of 4-{2-[(4′-chloro-biphenyl-4-carbonyl)-amino]-ethyl}-benzyl methanesulphonate and 26 μL (0.3 mmol) of cyclopropylmethylamine without using triethylamine.

Yield: 4 mg (3.8% of theory);

C₂₆H₂₇ClN₂O (M=418.97);

calc.: molar peak (M+H)⁺: 418/420 fnd.: molar peak (M+H)⁺: 418/420;

Retention time HPLC: 6.4 min (method A).

### EXAMPLE 2.99

### 4′-chloro-biphenyl-4-carboxylic acid {2-[4-(3.4-dihydro-1H-isoquinolin-2-ylmethyl)-phenyl]-ethyl}-amide

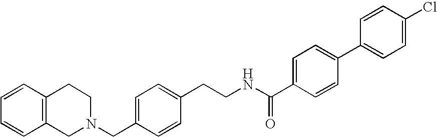

Prepared analogously to Example 2.95e from 111 mg (0.42 mmol) of 4-{2-[(4′-chloro-biphenyl-4-carbonyl)-amino]-ethyl}-benzyl methanesulphonate and 40 mg (0.3 mmol) of 1,2,3,4-tetrahydroisoquinoline without using triethylamine.

Yield: 21 mg (17.5% of theory);

C₂₆H₂₇ClN₂O (M=481.04);

calc.: molar peak (M+H)⁺: 481/483 fnd.: molar peak (M+H)⁺: 481/483;

Retention time HPLC: 6.8 min (method A).

### EXAMPLE 2.100

### 4′-chloro-biphenyl-4-carboxylic acid [2-(4-{[(2-hydroxy-ethyl)-methyl-amino]-methyl}-phenyl)-ethyl]-amide

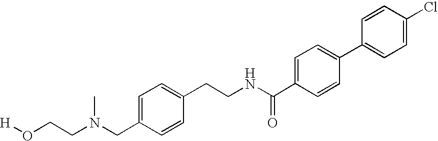

Prepared analogously to Example 2.95e from 111 mg (0.42 mmol) of 4-{2-[(4′-chloro-biphenyl-4-carbonyl)-amino]-ethyl}-benzyl methanesulphonate and 24 μL (0.3 mmol) of 2-methylamino-ethanol without using triethylamine.

Yield: 13 mg (12.3% of theory);

C₂₅H₂₇ClN₂O₂ (M=422.96);

calc.: molar peak (M+H)⁺: 423/425 fnd.: molar peak (M+H)⁺: 423/425;

Retention time HPLC: 5.8 min (method A).

### EXAMPLE 2.101

### 4′-chloro-biphenyl-4-carboxylic acid {2-[4-(2,6-dimethyl-piperidin-1-ylmethyl)-phenyl]-ethyl}-amide

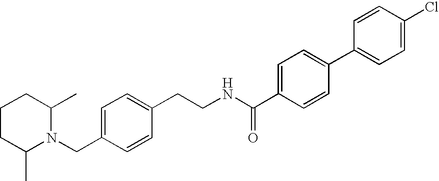

Prepared analogously to Example 2.95e from 111 mg (0.42 mmol) of 4-{2-[(4′-chloro-biphenyl-4-carbonyl)-amino]-ethyl}-benzyl methanesulphonate and 41 μL (0.3 mmol) of 2,6-dimethylpiperidine without using triethylamine.

Yield: 8 mg (6.9% of theory);

C₂₉H₃₃ClN₂O (M=461.05);

calc.: molar peak (M+H)⁺: 461/463 fnd.: molar peak (M+H)⁺: 461/463;

Retention time HPLC: 6.6 min (method A).

### EXAMPLE 2.102

### 4′-chloro-biphenyl-4-carboxylic acid [2-(4-azetidin-1-ylmethyl-phenyl)-ethyl]-amide

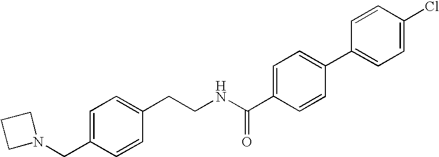

Prepared analogously to Example 2.95e from 111 mg (0.42 mmol) of 4-{2-[(4′-chloro-biphenyl-4-carbonyl)-amino]-ethyl}-benzyl methanesulphonate and 20 μL (0.3 mmol) of azetidine without using triethylamine.

Yield: 3 mg (3.0% of theory);

C₂₅H₂₅ClN₂O (M=404.94);

calc.: molar peak (M+H)⁺: 405/407 fnd.: molar peak (M+H)⁺: 405/407;

Retention time HPLC: 5.9 min (method A).

### EXAMPLE 2.103

### 4′-chloro-biphenyl-4-carboxylic acid {2-[4-(2,5-dihydro-pyrrol-1-ylmethyl)-phenyl]-ethyl}-amide

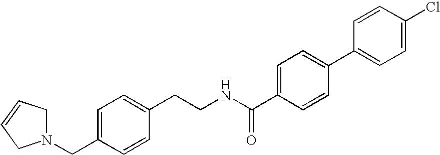

Prepared analogously to Example 2.95e from 50 mg (0.11 mmol) of 4-{2-[(4′-chloro-biphenyl-4-carbonyl)-amino]-ethyl}-benzyl methanesulphonate and 11 μL (0.14 mmol) of 2,5-dihydro-1H-pyrrole without using triethylamine.

Yield: 18 mg (38.2% of theory);

C₂₆H₂₅ClN₂O (M=416.95);

calc.: molar peak (M+H)⁺: 417/419 fnd.: molar peak (M+H)⁺: 417/419;

Retention time HPLC: 6.2 min (method A).

### EXAMPLE 2.104

### 4′-bromo-biphenyl-4-carboxylic acid {2-[4-(2,5-dihydro-pyrrol-1-ylmethyl)-phenyl]-ethyl}-amide

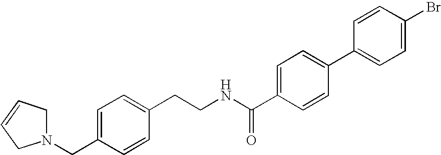

2.104a. ethyl 4′-bromo-biphenyl-4-carboxylate

Prepared analogously to Example 2.46b from 1.22 mL (7.47 mmol) of ethyl 4-bromo-benzoate and 1.8 g (8.96 mmol) of 4-bromophenyl-boric acid, refluxing for 72 h. The product is crystallised from acetonitrile.

Yield: 293 mg (12.8% of theory);

C₁₅H₁₃BrO₂ (M=305.17);

calc.: molar peak (M+H)⁺: 304/306 fnd.: molar peak (M+H)⁺: 304/306;

R_{f} value: 0.9 (silica gel, petroleum ether/EtOAc 6:4).

2.104b. 4′-bromo-biphenyl-4-carboxylic acid

1.24 mL 2M NaOH solution are added to a solution of 270 mg (0.89 mmol) of ethyl 4′-bromo-biphenyl-4-carboxylate in 10 mL EtOH and the reaction mixture is stirred for 2 h at RT. The pH is adjusted to 6-7 with 1M HCl, the precipitated product is filtered off and dried.

Yield: 205 mg (83.6% of theory);

C₁₃H₉BrO₂ (M=277.12);

calc.: molar peak (M−H)⁻: 275/277 fnd.: molar peak (M−H)⁻: 275/277;

Retention time HPLC: 8.5 min (method A).

2.104c. [4-(2-amino-ethyl)-phenyl]-methanol

580 mg of Raney Nickel are added to 5.8 g (39.41 mmol) of (4-hydroxymethyl-phenyl)-acetonitrile (cf. Example 1.1e.) in 116 mL methanolic NH₃ solution and the reaction mixture is hydrogenated at 50 psi H₂. After the end of the reaction the catalyst is filtered off, the solvent is removed and the residue is purified by chromatography (silica gel, EtOAc/MeOH/NH₃ 7:3:0.3)

Yield: 3.9 g (65.4% of theory);

C₉H₁₃NO (M=151.21);

calc.: molar peak (M+H)⁺: 152 fnd.: molar peak (M+H)⁺: 152;

R_{f} value: 0.18 (silica gel, EtOAc/MeOH/NH₃ 8:2:0.2).

2.104d. tert.butyl [2-(4-hydroxymethyl-phenyl)-ethyl]-carbaminate

17.36 mL of 1M BOC anhydride in CH₂Cl₂ are added at RT to a solution of 2.5 g (16.53 mmol) of [4-(2-amino-ethyl)-phenyl]-methanol in 50 mL CH₂Cl₂ and the reaction mixture is stirred overnight at RT. 100 mL of KHSO₄ solution are added, the organic phase is separated off, washed with dilute NaHCO₃ solution and water and dried over MgSO₄. After elimination of the drying agent and solvent the desired product is obtained.

Yield: 4.06 g (97.7% of theory);

C₁₄H₂₁NO₃ (M=251.33);

calc.: molar peak (M+H)⁺: 252 fnd.: molar peak (M+H)⁺: 252;

Retention time HPLC: 6.4 min (method A).

2.104e. tert.butyl [2-(4-chloromethyl-phenyl)-ethyl]-carbaminate

1 mL pyridine is added to a solution of 2.6 g (10.35 mmol) of tert.butyl [2-(4-hydroxymethyl-phenyl)-ethyl]-carbaminate in 50 mL CH₂Cl₂, cooled to 0° C. and 1.03 mL (12.41 mmol) of thionyl chloride are added. The mixture is kept for 1 h at 0° C. and then allowed to heat up to RT. The reaction mixture is washed with water, dilute KHSO₄ solution and again with water, dried with MgSO₄ and filtered through activated charcoal. After elimination of the solvent the product is obtained as an oil, which is reacted without further purification.

Yield: 1.8 g (64.5% of theory);

C₁₄H₂₀ClNO₂ (M=269.77);

calc.: molar peak (M−H)⁻: 268/270 fnd.: molar peak (M−H)⁻: 268/270;

R_{f} value: 0.62 (silica gel, petroleum ether/EtOAc 7:3).

2.104f. tert-butyl {2-[4-(2,5-dihydro-pyrrol-1-ylmethyl)-phenyl]-ethyl}-carbaminate

2.37 g (17.13 mmol) of K₂CO₃ and 0.8 mL (10.38 mmol) of 2,5-dihydro-1H-pyrrole are added to a solution of 1.4 g (5.19 mmol) of tert.butyl [2-(4-chloromethyl-phenyl)-ethyl]-carbaminate in 50 mL acetonitrile and the mixture is stirred overnight at RT. The reaction mixture is diluted with CH₂Cl₂, washed with water and dried over MgSO₄. After elimination of the drying agent and solvent the desired product is obtained.

Yield: 1.46 g (93.0% of theory);

C₁₈H₂₆N₂O₂ (M=302.42);

calc.: molar peak (M+H)⁺: 303 fnd.: molar peak (M+H)⁺: 303;

R_{f} value: 0.15 (silica gel, petroleum ether/EtOAc 7:3).

2.104g. 2-[4-(2,5-dihydro-pyrrol-1-ylmethyl)-phenyl]-ethylamine

5 mL trifluoroacetic acid are added to a solution of 1.21 g (4 mmol) of tert.butyl {2-[4-(2,5-dihydro-pyrrol-1-ylmethyl)-phenyl]-ethyl}-carbaminate in 50 mL CH₂Cl₂ and stirred for 2 h at RT. The reaction mixture is evaporated down in vacuo, the residue combined with water and CH₂Cl₂ and made alkaline with K₂CO₃ solution. The organic phase is separated off, washed with water and dried over MgSO₄. After elimination of the drying agent and solvent the desired product is obtained.

Yield: 0.35 g (43.3% of theory);

C₁₃H₁₈N₂ (M=202.30);

calc.: molar peak (M+H)⁺: 203 fnd.: molar peak (M+H)⁺: 203;

R_{f} value: 0.05 (silica gel, EtOAc/MeOH/NH₃ 9:1:0.1).

2.104h. 4′-bromo-biphenyl-4-carboxylic acid-{2-[4-(2,5-dihydro-pyrrol-1-ylmethyl)-phenyl]-ethyl}-amide

Prepared according to general working method I from 139 mg (0.50 mmol) of 4′-bromo-biphenyl-4-carboxylic acid and 101 mg (0.50 mmol) of 2-[4-(2,5-dihydro-pyrrol-1-ylmethyl)-phenyl]-ethylamine.

Yield: 21 mg (9.1% of theory);

C₂₆H₂₅BrN₂O (M=461.41);

calc.: molar peak (M+H)⁺: 461/463 fnd.: molar peak (M+H)⁺: 461/463;

Retention time HPLC: 6.46 min (method A).

### EXAMPLE 2.105

### 4′-chloro-biphenyl-4-carboxylic acid {2-[4-(1-ethyl-piperidin-2-yl)-phenyl]-ethyl}-amide

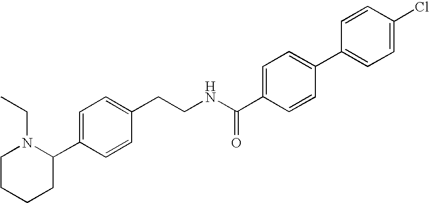

2.105a. (4-pyridin-2-yl-phenyl)-acetonitrile

Prepared analogously to Example 2.46b from 0.52 mL (5.40 mmol) of 2-bromo-pyridine and 1.0 g (5.96 mmol) of 4-cyanomethylphenyl-boric acid. After elimination of the drying agent and solvent the residue is triturated with diisopropylether and dried in the air.

Yield: 0.76 g (72.5% of theory);

C₁₃H₁₀N₂ (M=194.24);

calc.: molar peak (M+H)⁺: 195 fnd.: molar peak (M+H)⁺: 195;

Retention time HPLC: 3.56 min (method B).

2.105b. 2-(4-cyanomethyl-phenyl)-1-ethyl-pyridinium iodide

0.38 mL (4.7 mmol) of ethyl iodide are added to a solution of 760 mg (3.91 mmol) of (4-pyridin-2-yl-phenyl)-acetonitrile in 5 mL DMF and stirred overnight at RT. To complete the reaction the solution is treated for 20 min at 120° C. in the microwave. The solvent is evaporated down in vacuo, the residue is combined with water and extracted with EtOAc. The aqueous phase is evaporated down, the residue triturated with THF and the suspension cooled to 0° C. The product is suction filtered and dried at 50° C.

Yield: 800 mg (58.4% of theory);

C₁₅H₁₅IN₂ (M=350.21);

calc.: molar peak (M)⁺: 223 fnd.: molar peak (M)⁺: 223;

Retention time HPLC: 1.76 min (method A).

2.105c. 2-[4-(1-ethyl-piperidin-2-yl)-phenyl]-ethylamine

100 mg of Raney nickel are added to a solution of 800 mg (2.28 mmol) of 2-(4-cyanomethyl-phenyl)-1-ethyl-pyridinium iodide in 10 mL methanolic NH₃ and the reaction mixture is hydrogenated at 20 psi and RT 24 h in the autoclave. The catalyst is suction filtered, the reaction solution is combined with 100 mg PtO₂ and hydrogenated again at RT and 20 psi 30 h. After elimination of the catalyst the product is obtained (as the hydroiodide), which is reacted further without purification.

Yield: 700 mg (85.1% of theory);

C₁₅H₂₄IN₂ (M=360.28);

calc.: molar peak (M)⁺: 233 fnd.: molar peak (M)⁺: 233;

Retention time HPLC: 0.93 min (isocratic water:acetonitrile:formic acid 95:5:0.01 over 8 min).

2.105d. 4′-chloro-biphenyl-4-carboxylic acid {2-[4-(1-ethyl-piperidin-2-yl)-phenyl]-ethyl}-amide

Prepared according to general working method I from 480 mg (1.33 mmol) of 2-[4-(1-ethyl-piperidin-2-yl)-phenyl]-ethylamine (used as the hydroiodide) and 310 mg (1.33 mmol) of 4′-chloro-biphenyl-4-carboxylic acid.

Yield: 20 mg (3.4% of theory);

C₂₈H₃₁ClN₂O (M=447.03);

calc.: molar peak (M+H)⁺: 447/449 fnd.: molar peak (M+H)⁺: 447/449;

Retention time HPLC: 6.68 min (method A).

### EXAMPLE 2.106

### 4′-chloro-biphenyl-4-carboxylic acid [2-(1-pyrrolidin-1-yl-indan-5-yl)-ethyl]-amide

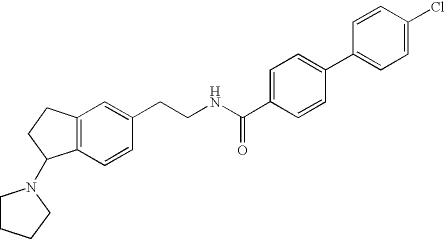

2.106a. ethyl (E)-3-(1-oxo-indan-5-yl)-acrylate

5.96 mL (55 mmol) of ethyl acrylate, 275 mg (1.21 mmol) of Pd(OAc)₂ and 704 mg (2.31 mmol) of tri-o-tolylphosphine are added to a solution of 4.64 g (21.99 mmol) of 5-bromo-indan-1-one in 110 mL triethylamine under N₂ and the reaction mixture is heated to 100° C. for 4 h. The solvent is distilled off, the residue is combined with 150 mL EtOAc and 100 mL ice water, acidified with conc. HCl, the organic phase is washed with 100 mL water and dried over MgSO₄. After elimination of the drying agent and solvent the residue is purified by chromatography (silica gel, hexane/EtOAc 9:1 towards 8:2)

Yield: 4.0 g (79.0% of theory);

melting point: 100-102° C.

2.106b. (E)-3-(1-oxo-indan-5-yl)-acrylic acid

10 mL 2 N NaOH are added to a solution of 4.0 g (17.0 mmol) of ethyl (E)-3-(1-oxo-indan-5-yl)-acrylate in 50 mL MeOH and the reaction mixture is refluxed for 30 min. Then it is combined with 11 mL 2 N HCl solution, MeOH is distilled off, the crystals are suction filtered and dried.

Yield: 3.0 g (87.3% of theory);

melting point: 240-244° C.

2.106c. 3-(1-oxo-indan-5-yl)-propionic acid

150 mg 10% Pd/C are added to a solution of 1.6 g (7.91 mmol) of (E)-3-(1-oxo-indan-5-yl)-acrylic acid in 50 mL MeOH and the reaction mixture is shaken in a Parr autoclave at RT and 3 bar H₂ until the theoretical uptake of H₂ has been achieved. 10 mL of 1 N NaOH are added and the solvent is removed. The residue is acidified with dilute HCl, exhaustively extracted with EtOAc and the organic phase is dried over MgSO₄. After elimination of the drying agent and solvent the residue is triturated with tert.-butylmethylether, the precipitate is suction filtered and dried.

Yield: 500 mg (31.0% of theory);

C₁₂H₁₂O₃ (M=204.23);

calc.: molar peak (M−H)⁻: 203 fnd.: molar peak (M−H)⁻: 203;

R_{f} value: 0.45 (silica gel, CH₂Cl₂/MeOH 9:1).

2.106d. tert.butyl [2-(1-oxo-indan-5-yl)-ethyl]-carbaminate

1.6 g (7.83 mmol) of 3-(1-oxo-indan-5-yl)-propionic acid are added to 25 ml tert. butanol and 2.5 mL triethylamine under an argon atmosphere. 2.22 mL (10.0 mmol) of diphenyl azido-phosphate are added to this solution and heated to 80° C. for 3 h. The reaction mixture is evaporated down in vacuo and the residue is purified by chromatography on silica gel.

Yield: 750 mg (34.8% of theory);

C₁₆H₂₁NO₃ (M=275.35);

calc.: molar peak (M)⁺: 275 fnd.: molar peak (M)⁺: 275;

R_{f} value: 0.65 (silica gel, CH₂Cl₂/MeOH 95:5).

2.106e. tert.butyl [2-(1-hydroxy-indan-5-yl)-ethyl]-carbaminate

700 mg (18.5 mmol) of NaBH₄ are added batchwise to a solution of 700 mg (2.54 mmol) of tert.butyl [2-(1-oxo-indan-5-yl)-ethyl]-carbaminate in 70 mL MeOH and stirred overnight at RT. The reaction solution is carefully combined with 10% KHSO₄ solution, diluted with water and exhaustively extracted with tert.-butylmethyl-ether. The organic phase is washed with water and dried over MgSO₄. After elimination of the drying agent and solvent the residue is purified by chromatography on silica gel.

Yield: 350 mg (49.7% of theory);

C₁₆H₂₃NO₃ (M=277.37);

calc.: molar peak (M)⁺: 277 fnd.: molar peak (M)⁺: 277;

R_{f} value: 0.30 (silica gel, petroleum ether/EtOAc 6:4).

2.106f. [2-(1-pyrrolidin-1-yl-indan-5-yl)-ethyl]-carbaminate tert.butyl

109 μL (1.5 mmol) of thionyl chloride (dissolved in a little CH₂Cl₂) are slowly added dropwise to a solution of 350 mg (1.26 mmol) of tert.butyl [2-(1-hydroxy-indan-5-yl)-ethyl]-carbaminate in 7.5 mL CH₂Cl₂ cooled to 0° C. Stirring is continued for a further 30 min at 10° C., the reaction solution is combined with ice-cold NaHCO₃ solution, the organic phase is separated off, washed with cold water and dried over MgSO₄. After elimination of the drying agent the filtrate is cooled to 0° C., 417 μL (5.0 mmol) of pyrrolidine are added dropwise and the reaction mixture is stirred overnight at RT. The reaction mixture is evaporated down and the residue is purified by chromatography on silica gel.

Yield: 120 mg (28.8% of theory);

C₂₀H₃₀N₂O₂ (M=330.47);

calc.: molar peak (M+H)⁺: 331 fnd.: molar peak (M+H)⁺: 331;

Retention time HPLC: 5.6 min (method A).

2.106g. 2-(1-pyrrolidin-1-yl-indan-5-yl)-ethylamine

100 μL trifluoroacetic acid are added with gentle cooling to a solution of 100 mg (0.3 mmol) of tert.butyl [2-(1-pyrrolidin-1-yl-indan-5-yl)-ethyl]-carbaminate in 10 mL CH₂Cl₂ and stirred for 1 h at RT. To complete the reaction a further 500 μL of trifluoroacetic acid are added with cooling and the mixture is stirred for 2 h at RT. The reaction mixture is evaporated down in vacuo and the product (as the bis-trifluoroacetate) is further reacted without purification.

Yield: 100 mg (72.7% of theory);

C₁₉H₂₄F₆N₂O₄ (M=458.51);

calc.: molar peak (M+H)⁺: 231 fnd.: molar peak (M+H)⁺: 231;

R_{f} value: 0.3 (silica gel, CH₂Cl₂/MeOH/NH₃ 9:1:0.1).

2.106h. 4′-chloro-biphenyl-4-carboxylic acid [2-(1-pyrrolidin-1-yl-indan-5-yl)-ethyl]-amide

Prepared according to general working method I from 100 mg (0.29 mmol) of 2-(1-pyrrolidin-1-yl-indan-5-yl)-ethylamine (used as the bis-trifluoroacetate) and 70 mg (0.3 mmol) of 4′-chloro-biphenyl-4-carboxylic acid.

Yield: 40 mg (30.0% of theory);

C₂₈H₂₉ClN₂O (M=445.01);

calc.: molar peak (M+H)⁺: 445/447 fnd.: molar peak (M+H)⁺: 445/447;

Retention time HPLC: 6.65 min (method A).

### EXAMPLE 2.107

### 4′-chloro-biphenyl-4-carboxylic acid [2-(3-bromo-4-pyrrolidin-1-ylmethyl-phenyl)-ethyl]-amide

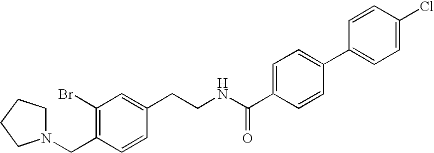

2.107a. methyl 2-bromo-4-cyanomethyl-benzoate

A solution of 98.55 g (0.32 mol) of methyl 2-bromo-4-bromomethyl-benzoate in 60 mL EtOH is added to a solution of 24.51 g (0.5 mol) of NaCN in 40 mL water and the reaction mixture is refluxed for 5 h. 1 L of tert.-butylmethylether and 500 mL water are added, the organic phase is separated off, washed several times with water and dried over MgSO₄. After elimination of the drying agent and solvent the residue is purified by chromatography on silica gel (petroleum ether/EtOAc 8:2).

Yield: 15.0 g (16.6% of theory);

C₁₀H₈BrNO₂ (M=254.09);

calc.: molar peak (M−H)⁻: 252/254;

fnd.: molar peak (M−H)⁻: 252/254.

2.107b. 2-bromo-4-cyanomethyl-benzoic acid

35 mL 1M NaOH solution are added to a solution of 7.9 g (31.0 mmol) of methyl 2-bromo-4-cyanomethyl-benzoate in 100 mL EtOH, the reaction mixture is refluxed for 1 h and then stirred overnight at RT. Ice water is added and the mixture is acidified with dilute KHSO₄ solution. The precipitate is suction filtered, washed with water and dried at 50° C.

Yield: 6.2 g (83.3% of theory);

C₉H₆BrNO₂ (M=240.06);

calc.: molar peak (M−H)⁻: 238/240 fnd.: molar peak (M−H)⁻: 238/240;

Retention time HPLC: 3.99 min (method B).

2.107c. (3-bromo-4-hydroxymethyl-phenyl)-acetonitrile

1.78 g (11 mmol) of CDI are added to a solution of 2.4 g (10 mmol) of 2-bromo-4-cyanomethyl-benzoic acid in 50 mL THF and the water bath is heated until the development of gas has ceased. Then this is added to a solution of 0.76 g (20 mmol) of NaBH₄ in 50 mL water, while the temperature should not exceed 30° C. Stirring is continued for a further 2 h at RT, the reaction mixture is carefully acidified with dilute KHSO₄ solution, extracted exhaustively with tert.-butylmethylether, the organic phase is washed with water and dried over MgSO₄. It is filtered through activated charcoal and the solvent is removed in vacuo.

Yield: 2.2 g (97.3% of theory);

C₉H₈BrNO (M=226.07);

calc.: molar peak (M−H)⁻: 224/226 fnd.: molar peak (M−H)⁻: 224/226;

R_{f} value: 0.6 (silica gel, CH₂Cl₂/MeOH 9:1).

2.107d. (3-bromo-4-pyrrolidin-1-ylmethyl-phenyl)-acetonitrile

1.25 mL (9 mmol) of triethylamine are added to a solution of 1.9 g (8.4 mmol) of (3-bromo-4-hydroxymethyl-phenyl)-acetonitrile in 50 mL CH₂Cl₂, cooled to 0° C. and a solution of 0.66 mL (8.5 mmol) of methanesulphonic acid chloride in 10 mL CH₂Cl₂ is added dropwise. The mixture is stirred for 1 h at 0° C. and then a solution of 1.4 mL (17 mmol) of pyrrolidine in 10 mL CH₂Cl₂ is added dropwise while cooling with ice. The reaction mixture is heated overnight to RT, combined with water, the organic phase is separated off, washed twice with water, filtered through activated charcoal and evaporated down in vacuo. The residue is co-evaporated twice with toluene and the product obtained is further reacted without purification.

Yield: 2.25 g (95.9% of theory);

C₁₃H₁₅BrN₂ (M=279.18);

calc.: molar peak (M+H)⁺: 279/281 fnd.: molar peak (M+H)⁺: 279/281;

R_{f} value: 0.5 (silica gel, CH₂Cl₂/MeOH/NH₃ 9:1:0.1).

2.107e. 2-(3-bromo-4-pyrrolidin-1-ylmethyl-phenyl)-ethylamine

20 mg of Raney nickel are added to a solution of 225 mg (0.81 mmol) of (3-bromo-4-pyrrolidin-1-ylmethyl-phenyl)-acetonitrile in 5 mL methanolic NH₃ and 5 mL EtOAc and shaken in a Parr autoclave for 1 h at RT and 5 psi H₂. The catalyst is filtered off, the solvent evaporated down in vacuo and the product further reacted without purification.

Yield: 225 mg (98.1% of theory);

C₁₃H₁₉BrN₂ (M=283.21);

calc.: molar peak (M+H)⁺: 283/285 fnd.: molar peak (M+H)⁺: 283/285;

R_{f} value: 0.08 (silica gel, CH₂Cl₂/MeOH/NH₃ 9:1:0.1).

2.107f. 4′-chloro-biphenyl-4-carboxylic acid [2-(3-bromo-4-pyrrolidin-1-ylmethyl-phenyl)-ethyl]-amide hydrochloride

Prepared according to general working method I from 220 mg (0.78 mmol) of 2-(3-bromo-4-pyrrolidin-1-ylmethyl-phenyl)-ethylamine and 186 mg (0.8 mmol) of 4′-chloro-biphenyl-4-carboxylic acid. After elimination of the drying agent and solvent the residue is taken up in isopropanol/tert.-butylmethylether, combined with ethereal HCl and evaporated down in vacuo. The residue is again taken up in 20 mL isopropanol, triturated, suction filtered, washed with a little isopropanol and dried at 50° C.

Yield: 165 mg (39.6% of theory);

C₂₆H₂₇BrCl₂N₂O (M=534.33);

calc.: molar peak (M+H)⁺: 497/499/501 fnd.: molar peak (M+H)⁺: 497/499/501;

R_{f} value: 0.35 (silica gel, CH₂Cl₂/MeOH/NH₃ 9:1:0.1).

### EXAMPLE 2.108

### 4′-chloro-biphenyl-4-carboxylic acid [2-(3-methyl-4-pyrrolidin-1-ylmethyl-phenyl)-ethyl]-amide

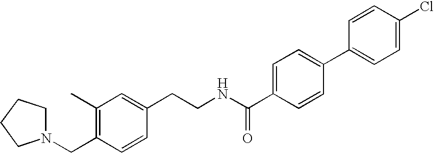

17.3 mg (0.28 mmol) of methylboric acid, 2.5 mL 2M Na₂CO₃ solution and 32 mg (0.03 mmol) of tetrakis-(triphenylphosphine)-palladium are added to a suspension of 150 mg (0.28 mmol) of 4′-chloro-biphenyl-4-carboxylic acid [2-(3-bromo-4-pyrrolidin-1-ylmethyl-phenyl)-ethyl]-amide hydrochloride in 5 mL dioxane and the reaction mixture is refluxed for 5 h. The hot suspension is suction filtered through a glass fibre filter, the filtrate is combined with semisaturated NaHCO₃ solution, exhaustively extracted with EtOAc and dried over MgSO₄. After elimination of the drying agent and solvent the residue is purified by chromatography on silica gel (CH₂Cl₂/MeOH 8:2).

Yield: 20 mg (16.4% of theory);

C₂₇H₂₉ClN₂O (M=433.0);

calc.: molar peak (M+H)⁺: 433/435 fnd.: molar peak (M+H)⁺: 433/435;

Retention time HPLC: 6.47 min (method A).

### EXAMPLE 2.109

### 4′-chloro-biphenyl-4-carboxylic acid [2-(2-bromo-4-pyrrolidin-1-ylmethyl-phenyl)-ethyl]-amide

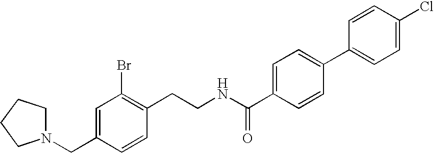

2.109a. ethyl 4-(2-amino-ethyl)-3-nitro-benzoate

5.78 g (57 mmol) of KNO₃ are added batchwise to a solution of 12.0 g (52 mmol) of ethyl 4-(2-amino-ethyl)-benzoate in 80 mL conc. H₂SO₄ cooled to −5° C. and stirred for 1 h at this temperature. The reaction solution is slowly added dropwise to ice water (the temperature should not exceed 0° C.) and stirred for 1 h. The precipitate is suction filtered, washed with water and dried at 50° C.

Yield: 8.2 g (66.2% of theory);

C₁₁H₁₄N₂O₄ (M=238.25);

calc.: molar peak (M+H)⁺: 239 fnd.: molar peak (M+H)⁺: 239;

Retention time HPLC: 3.64 min (method A).

2.109b. ethyl 4-{2-[(4′-chloro-biphenyl-4-carbonyl)-amino]-ethyl}-3-nitro-benzoate

Prepared according to general working method I from 8.2 g (34 mmol) of ethyl 4-(2-amino-ethyl)-3-nitro-benzoate and 7.91 g (34 mmol) of 4′-chloro-biphenyl-4-carboxylic acid.

Yield: 7.7 g (50.0% of theory);

C₂₄H₂₁ClN₂O₅ (M=452.90);

calc.: molar peak (M+H)⁺: 452/454 fnd.: molar peak (M+H)⁺: 452/454;

Retention time HPLC: 6.14 min (method B).

2.109c. ethyl 3-amino-4-{2-[(4′-chloro-biphenyl-4-carbonyl)-amino]-ethyl}-benzoate

0.5 g of Raney Nickel are added to a solution of 7.7 g (17 mmol) of ethyl 4-{2-[(4′-chloro-biphenyl-4-carbonyl)-amino]-ethyl}-3-nitro-benzoate in 200 mL EtOAc and the reaction mixture is shaken overnight in the autoclave at RT and 10 psi H₂. To complete the reaction 50 mL THF are added and the mixture is shaken for a further 2 h. The catalyst is suction filtered, washed thoroughly with THF, the solvent is evaporated down in vacuo, the residue is triturated with EtOAc, suction filtered again and dried in the air.

Yield: 5.0 g (69.5% of theory);

C₂₄H₂₃ClN₂O₃ (M=422.92);

calc.: molar peak (M+H)⁺: 423/425 fnd.: molar peak (M+H)⁺: 423/425;

Retention time HPLC: 5.71 min (method B).

2.109d. ethyl 3-bromo-4-{2-[(4′-chloro-biphenyl-4-carbonyl)-amino]-ethyl}-benzoate

20 mL 48% HBr are added to a solution of 5.0 g (7.69 mmol) of ethyl 3-amino-4-{2-[(4′-chloro-biphenyl-4-carbonyl)-amino]-ethyl}-benzoate in 20 mL water and cooled to 0° C. Then a solution of 0.9 g (13 mmol) of NaNO₂ in 5.2 mL water is added dropwise so that the temperature does not exceed 5° C. and the mixture is stirred for a further 10 min at 0° C. Then a solution of 1.87 g (13 mmol) of CuBr in 6.65 mL 48% HBr is immediately added dropwise at this temperature. The reaction mixture is then heated to 60° C. for 1 h. Water is added and the mixture is extracted exhaustively with EtOAc. The organic phase is washed with water and dried over MgSO₄. After elimination of the drying agent and solvent the residue is purified by chromatography on silica gel (petroleum ether/EtOAc 6:4).

Yield: 1.3 g (34.7% of theory);

C₂₄H₂₁BrClNO₃ (M=486.80);

calc.: molar peak (M+H)⁺: 486/488/490 fnd.: molar peak (M+H)⁺: 486/488/490;

R_{f} value: 0.55 (silica gel, petroleum ether/EtOAc 6:4).

2.109e. 3-bromo-4-{2-[(4′-chloro-biphenyl-4-carbonyl)-amino]-ethyl}-benzoic acid

6 mL 1N NaOH solution are added to a suspension of 1.3 g (2.67 mmol) of ethyl 3-bromo-4-{2-[(4′-chloro-biphenyl-4-carbonyl)-amino]-ethyl}-benzoate in 20 mL EtOH and 5 mL THF and the reaction mixture is stirred overnight at RT. It is evaporated down in vacuo, the residue is combined with water and neutralised with 1 N HCl, whereupon the product is precipitated. Stirring is continued for another hour while cooling with ice, the mixture is suction filtered, washed with water and the product is dried at 50° C.

Yield: 1.2 g (97.9% of theory);

C₂₂H₁₇BrClNO₃ (M=458.74);

calc.: molar peak (M+H)⁺: 456/458/460 fnd.: molar peak (M+H)⁺: 456/458/460;

Retention time HPLC: 5.51 min (method B).

2.109f. 4′-chloro-biphenyl-4-carboxylic acid [2-(2-bromo-4-hydroxymethyl-phenyl)-ethyl]-amide

0.64 g (3.92 mmol) of CDI are added to a solution of 1.2 g (2.62 mmol) of 3-bromo-4-{2-[(4′-chloro-biphenyl-4-carbonyl)-amino]-ethyl}-benzoic acid in 10 mL DMF and the mixture is heated to 50° C. until the development of gas has ceased. The reaction mixture is added to a solution of 0.3 g (7.85 mmol) of NaBH₄ in 10 mL water, stirred for 1 h at RT, acidified with dilute KHSO₄ solution and exhaustively extracted with EtOAc. The organic phase is washed with semisaturated NaHCO₃ solution and dried over MgSO₄. After elimination of the drying agent and solvent the residue is further reacted without purification.

Yield: 0.87 g (74.8% of theory);

C₂₂H₁₉BrClNO₂ (M=444.76);

calc.: molar peak (M+H)⁺: 444/446/448 fnd.: molar peak (M+H)⁺: 444/446/448;

Retention time HPLC: 8.07 min (method A).

2.109g. 4′-chloro-biphenyl-4-carboxylic acid [2-(2-bromo-4-chlormethyl-phenyl)-ethyl]-amide

0.24 mL (2.93 mmol) of pyridine are added to a solution of 0.87 g (1.96 mmol) of 4′-chloro-biphenyl-4-carboxylic acid [2-(2-bromo-4-hydroxymethyl-phenyl)-ethyl]-amide in 20 mL CH₂Cl₂ and cooled to 0° C. 0.21 mL (2.93 mmol) of thionyl chloride is added, the mixture is stirred for 1 h at this temperature and then allowed to warm up to RT. Water is added, the mixture is filtered through Celite, the aqueous phase is extracted with CH₂Cl₂ and the combined organic phases are dried over MgSO₄. After elimination of the drying agent and solvent the residue is further reacted without purification.

Yield: 0.66 g (72.8% of theory);

C₂₂H₁₈BrCl₂NO (M=463.21);

calc.: molar peak (M+H)⁺: 462/464/466 fnd.: molar peak (M+H)⁺: 462/464/466;

Retention time HPLC: 6.37 min (method B).

2.109h. 4′-chloro-biphenyl-4-carboxylic acid [2-(2-bromo-4-pyrrolidin-1-ylmethyl-phenyl)-ethyl]-amide

0.59 g (4.28 mmol) of K₂CO₃ and 0.24 mL (2.85 mmol) of pyrrolidine are added to a solution of 0.66 g (1.43 mmol) of 4′-chloro-biphenyl-4-carboxylic acid [2-(2-bromo-4-chloromethyl-phenyl)-ethyl]-amide in 20 mL acetonitrile and 6 mL DMF and stirred for 5 h at RT. Water is added, the mixture is extracted exhaustively with EtOAc, the organic phase is washed several times with water and dried over MgSO₄. After elimination of the drying agent and solvent the residue is purified by chromatography on silica gel (CH₂Cl₂/MeOH 9:1).

Yield: 0.2 g (28.2% of theory);

C₂₆H₂₆BrClN₂O (M=497.87);

calc.: molar peak (M+H)⁺: 497/499/501 fnd.: molar peak (M+H)⁺: 497/499/501;

Retention time HPLC: 4.39 min (method B).

### EXAMPLE 2.110

### 4′-chloro-biphenyl-4-carboxylic acid [2-(2-methyl-4-pyrrolidin-1-ylmethyl-phenyl)-ethyl]-amide

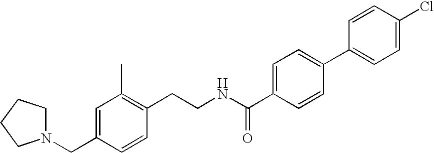

Prepared analogously to Example 2.108 from 200 mg (0.40 mmol) of 4′-chloro-biphenyl-4-carboxylic acid [2-(2-bromo-4-pyrrolidin-1-ylmethyl-phenyl)-ethyl]-amide and 27.3 mg (0.44 mmol) of methylboric acid, by refluxing for only 2 h and purifying the product by HPLC.

Yield: 62 mg (35.6% of theory);

C₂₇H₂₉ClN₂O (M=433.0);

calc.: molar peak (M+H)⁺: 433/435 fnd.: molar peak (M+H)⁺: 433/435;

Retention time HPLC: 6.15 min (method A).

### EXAMPLE 2.111

### 4′-chloro-biphenyl-4-carboxylic acid [2-(2-nitro-4-pyrrolidi n-1-ylmethyl-phenyl)-ethyl]-amide

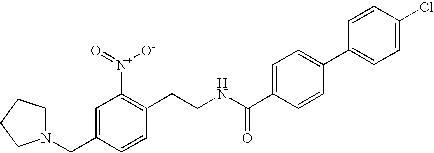

2.111 a. 4-{2-[(4′-chloro-biphenyl-4-carbonyl)-amino]-ethyl}-3-nitro-benzoic acid

2 mL 1N NaOH solution are added to a solution of 200 mg (0.44 mmol) of ethyl 4-{2-[(4′-chloro-biphenyl-4-carbonyl)-amino]-ethyl}-3-nitro-benzoate (Example 2.109b) in 10 mL EtOH and the reaction mixture is stirred for 1 h at RT. The mixture is evaporated down in vacuo, water and 2 mL 1N HCl solution are added to the residue and the suspension is stirred for 30 min in the ice bath. The product is suction filtered, washed with water and dried at 50° C.

Yield: 180 mg (95.9% of theory);

C₂₂H₁₇ClN₂O₅ (M=424.84);

calc.: molar peak (M+H)⁺: 425/427 fnd.: molar peak (M+H)⁺: 425/427;

R_{f} value: 0.07 (silica gel, EtOAc/MeOH/NH₃ 9:1:0.1).

2.111 b. 4′-chloro-biphenyl-4-carboxylic acid-[2-(4-hydroxymethyl-2-nitro-phenyl)-ethyl]-amide

Prepared analogously to Example 2.109f from 180 mg (0.42 mmol) of 4-{2-[(4′-chloro-biphenyl-4-carbonyl)-amino]-ethyl}-3-nitro-benzoic acid.

Yield: 110 mg (63.1% of theory);

C₂₂H₁₉ClN₂O₄ (M=410.86);

calc.: molar peak (M+H)⁺: 411/413 fnd.: molar peak (M+H)⁺: 411/413;

Retention time HPLC: 8.27 min (method A).

2.111 c. 4′-chloro-biphenyl-4-carboxylic acid [2-(2-nitro-4-pyrrolidin-1-ylmethyl-phenyl)-ethyl]-amide

23 μL methanesulphonic acid chloride are slowly added dropwise to a solution of 110 mg (0.27 mmol) of 4′-chloro-biphenyl-4-carboxylic acid-[2-(4-hydroxymethyl-2-nitro-phenyl)-ethyl]-amide and 48 μL triethylamine in 5 mL CH₂Cl₂ cooled to 5° C. The solution is heated for 1 h to 40° C., 5 mL DMF and 115 μL (1.34 mmol) of pyrrolidine are added and the mixture is heated to 80° C. for a further hour, during which time the CH₂Cl₂ is evaporated off. The reaction mixture is evaporated down in vacuo, the residue is combined with water, exhaustively extracted with EtOAc and the organic phase is dried over MgSO₄. After elimination of the drying agent and solvent the residue is purified by HPLC.

Yield: 11 mg (8.8% of theory);

C₂₆H₂₆ClN₃O₃ (M=463.97);

calc.: molar peak (M+H)⁺: 464/466 fnd.: molar peak (M+H)⁺: 464/466;

Retention time HPLC: 6.44 min (method A).

### EXAMPLE 2.112

### 4′-chloro-biphenyl-4-carboxylic acid [2-(2-methanesulphonylamino-4-pyrrolidin-1-ylmethyl-phenyl)-ethyl]-amide

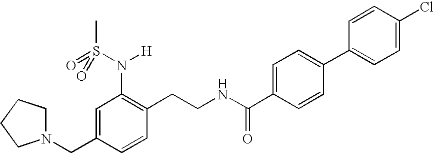

2.112a. ethyl 4-{2-[(4′-chloro-biphenyl-4-carbonyl)-amino]-ethyl}-3-methanesulphonylamino-benzoate

44 μL (0.57 mmol) of methanesulphonic acid chloride are slowly added dropwise to a solution of 200 mg (0.47 mmol) of ethyl 3-amino-4-{2-[(4′-chloro-biphenyl-4-carbonyl)-amino]-ethyl}-benzoate (Example 2.109c) in 5 mL pyridine cooled to 0° C. and the reaction mixture is stirred for 1 h at RT. It is combined with ice water, extracted exhaustively with EtOAc, the organic phase is washed several times with water and dried over MgSO₄. After elimination of the drying agent and solvent the residue is further reacted without purification.

Yield: 230 mg (97.1% of theory);

C₂₅H₂₅ClN₂O₅S (M=501.01);

calc.: molar peak (M+H)⁺: 501/503 fnd.: molar peak (M+H)⁺: 501/503;

Retention time HPLC: 5.66 min (method B).

2.112b. 4-{2-[(4′-chloro-biphenyl-4-carbonyl)-amino]-ethyl}-3-methanesulphonylamino-benzoic acid

Prepared analogously to Example 2.111 a from 230 mg (0.46 mmol) of ethyl 4-{2-[(4′-chloro-biphenyl-4-carbonyl)-amino]-ethyl}-3-methanesulphonylamino-benzoate.

Yield: 180 mg (82.9% of theory);

C₂₃H₂₁ClN₂O₅S (M=472.95);

calc.: molar peak (M−H)⁻: 471/473 fnd.: molar peak (M−H)⁻: 471/473;

Retention time HPLC: 7.67 min (method A).

2.112c. 4′-chloro-biphenyl-4-carboxylic acid [2-(4-hydroxymethyl-2-methanesulphonylamino-phenyl)-ethyl]-amide

Prepared analogously to Example 2.109f from 180 mg (0.38 mmol) of 4-{2-[(4′-chloro-biphenyl-4-carbonyl)-amino]-ethyl}-3-methanesulphonylamino-benzoic acid.

Yield: 150 mg (85.8% of theory);

C₂₃H₂₃ClN₂O₄S (M=458.97);

calc.: molar peak (M+H)⁺: 459/461 fnd.: molar peak (M+H)⁺: 459/461;

Retention time HPLC: 7.53 min (method A).

2.112d. 4′-chloro-biphenyl-4-carboxylic acid [2-(2-methanesulphonylamino-4-pyrrolidin-1-ylmethyl-phenyl)-ethyl]-amide

Prepared analogously to Example 2.111 c from 150 mg (0.33 mmol) of 4′-chloro-biphenyl-4-carboxylic acid [2-(4-hydroxymethyl-2-methanesulphonylamino-phenyl)-ethyl]-amide and 14 μL (1.64 mmol) of pyrrolidine.

After purification by HPLC the product is obtained as the formate salt.

Yield: 18 mg (9.9% of theory);

C₂₇H₃₀ClN₃O₃S*CH₂O₂ (M=558.10);

calc.: molar peak (M+H)⁺: 512/514 fnd.: molar peak (M+H)⁺: 512/514;

Retention time HPLC: 6.13 min (method A).

### EXAMPLE 2.113

### 4′-chloro-biphenyl-4-carboxylic acid [2-(3-pyridin-4-yl-4-pyrrolidin-1-ylmethyl-phenyl)-ethyl]-amide

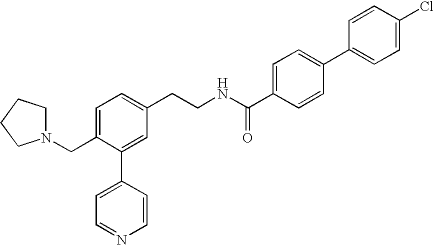

Prepared analogously to Example 2.108 from 200 mg (0.40 mmol) of 4′-chloro-biphenyl-4-carboxylic acid [2-(3-bromo-4-pyrrolidin-1-ylmethyl-phenyl)-ethyl]-amide and 74 mg (0.60 mmol) of pyridine-4-boric acid, purifying the product by HPLC.

Yield: 13 mg (6.5% of theory);

C₃₁H₃₀ClN₃O (M=496.06);

calc.: molar peak (M+H)⁺: 496/498 fnd.: molar peak (M+H)⁺: 496/498;

Retention time HPLC: 6.37 min (method A).

### EXAMPLE 2.114

### Methyl 5-{2-[(4′-chloro-biphenyl-4-carbonyl)-amino]-ethyl}-2-pyrrolidin-1-ylmethyl-benzoate

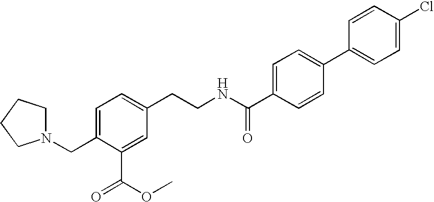

2.114a. methyl 5-cyanomethyl-2-pyrrolidin-1-ylmethyl-benzoate

0.5 mL triethylamine (3.58 mmol), 40 mg (0.18 mmol) of Pd(OAc)₂ and 99 mg (0.18 mmol) of 1,1′-diphenylphosphino-ferrocene are added to a solution of 500 mg (1.79 mmol) of (3-bromo-4-pyrrolidin-1-ylmethyl-phenyl)-acetonitrile (Example 2.107d) in 10 mL MeOH and 10 mL DMF. The reaction mixture is stirred for 15 h at 50° C. in an autoclave with 2 bar CO. To complete the reaction a further 0.5 mL triethylamine, 40 mg Pd(OAc)₂ and 99 mg 1,1′-diphenylphosphino-ferrocene are added, and the mixture is stirred for a further 10 h at 50° C. and 2 bar CO and overnight at 4 bar CO and 70° C. The solvents are evaporated down in vacuo, the residue is combined with EtOAc and extracted twice with water. The aqueous phase is saturated with K₂CO₃, exhaustively extracted with EtOAc and dried over MgSO₄. After elimination of the drying agent and solvent the product is left is a black oil which is further reacted without purification.

Yield: 380 mg (82.1% of theory).

C₁₅H₁₈N₂O₂ (M=258.32);

calc.: molar peak (M+H)⁺: 259 fnd.: molar peak (M+H)⁺: 259;

Retention time HPLC: 2.49 min (method B).

2.114b. methyl 5-(2-amino-ethyl)-2-pyrrolidin-1-ylmethyl-benzoate

100 mg of Raney nickel are added to a solution of 380 mg (1.47 mmol) of methyl 5-cyanomethyl-2-pyrrolidin-1-ylmethyl-benzoate in 20 mL methanolic NH₃ and the reaction mixture is hydrogenated at 20 psi H₂ for 27 h at RT. The catalyst is suction filtered, the solvent is eliminated and the residue is further reacted without purification.

Yield: 330 mg (85.5% of theory).

C₁₅H₂₂N₂O₂ (M=262.36);

calc.: molar peak (M+H)⁺: 263 fnd.: molar peak (M+H)⁺: 263;

Retention time HPLC: 1.40 min (method A).

2.114c. methyl 5-{2-[(4′-chloro-biphenyl-4-carbonyl)-amino]-ethyl}-2-pyrrolidin-1-ylmethyl-benzoate

Prepared according to general working method I from 330 mg (1.26 mmol) of methyl 5-(2-amino-ethyl)-2-pyrrolidin-1-ylmethyl-benzoate and 293 mg (1.26 mmol) of 4′-chloro-biphenyl-4-carboxylic acid.

Yield: 315 mg (52.5% of theory);

C₂₈H₂₉ClN₂O₃ (M=477.01);

calc.: molar peak (M+H)⁺: 477/479 fnd.: molar peak (M+H)⁺: 477/479;

Retention time HPLC: 6.82 min (method A).

### EXAMPLE 2.115

### 5-{2-[(4′-chloro-biphenyl-4-carbonyl)-amino]-ethyl}-2-pyrrolidin-1-ylmethyl-benzoic acid

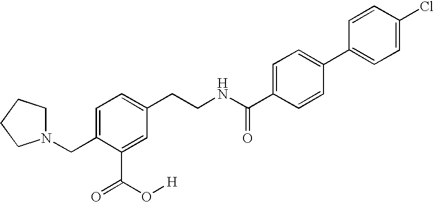

Prepared analogously to Example 2.111a from 310 mg (0.65 mmol) of methyl 5-{2-[(4′-chloro-biphenyl-4-carbonyl)-amino]-ethyl}-2-pyrrolidin-1-ylmethyl-benzoate

Yield: 85 mg (28.2% of theory);

C₂₇H₂₇ClN₂O₃ (M=462.98);

calc.: molar peak (M+H)⁺: 463/465 fnd.: molar peak (M+H)⁺: 463/465;

Retention time HPLC: 6.30 min (method A).

### EXAMPLE 2.116

### tert.butyl (5-{2-[(4′-chloro-biphenyl-4-carbonyl)-amino]-ethyl}-2-pyrrolidin-1-ylmethyl-phenyl)-carbaminate

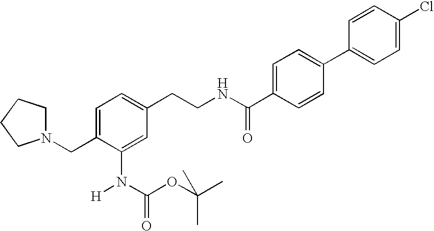

0.27 mL (1.92 mmol) of triethylamine and 0.41 mL (1.92 mmol) of diphenyl azido-phosphate are added to a solution of 740 mg (1.6 mmol) of 5-{2-[(4′-chloro-biphenyl-4-carbonyl)-amino]-ethyl}-2-pyrrolidin-1-ylmethyl-benzoic acid in 10 mL tert.butanol and the reaction mixture is refluxed for 5 h. It is evaporated down in vacuo, the residue is combined with CH₂Cl₂, extracted with 1N NaOH solution and the organic phase is dried over MgSO₄. After elimination of the drying agent and solvent the residue is purified by chromatography on silica gel.

Yield: 85 mg (28.2% of theory);

C₃₁H₃₆ClN₃O₃ (M=534.10);

calc.: molar peak (M+H)⁺: 534/536 fnd.: molar peak (M+H)⁺: 534/536;

Retention time HPLC: 4.82 min (method B).

### EXAMPLE 2.117

### 4′-chloro-biphenyl-4-carboxylic acid [2-(3-ethyl-4-pyrrolidin-1-ylmethyl-phenyl)-ethyl]-amide

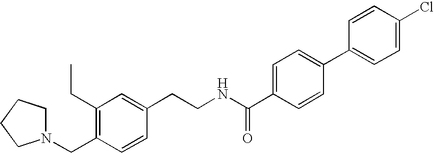

2.117a. (4-pyrrolidin-1-ylmethyl-3-trimethylsilanylethinyl-phenyl)-acetonitrile

A suspension of 0.36 g (1.29 mmol) of (3-bromo-4-pyrrolidin-1-ylmethyl-phenyl)-acetonitrile (Example 2.107d), 0.36 mL (2.58 mmol) of trimethylsilylacetylene, 0.36 mL (2.58 mmol) of triethylamine, 25 mg (0.13 mmol) of CuI and 0.15 g (0.13 mmol) of tetrakis-(triphenylphosphine)-palladium in 3 mL DMF is stirred in the microwave (CEM) for 15 min at 100° C. and 200 Watt. After cooling of the reaction mixture saturated NaCl solution is added, the mixture is exhaustively extracted with EtOAc and the organic phase is dried over MgSO₄. After elimination of the drying agent and solvent the residue is purified by chromatography on silica gel (EtOAc).

Yield: 50 mg (13.1% of theory);

C₁₈H₂₄N₂Si (M=296.49);

calc.: molar peak (M+H)⁺: 297 fnd.: molar peak (M+H)⁺: 297;

Retention time HPLC: 6.39 min (method A).

2.117b. 2-(3-ethyl-4-pyrrolidin-1-ylmethyl-phenyl)-ethylamine

20 mg of Raney nickel are added to a solution of 50 mg (0.17 mmol) of (4-pyrrolidin-1-ylmethyl-3-trimethylsilanylethinyl-phenyl)-acetonitrile in 5 mL methanolic NH₃ and the reaction mixture is shaken for 22 h at RT and 3 bar H₂. The catalyst is suction filtered and the solvent is eliminated in vacuo. The crude product is further reacted without purification.

Yield: 39 mg (100% of theory);

C₁₅H₂₄N₂ (M=232.37);

calc.: molar peak (M+H)⁺: 233 fnd.: molar peak (M+H)⁺: 233.

2.117c. 4′-chloro-biphenyl-4-carboxylic acid [2-(3-ethyl-4-pyrrolidin-1-ylmethyl-phenyl)-ethyl]-amide

Prepared according to general working method I from 40 mg (0.17 mmol) of 2-(3-ethyl-4-pyrrolidin-1-ylmethyl-phenyl)-ethylamine and 48 mg (0.21 mmol) of 4′-chloro-biphenyl-4-carboxylic acid.

Yield: 2 mg (2.6% of theory);

C₂₈H₃₁ClN₂O (M=447.03);

calc.: molar peak (M+H)⁺: 447/449 fnd.: molar peak (M+H)⁺: 447/449;

Retention time HPLC: 6.87 min (method A).

### EXAMPLE 2.118

### 4′-chloro-biphenyl-4-carboxylic acid [2-(6-pyrrolidin-1-ylmethyl-pyridin-3-yl)-ethyl]-amide

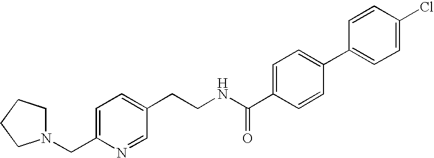

2.118a. methyl 6-dibromomethyl-nicotinate

53.4 g (0.3 mol) of NBS and 2 g dibenzoylperoxide are added to a solution of 38.96 g (0.25 mol) of methyl 6-methyl-nicotinate in 1 L CCl₄ and the reaction mixture is refluxed overnight. Then another 26.7 g (0.15 mol) of NBS and 1 g dibenzoylperoxide are added and the mixture is again refluxed for 24 h. After cooling of the reaction mixture the precipitate is suction filtered, the solvent is eliminated and the residue is purified by chromatography.

Yield: 15.0 g (19.4% of theory);

C₈H₇Br₂NO₂ (M=308.96);

calc.: molar peak (M+H)⁺: 308/310/312 fnd.: molar peak (M+H)⁺: 308/310/312;

R_{f} value: 0.6 (silica gel, petroleum ether/EtOAc 8:2).

2.118b. methyl 6-dimethoxymethyl-nicotinate

13.9 mL of NaOMe in MeOH (30%, 75 mmol) in 100 mL MeOH are heated to boiling. A solution of 11.0 g (34.1 mmol) of methyl 6-dibromomethyl-nicotinate in 10 mL MeOH is added dropwise to the boiling solution and refluxed overnight. To complete the reaction a further 1.5 mL (8.1 mmol) of the NaOMe solution are added and the mixture is refluxed again for 24 h. The reaction mixture is evaporated down in vacuo, the residue is combined with dilute KHSO₄ solution, neutralised with dilute NaHCO₃ solution, exhaustively extracted with EtOAc, the organic phase is washed with water and dried over MgSO₄. After elimination of the drying agent and solvent the residue is further reacted without purification.

Yield: 5.0 g (69.5% of theory);

C₁₀H₁₃NO₄ (M=211.22);

calc.: molar peak (M+H)⁺: 212 fnd.: molar peak (M+H)⁺: 212;

R_{f} value: 0.44 (silica gel, petroleum ether/EtOAc 6:4).

2.118c. 6-dimethoxymethyl-nicotinic acid

15 mL 1N NaOH solution are added to a solution of 2.8 g (13.26 mmol) of methyl 6-dimethoxymethyl-nicotinate in 50 ml MeOH and stirred for 24 h at RT. The reaction mixture is neutralised with 15 mL 1N HCl, evaporated down in vacuo, the residue is triturated with MeOH/THF, the precipitate is suction filtered and the filtrate is evaporated down. The product obtained is further reacted without purification.

Yield: 2.6 g (99.4% of theory);

C₉H₁₁NO₄ (M=197.19);

calc.: molar peak (M+H)⁺: 198 fnd.: molar peak (M+H)⁺: 198;

Retention time HPLC: 3.65 min (method A).

2.118d. (6-dimethoxymethyl-pyridin-3-yl)-methanol

Prepared analogously to Example 2.109f from 2.7 g (13.7 mmol) of 6-dimethoxymethyl-nicotinic acid, using THF as solvent and tert.butylmethylether for the extraction.

Yield: 2.1 g (83.7% of theory);

C₉H₁₃NO₃ (M=183.21);

calc.: molar peak (M+H)⁺: 184 fnd.: molar peak (M+H)⁺: 184;

Retention time HPLC: 2.85 min (method A).

2.118e. 5-chloromethyl-2-dimethoxymethyl-pyridine

0.3 mL (4.14 mmol) of thionyl chloride, dissolved in a little CH₂Cl₂, are slowly added dropwise to a solution of 500 mg (2.73 mmol) of (6-dimethoxymethyl-pyridin-3-yl)-methanol in 10 ml CH₂Cl₂ cooled to 0° C. and stirred for a further 30 min at this temperature. The reaction mixture is diluted with CH₂Cl₂, washed with cold NaHCO₃ solution and dried over MgSO₄. After elimination of the drying agent and solvent the residue is further reacted without purification.

Yield: 500 mg (90.8% of theory);

C₉H₁₂ClNO₂ (M=201.65);

calc.: molar peak (M+H)⁺: 202/204 fnd.: molar peak (M+H)⁺: 202/204;

R_{f} value: 0.3 (silica gel, petroleum ether/EtOAc 6:4).

2.118f. (6-dimethoxymethyl-pyridin-3-yl)-acetonitrile

20 mL DMSO are added to 5.21 g (80 mmol) of KCN in 5.2 mL water and at 80° C. a solution of 500 mg (2.48 mmol) of 5-chloromethyl-2-dimethoxymethyl-pyridine in 10 mL DMSO is added dropwise and the reaction mixture is kept for a further hour at 80° C. It is poured onto 200 mL water, saturated with NaCl, extracted exhaustively with EtOAc, the organic phase is dried over MgSO₄ and filtered through activated charcoal. The filtrate is evaporated down and the residue is purified by chromatography on silica gel (CH₂Cl₂/MeOH 9:1).

Yield: 330 mg (69.2% of theory);

C₁₀H₁₂N₂O₂ (M=192.22);

calc.: molar peak (M+H)⁺: 193 fnd.: molar peak (M+H)⁺: 193;

R_{f} value: 0.48 (silica gel, CH₂Cl₂/MeOH 9:1).

2.118g. 2-(6-dimethoxymethyl-pyridin-3-yl)-ethylamine

50 mg of Raney nickel are added to a solution of 330 mg (1.72 mmol) of (6-dimethoxymethyl-pyridin-3-yl)-acetonitrile in 10 mL methanolic NH₃ and the reaction mixture is hydrogenated in a Parr autoclave at 30° C. 15 h under 3 bar H₂.

The catalyst is filtered off, the solvent is evaporated down in vacuo and the residue is further reacted without purification.

Yield: 340 mg (100% of theory);

C₁₀H₁₆N₂O₂ (M=196.25);

calc.: molar peak (M+H)⁺: 197 fnd.: molar peak (M+H)⁺: 197;

Retention time HPLC: 1.3 min (method A).

2.118h. 4′-chloro-biphenyl-4-carboxylic acid [2-(6-dimethoxymethyl-pyridin-3-yl)-ethyl]-amide

Prepared according to general working method I from 340 mg (1.73 mmol) of 2-(6-dimethoxymethyl-pyridin-3-yl)-ethylamine and 419 mg (1.80 mmol) of 4′-chloro-biphenyl-4-carboxylic acid.

Yield: 210 mg (28.4% of theory);

C₂₃H₂₃ClN₂O₃ (M=410.90);

calc.: molar peak (M+H)⁺: 411/413 fnd.: molar peak (M+H)⁺: 411/413;

R_{f} value: 0.4 (silica gel, CH₂Cl₂/MeOH/NH₃ 9:1:0.1).

2.118i. 4′-chloro-biphenyl-4-carboxylic acid [2-(6-formyl-pyridin-3-yl)-ethyl]-amide

5 mL 12% HCl are added to a solution of 205 mg (0.5 mmol) of 4′-chloro-biphenyl-4-carboxylic acid [2-(6-dimethoxymethyl-pyridin-3-yl)-ethyl]-amide in 10 mL MeOH and the reaction mixture is stirred for 4 h at RT and heated to 80° C. overnight. Another 2.5 mL of 12% HCl are added, the mixture is heated for a further 8 h at 80° C. and overnight at 100° C. The reaction mixture is combined with 50 mL water, adjusted to pH 8 with Na₂CO₃ solution, exhaustively extracted with CH₂Cl₂ and the organic phase is dried over MgSO₄. After elimination of the drying agent and solvent the residue is further reacted without purification.

Yield: 180 mg (98.7% of theory);

C₂₁H₁₇ClN₂O₂ (M=364.84);

calc.: molar peak (M+H)⁺: 365/367 fnd.: molar peak (M+H)⁺: 365/367;

Retention time HPLC: 5.25 min (method A).

2.118k. 4′-chloro-biphenyl-4-carboxylic acid [2-(6-pyrrolidin-1-ylmethyl-pyridin-3-yl)-ethyl]-amide

50 μL (0.6 mmol) of pyrrolidine, 37.7 mg (0.6 mmol) of NaBH₃CN and 2 mL MeOH are added to a solution of 180 mg (0.49 mmol) of 4′-chloro-biphenyl-4-carboxylic acid [2-(6-formyl-pyridin-3-yl)-ethyl]-amide in 5 mL acetonitrile, the pH value is adjusted to 5-6 with glacial acetic acid and the mixture is stirred for 5 h at RT. The reaction mixture is acidified with 1M KHSO₄ solution, made alkaline with 2M Na₂CO₃ solution, exhaustively extracted with CH₂Cl₂ and the organic phase is dried over MgSO₄. After elimination of the drying agent and solvent the residue is purified by chromatography on silica gel (CH₂Cl₂/MeOH/NH₃ 9:1:0.1).

Yield: 25 mg (12.1% of theory);

C₂₅H₂₆ClN₃O (M=419.96);

calc.: molar peak (M+H)⁺: 420/422 fnd.: molar peak (M+H)⁺: 420/422;

R_{f} value: 0.2 (silica gel, CH₂Cl₂/MeOH/NH₃ 9:1:0.1).

### EXAMPLE 2.119

### 4′-chloro-biphenyl-4-carboxylic acid [2-(5-pyrrolidin-1-ylmethyl-pyridin-2-yl)-ethyl]-amide

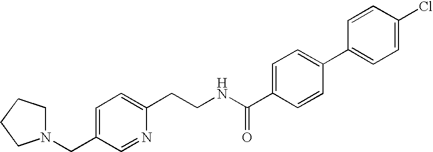

2.119a. methyl 6-hydroxymethyl-nicotinate

Prepared analogously to Example 2.109f from 5.0 g (27.6 mmol) of 5-methyl pyridine-2,5-dicarboxylate, using THF as solvent and tert-butylmethylether for the extraction.

Yield: 2.0 g (43.3% of theory);

C₈H₉NO₃ (M=167.17);

calc.: molar peak (M+H)⁺: 168 fnd.: molar peak (M+H)⁺: 168;

R_{f} value: 0.2 (silica gel, CH₂Cl₂/MeOH 95:5).

2.119b. methyl 6-chloromethyl-nicotinate

1.06 mL (13 mmol) of pyridine added and slowly 1.08 mL (13 mmol) of thionyl chloride are added dropwise to a solution of 2.0 g (11.96 mmol) of methyl 6-hydroxymethyl-nicotinate in 100 mL CH₂Cl₂ cooled to 0° C. This is stirred for a further hour at 0° C. and then slowly heated to RT. To complete the reaction a further 1 mL (12 mmol) of thionyl chloride is added and the mixture is stirred for 1 h at RT. The reaction mixture water is added, the organic phase is separated off, washed with dilute NaHCO₃ solution and water and dried over MgSO₄. This is filtered through activated charcoal and the solvent is evaporated down in vacuo. The product obtained is further reacted without purification.

Yield: 1.7 g (65.1% of theory);

C₈H₈ClNO₂ (M=185.61);

calc.: molar peak (M+H)⁺: 186/188 fnd.: molar peak (M+H)⁺: 186/188;

Retention time HPLC: 6.7 min (method A).

2.119c. methyl 6-cyanomethyl-nicotinate

Prepared analogously to Example 2.118f from 1.5 g (8.08 mmol) of methyl 6-chloromethyl-nicotinate and 5.2 g (80 mmol) of KCN, using cyclohexane/EtOAc 8:2 as eluant for the purification by chromatography on silica gel.

Yield: 220 mg (15.5% of theory);

C₉H₈N₂O₂ (M=176.18);

calc.: molar peak (M+H)⁺: 177 fnd.: molar peak (M+H)⁺: 177;

R_{f} value: 0.6 (silica gel, petroleum ether/EtOAc 1:1).

2.119d. methyl 6-(2-amino-ethyl)-nicotinate

20 mg of Raney nickel are added to a solution of 75 mg (0.43 mmol) of methyl 6-cyanomethyl-nicotinate in 5 mL methanolic NH₃ and the reaction mixture is hydrogenated in a Parr autoclave at 30° C. for 6 h under 3 bar H₂. The catalyst is filtered off, the solvent is evaporated down in vacuo and the residue is further reacted without purification.

Yield: 70 mg (90.3% of theory);

C₉H₁₂N₂O₂ (M=180.21);

calc.: molar peak (M+H)⁺: 181 fnd.: molar peak (M+H)⁺: 181;

Retention time HPLC: 2.5 min (method A).

2.119e. methyl 6-{2-[(4′-chloro-biphenyl-4-carbonyl)-amino]-ethyl}-nicotinate

Prepared according to general working method I from 70 mg (0.39 mmol) of methyl 6-(2-amino-ethyl)-nicotinate and 100 mg (0.43 mmol) of 4′-chloro-biphenyl-4-carboxylic acid.

Yield: 150 mg (88.3% of theory);

C₂₂H₁₉ClN₂O₃ (M=394.86);

calc.: molar peak (M+H)⁺: 395/397 fnd.: molar peak (M+H)⁺: 395/397;

Retention time HPLC: 8.6 min (method A).

2.119f. 6-{2-[(4′-chloro-biphenyl-4-carbonyl)-amino]-ethyl}-nicotinic acid

0.8 mL 1 M NaOH solution are added to a solution of 150 mg (0.38 mmol) of methyl 6-{2-[(4′-chloro-biphenyl-4-carbonyl)-amino]-ethyl}-nicotinate in 25 mL MeOH and the reaction mixture is refluxed for 1 h. It is neutralised with 0.8 mL of 1 N HCl, evaporated down in vacuo, the residue is stirred with water and the precipitate is removed by suction filtering. It is dissolved in THF, the solution is dried with MgSO₄, filtered and evaporated down in vacuo. The residue is further reacted without purification.

Yield: 90 mg (62.2% of theory);

C₂₁H₁₇ClN₂O₃ (M=380.83);

calc.: molar peak (M+H)⁺: 381/383 fnd.: molar peak (M+H)⁺: 381/383;

Retention time HPLC: 6.9 min (method A).

2.119g. 4′-chloro-biphenyl-4-carboxylic acid [2-(5-hydroxymethyl-pyridin-2-yl)-ethyl]-amide

Prepared analogously to Example 2.109f from 90 mg (0.24 mmol) of 6-{2-[(4′-chloro-biphenyl-4-carbonyl)-amino]-ethyl}-nicotinic acid, using THF as solvent and tert-butylmethylether for the extraction.

Yield: 50 mg (56.8% of theory);

C₂₁H₁₉ClN₂O₂ (M=366.85);

calc.: molar peak (M+H)⁺: 367/369 fnd.: molar peak (M+H)⁺: 367/369;

R_{f} value: 0.5 (silica gel, CH₂Cl₂/MeOH 9:1).

2.119h. 4′-chloro-biphenyl-4-carboxylic acid [2-(5-pyrrolidin-1-ylmethyl-pyridin-2-yl)-ethyl]-amide

22 μL thionyl chloride are added dropwise to a solution of 50 mg (0.14 mmol) of 4′-chloro-biphenyl-4-carboxylic acid [2-(5-hydroxymethyl-pyridin-2-yl)-ethyl]-amide in 5 mL CH₂Cl₂ cooled to 0° C. and the reaction mixture is slowly allowed to warm up to RT. After 1 h at RT a further 22 μL thionyl chloride are added dropwise to complete the reaction and stirring is continued for 1 h. The reaction mixture is diluted with 30 mL CH₂Cl₂, combined with ice water, made alkaline with NaHCO₃ solution, the organic phase is separated off, washed with water and dried over MgSO₄. After elimination of the drying agent 50 μL (0.6 mmol) of pyrrolidine are added to this solution and the reaction mixture is stirred overnight at RT. It is evaporated down in vacuo and the residue is purified by HPLC chromatography.

Yield: 2.4 mg (4.1% of theory);

C₂₅H₂₆ClN₃O (M=419.96);

calc.: molar peak (M+H)⁺: 420/422 fnd.: molar peak (M+H)⁺: 420/422;

R_{f} value: 0.3 (silica gel, CH₂Cl₂/MeOH 9:1).

Retention time HPLC: 6.0 min (method A).

### EXAMPLE 2.120

### 4′-chloro-biphenyl-4-carboxylic acid {2-[4-(1-pyrrolidin-1-yl-ethyl)-phenyl]-ethyl}-amide

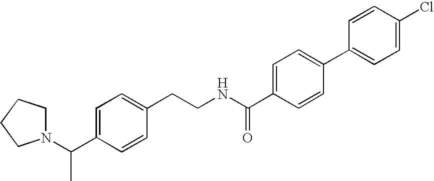

2.120a. tert.butyl [2-(4-acetyl-phenyl)-ethyl]-carbaminate

5.46 g (25 mmol) of BOC-anhydride are added to a solution of 4.99 g (25 mmol) of 1-[4-(2-amino-ethyl)-phenyl]-ethanone (used as the hydrochloride) in 100 ml CH₂Cl₂ and at RT 25 mL of 1N NaOH solution are slowly added dropwise and after the addition has ended the mixture is stirred for 2 h at RT. The reaction mixture is filtered through Celite, washed twice with water and dried over MgSO₄. It is filtered through activated charcoal, evaporated down in vacuo and the product is further reacted without purification.

Yield: 6.4 g (97.2% of theory);

C₁₅H₂₁NO₃ (M=263.34);

calc.: molar peak (M+H)⁺: 262 fnd.: molar peak (M+H)⁺: 262;

R_{f} value: 0.88 (silica gel, CH₂Cl₂/MeOH/NH₃ 9:1:0.1).

2.120b. tert.butyl {2-[4-(1-hydroxy-ethyl)-phenyl]-ethyl}-carbaminate

4.72 g (125 mmol) of NaBH₄ are added batchwise at RT to a solution of 6.58 g (25 mmol) of tert.butyl [2-(4-acetyl-phenyl)-ethyl]-carbaminate in 250 mL MeOH and the reaction mixture is stirred over the weekend. It is carefully acidified with KHSO₄ solution, extracted exhaustively with tert-butylmethylether, the organic phase is washed with saturated NaCl solution and dried over MgSO₄. After elimination of the drying agent and solvent the product is left as a slightly yellowish oil which crystallises out when left to stand.

Yield: 5.4 g (81.4% of theory);

C₁₅H₂₃NO₃ (M=265.36);

calc.: molar peak (M+H)⁺: 266 fnd.: molar peak (M+H)⁺: 266;

R_{f} value: 0.4 (silica gel, petroleum ether/EtOAc 6:4).

2.120c. tert.butyl {2-[4-(1-pyrrolidin-1-yl-ethyl)-phenyl]-ethyl}-carbaminate

0.66 mL (8.5 mmol) of methanesulphonic acid chloride, dissolved in 10 mL CH₂Cl₂, are added dropwise to a solution of 2.89 g (10.89 mmol) of tert.butyl {2-[4-(1-hydroxy-ethyl)-phenyl]-ethyl}-carbaminate in 50 mL CH₂Cl₂ and 1.25 ml triethylamine cooled to 0° C. Stirring is continued for 1 h at this temperature and then a solution of 1.4 mL (17 mmol) of pyrrolidine in 10 mL CH₂Cl₂ is slowly added dropwise. The reaction mixture is stirred overnight at RT, combined with dilute KHSO₄ solution, the organic phase is separated off, washed twice with dilute KHSO₄ solution, the combined aqueous phases are made basic with K₂CO₃ solution and exhaustively extracted with tert-butylmethylether. The combined organic phases are washed several times with a little water and dried over MgSO₄. After elimination of the drying agent and solvent the product is further reacted without purification.

Yield: 0.3 g (8.7% of theory);

C₁₉H₃₀N₂O₂ (M=318.46);

calc.: molar peak (M+H)⁺: 319 fnd.: molar peak (M+H)⁺: 319;

R_{f} value: 0.22 (silica gel, CH₂Cl₂/MeOH/NH₃ 9:1:0.1).

2.120d. 2-[4-(1-pyrrolidin-1-yl-ethyl)-phenyl]-ethylamine

0.72 mL trifluoroacetic acid are added to a solution of 300 mg (0.94 mmol) of tert.butyl {2-[4-(1-pyrrolidin-1-yl-ethyl)-phenyl]-ethyl}-carbaminate in 20 mL CH₂Cl₂ and stirred for 1 h at RT. To complete the reaction a further 0.72 mL of trifluoroacetic acid are added and the reaction mixture is kept for 1 h at RT. The solvent is evaporated down in vacuo, the residue is taken up in water, made alkaline with 2 N NaOH, exhaustively extracted with EtOAc and the organic phase is dried over MgSO₄. After elimination of the drying agent and solvent the product is further reacted without purification.

Yield: 150 mg (72.9% of theory);

C₁₄H₂₂N₂ (M=218.35);

calc.: molar peak (M+H)⁺: 219 fnd.: molar peak (M+H)⁺: 219;

R_{f} value: 0.15 (silica gel, CH₂Cl₂/MeOH/NH₃ 8:2:0.2).

2.120e. 4′-chloro-biphenyl-4-carboxylic acid {2-[4-(1-pyrrolidin-1-yl-ethyl)-phenyl]-ethyl}-amide

Prepared according to general working method I from 150 mg (0.69 mmol) of 2-[4-(1-pyrrolidin-1-yl-ethyl)-phenyl]-ethylamine and 176 mg (0.76 mmol) of 4′-chloro-biphenyl-4-carboxylic acid.

Yield: 150 mg (88.3% of theory);

C₂₇H₂₉ClN₂O (M=433.0);

calc.: molar peak (M+H)⁺: 433/435 fnd.: molar peak (M+H)⁺: 433/435;

Retention time HPLC: 6.33 min (method A).

### EXAMPLE 2.121

### 4′-chloro-biphenyl-4-carboxylic acid {2-[3-bromo-4-(2,5-dihydro-pyrrol-1-ylmethyl)-phenyl]-ethyl}-amide

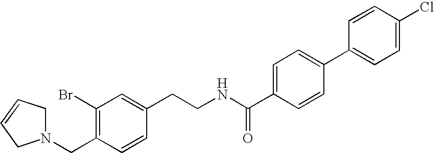

2.121 a. [4-(2-amino-ethyl)-2-bromo-phenyl]-methanol

100 mg of Raney nickel are added to a solution of 4 g (17.68 mmol) of (3-bromo-4-hydroxymethyl-phenyl)-acetonitrile (cf. Example 2.107c.) in 100 mL THF and 50 mL methanolic NH₃ and the reaction mixture is shaken in a Parr autoclave for 5 h at RT and 5 psi H₂. The catalyst is filtered off, the solvent removed and the product is further reacted without purification.

Yield: 3.8 g (93.4% of theory);

C₉H₁₂BrNO (M=230.11);

calc.: molar peak (M+H)⁺: 230/232 fnd.: molar peak (M+H)⁺: 230/232;

Retention time HPLC: 1.85 min (method A).

2.121b. tert.butyl [2-(3-bromo-4-hydroxymethyl-phenyl)-ethyl]-carbaminate

17 mL of a 1 M BOC-anhydride solution in CH₂Cl₂ are added to a solution of 3.8 g (16.51 mmol) of [4-(2-amino-ethyl)-2-bromo-phenyl]-methanol in 50 mL CH₂Cl₂ and the reaction mixture is stirred overnight at RT. It is diluted with 100 mL of dilute KHSO₄ solution, the organic phase is separated off, washed with dilute NaHCO₃ solution and water and dried over MgSO₄. After elimination of the drying agent and solvent the residue is purified by chromatography on silica gel.

Yield: 2.3 g (42.2% of theory);

C₁₄H₂₀BrNO₃ (M=330.22);

R_{f} value: 0.44 (silica gel, petroleum ether/EtOAc 6:4).

2.121c. tert.butyl [2-(3-bromo-4-chloromethyl-phenyl)-ethyl]-carbaminate

0.54 mL (6.5 mmol) of thionyl chloride are slowly added dropwise to a solution of 1.98 g (6.0 mmol) of tert.butyl [2-(3-bromo-4-hydroxymethyl-phenyl)-ethyl]-carbaminate in 50 mL CH₂Cl₂ and 0.53 mL pyridine cooled to 0° C., stirred for a further hour at 0° C. and then heated to RT. Water is added to the reaction mixture, the organic phase is washed with dilute KHSO₄ solution and water and dried over MgSO₄. After filtration through activated charcoal and elimination of the solvent the product is further reacted without purification.

Yield: 2.0 g (95.6% of theory);

C₁₄H₁₉BrClNO₂ (M=348.67);

calc.: molar peak (M+H)⁺: 348/350/352 fnd.: molar peak (M+H)⁺: 348/350/352;

R_{f} value: 0.6 (silica gel, petroleum ether/EtOAc 6:4).

2.121d. tert.butyl {2-[3-bromo-4-(2,5-dihydro-pyrrol-1-ylmethyl)-phenyl]-ethyl}-carbaminate

0.84 mL (11 mmol) of 2,5-dihydro-1H-pyrrole are added to a suspension of 1.9 g (5.45 mmol) of tert.butyl [2-(3-bromo-4-chloromethyl-phenyl)-ethyl]-carbaminate and 2.5 g (18.1 mmol) of K₂CO₃ in 50 mL acetonitrile and the reaction mixture is stirred overnight at RT. The suspension is filtered, the filtrate evaporated down in vacuo and the residue purified by chromatography on silica gel.

Yield: 0.5 g (24.1% of theory);

C₁₈H₂₅BrN₂O₂ (M=381.32);

calc.: molar peak (M+H)⁺: 381/383 fnd.: molar peak (M+H)⁺: 381/383;

R_{f} value: 0.58 (silica gel, CH₂Cl₂/MeOH 8:2).

2.121e. 2-[3-bromo-4-(2,5-dihydro-pyrrol-1-ylmethyl)-phenyl]-ethylamine

5 mL trifluoroacetic acid are added to a solution of 500 mg (1.31 mmol) of tert.butyl {2-[3-bromo-4-(2,5-dihydro-pyrrol-1-ylmethyl)-phenyl]-ethyl}-carbaminate in 50 mL CH₂Cl₂ and the reaction mixture is stirred for 2 h at RT.

It is evaporated down in vacuo, combined with water and CH₂Cl₂, adjusted to an alkaline pH with K₂CO₃ solution, the organic phase is separated off and washed again with water. This is evaporated down in vacuo and the product is purified by chromatography on silica gel.

Yield: 350 mg (95.0% of theory);

C₁₃H₁₇BrN₂ (M=281.20);

calc.: molar peak (M+H)⁺: 281/283 fnd.: molar peak (M+H)⁺: 281/283;

R_{f} value: 0.08 (silica gel, CH₂Cl₂/MeOH/NH₃ 95:5:0.5).

2.121 f. 4′-chloro-biphenyl-4-carboxylic acid {2-[3-bromo-4-(2,5-dihydro-pyrrol-1-ylmethyl)-phenyl]-ethyl}-amide

Prepared according to general working method I from 141 mg (0.5 mmol) of 2-[3-bromo-4-(2,5-dihydro-pyrrol-1-ylmethyl)-phenyl]-ethylamine and 116 mg (0.5 mmol) of 4′-chloro-biphenyl-4-carboxylic acid.

Yield: 140 mg (56.5% of theory);

C₂₆H₂₄BrClN₂O (M=495.85);

calc.: molar peak (M+H)⁺: 495/497/499 fnd.: molar peak (M+H)⁺: 495/497/499;

Retention time HPLC: 6.6 min (method A).

### EXAMPLE 2.122

### 4′-bromo-3-fluoro-biphenyl-4-carboxylic acid {2-[3-bromo-4-(2,5-dihydro-pyrrol-1-ylmethyl)-phenyl]-ethyl}-amide

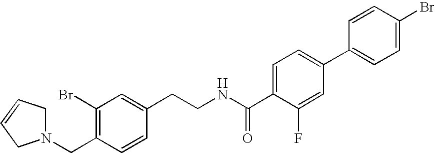

2.122a. 4′-bromo-3-fluoro-biphenyl-4-carboxylic acid

1.04 g (5 mmol) of 4-bromophenylboric acid, 115 mg (0.1 mmol) of tetrakis-(triphenylphosphine)-palladium and 2 ml 2M Na₂CO₃ solution are added successively to a solution of 1.1 g (5 mmol) of 4-bromo-2-fluorobenzoic acid in 5 mL DMF and 5 mL dioxane and the reaction mixture is refluxed for 2 h. To complete the reaction a further 250 mg (1.25 mmol) of 4-bromophenylboric acid are added and the mixture is refluxed for a further 2 h. The reaction mixture is filtered hot through a glass fibre filter, washed with water, acidified with dilute KHSO₄ solution, the precipitate formed is suction filtered and washed with water. The residue is triturated with acetonitrile and a little MeOH, filtered to remove insoluble matter, the filtrate is evaporated down, the residue is triturated with MeOH and the product is suction filtered.

Yield: 140 mg (9.5% of theory);

C₁₃H₈BrFO₂ (M=295.11);

calc.: molar peak (M+H)⁺: 293/295 fnd.: molar peak (M+H)⁺: 293/295;

R_{f} value: 0.5 (silica gel, CH₂Cl₂/MeOH 9:1).

2.122b. 4′-bromo-3-fluoro-biphenyl-4-carboxylic acid {2-[3-bromo-4-(2,5-dihydro-pyrrol-1-ylmethyl)-phenyl]-ethyl}-amide

Prepared according to general working method I from 141 mg (0.5 mmol) of 2-[3-bromo-4-(2,5-dihydro-pyrrol-1-ylmethyl)-phenyl]-ethylamine and 140 mg (0.47 mmol) of 4′-bromo-3-fluoro-biphenyl-4-carboxylic acid.

Yield: 10 mg (3.8% of theory);

C₂₆H₂₃Br₂FN₂O (M=558.29);

calc.: molar peak (M+H)⁺: 557/559/561 fnd.: molar peak (M+H)⁺: 557/559/561;

Retention time HPLC: 7.0 min (method A).

### EXAMPLE 2.123

### 4′-chloro-biphenyl-4-carboxylic acid [2-(3-amino-4-pyrrolidin-1-ylmethyl-phenyl)-ethyl]-amide

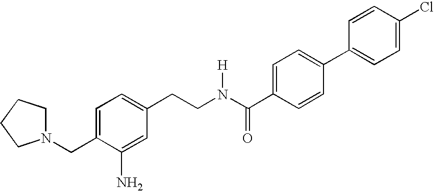

0.12 mL trifluoroacetic acid are added to a solution of 40 mg (0.08 mmol) of tert.butyl (5-{2-[(4′-chloro-biphenyl-4-carbonyl)-amino]-ethyl}-2-pyrrolidin-1-ylmethyl-phenyl)-carbaminate (cf. Example 2.116) in 3 mL CH₂Cl₂ and the reaction mixture is stirred at RT over the weekend. It is evaporated down in vacuo, combined with semisaturated NaHCO₃ solution, extracted with EtOAc and the organic phase is dried over MgSO₄. After elimination of the drying agent and solvent the residue is purified by HPLC.

Yield: 3 mg (7.3% of theory);

C₂₆H₂₈ClN₃O*C₂HF₃O₂ (M=548.01);

calc.: molar peak (M+H)⁺: 434/436 fnd.: molar peak (M+H)⁺: 434/436;

Retention time HPLC: 5.35 min (Stable Bond C18; 3.5 μM; water:acetonitrile:formic acid 6:4:0.015).

### EXAMPLE 2.124

### 4′-chloro-biphenyl-4-carboxylic acid-ethyl-[2-(4-pyrrolidin-1-ylmethyl-phenyl)-ethyl]-amide

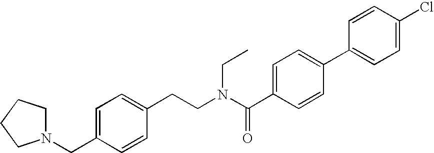

2.124a. ethyl-[2-(4-pyrrolidin-1-ylmethyl-phenyl)-ethyl]-amine

A solution of 89 μL (1.1 mmol) of ethyl iodide in 1 mL THF is added dropwise to a solution of 204 mg (1.0 mmol) of 2-(4-pyrrolidin-1-ylmethyl-phenyl)-ethylamine and 0.17 mL triethylamine in 5 mL THF and the reaction mixture is stirred for 24 h at RT. It is combined with saturated NaHCO₃ solution, extracted with EtOAc and the organic phase is dried over MgSO₄. After elimination of the drying agent and solvent the residue is further reacted without purification.

Yield: 70 mg (30.1% of theory).

2.124b. 4′-chloro-biphenyl-4-carboxylic acid-ethyl-[2-(4-pyrrolidin-1-ylmethyl-phenyl)-ethyl]-amide

Prepared according to general working method I from 70 mg (0.3 mmol) of ethyl-[2-(4-pyrrolidin-1-ylmethyl-phenyl)-ethyl]-amine and 81 mg (0.35 mmol) of 4′-chloro-biphenyl-4-carboxylic acid.

Yield: 20 mg (14.9% of theory);

C₂₈H₃₁ClN₂O (M=447.03);

calc.: molar peak (M+H)⁺: 447/449 fnd.: molar peak (M+H)⁺: 447/449;

Retention time HPLC: 6.92 min (method A).

### EXAMPLE 2.125

### 4′-chloro-biphenyl-4-carboxylic acid-isobutyl-[2-(4-pyrrolidin-1-ylmethyl-phenyl)-ethyl]-amide

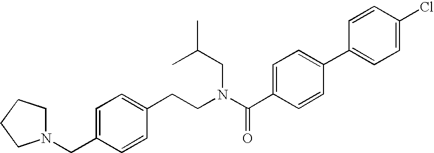

2.125a. isobutyl-[2-(4-pyrrolidin-1-ylmethyl-phenyl)-ethyl]-amine

A solution of 204 mg (1.0 mmol) of 2-(4-pyrrolidin-1-ylmethyl-phenyl)-ethylamine and 91 μL (1.0 mmol) of isobutyraldehyde in 20 mL THF is acidified slightly with glacial acetic acid, combined with 253 mg (1.2 mmol) of NaBH(OAc)₃ and stirred overnight at RT. The reaction mixture is combined with semisaturated NaHCO₃ solution, exhaustively extracted with EtOAc; the aqueous phase is saturated with K₂CO₃ and extracted with EtOAc. The combined organic phases are dried over MgSO₄. After elimination of the drying agent and solvent the residue is further reacted without purification.

Yield: 250 mg (96.0% of theory).

C₁₇H₂₈N₂ (M=260.43);

calc.: molar peak (M+H)⁺: 261 fnd.: molar peak (M+H)⁺: 261;

R_{f} value: 0.4 (silica gel, CH₂Cl₂/MeOH/NH₃ 8:2:0.2).

2.125b. 4′-chloro-biphenyl-4-carboxylic acid-isobutyl-[2-(4-pyrrolidin-1-ylmethyl-phenyl)-ethyl]-amide

Prepared according to general working method I from 250 mg (0.96 mmol) of isobutyl-[2-(4-pyrrolidin-1-ylmethyl-phenyl)-ethyl]-amine and 244 mg (1.05 mmol) of 4′-chloro-biphenyl-4-carboxylic acid.

Yield: 67 mg (14.7% of theory);

C₃₀H₃₅ClN₂O (M=475.08);

calc.: molar peak (M+H)⁺: 475/477 fnd.: molar peak (M+H)⁺: 475/477;

Retention time HPLC: 7.67 min (method A).

### EXAMPLE 2.126

### 4′-chloro-biphenyl-4-carboxylic acid-cyclohex-3-enylmethyl-[2-(4-pyrrolidin-1-ylmethyl-phenyl)-ethyl]-amide

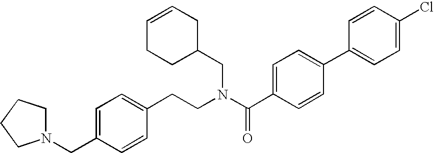

2.126a. cyclohex-3-enylmethyl-[2-(4-pyrrolidin-1-ylmethyl-phenyl)-ethyl]-amine

Prepared analogously to Example 2.125a. from 204 mg (1.0 mmol) of 2-(4-pyrrolidin-1-ylmethyl-phenyl)-ethylamine and 114 μL (1.0 mmol) of 1,2,3,6-tetrahydrobenzaldehyde.

Yield: 100 mg (33.5% of theory).

C₂₀H₃₀N₂ (M=298.48);

R_{f} value: 0.2 (silica gel, CH₂Cl₂/MeOH/NH₃ 8:2:0.2).

2.126b. 4′-chloro-biphenyl-4-carboxylic acid-cyclohex-3-enylmethyl-[2-(4-pyrrolidin-1-ylmethyl-phenyl)-ethyl]-amide

Prepared according to general working method I from 100 mg (0.34 mmol) of cyclohex-3-enylmethyl-[2-(4-pyrrolidin-1-ylmethyl-phenyl)-ethyl]-amine and 86 mg (0.37 mmol) of 4′-chloro-biphenyl-4-carboxylic acid.

Yield: 46 mg (26.8% of theory);

C₃₃H₃₇ClN₂O (M=513.13);

calc.: molar peak (M+H)⁺: 513/515 fnd.: molar peak (M+H)⁺: 513/515;

Retention time HPLC: 8.20 min (method A).

### EXAMPLE 2.127

### 4′-chloro-biphenyl-4-carboxylic acid-benzyl-[2-(4-pyrrolidin-1-ylmethyl-phenyl)-ethyl]-amide

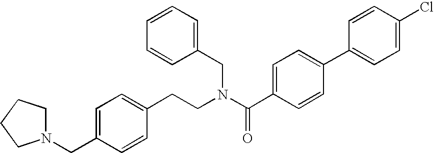

2.127a. benzyl-[2-(4-pyrrolidin-1-ylmethyl-phenyl)-ethyl]-amine

Prepared analogously to Example 2.125a. from 204 mg (1.0 mmol) of 2-(4-pyrrolidin-1-ylmethyl-phenyl)-ethylamine and 102 μL (1.0 mmol) of benzaldehyde.

Yield: 160 mg (54.3% of theory).

C₂₀H₂₆N₂ (M=294.44);

R_{f} value: 0.28 (silica gel, CH₂Cl₂/MeOH/NH₃ 8:2:0.2).

2.127b. 4′-chloro-biphenyl-4-carboxylic acid-benzyl-[2-(4-pyrrolidin-1-ylmethyl-phenyl)-ethyl]-amide

Prepared according to general working method I from 160 mg (0.54 mmol) of benzyl-[2-(4-pyrrolidin-1-ylmethyl-phenyl)-ethyl]-amine and 140 mg (0.60 mmol) of 4′-chloro-biphenyl-4-carboxylic acid.

Yield: 16 mg (5.8% of theory);

C₃₃H₃₃ClN₂O (M=509.10);

calc.: molar peak (M+H)⁺: 509/511 fnd.: molar peak (M+H)⁺: 509/511;

Retention time HPLC: 7.51 min (method A).

### EXAMPLE 2.128

### 4′-chloro-biphenyl-4-carboxylic acid-cyclohexylmethyl-[2-(4-pyrrolidin-1-ylmethyl-phenyl)-ethyl]-amide

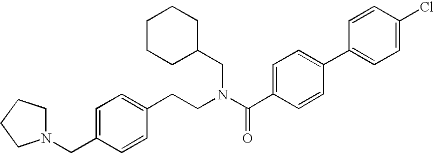

2.128a. cyclohexylmethyl-[2-(4-pyrrolidin-1-ylmethyl-phenyl)-ethyl]-amine

Prepared analogously to Example 2.125a. from 204 mg (1.0 mmol) of 2-(4-pyrrolidin-1-ylmethyl-phenyl)-ethylamine and 121 μL (1.0 mmol) of cyclohexanecarbaldehyde.

Yield: 100 mg (33.3% of theory).

C₂₀H₃₂N₂ (M=300.49);

R_{f} value: 0.18 (silica gel, CH₂Cl₂/MeOH/NH₃ 8:2:0.2).

2.128b. 4′-chloro-biphenyl-4-carboxylic acid-cyclohexylmethyl-[2-(4-pyrrolidin-1-ylmethyl-phenyl)-ethyl]-amide

Prepared according to general working method I from 100 mg (0.33 mmol) of cyclohexylmethyl-[2-(4-pyrrolidin-1-ylmethyl-phenyl)-ethyl]-amine and 86 mg (0.37 mmol) of 4′-chloro-biphenyl-4-carboxylic acid.

Yield: 70 mg (40.8% of theory);

C₃₃H₃₃ClN₂O (M=515.15);

calc.: molar peak (M+H)⁺: 515/517 fnd.: molar peak (M+H)⁺: 515/517;

Retention time HPLC: 8.63 min (method A).

### EXAMPLE 2.129

### 4′-chloro-biphenyl-4-carboxylic acid-cyclopropylmethyl-[2-(4-pyrrolidin-1-ylmethyl-phenyl)-ethyl]-amide

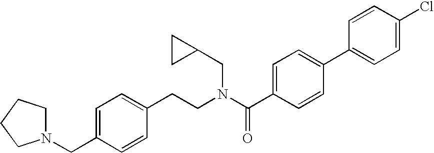

2.129a. cyclopropylmethyl-[2-(4-pyrrolidin-1-ylmethyl-phenyl)-ethyl]-amine

Prepared analogously to Example 2.125a. from 204 mg (1.0 mmol) of 2-(4-pyrrolidin-1-ylmethyl-phenyl)-ethylamine and 75 μL (1.0 mmol) of cyclopropanecarbaldehyde.

Yield: 100 mg (38.7% of theory).

C₁₇H₂₆N₂ (M=258.41);

R_{f} value: 0.30 (silica gel, CH₂Cl₂/MeOH/NH₃ 8:2:0.2).

2.129b. 4′-chloro-biphenyl-4-carboxylic acid-cyclopropylmethyl-[2-(4-pyrrolidin-1-ylmethyl-phenyl)-ethyl]-amide

Prepared according to general working method I from 100 mg (0.39 mmol) of cyclopropylmethyl-[2-(4-pyrrolidin-1-ylmethyl-phenyl)-ethyl]-amine and 100 mg (0.43 mmol) of 4′-chloro-biphenyl-4-carboxylic acid.

Yield: 23 mg (12.6% of theory);

C₃₀H₃₃ClN₂O (M=473.06);

calc.: molar peak (M+H)⁺: 473/475 fnd.: molar peak (M+H)⁺: 473/475;

Retention time HPLC: 7.45 min (method A).

### EXAMPLE 2.130

### 4-pentyl-N-[2-(4-pyrrolidin-1-ylmethyl-phenyl)-ethyl]-benzamide

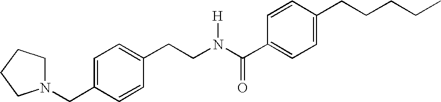

Prepared according to general working method I from 2-(4-pyrrolidin-1-ylmethyl-phenyl)-ethylamine (102 mg, 0.50 mmol) and 4-pentyl-benzoic acid (96 mg, 0.50 mmol).

Yield: 75 mg (39.6% of theory);

C₂₅H₃₄N₂O (M=378.56);

calc.: molar peak (M+H)⁺: 379 fnd.: molar peak (M+H)⁺: 379;

Retention time HPLC: 6.5 min (method A).

### EXAMPLE 2.131

### 4-butyl-N-[2-(4-pyrrolidin-1-ylmethyl-phenyl)-ethyl]-benzamide

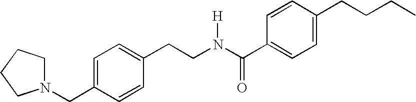

Prepared according to general working method I from 2-(4-pyrrolidin-1-ylmethyl-phenyl)-ethylamine (102 mg, 0.50 mmol) and 4-butyl-benzoic acid (89 mg, 0.50 mmol).

Yield: 60 mg (32.9% of theory);

C₂₄H₃₂N₂O (M=364.54);

calc.: molar peak (M+H)⁺: 365 fnd.: molar peak (M+H)⁺: 365;

Retention time HPLC: 6.0 min (method A).

### EXAMPLE 2.132

### 4-butylamino-N-[2-(4-pyrrolidin-1-ylmethyl-phenyl)-ethyl]-benzamide

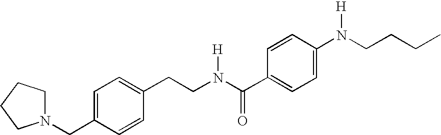

Prepared according to general working method I from 2-(4-pyrrolidin-1-ylmethyl-phenyl)-ethylamine (204 mg, 1.0 mmol) and 4-butylamino-benzoic acid (155 mg, 0.80 mmol).

Yield: 30 mg (9.9% of theory);

C₂₄H₃₃N₃O (M=379.55);

calc.: molar peak (M+H)⁺: 380 fnd.: molar peak (M+H)⁺: 380;

Retention time HPLC: 6.0 min (method A).

### EXAMPLE 2.133

### 4-(1-methyl-butyl)-N-[2-(4-pyrrolidin-1-ylmethyl-phenyl)-ethyl]-benzamide

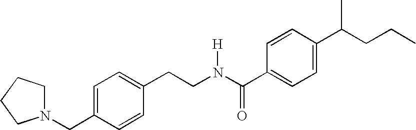

Prepared according to general working method I from 2-(4-pyrrolidin-1-ylmethyl-phenyl)-ethylamine (82 mg, 0.40 mmol) and 4-(1-methyl-butyl)-benzoic acid (75 mg, 0.39 mmol).

Yield: 40 mg (27.1% of theory);

C₂₄H₃₂N₂O (M=378.56);

calc.: molar peak (M+H)⁺: 379 fnd.: molar peak (M+H)⁺: 379;

Retention time HPLC: 4.3 min (method B).

### EXAMPLE 2.134

### N-[2-(4-pyrrolidin-1-ylmethyl-phenyl)-ethyl]-4-(4,4,4-trifluoro-butoxy)-benzamide

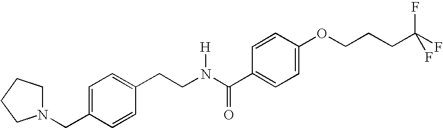

2.134a. methyl 4-(4,4,4-trifluoro-butoxy)-benzoate

608 mg (4.4 mmol) of K₂CO₃ are added to a solution of 304 mg (2.0 mmol) of methyl 4-hydroxybenzoate in 10 mL DMF and then 382 mg (2.0 mmol) of 1-bromo-4,4,4-trifluorobutane. The mixture is stirred overnight at RT, again combined with 1-bromo-4,4,4-trifluorobutane and stirred for a further 24 h at RT. The reaction solution is diluted with water and exhaustively extracted twice with EtOAc. The combined org. extracts are dried over MgSO₄ and evaporated down i. vac. The crude product is used without further purification in the next reaction step.

Yield: 500 mg (95.3% of theory);

C₁₂H₁₃F₃O₃ (M=262.23);

calc.: molar peak (M+H)⁺: 263 fnd.: molar peak (M+H)⁺: 263;

R_{f} value: 0.9 (silica gel, petroleum ether/EtOAc 6:4).

2.134b. 4-(4,4,4-trifluoro-butoxy)-benzoic acid

10.0 mL (10.0 mmol) of 1M sodium hydroxide solution are added to a solution of 500 mg (1.9 mmol) of methyl 4-(4,4,4-trifluoro-butoxy)-benzoate in 7 mL THF. The mixture is stirred for 8 h under reflux. THF is removed i. vac. and the residue is acidified with hydrochloric acid. After filtration the precipitate formed is dried in the air.

Yield: 350 mg (73.9% of theory);

C₁₁H₁₁F₃O₃ (M=248.20);

calc.: molar peak (M−H)—: 247 fnd.: molar peak (M−H)—: 247;

Retention time HPLC: 7.5 min (method A).

2.134c. N-[2-(4-pyrrolidin-1-ylmethyl-phenyl)-ethyl]-4-(4,4,4-trifluoro-butoxy)-benzamide

Prepared according to general working method I from 2-(4-pyrrolidin-1-ylmethyl-phenyl)-ethylamine (102 mg, 0.50 mmol) and 4-(4,4,4-trifluoro-butoxy)-benzoic acid (124 mg, 0.50 mmol).

Yield: 37 mg (17.0% of theory);

C₂₄H₂₉F₃N₂O₂ (M=434.51);

calc.: molar peak (M+H)⁺: 435 fnd.: molar peak (M+H)⁺: 435;

Retention time HPLC: 5.8 min (method A).

### EXAMPLE 2.135

### 3-methyl-4-pent-1-ynyl-N-[2-(4-pyrrolidin-1-ylmethyl-phenyl)-ethyl]-benzamide

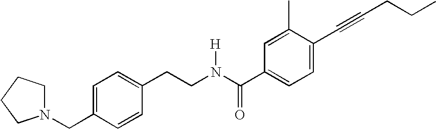

2.135a. methyl 3-methyl-4-pent-1-ynyl-benzoate

0.39 mL (4.0 mmol) of pentyne, 0.56 mL (4.0 mmol) of triethylamine, 70 mg (0.1 mmol) of bis-(triphenylphosphine)-palladium(II)-chloride and 19 mg (0.1 mmol) of copper(I)-iodide are added successively to a solution of 458 mg (2.0 mmol) of methyl 4-bromo-3-methyl-benzoate in 3.0 mL DMF. The reaction solution is stirred in the microwave for 10 min at 200 Watt and 65° C. A further 0.20 mL (2.0 mmol) of pentyne are added and the reaction solution is stirred for a further 20 min in the microwave at 200 Watt and 70° C. The mixture is diluted with 30 mL EtOAc, filtered through Celite and the filtrate is washed three times with 50 mL water. The combined organic extracts are dried over MgSO₄, filtered through activated charcoal and the solvent is eliminated i. vac. The purification is carried out by column chromatography on silica gel (cyclohexane after cyclohexane/ethyl acetate 9:1).

Yield: 200 mg (46.2% of theory);

C₁₄H₁₆O₂ (M=216.28);

calc.: molar peak (M+H)⁺: 217 fnd.: molar peak (M+H)⁺: 217;

Retention time HPLC: 6.8 min (method B).

2.135b. 3-methyl-4-pent-1-ynyl-benzoic acid

3.0 mL (3.0 mmol) of 1M sodium hydroxide solution are added to a solution of 200 mg (0.93 mmol) of methyl 3-methyl-4-pent-1-ynyl-benzoate in 3 mL methanol. The mixture is refluxed for 3 h. The reaction solution is diluted with water and extracted once with 40 mL of EtOAc. The aqueous phase is acidified with 1M KHSO₄ solution and extracted twice with 40 mL EtOAC. The combined organic phases are dried over MgSO₄. After elimination of the drying agent and solvent the crude product is used in the next reaction step without further purification.

Yield: 50 mg (26.7% of theory);

C₁₃H₁₄O₂ (M=202.26);

calc.: molar peak (M−H)⁻: 201 fnd.: molar peak (M−H)⁻: 201;

Retention time HPLC: 5.6 min (method B).

2.135c. 3-methyl-4-pent-1-ynyl-N-[2-(4-pyrrolidin-1-ylmethyl-phenyl)-ethyl]-benzamide

Prepared according to general working method I from 2-(4-pyrrolidin-1-ylmethyl-phenyl)-ethylamine (51 mg, 0.25 mmol) and 3-methyl-4-pent-1-ynyl-benzoic acid (50 mg, 0.25 mmol).

Yield: 22 mg (22.9% of theory);

C₂₆H₃₂N₂O (M=388.558);

calc.: molar peak (M+H)⁺: 389 fnd.: molar peak (M+H)⁺: 389;

Retention time HPLC: 6.9 min (method A).

### EXAMPLE 2.136

### 4-pent-1-ynyl-N-[2-(4-pyrrolidin-1-ylmethyl-phenyl)-ethyl]-benzamide

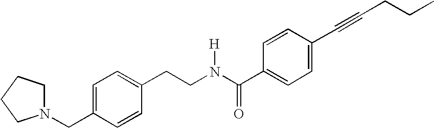

2.136a. ethyl 4-pent-1-ynyl-benzoate

0.39 mL (4 mmol) of 1-pentyne, 0.56 mL triethylamine, 70 mg (0.1 mmol) of bis-(triphenylphosphine)-palladium(II)-chloride and 19 mg (0.1 mmol) of CuI are added successively to a solution of 552 mg (2.0 mmol) of ethyl 4-iodobenzoate in 3 mL DMF. The reaction solution is stirred for 4 h at 80° C. The mixture is diluted with 30 mL EtOAc, filtered through Celite, the filtrate is washed three times with 50 mL water in each case and dried over MgSO₄. After filtration through activated charcoal the solvent is eliminated in vacuo. The purification is carried out by column chromatography on silica gel (cyclohexane after cyclohexane/ethyl acetate 9:1).

Yield: 150 mg (34.7% of theory);

C₁₄H₁₆O₂ (M=216.282);

calc.: molar peak (M+H)⁺: 217 fnd.: molar peak (M+H)⁺: 217;

Retention time HPLC: 6.8 min (method B).

2.136b. 4-pent-1-ynyl-benzoic acid

5.0 mL (5.0 mmol) of 1M sodium hydroxide solution are added to a solution of 150 mg (0.69 mmol) of ethyl 4-pent-1-ynyl-benzoate in 3 mL methanol. The mixture is stirred for 3 h under reflux. The reaction solution is diluted with water and extracted once with 40 mL EtOAc. The aqueous phase is acidified with 1M KHSO₄ solution and extracted twice with 40 mL EtOAc. The combined organic extracts are dried over magnesium sulphate and the solvent is eliminated i. vac. The crude product was used in the next reaction step without further purification.

Yield: 150 mg (115% of theory);

C₁₂H₁₂O₂ (M=188.23);

calc.: molar peak (M−H)⁻: 187 fnd.: molar peak (M−H)⁻: 187;

R_{f} value: 0.2 (silica gel, petroleum ether/EtOAc 8:2).

2.136c. 4-pent-1-ynyl-N-[2-(4-pyrrolidin-1-ylmethyl-phenyl)-ethyl]-benzamide

Prepared according to general working method I from 2-(4-pyrrolidin-1-ylmethyl-phenyl)-ethylamine (163 mg, 0.80 mmol) and 4-pent-1-ynyl-benzoic acid (150 mg, 0.80 mmol).

Yield: 122 mg (40.9% of theory);

C₂₅H₃₀N₂O (M=374.53);

calc.: molar peak (M+H)⁺: 375 fnd.: molar peak (M+H)⁺: 375;

R_{f} value: 0.35 (silica gel, EtOAc/methanol/NH₃ 9:1:0.1).

### EXAMPLE 2.137

### (4-pent-1-enyl)-N-[2-(4-pyrrolidin-1-ylmethyl-phenyl)-ethyl]-benzamide

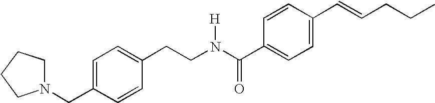

2.137a. methyl 4-pent-1-enyl-benzoate

246 mg (2.2 mmol) of potassium-tert-butoxide are added at 0° C. to a solution of 1.08 g (2.2 mmol) of (4-methoxycarbonyl-benzyl)-triphenyl-phosphonium-bromide in 20 mL of THF under an argon atmosphere. The orange solution is stirred for a further 15 min at 0° C. and then combined with 0.18 mL (2.0 mmol) of butyraldehyde. The reaction solution is refluxed for 3 h and then diluted with EtOAc. The organic phase is washed twice with water, dried over magnesium sulphate and the solvent is removed i. vac. The residue is triturated with diisopropylether, filtered and the filtrate is evaporated down. The further purification is carried out by column chromatography on silica gel (petroleum ether/EtOAc 6:4). Methyl 4-pent-1-enyl-benzoate is obtained as a 2:1 mixture of E/Z isomers.

Yield: 350 mg (56.5% of theory);

C₁₃H₁₆O₂ (M=204.27);

calc.: molar peak (M+H)⁺: 204 fnd.: molar peak (M+H)⁺: 204;

R_{f} value: 0.90 (silica gel, petroleum ether/EtOAc 6:4).

2.137b. 4-pent-1-enyl-benzoic acid

5.0 mL (5.0 mmol) of 1M sodium hydroxide solution are added to a solution of 350 mg (1.71 mmol) of ethyl 4-pent-1-enyl-benzoate in 4 mL methanol. The mixture was refluxed for 2 h. The solvent is removed i. vac. and the residue is combined with 6M hydrochloric acid solution. The precipitate formed is suction filtered and dried at 35° C. in the circulating air dryer. The further purification is carried out by filtration through a silica gel column (petroleum ether/EtOAc 6:4).

Yield: 300 mg (92.1% of theory);

C₁₂H₁₄O₂ (M=190.24);

calc.: molar peak (M−H)⁻: 189 fnd.: molar peak (M−H)⁻: 189;

R_{f} value: 0.4 (silica gel, petroleum ether/EtOAc 6:4).

2.137c. (4-pent-1-enyl)-N-[2-(4-pyrrolidin-1-ylmethyl-phenyl)-ethyl]-benzamide

Prepared according to general working method I from 2-(4-pyrrolidin-1-ylmethyl-phenyl)-ethylamine (306 mg, 1.50 mmol) and 4-pent-1-enyl-benzoic acid (300 mg, 1.56 mmol) as a 2:1 mixture of E/Z isomers.

Yield: 130 mg (23.0% of theory);

C₂₅H₃₂N₂O (M=376.547);

calc.: molar peak (M+H)⁺: 377 fnd.: molar peak (M+H)⁺: 377;

Retention time HPLC: 6.9 min (method A).

### EXAMPLE 2.138

### 3-chloro-4-cyclohexyl-N-[2-(4-pyrrolidin-1-ylmethyl-phenyl)-ethyl]-benzamide

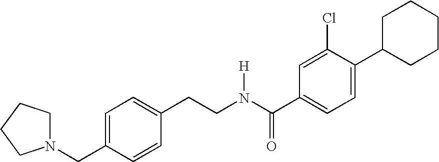

Prepared according to general working method I from 2-(4-pyrrolidin-1-ylmethyl-phenyl)-ethylamine (102 mg, 0.50 mmol) and 3-chloro-4-cyclohexyl-benzoic acid (119 mg, 0.50 mmol).

Yield: 46 mg (21.6% of theory);

C₂₆H₃₃ClN₂O (M=425.019);

calc.: molar peak (M+H)⁺: 425/427 fnd.: molar peak (M+H)⁺: 425/427;

Retention time HPLC: 4.7 min (method B).

Some test methods for determining an MCH-receptor antagonistic activity will now be described. In addition, other test methods known to the skilled man are used, e.g. by inhibiting the MCH-receptor-mediated inhibition of cAMP production, as described by Hoogduijn M et al. in “Melanin-concentrating hormone and its receptor are expressed and functional in human skin”, Biochem. Biophys. Res Commun. 296 (2002) 698-701 and by biosensory measurement of the binding of MCH to the MCH receptor in the presence of antagonistic substances by plasmon resonance, as described by Karlsson OP and Lofas S. in “Flow-Mediated On-Surface Reconstitution of G-Protein Coupled Receptors for Applications in Surface Plasmon Resonance Biosensors”, Anal. Biochem. 300 (2002), 132-138. Other methods of testing antagonistic activity to MCH receptors are contained in the references and patent documents mentioned hereinbefore, and the description of the test methods used is hereby incorporated in this application.

MCH-1 receptor binding test

| | |
|---|---|
| Method: | MCH binding to hMCH-1R transfected cells |
| Species: | Human |
| Test cell: | hMCH-1R stably transfected into |
| | CHO/Galpha16 cells |
| Results: | IC50 values |

Membranes from CHO/Galpha16 cells stably transfected with human hMCH-1R are resuspended using a syringe (needle 0.6×25 mm) and diluted in test buffer (50 mM HEPES, 10 mM MgCl₂, 2 mM EGTA, pH 7.00; 0.1% bovine serum albumin (protease-free), 0.021% bacitracin, 1 μg/ml aprotinin, 1 μg/ml leupeptin and 1 μM phosphoramidone) to a concentration of 5 to 15 μg/ml. 200 microliters of this membrane fraction (contains 1 to 3 μg of protein) are incubated for 60 minutes at ambient temperature with 100 pM of ¹²⁵I-tyrosyl melanin concentrating hormone (¹²⁵I-MCH commercially obtainable from NEN) and increasing concentrations of the test compound in a final volume of 250 microliters. After the incubation the reaction is filtered using a cell harvester through 0.5% PEI treated glass fibre filters (GF/B, Unifilter Packard). The membrane-bound radioactivity retained on the filter is then determined after the addition of scintillator substance (Packard Microscint 20) in a measuring device (TopCount of Packard).

The non-specific binding is defined as bound radioactivity in the presence of 1 micromolar MCH during the incubation period.

The analysis of the concentration binding curve is carried out on the assumption of one receptor binding site.

Standard:

Non-labelled MCH competes with labelled ¹²⁵I-MCH for the receptor binding with an IC50 value of between 0.06 and 0.15 nM.

The KD value of the radioligand is 0.156 nM.

MCH-1 receptor-coupled Ca²⁺ mobilisation test

| Method: | Calcium mobilisation test with human MCH (FLIPR³⁸⁴) | |
|---|---|---|
| Species: | Human | |
| Test cells: | CHO/Galpha 16 cells stably transfected with hMCH-R1 | |
| Results: | 1st measurement: % stimulation of | |
| | the reference (MCH 10⁻⁶M) | |
| | 2nd measurement: pKB value | |
| Reagents: | HBSS (10×) | (GIBCO) |
| | HEPES buffer (1M) | (GIBCO) |
| | Pluronic F-127 | (Molecular Probes) |
| | Fluo-4 | (Molecular Probes) |
| | Probenecid | (Sigma) |
| | MCH | (Bachem) |
| | bovine serum albumin | (Serva) |
| | (protease-free) | |
| | DMSO | (Serva) |
| | Ham's F12 | (BioWhittaker) |
| | FCS | (BioWhittaker) |
| | L-Glutamine | (GIBCO) |
| | Hygromycin B | (GIBCO) |
| | PENStrep | (BioWhittaker) |
| | Zeocin | (Invitrogen) |

Clonal CHO/Galpha16 hMCH-R1 cells are cultivated in Ham's F12 cell culture medium (with L-glutamine; BioWhittaker; Cat.No.: BE12-615F). This contains per 500 ml 10% FCS, 1% PENStrep, 5 ml L-glutamine (200 mM stock solution), 3 ml hygromycin B (50 mg/ml in PBS) and 1.25 ml zeocin (100 μg/ml stock solution). One day before the experiment the cells are plated on a 384-well microtitre plate (black-walled with a transparent base, made by Costar) in a density of 2500 cells per cavity and cultivated in the above medium overnight at 37° C., 5% CO₂ and 95% relative humidity. On the day of the experiment the cells are incubated with cell culture medium to which 2 mM Fluo-4 and 4.6 mM Probenicid have been added, at 37° C. for 45 minutes. After charging with fluorescent dye the cells are washed four times with Hanks buffer solution (1×HBSS, 20 mM HEPES), which is combined with 0.07% Probenicid. The test substances are diluted in Hanks buffer solution, combined with 2.5% DMSO. The background fluorescence of non-stimulated cells is measured in the presence of substance in the 384-well microtitre plate five minutes after the last washing step in the FLIPR³⁸⁴ apparatus (Molecular Devices; excitation wavelength: 488 nm; emission wavelength: bandpass 510 to 570 nm). To stimulate the cells MCH is diluted in Hanks buffer with 0.1% BSA, pipetted into the 384-well cell culture plate 35 minutes after the last washing step and the MCH-stimulated fluorescence is then measured in the FLIPR³⁸⁴ apparatus.

Data Analysis:

1 st measurement: The cellular Ca²⁺ mobilisation is measured as the peak of the relative fluorescence minus the background and is expressed as the percentage of the maximum signal of the reference (MCH 10⁻⁶M). This measurement serves to identify any possible agonistic effect of a test substance.

2nd measurement: The cellular Ca²⁺ mobilisation is measured as the peak of the relative fluorescence minus the background and is expressed as the percentage of the maximum signal of the reference (MCH 10⁻⁶M, signal is standardised to 100%). The EC50 values of the MCH dosage activity curve with and without test substance (defined concentration) are determined graphically by the GraphPad Prism 2.01 curve program. MCH antagonists cause the MCH stimulation curve to shift to the right in the graph plotted.

The inhibition is expressed as a pKB value:

pKB=log(EC_{50(testsubstance+MCH)}/EC_{50(MCH)}−1)−log c_{(testsubstance)}

The compounds according to the invention, including their salts, exhibit an MCH-receptor antagonistic activity in the tests mentioned above. Using the MCH-1 receptor binding test described above an antagonistic activity is obtained in a dosage range from about 10⁻¹⁰ to 10⁻⁵ M, particularly from 10⁻⁹ to 10⁻⁶ M.

The following IC50 values were determined using the MCH-1 receptor binding test described above:

| Compound according | |
|---|---|
| to Example No. | IC50 value |
| 1.14 | 2.1 nM |
| 2.4 | 3.5 nM |
| 2.12 | 30.5 nM |

Some examples of formulations will be described hereinafter, wherein the term “active substance” denotes one or more compounds according to the invention, including their salts. In the case of one of the combinations with one or more active substances described, the term “active substance” also includes the additional active substances.

### EXAMPLE 3

### Capsules for Powder Inhalation Containing 1 mg Active Substance

Composition:

1 capsule for powder inhalation contains:

| | |
|---|---|
| active substance | 1.0 mg |
| lactose | 20.0 mg |
| hard gelatine capsules | 50.0 mg |
| | 71.0 mg |

Method of Preparation:

The active substance is ground to the particle size required for inhalation. The ground active substance is homogeneously mixed with the lactose. The mixture is packed into hard gelatine capsules.

### EXAMPLE 4

### Inhalable Solution for Respimat® Containing 1 mg Active Substance

Composition:

1 spray contains:

| | | |
|---|---|---|
| active substance | 1.0 | mg |
| benzalkonium chloride | 0.002 | mg |
| disodium edetate | 0.0075 | mg |
| purified water ad | 15.0 | μl |

Method of Preparation:

The active substance and benzalkonium chloride are dissolved in water and packed into Respimat® cartridges.

### EXAMPLE 5

### Inhalable Solution for Nebulisers Containing 1 mg Active Substance

Composition:

1 vial contains:

| | | |
|---|---|---|
| active substance | 0.1 | g |
| sodium chloride | 0.18 | g |
| benzalkonium chloride | 0.002 | g |
| purified water ad | 20.0 | ml |

Method of Preparation:

The active substance, sodium chloride and benzalkonium chloride are dissolved in water.

### EXAMPLE 6

### Propellant Type Metered Dose Aerosol Containing 1 mg Active Substance

Composition:

1 spray contains:

| | |
|---|---|
| active substance | 1.0 mg |
| lecithin | 0.1% |
| propellant gas ad | 50.0 μl |

Method of Preparation:

The micronised active substance is homogeneously suspended in the mixture of lecithin and propellant gas. The suspension is transferred into a pressurised contained with a metering valve.

### EXAMPLE 7

### Nasal Spray Containing 1 mg Active Substance

Composition:

| | | |
|---|---|---|
| active substance | 1.0 | mg |
| sodium chloride | 0.9 | mg |
| benzalkonium chloride | 0.025 | mg |
| disodium edetate | 0.05 | mg |
| purified water ad | 0.1 | ml |

Method of Preparation:

The active substance and the excipients are dissolved in water and transferred into a corresponding container.

### EXAMPLE 8

### Injectable Solution Containing 5 mg of Active Substance per 5 ml

Composition:

| | | |
|---|---|---|
| active substance | 5 | mg |
| glucose | 250 | mg |
| human serum albumin | 10 | mg |
| glycofurol | 250 | mg |
| water for injections ad | 5 | ml |

Preparation:

Glycofurol and glucose are dissolved in water for injections (WfI); human serum albumin is added; active ingredient is dissolved with heating; made up to specified volume with WfI; transferred into ampoules under nitrogen gas.

### EXAMPLE 9

### Injectable Solution Containing 100 mg of Active Substance per 20 ml

Composition:

| | | |
|---|---|---|
| active substance | 100 | mg |
| monopotassium dihydrogen phosphate = | 12 | mg |
| KH₂PO₄ | | |
| disodium hydrogen phosphate = | 2 | mg |
| Na₂HPO₄.2H₂O | | |
| sodium chloride | 180 | mg |
| human serum albumin | 50 | mg |
| Polysorbate 80 | 20 | mg |
| water for injections ad | 20 | ml |

Preparation:

Polysorbate 80, sodium chloride, monopotassium dihydrogen phosphate and disodium hydrogen phosphate are dissolved in water for injections (WfI); human serum albumin is added; active ingredient is dissolved with heating; made up to specified volume with WfI; transferred into ampoules.

### EXAMPLE 10

### Lyophilisate Containing 10 mg of Active Substance

Composition:

| | |
|---|---|
| Active substance | 10 mg |
| Mannitol | 300 mg |
| human serum albumin | 20 mg |

Preparation:

Mannitol is dissolved in water for injections (WfI); human serum albumin is added; active ingredient is dissolved with heating; made up to specified volume with WfI; transferred into vials; freeze-dried.

Solvent for Lyophilisate:

| | | |
|---|---|---|
| Polysorbate 80 = Tween 80 | 20 | mg |
| mannitol | 200 | mg |
| water for injections ad | 10 | ml |

Preparation:

Polysorbate 80 and mannitol are dissolved in water for injections (WfI); transferred into ampoules.

### EXAMPLE 11

### Tablets Containing 20 mg of Active Substance

Composition:

| | |
|---|---|
| active substance | 20 mg |
| lactose | 120 mg |
| maize starch | 40 mg |
| magnesium stearate | 2 mg |
| Povidone K 25 | 18 mg |

Preparation:

Active substance, lactose and maize starch are homogeneously mixed; granulated with an aqueous solution of Povidone; mixed with magnesium stearate; compressed in a tablet press; weight of tablet 200 mg.

### EXAMPLE 12

### Capsules Containing 20 mg Active Substance

Composition:

| | |
|---|---|
| active substance | 20 mg |
| maize starch | 80 mg |
| highly dispersed silica | 5 mg |
| magnesium stearate | 2.5 mg |

Preparation:

Active substance, maize starch and silica are homogeneously mixed; mixed with magnesium stearate; the mixture is packed into size 3 hard gelatine capsules in a capsule filling machine.

### EXAMPLE 13

### Suppositories Containing 50 mg of Active Substance

Composition:

| | |
|---|---|
| active substance | 50 mg |
| hard fat (Adeps solidus) q.s. ad | 1700 mg |

Preparation:

Hard fat is melted at about 38° C.; ground active substance is homogeneously dispersed in the molten hard fat; after cooling to about 35° C. it is poured into chilled moulds.

### EXAMPLE 14

### Injectable Solution Containing 10 mg of Active Substance per 1 ml

Composition:

| | | |
|---|---|---|
| active substance | 10 | mg |
| mannitol | 50 | mg |
| human serum albumin | 10 | mg |
| water for injections ad | 1 | ml |

Preparation:

Mannitol is dissolved in water for injections (WfI); human serum albumin is added; active ingredient is dissolved with heating; made up to specified volume with WfI; transferred into ampoules under nitrogen gas.

What is claimed is:

1. Carboxamide compounds comprised of general formula I 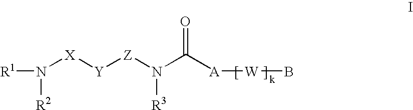
wherein
R¹, R² independently of one another denote H, a C₁₋₈-alkyl or C₃₋₇-cycloalkyl group optionally substituted by the group R¹¹ or a phenyl group optionally mono- or polysubstituted by the group R¹² and/or monosubstituted by nitro, or
R¹ and R² form a C₂₋₈-alkylene bridge wherein
one or two —CH₂-groups may be replaced independently of one another by —CH═N— or —CH═CH— and/or
one or two —CH₂-groups may be replaced independently of one another by —O—, —S—, —CO—, —C(═CH₂)— or —NR¹³— so that heteroatoms are not directly connected to one another,
while in the alkylene bridge defined above one or more H atoms may be replaced by R¹⁴, and/or
the alkylene bridge defined above may be substituted by one or two identical or different carbo- or heterocyclic groups Cy in such a way that the bond between the alkylene bridge and the group Cy is formed
via a single or double bond,
via a common C atom forming a spirocyclic ring system,
via two common, adjacent C and/or N atoms forming a fused bicyclic ring system or
via three or more C and/or N atoms forming a bridged ring system,
R³ denotes H, C₁₋₆-alkyl, C₃₋₇-cycloalkyl, C₃₋₇-cycloalkyl-C₁₋₄-alkyl-, C₃₋₇-cycloalkenyl, C₃₋₇-cycloalkenyl-C₁₋₄-alkyl-, phenyl, phenyl-C₁₋₄-alkyl-, C₁₋₃-alkoxy-C₂₋₆-alkyl-, amino-C₂₋₆-alkyl-, C₁₋₃-alkyl-amino-C₂₋₆-alkyl- or di-(C₁₋₃-alkyl)-amino-C₂₋₆-alkyl-,
X denotes a single bond or a C₁₋₈-alkylene bridge wherein
one or two —CH₂-groups may be replaced independently of one another by —CH═CH— or —C≡C— and/or
one or two —CH₂-groups may be replaced independently of one another by —O—, —S—, —(SO)—, —(SO₂)—, —CO— or —NR⁴— in such a way that in each case two O, S or N atoms or one O atom and an S atom are not directly connected with one another,
while one or two C atoms independently of one another may be substituted by a hydroxy, ω-hydroxy-C₁₋₃-alkyl-, ω-(C₁₋₃-alkoxy)-C₁₋₃-alkyl- and/or C₁₋₃-alkoxy group and/or in each case with one or two identical or different C₁₋₆-alkyl groups, and/or
the alkylene bridge may be connected to R¹ so as to include the N atom connected to R¹ and X, forming a heterocyclic group,
Z denotes a C₁₋₄-alkylene bridge, wherein two adjacent C atoms with an additional C₁₋₄-alkylene bridge may be connected to one another, while in group Z a —CH₂-group may be replaced by —O— or —NR⁵—,
and one or two C atoms of the alkylene bridge may be substituted independently of one another with a hydroxy, ω-hydroxy-C₁₋₃-alkyl-,
ω-(C₁₋₃-alkoxy)-C₁₋₃-alkyl-, C₁₋₃-alkoxy group, amino-C₁₋₃-alkyl-, C₁₋₃-alkyl-amino-C₁₋₃-alkyl- or di-(C₁₋₃-alkyl)-amino-C₁₋₃-alkyl- and/or with one or two identical or different C₁₋₆-alkyl groups, and/or
R³ may be connected to Z so as to include the N atom connected to R³, forming a heterocyclic group,
A, Y independently of one another have one of the meanings given for Cy,
while R¹ may be connected to Y so as to include the group X and the N atom connected to R¹ and X, forming a heterocyclic group fused to Y, and/or
R³ may be connected to Y so as to include the group Z and the N atom connected to R³ and Z, forming a saturated or partially unsaturated heterocyclic group fused to Y, or
A and R³ may be connected to one another in such a way that the group 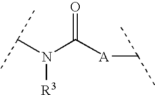
of formula I
denotes a group of partial formula II 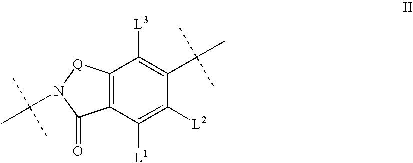
and
Q denotes a group, selected from the partial formulae IIIa to IIIg
—CR⁶R⁷— IIIa
—CR⁶═CR⁷— IIIb
—N═CR⁸— IIIc
—N═N— IIId
—CO—NR⁹— IIIe
—CR⁸═N— IIIf
—CO— IIIg
L¹, L², L³ independently of one another have one of the meanings given for R²⁰,
B denotes C₁₋₆-alkyl, C₁₋₆-alkenyl, C₁₋₆-alkynyl, C₃₋₇-cycloalkyl-C₁₋₃-alkyl-, C₃₋₇-cycloalkenyl-C₁₋₃-alkyl-, C₃₋₇-cycloalkyl-C₁₋₃-alkenyl- or C₃₋₇-cycloalkyl-C₁₋₃-alkynyl-, wherein one or more C atoms may be mono- or polysubstituted by halogen and/or monosubstituted by hydroxy or cyano and/or cyclic groups may be mono- or polysubstituted by R²⁰, or
has one of the meanings given for Cy, while the bond to the group W or optionally directly to the group A is formed via a C atom of the carbocyclic moiety or of the optionally fused-on phenyl or pyridine ring or via an N or C atom of the heterocyclic moiety,
while when k=0 the group B and the group A may be connected to one another via a common C atom forming a spirocyclic ring system or
via two common, adjacent atoms forming a fused, bicyclic ring system,
W denotes a single bond, —O—, a C₁₋₄-alkylene, C₂₋₄-alkenylene, C₂₋₄-alkynylene, C₁₋₄-alkylenoxy-, Oxy-C₁₋₄-alkylene-, C₁₋₃-alkylene-oxy-C₁₋₃-alkylene-, imino, N—(C₁₋₃-alkyl)-imino-, imino-C₁₋₄-alkylene-, N—(C₁₋₃-alkyl)-imino-C₁₋₄-alkylene-, C₁₋₄-alkylene-imino- or C₁₋₄-alkylene-N—(C₁₋₃-alkyl)-imino-group,
while one or two C atoms independently of one another may be substituted by a hydroxy, ω-hydroxy-C₁₋₃-alkyl, ω-(C₁₋₃-alkoxy)-C₁₋₃-alkyl- and/ or C₁₋₃-alkoxy group and/or with one or two identical or different C₁₋₆-alkyl groups, and/or
W with the definitions alkylene, oxyalkylene and alkyleneoxyalkylene may also be connected to B via a double bond,
k denotes 0 or 1,
Cy denotes a carbo- or heterocyclic group selected from one of the following meanings
a saturated 3- to 7-membered carbocyclic group,
an unsaturated 5- to 7-membered carbocyclic group,
a phenyl group,
a saturated 4- to 7-membered or unsaturated 5- to 7-membered heterocyclic group with an N, O or S atom as heteroatom,
a saturated or unsaturated 5- to 7-membered heterocyclic group with two or more N atoms or with one or two N atoms and an O or S atom as heteroatoms,
an aromatic heterocyclic 5- or 6-membered group with one or more identical or different heteroatoms selected from N, O and/or S,
while the above mentioned 4, 5, 6 or 7-membered groups may be connected via two common, adjacent C atoms, fused with a phenyl or pyridine ring, and
in the above mentioned 5-, 6- or 7-membered groups one or two non-adjacent —CH₂-groups may be replaced by a —CO—, —C(═CH₂)—, —(SO)— or —(SO₂)-group, and
the above mentioned saturated 6- or 7-membered groups may also be present as bridged ring systems with an imino, N—(C₁₋₄-alkyl)-imino, methylene, C₁₋₄-alkyl-methylene or di-(C₁₋₄-alkyl)-methylene bridge, and
the above mentioned cyclic groups may be mono- or polysubstituted at one or more C atoms with R²⁰, and in the case of a phenyl group also additionally monosubstituted by nitro, and/or substituted by R²¹ at one or more N atoms,
R⁴, R⁵ independently of one another have one of the meanings given for R¹⁶,
R⁶, R⁷,
R⁸, R⁹ independently of one another denote H, a C₁₋₆-alkyl, ω-C₁₋₃-alkoxy-C₁₋₃-alkyl- or ω-hydroxy-C₁₋₃-alkyl-group and R⁶, R⁷, R⁸ independently of one another also denote halogen,
R¹¹ denotes R¹⁵—O—, R¹⁵—O—CO—, R¹⁶R¹⁷N—, R¹⁸R¹⁹N—CO— or Cy-,
R¹² has one of the meanings given for R²⁰,
R¹³ has one of the meanings given for R¹⁷,
R¹⁴ denotes halogen, C₁₋₆-alkyl, R¹⁵—O—, R¹⁵—O—CO—, R¹⁶R¹⁷N—, R¹⁸R¹⁹N—CO—, R¹⁵—O—C₁₋₃-alkyl-, R¹⁵—O—CO—C₁₋₃-alkyl-, R¹⁶R¹⁷N—C₁₋₃-alkyl-, R¹⁸R¹⁹N—CO—C₁₋₃-alkyl- or Cy-C₁₋₃-alkyl-,
R¹⁵ denotes H, C₁₋₄-alkyl, C₃₋₇-cycloalkyl, C₃₋₇-cycloalkyl-C₁₋₃-alkyl-, phenyl, phenyl-C₁₋₃-alkyl- or pyridinyl,
R¹⁶ denotes H, C₁₋₆-alkyl, C₃₋₇-cycloalkyl, C₃₋₇-cycloalkyl-C₁₋₃-alkyl-, C₄₋₇-cycloalkenyl, C₄₋₇-cycloalkenyl-C₁₋₃-alky-l, ω-hydroxy-C₂₋₃-alkyl-, ω-(C₁₋₃-alkoxy)-C₂₋₃-alkyl-, amino-C₁₋₆-alkyl-, C₁₋₃-alkyl-amino-C₁₋₆-alkyl- or di-(C₁₋₃-alkyl)-amino-C₁₋₆-alkyl-,
R¹⁷ has one of the meanings given for R¹⁶ or denotes phenyl, phenyl-C₁₋₃-alkyl-, pyridinyl, dioxolan-2-yl, C₁₋₃-alkylcarbonyl, hydroxycarbonyl-C₁₋₃-alkyl-, C₁₋₄-alkoxycarbonyl, C₁₋₃-alkylcarbonylamino-C₂₋₃-alkyl-, C₁₋₃-alkylsulphonyl- or C₁₋₃-alkylsulphonylamino-C₂₋₃-alkyl-,
R¹⁸, R¹⁹ independently of one another denote H or C₁₋₆-alkyl,
R²⁰ denotes halogen, hydroxy, cyano, C₁₋₄-alkyl, C₃₋₇-cycloalkyl, hydroxy-C₁₋₃-alkyl, R²²—C₁₋₃-alkyl- or has one of the meanings given for R²²,
R²¹ denotes C₁₋₃-alkyl, ω-hydroxy-C₂₋₃-alkyl-, phenyl, phenyl-C₁₋₃-alkyl-, C₁₋₃-alkyl-carbonyl, carboxy, C₁₋₄-alkoxy-carbonyl, C₁₋₃-alkylsulphonyl, phenylcarbonyl or phenyl-C₁₋₃-alkyl-carbonyl,
R²² denotes pyridinyl, phenyl, phenyl-C₁₋₃-alkoxy-, C₁₋₃-alkoxy, C₁₋₃-alkylthio, carboxy, H—CO—, C₁₋₃-alkylcarbonyl, C₁₋₄-alkoxycarbonyl, aminocarbonyl, C₁₋₃-alkylaminocarbonyl, di-(C₁₋₃-alkyl)-aminocarbonyl, C₁₋₃-alkyl-sulphonyl-, C₁₋₃-alkyl-sulphinyl-, C₁₋₃-alkyl-sulphonylamino-, amino, C₁₋₃-alkylamino-, di-(C₁₋₃-alkyl)-amino-, phenyl-C₁₋₃-alkylamino- or N—(C₁₋₃-alkyl)-phenyl-C₁₋₃-alkylamino-, acetylamino-, propionylamino-, phenylcarbonyl, phenylcarbonylamino-, phenylcarbonylmethylamino-, hydroxyalkylaminocarbonyl, (4-morpholinyl)carbonyl, (1-pyrrolidinyl)-carbonyl, (1-piperidinyl)carbonyl, (hexahydro-1-azepinyl)carbonyl, (4-methyl-1-piperazinyl)carbonyl, methylenedioxy-, aminocarbonylamino- or alkylaminocarbonylamino-,
while in the groups and residues A, B, W, X, Y, Z, R¹ to R⁹ and R¹¹ to R²² in each case one or more C atoms may be mono- or polysubstituted by F and/or in each case one or two C atoms independently of one another may be monosubstituted by Cl or Br, and/or in each case one or more phenyl rings independently of one another additionally have one, two or three substituents selected from the group F, Cl, Br, I, C₁₋₄-alkyl, C₁₋₄-alkoxy, difluoromethyl, trifluoromethyl, hydroxy, amino, C₁₋₃-alkylamino-, di-(C₁₋₃-alkyl)-amino-, acetylamino-, aminocarbonyl, CN, difluoromethoxy, trifluoromethoxy, amino-C₁₋₃-alkyl-, C₁₋₃-alkylamino-C₁₋₃-alkyl- and di-(C₁₋₃-alkyl)-amino-C₁₋₃-alkyl- and/or may be monosubstituted by nitro, and
the H atom of any carboxy group present or an H atom bound to an N atom may be replaced in each case by a group which can be cleaved in vivo,
the tautomers, diastereomers, enantiomers, mixtures thereof and the salts thereof.

2. The carboxamide compounds according to claim 1, wherein group A has the meanings given for Cy in claim 1.

3. The carboxamide compounds according to claim 1, wherein
R³ denotes H, C₁₋₆alkyl, C₃₋₇-cycloalkyl, C₃₋₇-cycloalkyl-C₁₋₄-alkyl-, C₁₋₃-alkoxy-C₂₋₆-alkyl-, amino-C₂₋₆-alkyl-, C₁₋₃-alkyl-amino-C₂₋₆-alkyl- or di-(C₁₋₃-alkyl)-amino-C₂₋₆-alkyl-,
B has one of the meanings given for Cy, while the bond to the group W or optionally directly to the group A is formed via a C atom of the carbocyclic moiety or of the optionally fused-on phenyl or pyridine ring or via an N or C atom of the heterocyclic moiety,
while if k=0 the group B and the group A may be connected to one another via a common C atom forming a spirocyclic ring system or
via two common, adjacent atoms forming a fused, bicyclic ring system,
R¹⁵ denotes H, C₁₋₄-alkyl, C₃₋₇-cycloalkyl, C₃₋₇-cycloalkyl-C₁₋₃-alkyl-, phenyl or phenyl-C₁₋₃-alkyl-,
R¹⁷ has one of the meanings given for R¹⁶ or denotes phenyl, phenyl-C₁₋₃-alkyl-, dioxolan-2-yl, C₁₋₃-alkylcarbonyl, hydroxycarbonyl-C₁₋₃-alkyl, C₁₋₃-alkylcarbonylamino-C₂₋₃-alkyl-, C₁₋₃-alkylsulphonyl- or C₁₋₃-alkylsulphonylamino-C₂₋₃-alkyl-,
R²² denotes phenyl, phenyl-C₁₋₃-alkoxy-, C₁₋₃-alkoxy, C₁₋₃-alkylthio, carboxy, C₁₋₃-alkylcarbonyl, C₁₋₃-alkoxycarbonyl, aminocarbonyl, C₁₋₃-alkylaminocarbonyl, di-(C₁₋₃-alkyl)-aminocarbonyl, C₁₋₃-alkyl-sulphonyl, C₁₋₃-alkyl-sulphinyl, C₁₋₃-alkyl-sulphonylamino-, amino, C₁₋₃-alkylamino-, di-(C₁₋₃-alkyl)-amino-, phenyl-C₁₋₃—alkylamino- or N—(C₁₋₃-alkyl)-phenyl-C₁₋₃-alkylamino-, acetylamino-, propionylamino-, phenylcarbonyl, phenylcarbonylamino-, phenylcarbonylmethylamino-, hydroxyalkylaminocarbonyl, (4-morpholinyl)carbonyl, (1-pyrrolidinyl)carbonyl, (1-piperidinyl)carbonyl, (hexahydro-1-azepinyl)carbonyl, (4-methyl-1-piperazinyl)carbonyl, methylenedioxy, aminocarbonylamino- or alkylaminocarbonylamino-,
while in the groups A, B, W, X, Y, Z, R¹ to R⁹ and R¹¹ to R²² in each case one or more C atoms may be mono- or polysubstituted by F and/or in each case one or two C atoms independently of one another may be monosubstituted by Cl or Br, and
the groups A, W, X, Y, Z, R¹, R², R⁴ to R⁹, R¹¹ to R¹⁴, R¹⁶ and R¹⁸ to R²¹ and k have the meanings according to claim 1, and
the H atom of any carboxy group present or an H atom bound to an N atom in each case by a group which can be cleaved in vivo may be replaced,
the tautomers, diastereomers, enantiomers, mixtures thereof and the salts thereof.

4. Carboxamide compounds according to claim 3, wherein group A has the meanings given for Cy in claim 3.

5. The carboxamide compounds according to claim 1, wherein A and R³ are connected to one another such that
the group 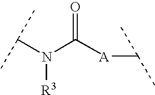
of formula I
denotes a group of partial formula II 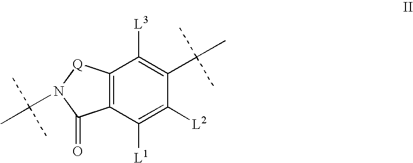
and
Q denotes a group, selected from the partial formulae IIIa to IIIg
—CR⁶R⁷— IIIa
—CR⁶═CR⁷— IIIb
—N═CR⁸— IIIc
—N═N— IIId
—CO—NR⁹— IIIe
—CR⁸═N— IIIf
—CO— IIIg
and
L¹, L², L³, R⁶, R⁷, R⁸ and R⁹ have the meanings given in claim 1.

6. The carboxamide compounds according to claim 1, wherein R¹, R² independently of one another denote H, C₁₋₆-alkyl, C₃₋₇-cycloalkyl, C₃₋₇-cycloalkyl-C₁₋₃-alkyl-, ω-hydroxy-C₂₋₃-alkyl, ω-(C₁₋₃-alkoxy)-C₂₋₃-alkyl-, C₁₋₄-alkoxy-carbonyl-C₁₋₃-alkyl, amino-C₂₋₄-alkyl-, C₁₋₃-alkyl-amino-C₂₋₄-alkyl- or di-(C₁₋₃-alkyl)-amino-C₂₋₄-alkyl-, phenyl or phenyl-C₁₋₃-alkyl-,
while in the above mentioned groups and residues one or more C atoms may be mono- or polysubstituted by F and/or one or two C atoms independently of one another may be monosubstituted by Cl or Br and
the phenyl group may be mono- or polysubstituted by the group R¹² defined in claim 1 and/or monosubstituted by nitro.

7. Carboxamide compounds according to claim 1, wherein R¹ and R² form an alkylene bridge according to claim 1 in such a way that R¹R²N— forms a group selected from azetidine, pyrrolidine, piperidine, azepan, 2,5-dihydro-1H-pyrrole, 1,2,3,6-tetrahydro-pyridine, 2,3,4,7-tetrahydro-1H-azepinyl, 2,3,6,7-tetrahydro-1H-azepine, morpholine, thiomorpholine, and piperazine, wherein the free imine function may be substituted with R¹³,
wherein one or more H atoms may be replaced by R¹⁴, and/or may be substituted in the manner specified in claim 1 with one or two identical or different carbo- or heterocyclic groups Cy,
where R¹³, R¹⁴ and Cy have the meanings given in claim 1.

8. The carboxamide compounds according to claim 1, wherein the group 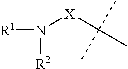
has a meaning according to one of the following partial formulae 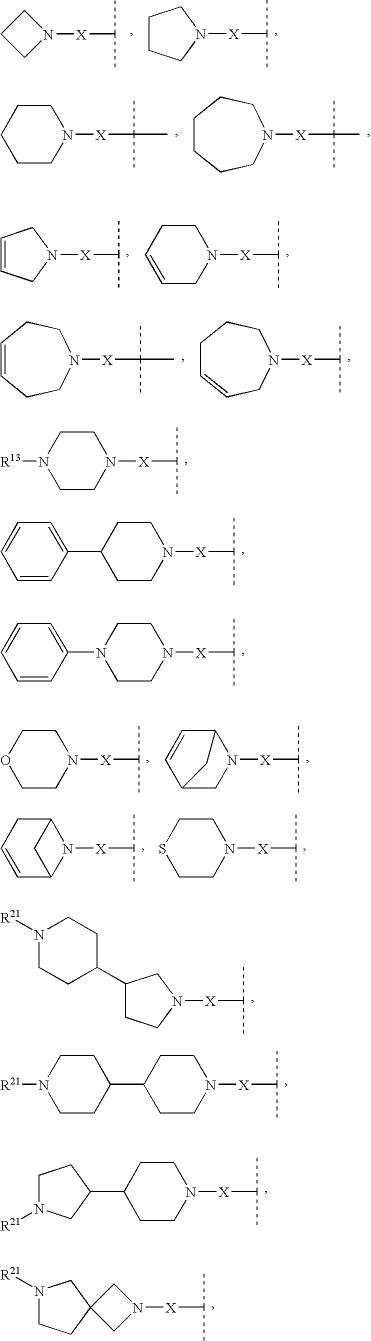 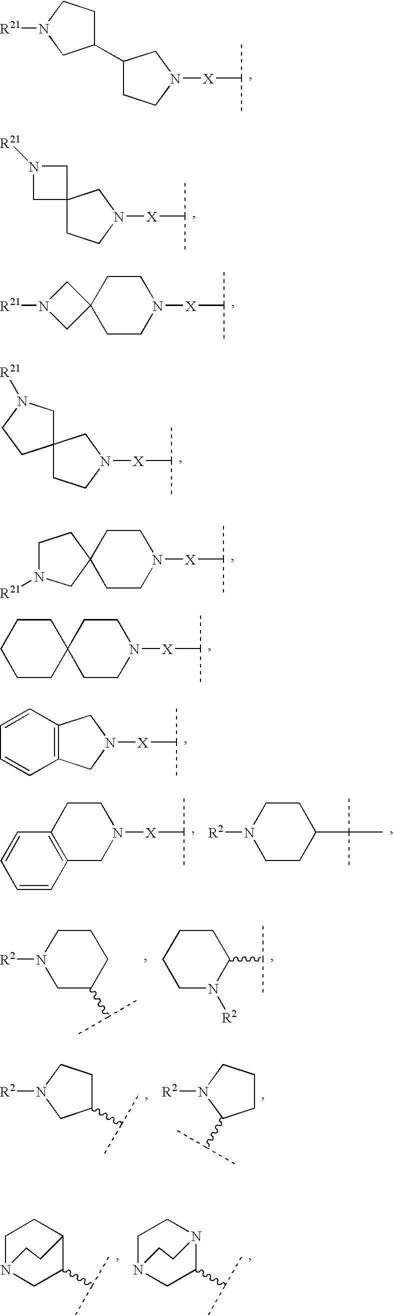
wherein one or more H atoms of the heterocycle formed by the group R¹R²N— may be replaced by R¹⁴ and the ring connected to the heterocycle formed by the group R¹R²N may be mono- or polysubstituted at one or more C atoms with R²⁰, and in the case of a phenyl ring may also additionally be monosubstituted by nitro and
wherein R¹³, R¹⁴, R²⁰, R²¹ and X have the meanings given in claim 1.

9. The carboxamide compounds according to claim 1, wherein X denotes a single bond or an unbranched bridge selected from C₁₋₆-alkylene, C₂₋₆-alkenylene, C₂₋₆-alkynylene, C₁₋₆-alkylenoxy, carbonyl, carbonyl-C₁₋₆-alkylene or C₁₋₆-alkylene-amino, wherein the amino group may be substituted with R⁴,
while one or two C atoms may be substituted as specified in claim 1 and/or the alkylene bridge may be connected to R¹ as specified in claim 1.

10. The carboxamide compounds according to claim 1, wherein X denotes a single bond, carbonyl or an alkylene bridge selected from methylene, 1,2-ethylene, 1,3-propylene and 1,4-butylene,
wherein one or two C atoms independently of one another may be substituted with a hydroxy, ω-hydroxy-C₁₋₃-alkyl, ω-(C₁₋₃-alkoxy)-C₁₋₃-alkyl-and/or C₁₋₃-alkoxy group and/or in each case may be substituted with one or two identical or different C₁₋₄-alkyl groups, and
in each case one or more C atoms may be mono- or polysubstituted by F and/or in each case one or two C atoms independently of one another may be monosubstituted by Cl or Br.

11. The carboxamide compounds according to claim 1, wherein Z denotes methylene, 1,2-ethylene, 1,3-propylene, 1,4-butylene, methyleneoxy, 1,2-ethyleneoxy, 1,3-propyleneoxy or 1,4-butyleneoxy,
wherein one or two C atoms independently of one another may be substituted with a hydroxy, ω-hydroxy-C₁₋₃-alkyl, ω-(C₁₋₃-alkoxy)-C₁₋₃-alkyl-and/or C₁₋₃-alkoxy group and/or in each case may be substituted with one or two identical or different C₁₋₄-alkyl groups, and
in each case one or more C atoms may be mono- or polysubstituted by F and/or in each case one or two C atoms independently of one another may be monosubstituted by Cl or Br and
R³ may be connected to Z so as to include the N atoms linked to R³ forming a heterocyclic group.

12. The carboxamide compounds according to claim 11, wherein Z is selected from the group of the bridges —CH₂—, —CH₂—CH₂—, —CH₂—CH(CH₃)—, —CH₂—C(CH₃)₂—, —CH(CH₃)—CH₂—, —C(CH₃)₂—CH₂— and —CH₂—O— or Z is linked to R³ in such a way that the group of partial formula 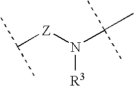
has a meaning selected from 1,3-pyrrolidinylene, 1,3-piperidinylene, 1,2,5,6-tetrahydropyridin-1,3-ylene and 3-hydroxy-1,3-piperidinylene.

13. The carboxamide compounds according to claim 1, wherein R³ is selected from among methyl, ethyl, n-propyl, iso-propyl, 2-hydroxyethyl, 3-hydroxy-n-propyl and 2-hydroxy-1-methyl-ethyl-, while in the above mentioned groups one, two or three H atoms may be replaced by F, or is selected from among H, amino-C₂₋₃-alkyl-, C₁₋₃-alkyl-amino-C₂₋₃-alkyl- and di-(C₁₋₃-alkyl)-amino-C₂₋₃-alkyl-.

14. The carboxamide compounds according to claim 1, wherein the group Y is selected from among the bivalent cyclic groups 1,2-cyclopropylene, 1,3-cyclobutylene, 1,3-cyclopentylene, 1,3-cyclopentenylene, 1,3- and 1,4-cyclohexylene, 1,3-phenylene, 1,4-phenylene, 1,3- and 1,4-cyclohexenylene, 1,4-cycloheptylene, 1,4-cycloheptenylene, 1,3-pyrrolidinylene, 1,3-pyrrolinylene, 1,3-pyrrolylene, 1,4-piperidinylene, 1,4-tetrahydropyridinylene, 1,4-dihydropyridinylene, 2,4- and 2,5-pyridinylene or 1,4-piperazinylene,
while the above mentioned 5-, 6- or 7-membered groups may be connected through two common, adjacent C atoms fused with a phenyl or pyridine ring,
the above mentioned cyclic groups may be mono- or polysubstituted at one or more C atoms with R²⁰, in the case of a phenyl group they may also additionally be monosubstituted by nitro, and/or may be substituted with R²¹ at one or more N atoms,
while R¹ may be connected to Y and/or R³ to Y as specified in claim 1, and
R¹, R³, R²⁰ and R²¹ have the meanings given in claim 1.

15. The carboxamide compounds according to claim 14, wherein R¹ is linked to Y in such a way that the group of partial formula 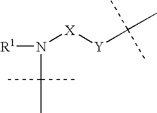
has a meaning selected from the following partial formulae

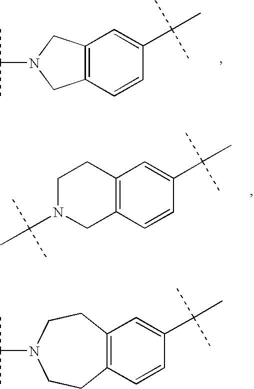

16. The carboxamide compounds according to claim 1, wherein group A is selected from among the bivalent cyclic groups 1,2-cyclopropylene, 1,3-cyclobutylene, 1,3-cyclopentylene, 1,3-cyclopentenylene, 1,3- and 1,4-cyclohexylene, 1,3- and 1,4-phenylene, 1,3- and 1,4-cyclohexenylene, 1,4-cycloheptylene, 1,4-cycloheptenylene, 1,3-pyrrolidinylene, 1,3-pyrrolinylene, 1,3-pyrrolylene, 1,4-piperidinylene, 1,4-tetrahydropyridinylene, 1,4-dihydropyridinylene, 2,4- and 2,5-pyridinylene, 1,4-piperazinylene, 7-aza-bicyclo[2.2.1]heptan-2,7-diyl and 8-aza-bicyclo[3.2.1]octan-3,8-diyl,
while the above mentioned 5-, 6- or 7-membered groups may be linked by two common, adjacent C atoms fused with a phenyl or pyridine ring, and
the above mentioned cyclic groups may be mono- or polysubstituted at one or more C atoms with R²⁰, in the case of a phenyl ring they may also additionally be monosubstituted by nitro, and/or they may be substituted at one or more N atoms with R²¹, and
R²⁰, R²¹ and Y have the meanings given in claim 1.

17. The carboxamide compounds according to claim 1, wherein group B is selected from among cyclopropyl, cyclobutyl, cyclopentyl, cyclopentenyl, cyclohexyl, cyclohexanonyl, cyclohexenyl, phenyl, cycloheptyl, cycloheptenyl, aziridinyl, azetidinyl, pyrrolidinyl, pyrrolinyl, pyrrolyl, piperidinyl, tetrahydropyridinyl, dihydropyridinyl, pyridinyl, azepanyl, piperazinyl, 1H-pyrazolyl, imidazolyl, triazolyl, tetrazolyl, morpholinyl, thiomorpholinyl, indolyl, isoindolyl, quinolinyl, benzoimidazolyl, isoquinolinyl, furanyl and thienyl,
while the bond to the group W or optionally directly to the group A is made via a C atom of the carbocyclic moiety or of the optionally fused-on phenyl or pyridine ring or via an N or C atom of the heterocyclic moiety,
or B together with the group W connected by a double bond is selected from the group cyclopentylidene-methyl, cyclohexylidene-methyl and cyclohexanone-4-ylidene-methyl, and
the above mentioned cyclic groups may be mono- or polysubstituted at one or more C atoms with R²⁰, in the case of a phenyl group may also additionally be monosubstituted by nitro, and/or may be substituted with R²¹ at one or more N atoms, and
R²⁰ and R²¹ have the meanings given in claim 1.

18. Carboxamide compounds according to claim 1, wherein group B is selected from among C₁₋₆-alkyl, C₁₋₆-alkenyl, C₁₋₆-alkynyl, C₃₋₇-cycloalkyl-C₁₋₃-alkyl-, C₃₋₇-cycloalkenyl-C₁₋₃-alkyl-, C₃₋₇-cycloalkyl-C₁₋₃-alkenyl- or C₃₋₇-cycloalkyl-C₁₋₃-alkynyl-, wherein one or more C atoms may be mono- or polysubstituted by halogen and/or monosubstituted by hydroxy or cyano and/or cyclic groups may be mono- or polysubstituted by R²⁰, and
W denotes a single bond, —O—, a C₁₋₄-alkylene, C₂₋₄-alkenylene, C₂₋₄-alkynylene, C₁₋₄-alkyleneoxy, oxy-C₁₋₄-alkylene-, C₁₋₃-alkylene-oxy-C₁₋₃-alkylene-, imino, N—(C₁₋₃-alkyl)-imino-, imino-C₁₋₄-alkylene-, N—(C₁₋₃-alkyl)-imino-C₁₋₄-alkylene-, C₁₋₄-alkylene-imino- or C₁₋₄-alkylene-N—(C₁₋₃-alkyl)-imino-group, while one or two C atoms independently of one another may be substituted with a hydroxy, ω-hydroxy-C₁₋₃-alkyl, ω-(C₁₋₃-alkoxy)-C₁₋₃-alkyl- and/ or C₁₋₃-alkoxy group and/or with one or two identical or different C₁₋₄-alkyl groups, and
k denotes 0 or 1 and
R²⁰ has the meanings given in claim 1.

19. Carboxamide compounds according to claim 1, wherein k=0 and the group A is linked to the group B through a common C atom forming a spirocyclic ring system,
while the group A represents a saturated 5- to 7-membered carbo- or heterocyclic group and the group B denotes a saturated 4- to 7-membered carbo- or heterocyclic group, and
the heterocyclic groups in each case contain an N, O or S atom, and a phenyl or pyridine ring may be fused to a 5- to 7-membered group B through two adjacent C atoms, and
the above mentioned cyclic groups may be mono- or polysubstituted at one or more C atoms with R²⁰, in the case of a fused-on phenyl ring they may additionally be monosubstituted by nitro, and/or may be substituted with R²¹ at one or more N atoms, while R²⁰ and R²¹ have the meanings given in claim 1.

20. The carboxamide compounds according to claim 1, wherein k=0 and the group B is linked to the group A through two common, adjacent atoms forming a fused, bicyclic saturated, unsaturated or aromatic, 8- to 12-membered carbo- or heterocyclic ring system,
while the heterocyclic ring system has one or more identical or different heteroatoms selected from N, O and/or S, and
the bicyclic ring system may be mono- or polysubstituted at one or more C atoms with R²⁰, in the case of a fused-on phenyl ring it may also additionally be monosubstituted by nitro, and/or may be substituted with R²¹ at one or more N atoms, while R²⁰ and R²¹ have the meanings given in claim 1.

21. The carboxamide compounds according to claim 1, wherein W is a single bond, —CH₂— or —CH═.

22. Carboxamide compounds according to claim 1, wherein
Y, A independently of one another are selected from among the bivalent cyclic groups 1,4-phenylene, 1,4-cyclohexylene, 1,4-cyclohexenylene, 1,4-piperidinylene, 1,2,3,6-tetrahydro-pyridin-1,4-ylene, 2,5-pyridinylene and 1,4-piperazinylene, while A may also be connected to R³ according to claim 3, and the above mentioned cyclic groups may be mono- or polysubstituted by R²⁰ at one or more C atoms, in the case of a phenyl group they may also additionally be monosubstituted by nitro, and/or may be substituted by R²¹ at one or more N atoms,
B denotes phenyl or cyclohexyl, while the above mentioned groups may be mono- or polysubstituted by R²⁰ and/or the phenyl ring may additionally be monosubstituted by nitro, while R²⁰ has the meanings given in claim 1, and
W is a single bond, —CH₂— or —CH═, and
Z denotes —CH₂—CH₂—, —CH₂—CH(CH₃)—, —CH₂—C(CH₃)₂—, —CH(CH₃)—CH₂—, —C(CH₃)₂—CH₂— or —CH₂—O— or
is linked to R³ in such a way that the group of partial formula 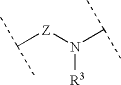
of formula I has a meaning selected from 1,3-pyrrolidinylene and 1,3-piperidinylene and
R³, R²⁰ and R²¹ have the meanings given in claim 1.

23. Carboxamide compounds according to claim 1 from among formulae I.1 to I.14 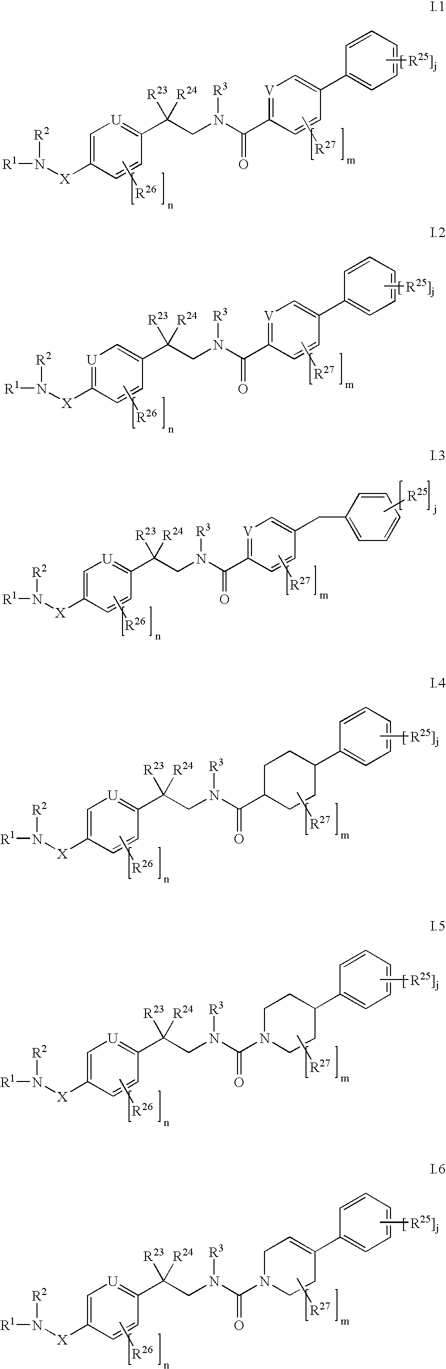 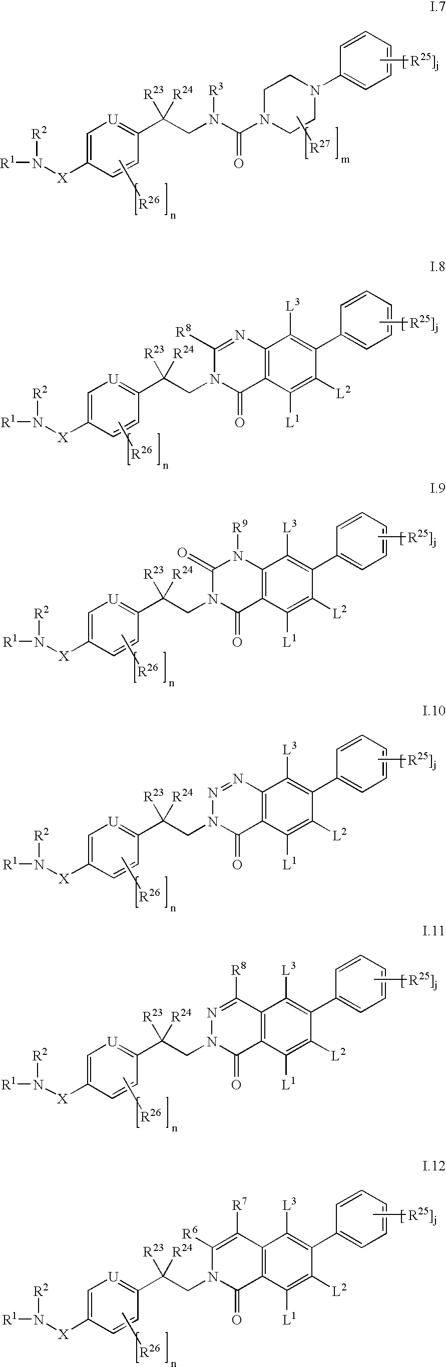 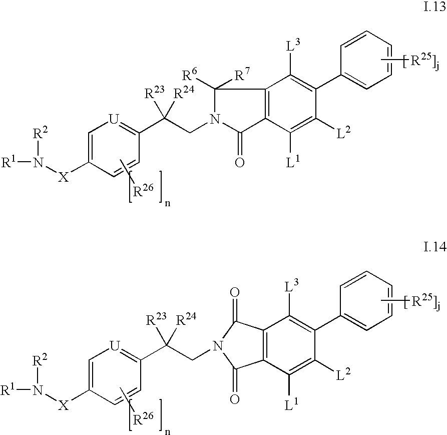
wherein
U, V independently of one another denote C or N,
R²³, R²⁴ independently of one another denote H, F, methyl, trifluoromethyl, ethyl, iso-propyl or n-propyl,
while in formulae I.1 to I.6 R²⁴ may be connected to R³ in such a way that the group of partial formula 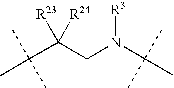
has a meaning selected from 1,3-pyrrolidinylene and 1,3-piperidinylene, and
R²⁵,
R²⁶, R²⁷ independently of one another have one of the meanings given for R²⁰ according to claim 1 or in the case of a phenyl group also simply denote nitro, while repeatedly occurring groups R²⁵, R²⁶, R²⁷ may have identical or different meanings, and
j is 0, 1, 2, 3 or 4 and
m, n independently of one another denote 0, 1 or 2 and
L¹, L², L³, R¹, R², R³, R⁶, R⁷, R⁸, R⁹, R²⁰ and X have the meanings given in claim 1.

24. Carboxamide compounds according to claim 1, characterised by the formula I.15 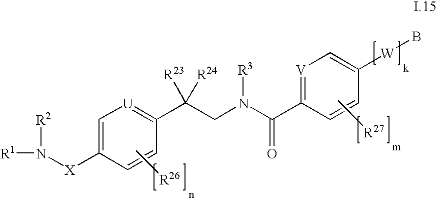
wherein
B is selected from among C₁₋₆alkyl, C₁₋₆-alkenyl, C₁₋₆-alkynyl, C₃₋₇-cycloalkyl-C₁₋₃-alkyl-, C₃₋₇-cycloalkenyl-C₁₋₃-alkyl-, C₃₋₇-cycloalkyl-C₁₋₃-alkenyl- or C₃₋₇-cycloalkyl-C₁₋₃-alkynyl-, wherein one or more C atoms may be mono- or polysubstituted by halogen and/or monosubstituted by hydroxy or cyano and/or cyclic groups may be mono- or polysubstituted by R²⁰, and
W denotes a single bond, —O—, a C₁₋₄-alkylene, C₂₋₄-alkenylene, C₂₋₄-alkynylene, C₁₋₄-alkylenoxy-, oxy-C₁₋₄-alkylene-, C₁₋₃-alkylene-oxy-C₁₋₃-alkylene-, imino, N—(C₁₋₃-alkyl)-imino-, imino-C₁₋₄-alkylene-, N—(C₁₋₃-alkyl)-imino-C₁₋₄-alkylene-, C₁₋₄-alkylene-imino- or C₁₋₄-alkylene-N—(C₁₋₃-alkyl)-imino group, while one or two C atoms independently of one another may be substituted by a hydroxy, ω-hydroxy-C₁₋₃-alkyl, ω-(C₁₋₃-alkoxy)-C₁₋₃-alkyl and/or C₁₋₃-alkoxy group and/or by one or two identical or different C₁₋₄-alkyl groups, and
k denotes 0 or 1 and
U, V, R²³, R²⁴, R²⁶, R²⁷, m, n, L¹, L², L³, R¹, R², R³, R⁶, R⁷, R⁸, R⁹, R²⁰ and X have the meanings given in claim 23.

25. Carboxamide compounds according to claim 24, wherein U and V in each case represent a C atom.

26. Carboxamide compounds according to claim 1, wherein X is —CH₂—, —CH(CH₃)— or —C(CH₃)_{2—.}

27. Carboxamide compounds according to claim 26, wherein
R²⁵,
R²⁶, R²⁷ independently of one another denote F, Cl, Br, I, OH, cyano, methyl, difluoromethyl, trifluoromethyl, ethyl, n-propyl, iso-propyl, methoxy, difluoromethoxy, trifluoromethoxy, ethoxy, n-propoxy or iso-propoxy, in the case of a substitution of a phenyl group they may also denote nitro, while repeatedly occurring groups R²⁵, R²⁶, R²⁷ may have identical or different meanings, and
j is 0, 1 or 2, and
m, n independently of one another are 0 or 1.

28. Carboxamide compounds according to claim 1, wherein R⁶, R⁷, R⁸ and/or R⁹ independently of one another denote H, methyl, trifluoromethyl, ethyl, iso-propyl or n-propyl, if R⁶, R⁷ also represent F.

29. Carboxamide compounds according to claim 1 selected from among the formulae:
(1) 7-(4-chloro-phenyl)-3-[2-(4-pyrrolidin-1-ylmethyl-phenyl)-ethyl]-3H-quinazolin-4-one
(2) 3-[2-(4-pyrrolidin-1-ylmethyl-phenyl)-ethyl]-7-p-tolyl-3H-quinazolin-4-one
(3) 3-[2-(4-pyrrolidin-1-ylmethyl-phenyl)-ethyl]-7-(4-trifluoromethyl-phenyl)-3H-quinazolin-4-one
(4) 7-(4-methoxy-phenyl)-3-[2-(4-pyrrolidin-1-ylmethyl-phenyl)-ethyl]-3H-quinazolin-4-one
(5) 7-(3.4-dichloro-phenyl)-3-[2-(4-pyrrolidin-1-ylmethyl-phenyl)-ethyl]-3H-quinazolin-4-one
(6) 7-(4-fluoro-phenyl)-3-[2-(4-pyrrolidin-1-ylmethyl-phenyl)-ethyl]-3H-quinazolin-4-one
(7) 7-(4-ethyl-phenyl)-3-[2-(4-pyrrolidin-1-ylmethyl-phenyl)-ethyl]-3H-quinazolin-4-one
(8) 2-methyl-3-[2-(4-pyrrolidin-1-ylmethyl-phenyl)-ethyl]-7-(4-trifluoromethyl-phenyl)-3H-quinazolin-4-one
(9) 2-methyl-3-[2-(4-pyrrolidin-1-ylmethyl-phenyl)-ethyl]-7-p-tolyl-3H-quinazolin-4-one
(10) 7-(4-chloro-phenyl)-2-methyl-3-[2-(4-pyrrolidin-1-ylmethyl-phenyl)-ethyl]-3H-quinazolin-4-one
(11) 7-(4-chloro-phenyl)-3-[2-(4-pyrrolidin-1-ylmethyl-phenyl)-ethyl]-1H-quinazolin-2,4-dione
(12) 7-(4-chloro-phenyl)-3-{2-[4-((S)-2-methoxymethyl-pyrrolidin-1-ylmethyl)-phenyl]-ethyl}-3H-quinazolin-4-one
(13) 7-(4-chloro-phenyl)-3-[2-(4-dimethylaminomethyl-phenyl)-ethyl]-3H-quinazolin-4-one
(14) 7-(4-chloro-phenyl)-3-[2-(4-piperidin-1-ylmethyl-phenyl)-ethyl]-3H-quinazolin-4-one
(15) 7-(4-chloro-phenyl)-3-[2-(4-morpholin-4-ylmethyl-phenyl)-ethyl]-3H-quinazolin-4-one
(16) 7-(4-chloro-phenyl)-3-[2-(4-pyrrolidin-1-ylmethyl-phenyl)-ethyl]-3H-benzo[d][1,2,3]triazin-4-one
(17) 5-(4-fluoro-phenyl)-2-[2-(4-pyrrolidin-1-ylmethyl-phenyl)-ethyl]-isoindol-1,3-dione
(18) 4′-chloro-biphenyl-4-carboxylic acid [2-(4-pyrrolidin-1-ylmethyl-phenyl)-ethyl]-amide
(19) 4′-chloro-biphenyl-4-carboxylic acid-[2-(4-diethylaminomethyl-phenyl)-ethyl]-amide
(20) 4′-chloro-biphenyl-4-carboxylic acid-[2-(4-piperidin-1-ylmethyl-phenyl)-ethyl]-amide
(21) 4′-methoxy-biphenyl-4-carboxylic acid-[2-(4-diethylaminomethyl-phenyl)-ethyl]-amide
(22) 4′-chloro-biphenyl-4-carboxylic acid-[2-(4-diethylaminomethyl-phenyl)-ethyl]-methyl-amide
(23) 4-(4-chloro-phenyl)-cyclohexanecarboxylic acid-[2-(4-pyrrolidin-1-ylmethyl-phenyl)-ethyl]-amide
(24) 4-methylphenyl-piperidine-1-carboxylic acid-[2-(4-pyrrolidin-1-ylmethyl-phenyl)-ethyl]-amide
(25) 4-(4-chloro-phenyl)-3.6-dihydro-2H-pyridine-1-carboxylic acid-[2-(4-pyrrolidin-1-ylmethyl-phenyl)-ethyl]-amide
(26) 4-(4-chloro-phenyl)-piperidine-1-carboxylic acid-[2-(4-pyrrolidin-1-ylmethyl-phenyl)-ethyl]-amide
(27) 4′-chloro-biphenyl-4-carboxylic acid-[2-(4-pyrrolidin-1-ylmethyl-phenyl)-propyl]-amide
(28) 4′-chloro-biphenyl-4-carboxylic acid-(4-pyrrolidin-1-ylmethyl-benzyloxy)-amide
(29) 4-cyclohexyl-N-[2-(4-pyrrolidin-1-ylmethyl-phenyl)-ethyl]-benzamide
(30) 4′-chloro-biphenyl-4-carboxylic acid-[2-(3-methoxy-4-pyrrolidin-1-ylmethyl-phenyl)-ethyl]-amide
(31) 7-(4-chloro-phenyl)-3-{2-[6-(4-methyl-piperazin-1-yl)-pyridin-3-yl]-ethyl}-3H-quinazolin-4-one
(32) 4′-chloro-biphenyl-4-carboxylic acid-{2-[6-(4-methyl-piperazin-1-yl)-pyridin-3-yl]-ethyl}-amide
(33) 7-(3-methoxy-phenyl)-3-[2-(4-pyrrolidin-1-ylmethyl-phenyl)-ethyl]-3H-quinazolin-4-one
(34) 4-(4-oxo-cyclohexyl)-N-[2-(4-pyrrolidin-1-ylmethyl-phenyl)-ethyl]-benzamide
(35) 4-cyclohexyl-1-cylohexylcarboxylic acid-[2-(4-pyrrolidin-1-ylmethyl-phenyl)-ethyl]-amide
(36) 4-benzyl-piperidine-1-carboxylic acid-[2-(4-pyrrolidin-1-ylmethyl-phenyl)-ethyl]-amide
(37) 4-cyclohexyl-piperidine-1-carboxylic acid-[2-(4-pyrrolidin-1-ylmethyl-phenyl)-ethyl]-amide
(38) 4-(4-chloro-phenyl)-piperazine-1-carboxylic acid-[2-(4-pyrrolidin-1-ylmethyl-phenyl)-ethyl]-amide
(39) 4-(4-fluoro-phenyl)-piperidine-1-carboxylic acid-[2-(4-pyrrolidin-1-ylmethyl-phenyl)-ethyl]-amide
(40) 4-(4-methoxy-phenyl)-piperazine-1-carboxylic acid-[2-(4-pyrrolidin-1-ylmethyl-phenyl)-ethyl]-amide
(41) 4-phenyl-piperidine-1-carboxylic acid-[2-(4-pyrrolidin-1-ylmethyl-phenyl)-ethyl]-amide
(42) (4′-chloro-biphenyl-4-yl)-[3-(4-pyrrolidin-1-ylmethyl-phenyl)-piperidin-1-yl]-methanone
(43) 4′-chloro-biphenyl-4-carboxylic acid-[2-methyl-2-(4-pyrrolidin-1-ylmethyl-phenyl)-propyl]-amide
(44) 4′-chloro-biphenyl-4-carboxylic acid-[2-(4-pyrrolidin-1-ylmethyl-cyclohexyl)-ethyl]-amide
(45) 4-benzyl-N-[2-(4-pyrrolidin-1-ylmethyl-phenyl)-ethyl]-benzamide
(46) 4-(4-oxo-cyclohexylidenemethyl)-N-[2-(4-pyrrolidin-1-ylmethyl-phenyl)-ethyl]-benzamide
(47) 4′-chloro-biphenyl-4-carboxylic acid-[2-(2-fluoro-4-pyrrolidin-1-ylmethyl-phenyl)-ethyl]-amide
(48) 5-(4-chloro-phenyl)-2-[2-(4-pyrrolidin-1-ylmethyl-phenyl)-ethyl]-2.3-dihydro-isoindol-1-one
(49) 4-piperidin-1-yl-N-[2-(4-pyrrolidin-1-ylmethyl-phenyl)-ethyl]-benzamide
(50) 7-(4-chloro-phenyl)-3-{2-[4-(4-hydroxy-4-phenyl-piperidin-1-ylmethyl)-phenyl]-ethyl}-3H-benzo[d][1,2,3]triazin-4-one
(51) 7-(4-chloro-phenyl)-3-{2-[4-(3-aza-spiro[5.5]undec-3-ylmethyl)-phenyl]-ethyl}-3H-quinazolin-4-one
(52) 7-(4-chloro-phenyl)-3-{2-[4-(3-aza-spiro[5.5]undec-3-ylmethyl)-phenyl]-ethyl}-3H-benzo[d][1,2,3]triazin-4-one
(53) 7-(4-chloro-phenyl)-3-{2-[4-(4-hydroxy-4-phenyl-piperidin-1-ylmethyl)-phenyl]-ethyl}-3H-quinazolin-4-one
(54) 7-(4-chloro-phenyl)-3-(2-{4-[4-(pyridin-2-yloxy)-piperidin-1-ylmethyl]-phenyl}-ethyl)-3H-quinazolin-4-one
(55) 6-(4-chloro-phenyl)-2-[2-(4-pyrrolidin-1-ylmethyl-phenyl)-ethyl]-2H-isoquinolin-1-one
(56) 4′-chloro-biphenyl-4-carboxylic acid [2-(3-bromo-4-pyrrolidin-1-ylmethyl-phenyl)-ethyl]-amide
(57) 4′-chloro-biphenyl-4-carboxylic acid [2-(3-methyl-4-pyrrolidin-1-ylmethyl-phenyl)-ethyl]-amide
(58) 4′-chloro-biphenyl-4-carboxylic acid {2-[4-(1-ethyl-piperidin-2-yl)-phenyl]-ethyl}-amide
(59) 4′-chloro-biphenyl-4-carboxylic acid{2-[4-(4-acetyl-piperazin-1-ylmethyl)-phenyl]-ethyl}-amide
(60) 4′-chloro-biphenyl-4-carboxylic acid{2-[4-(2-aza-bicyclo[2.2.1]hept-5-en-2-ylmethyl)-phenyl]-ethyl}-amide
(61) 4′-chloro-biphenyl-4-carboxylic acid {2-[4-(1,3-dihydro-isoindol-2-ylmethyl)-phenyl]-ethyl}-amide
(62) 4′-chloro-biphenyl-4-carboxylic acid (2-{4-[(diisopropylamino)-methyl]-phenyl}-ethyl)-amide
(63) 4′-chloro-biphenyl-4-carboxylic acid {2-[3-bromo-4-(2,5-dihydro-pyrrol-1-ylmethyl)-phenyl]-ethyl}-amide
(64) 4′-chloro-biphenyl-4-carboxylic acid{2-[4-(2-dimethylaminomethyl-pyrrolidin-1-ylmethyl)-phenyl]-ethyl}-amide
(65) 4′-chloro-biphenyl-4-carboxylic acid{2-[4-(3-dimethylamino-pyrrolidin-1-ylmethyl)-phenyl]-ethyl}-amide
(66) 4′-chloro-biphenyl-4-carboxylic acid [2-(2-bromo-4-pyrrolidin-1-ylmethyl-phenyl)-ethyl]-amide
(67) 4-pent-1-ynyl-N-[2-(4-pyrrolidin-1-ylmethyl-phenyl)-ethyl]-benzamide
(68) 4′-chloro-biphenyl-4-carboxylic acid [2-(6-pyrrolidin-1-ylmethyl-pyridin-3-yl)-ethyl]-amide
(69) 4′-chloro-biphenyl-4-carboxylic acid [2-(1-pyrrolidin-1-yl-indan-5-yl)-ethyl]-amide
(70) 4′-chloro-biphenyl-4-carboxylic acid [2-(2-nitro-4-pyrrolidin-1-ylmethyl-phenyl)-ethyl]-amide
(71) 2′,4′-dichloro-biphenyl-4-carboxylic acid [2-(4-pyrrolidin-1-ylmethyl-phenyl)-ethyl]-amide
(72) 4′-chloro-biphenyl-4-carboxylic acid{2-[4-(3-amino-pyrrolidin-1-ylmethyl)-phenyl]-ethyl}-amide
(73) 4′-chloro-biphenyl-4-carboxylic acid{2-[4-(2-aminomethyl-pyrrolidin-1-ylmethyl)-phenyl]-ethyl}-amide
(74) 4′-chloro-biphenyl-4-carboxylic acid{2-[4-(2-methyl-2,6-diaza-spiro[3.4]oct-6-ylmethyl)-phenyl]-ethyl}-amide
(75) 4′-chloro-biphenyl-4-carboxylic acid [2-(5-pyrrolidin-1-ylmethyl-pyridin-2-yl)-ethyl]-amide
(76) 4′-chloro-biphenyl-4-carboxylic acid [2-(3-ethyl-4-pyrrolidin-1-ylmethyl-phenyl)-ethyl]-amide
(77) 4′-bromo-biphenyl-4-carboxylic acid {2-[4-(2,5-dihydro-pyrrol-1-ylmethyl)-phenyl]-ethyl}-amide
(78) 4-(5-chloro-thiophen-2-yl)-N-[2-(4-pyrrolidin-1-ylmethyl-phenyl)-ethyl]-benzamide
(79) 4′-chloro-biphenyl-4-carboxylic acid [2-(2-methyl-4-pyrrolidin-1-ylmethyl-phenyl)-ethyl]-amide
(80) 4′-bromo-3-fluoro-biphenyl-4-carboxylic acid {2-[3-bromo-4-(2,5-dihydro-pyrrol-1-ylmethyl)-phenyl]-ethyl}-amide
(81) 4′-chloro-2-fluoro-biphenyl-4-carboxylic acid [2-(4-pyrrolidin-1-ylmethyl-phenyl)-ethyl]-amide
(82) 4′-ethyl-biphenyl-4-carboxylic acid [2-(4-pyrrolidin-1-ylmethyl-phenyl)-ethyl]-amide
(83) tert.butyl [1-(4-{2-[(4′-chloro-biphenyl-4-carbonyl)-amino]-ethyl}-benzyl)-pyrrolidin-2-ylmethyl]-carbaminate
(84) 4′-chloro-biphenyl-4-carboxylic acid {2-[4-(2-methyl-piperidin-1-ylmethyl)-phenyl]-ethyl}-amide
(85) 4′-chloro-biphenyl-4-carboxylic acid {2-[4-(2-methyl-pyrrolidin-1-ylmethyl)-phenyl]-ethyl}-amide
(86) 4′-chloro-biphenyl-4-carboxylic acid (2-{4-[(cyclopropylmethyl-amino)-methyl]-phenyl}-ethyl)-amide
(87) 4′-chloro-biphenyl-4-carboxylic acid {2-[4-(3.4-dihydro-1H-isoquinolin-2-ylmethyl)-phenyl]-ethyl}-amide
(88) 4′-chloro-biphenyl-4-carboxylic acid [2-(4-{[(2-hydroxy-ethyl)-methyl-amino]-methyl}-phenyl)-ethyl]-amide
(89) tert.butyl [1-(4-{2-[(4′-chloro-biphenyl-4-carbonyl)-amino]-ethyl}-benzyl)-pyrrolidin-3-yl]-carbaminate
(90) 4′-chloro-biphenyl-4-carboxylic acid {2-[4-(2,6-dimethyl-piperidin-1-ylmethyl)-phenyl]-ethyl}-amide
(91) 4′-chloro-biphenyl-4-carboxylic acid [2-(4-azetidin-1-ylmethyl-phenyl)-ethyl]-amide
(92) 3,4′-dichloro-biphenyl-4-carboxylic acid [2-(4-pyrrolidin-1-ylmethyl-phenyl)-ethyl]-amide
(93) 4′-fluoro-biphenyl-4-carboxylic acid [2-(4-pyrrolidin-1-ylmethyl-phenyl)-ethyl]-amide
(94) 4′-chloro-3-fluoro-biphenyl-4-carboxylic acid [2-(4-pyrrolidin-1-ylmethyl-phenyl)-ethyl]-amide
(95) 2′-fluoro-4′-chloro-biphenyl-4-carboxylic acid [2-(4-pyrrolidin-1-ylmethyl-phenyl)-ethyl]-amide
(96) 5-(4-chloro-phenyl)-pyridine-2-carboxylic acid [2-(4-pyrrolidin-1-ylmethyl-phenyl)-ethyl]-amide
(97) 4′-chloro-biphenyl-4-carboxylic acid {2-[4-(2,5-dihydro-pyrrol-1-ylmethyl)-phenyl]-ethyl}-amide
(98) 4′-bromo-biphenyl-4-carboxylic acid [2-(4-pyrrolidin-1-ylmethyl-phenyl)-ethyl]-amide
(99) 4′-chloro-biphenyl-4-carboxylic acid {2-[4-(1-pyrrolidin-1-yl-ethyl)-phenyl]-ethyl}-amide.

30. Carboxamide compounds according to claim 29 selected from among the formulae (1), (2), (3), (4), (5), (6), (7), (8), (9), (10), (11), (12), (13), (14), (15), (16), (17), (18), (19), (20), (21), (22), (23), (24), (25), (25), (26), (27), (28), (29), (30), (47) and (50) to (99).

31. Process for preparing carboxamide compounds of formula I 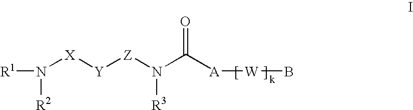
wherein A, B, W, X, Y, Z, R¹, R², R³ and k have one of the meanings specified in claim 1 and wherein
if A denotes a group R³ which is not connected to the group A:
a) in the event that A denotes a nitrogen-heterocyclic group connected to the carboxamide group via a nitrogen atom which may also have in addition to the nitrogen atom one or more heteroatoms selected from N, O and S, at least one amine compound of formula 1-1 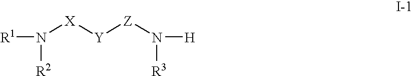
wherein R¹, R², R³, X, Y and Z have the meanings given hereinbefore,
reacting with CDT (1,1′-carbonyldi-(1,2,4-triazole)), at least one secondary amine compound of formula I-2
A[W]ₖB I-2
wherein A, B, W and k have the meanings given hereinbefore and the group A has the sec. amine function,
in a solvent or mixture of solvents in the presence of at least one base, and
b) for the other cases at least one carboxylic acid compound of formula I-3 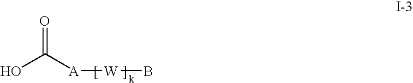
wherein A, B, W and k have the meanings given hereinbefore,
reacting with TBTU (2-(1H-benzotriazol-1-yl)-1,1,3,3-tetramethyluronium-tetrafluoroborate) and at least one amine compound of formula I-1 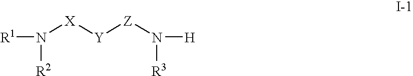
wherein R¹, R², R³, X, Y and Z have the meanings given hereinbefore,
in a solvent or mixture of solvents in the presence of at least one base, and
if B is a croup R³ connected to the group A:
a) in the event of a group Q having the meaning —CR⁶R⁷— (IIIa), while R⁶ and R⁷ are as hereinbefore defined, an amine compound of formula Ia.1 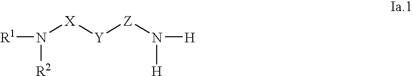
wherein R¹, R², X, Y and Z have the meanings specified,
reacting with an o-bromomethyl-benzoic acid ester derivative of formula Ia.2 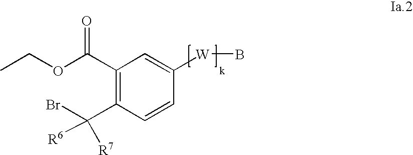
wherein R⁶, R⁷, W, B and k have the meanings specified,
b) in the event of a group Q having the meaning —CR⁶═CR⁷— (IIIb), wherein R⁶ and R⁷ are as hereinbefore defined, an isoquinolinone derivative of formula Ib.2 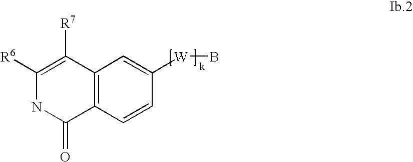
wherein R⁶, R⁷, W, B and k have the meanings specified,
reacting with an electrophilic compound of formula Ib.3 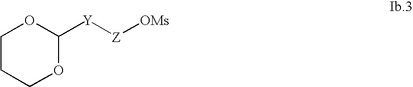
wherein Y and Z have the meanings specified and OMs denotes a suitable leaving group, preferably mesylate, to obtain an isoquinoline derivative of formula Ib.4 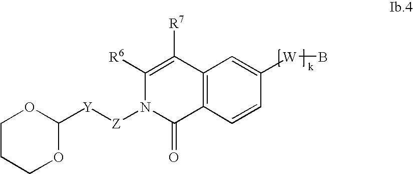
wherein R⁶, R⁷, W, B, Y, Z and k have the meanings specified, and the isoquinoline derivative of formula Ib.4 is further derivatised by known methods to form the compound of formula I,
c) in the event of a group Q having the meaning —N═CR⁸— (IIIc), wherein R⁸ is as hereinbefore defined, a phthalazinone derivative of formula Ic.4 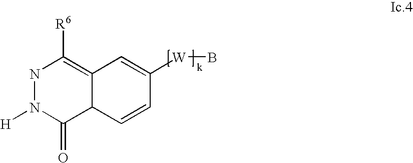
wherein R⁸, W, B and k have the meanings specified,
reacting with an electrophilic compound of formula Ic.5 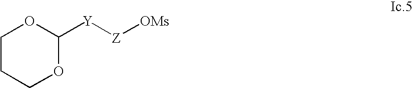
wherein Y and Z have the meanings specified and OMs denotes a leaving group, preferably mesylate, to form a phthalazinone derivative of formula Ic.6 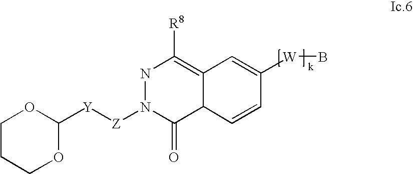
wherein R⁸, W, B, Y, Z and k have the meanings specified, and the phthalazinone derivative of formula Ic.6 thus obtained is further derivatised by known methods to form the compound of formula I wherein Q denotes —N═CR⁸— (IIIc),
d) in the event of a group Q having the meaning —N═N— (IIId) an o-amino-benzamide derivative of formula Id.1 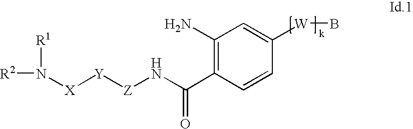
wherein R¹, R², W, B, X, Y, Z and k have the meanings specified, reacting in the presence of a suitable nitrite compound and an acid to form the compound of formula I wherein Q denotes —N═N—,
e) in the event of a group Q having the meaning —CO—NR⁹— (IIIe), wherein R⁹ is as hereinbefore defined, an o-amino-benzamide derivative of formula Ie.1 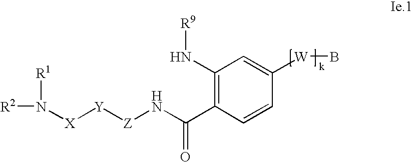
wherein R¹, R², R⁹, W, B, X, Y, Z and k have the meanings specified, reacting in the presence of CDI (carbonyldiimidazole) to form the compound of formula I wherein Q denotes —CO—NR⁹—,
f) in the event of a group Q having the meaning —CR⁸═N— (IIIf), wherein R⁸ is as hereinbefore defined, an o-amino-benzamide derivative of formula If.1 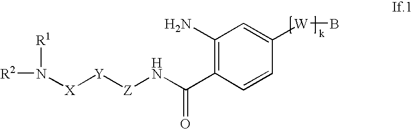
wherein R¹, R², W, B, X, Y, Z and k have the meanings specified, reacting with a carboxylic acid R⁸COOH having the meaning specified for R⁸ and/or a corresponding activated carboxylic acid derivative to form the quinazolinone derivative of formula I wherein Q denotes —CR⁸═N—,
g) in the event of a group Q having the meaning —CO— (IIIg) an isobenzofurandione derivative of formula Ig.2 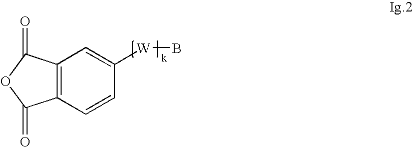
wherein W, B and k have the meanings specified,
reacting with an amine of formula Ig.1 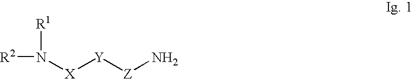
wherein R¹, R², X, Y and Z have the meanings specified, to form the compound of formula I wherein Q denotes —CO—.

32. The physiologically acceptable salts of the carboxamide compounds according to claim 1.

33. Method of influencing the eating behavior of a mammal comprised of administering to a mammal a pharmaceutically effective amount of carboxamide compound according to claim 32.

34. A composition comprised of at least one carboxamide compound according to claim 1 further comprised of one or more physiologically acceptable excipients.

35. Pharmaceutical composition, containing at least one carboxamide compound according to claim 34 optionally together with one or more inert carriers and/or diluents.

36. Method of influencing the eating behaviour of a mammal comprised of administering to a mammal a pharmaceutically effective amount of a compound according to claim 1.

37. A method of reducing the body weight and/or prevention of an increase of body weight of a mammal comprised of the administration of a pharmaceutically effective amount of carboxamide compound according to claim 1 to a mammal.

38. Use of at least one carboxamide compound according to claim 1 for preparing a pharmaceutical composition with an MCH-receptor-antagonist activity.

39. Method of preventing and/or treating symptoms and/or diseases which are caused by MCH or otherwise casually connected with MCH, comprised of the administration to a patient in need thereof a pharmaceutically effective amount of a compound according to claim 1.

40. A method of treating metabolic disorders and/or eating disorders, obesity, bulimia, bulimia nervosa, cachexia, anorexia nervosa and hyperphagia comprised of the administration to a patient in need thereof of a pharmaceutically effective amount of a carboxamide compound according to claim 1.

41. Method of preventing and/or treating diseases and/or disorders associated with obesity, particularly diabetes, especially type II diabetes, complications of diabetes including diabetic retinopathy, diabetic neuropathy, diabetic nephropathy, insulin resistance, pathological glucose tolerance, encephalorrhagia, cardiac insufficiency, cardiovascular diseases, particularly arteriosclerosis and high blood pressure, arthritis and gonitis comprised of administering to a patient in need thereof a pharmaceutically effective amount of a compound according to claim 1.

42. Method of preventing or treating hyperlipidaemia, cellulitis, fat accumulation, malignant mastocytosis, systemic mastocytosis, emotional disorders, affective disorders, depression, anxiety, sleep disorders, reproductive disorders, sexual disorders, memory disorders, epilepsy, forms of dementia and hormonal disorders comprised of administering to a patient in need thereof a pharmaceutically effective amount of a carboxamide compound according to claim 1.

43. A method of preventing and/or treating micturition disorders, selected from a list consisting of urinary incontinence, hyperactive urinary bladder, urgency, nycturia and enuresis comprised of administering to a patient in need thereof a pharmaceutically effective amount of a compound according to claim 1.

44. Pharmaceutical compositions, containing a first active substance selected from the carboxamide compounds according to claim 1 and a second active substance selected from the group consisting of active substances for the treatment of diabetes, active substances for the treatment of diabetic complications, active substances for the treatment of obesity, preferably other than MCH antagonists, active substances for the treatment of high blood pressure, active substances for the treatment of hyperlipidaemia, including arteriosclerosis, active substances for the treatment of arthritis, active substances for the treatment of anxiety states and active substances for the treatment of depression, optionally together with one or more inert carriers and/or diluents.
